(19) United States
(12) Patent Application Publication
(10) Pub. No.: US 2024/0101990 A1
(43) Pub. Date: Mar. 28, 2024

(54) CRISPR DNA TARGETING ENZYMES AND SYSTEMS

(71) Applicant: Arbor Biotechnologies, Inc., Cambridge, MA (US)

(72) Inventors: David A. Scott, San Francisco, CA (US); David R. Cheng, Boston, MA (US); Winston X. Yan, Boston, MA (US); Tia M. DiTommaso, Waltham, MA (US)

(21) Appl. No.: 18/465,755

(22) Filed: Sep. 12, 2023

Related U.S. Application Data
(63) Continuation of application No. 17/820,444, filed on Aug. 17, 2022, now Pat. No. 11,795,442.
Application No. 17/820,444 is a continuation of application No. 17/505,578, filed on Oct. 19, 2021, now Pat. No. 11,453,867.
Application No. 17/505,578 is a continuation of application No. PCT/US2020/049923, filed on Sep. 9, 2020.
(60) Provisional application No. 62/897,859, filed on Sep. 9, 2019.

(51) Int. Cl. C12N 9/22; C12N 1/20; C12N 5/071; C12N 15/11; C12N 15/70; C12N 15/85; C12N 15/90; C12Q 1/6823

(52) U.S. Cl. CPC C12N 9/22; C12N 1/20; C12N 5/0687; C12N 15/11; C12N 15/111; C12N 15/70; C12N 15/85; C12N 15/907; C12Q 1/6823; C12N 2310/20; C12N 2800/10; C12N 2800/80

(57) ABSTRACT

The disclosure describes novel systems, methods, and compositions for the manipulation of nucleic acids in a targeted fashion. The disclosure describes non-naturally occurring, engineered CRISPR systems, components, and methods for targeted modification of nucleic acids. Each system includes one or more protein components and one or more nucleic acid components that together target nucleic acids.

### RELATED APPLICATION

This application is a continuation of U.S. patent application Ser. No. 17/820,444, filed Aug. 17, 2022, now issued as U.S. Pat. No. 11,795,442 on Oct. 24, 2023, which is a continuation of U.S. patent application Ser. No. 17/505,578, filed Oct. 19, 2021, now issued as U.S. Pat. No. 11,453,867 on Sep. 27, 2022, which is a continuation of International Application No. PCT/US2020/049923, filed Sep. 9, 2020, which claims priority to U.S. Provisional Application 62/897,859 filed on Sep. 9, 2019, the entire contents of each of which is hereby incorporated by reference.

### SEQUENCE LISTING

The instant application contains a Sequence Listing which has been submitted electronically in XML format and is hereby incorporated by reference in its entirety. Said XML copy, created on Dec. 15, 2023, is named 0030US3_C1_A2186-702823_SL_Update.xml and is 455,508 bytes in size.

### FIELD OF THE INVENTION

The present disclosure relates to systems and methods for genome editing and modulation of gene expression using novel Clustered Regularly Interspaced Short Palindromic Repeats (CRISPR) and CRISPR-associated (Cas) genes.

### BACKGROUND

Recent advances in genome sequencing technologies and analyses have yielded significant insight into the genetic underpinnings of biological activities in many diverse areas of nature, ranging from prokaryotic biosynthetic pathways to human pathologies. To fully understand and evaluate the vast quantities of information yielded, equivalent increases in the scale, efficacy, and ease of sequence technologies for genome and epigenome manipulation are needed. These novel technologies will accelerate the development of novel applications in numerous areas, including biotechnology, agriculture, and human therapeutics.

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements. CRISPR-Cas systems comprise an extremely diverse group of proteins effectors, non-coding elements, and loci architectures, some examples of which have been engineered and adapted to produce important biotechnological advances.

The components of the system involved in host defense include one or more effector proteins capable of modifying a nucleic acid and an RNA guide element that is responsible for targeting the effector protein(s) to a specific sequence on a phage nucleic acid. The RNA guide is composed of a CRISPR RNA (crRNA) and may require an additional trans-activating RNA (tracrRNA) to enable targeted nucleic acid manipulation by the effector protein(s). The crRNA consists of a direct repeat responsible for protein binding to the crRNA and a spacer sequence that is complementary to the desired nucleic acid target sequence. CRISPR systems can be reprogrammed to target alternative DNA or RNA targets by modifying the spacer sequence of the crRNA.

CRISPR-Cas systems can be broadly classified into two classes: Class 1 systems are composed of multiple effector proteins that together form a complex around a crRNA, and Class 2 systems consists of one effector protein that complexes with the RNA guide to target nucleic acid substrates. The single-subunit effector composition of the Class 2 systems provides a simpler component set for engineering and application translation and have thus far been an important source of programmable effectors. Nevertheless, there remains a need for additional programmable effectors and systems for modifying nucleic acids and polynucleotides (i.e., DNA, RNA, or any hybrid, derivative, or modification) beyond the current CRISPR-Cas systems, such as smaller effectors and/or effectors having unique PAM sequence requirements, that enable novel applications through their unique properties.

### SUMMARY

This disclosure provides non-naturally-occurring, engineered systems and compositions for novel single-effector Class 2 CRISPR-Cas systems, which were first identified computationally from genomic databases and subsequently engineered and experimentally validated. In particular, identification of the components of these CRISPR-Cas systems allows for their use in non-natural environments, e.g., in bacteria other than those in which the systems were initially discovered or in eukaryotic cells, such as mammalian cells. These new effectors are divergent in sequence and function compared to orthologs and homologs of existing Class 2 CRISPR effectors.

In one aspect, the disclosure provides engineered, non-naturally occurring Clustered Regularly Interspaced Short Palindromic Repeat (CRISPR)—Cas systems of CLUST.091979 including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-56; and an RNA guide including a direct repeat sequence and a spacer sequence capable of hybridizing to a target nucleic acid; wherein the CRISPR-associated protein is capable of binding to the RNA guide and of modifying the target nucleic acid sequence complementary to the spacer sequence. In one aspect, the disclosure provides engineered, non-naturally occurring Clustered Regularly Interspaced Short Palindromic Repeat (CRISPR)—Cas systems of CLUST.091979 including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-56; and an RNA guide including a direct repeat sequence and a spacer sequence capable of hybridizing to a target nucleic acid, or a nucleic acid encoding the RNA guide; wherein the CRISPR-associated protein is capable of binding to the RNA guide and of modifying the target nucleic acid sequence complementary to the spacer sequence.

In some aspects, the disclosure provides an engineered, non-naturally occurring Clustered Regularly Interspaced Short Palindromic Repeat (CRISPR)—Cas system of CLUST.091979 comprising a CRISPR-associated protein or a nucleic acid encoding the CRISPR-associated protein, wherein the CRISPR-associated protein comprises an amino acid sequence of SEQ ID NO: 241; and an RNA guide comprising a direct repeat sequence and a spacer sequence capable of hybridizing to a target nucleic acid; wherein the CRISPR-associated protein is capable of binding to the RNA guide and of modifying the target nucleic acid sequence complementary to the spacer sequence. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in SEQ ID NO: 4, SEQ ID NO: 10, SEQ ID NO: 12, or SEQ ID NO: 14.

In some embodiments of any of the systems described herein, the CRISPR-associated protein includes at least one (e.g., one, two, or three) RuvC domain or at least one split RuvC domain.

In some embodiments of any of the systems described herein, the CRISPR-associated protein comprises one or more of the following sequences: (a) PX₁X₂X₃X₄F (SEQ ID NO: 216), wherein X₁ is L or M or I or C or F, X₂ is Y or W or F, X₃ is K or T or C or R or W or Y or H or V, and X₄ is I or L or M; (b) RX₁X₂X₃L (SEQ ID NO: 217), wherein X₁ is I or L or M or Y or T or F, X₂ is R or Q or K or E or S or T, and X₃ is L or I or T or C or M or K; (c) NX₁YX₂(SEQ ID NO: 218), wherein X₁ is I or L or F and X₂ is K or R or V or E; (d) KX₁X₂X₃FAX₄X₅KD (SEQ ID NO: 219), wherein X₁ is T or I or N or A or S or F or V, X₂ is I or V or L or S, X₃ is H or S or G or R, X₄ is D or S or E, and X₅ is I or V or M or T or N; (e) LX₁NX₂ (SEQ ID NO: 220), wherein X₁ is G or S or C or T and X₂ is N or Y or K or S; (f) PX₁X₂X₃X₄SQX₅DS (SEQ ID NO: 221), wherein X₁ is S or P or A, X₂ is Y or S or A or P or E or Y or Q or N, X₃ is F or Y or H, X₄ is T or S, and X₅ is M or T or I; (g) KX₁X₂VRX₃X₄QEX₅H (SEQ ID NO: 222), wherein X₁ is N or K or W or R or E or T or Y, X₂ is M or R or L or S or K or V or E or T or I or D, X₃ is L or R or H or P or T or K or Q of P or S or A, X₄ is G or Q or N or R or K or E or I or T or S or C, and X₅ is R or W or Y or K or T or F or S or Q; and (h) X₁NGX₂X₃X₄DX₅NX₆X₇X₈N (SEQ ID NO: 223), wherein X₁ is I or K or V or L, X₂ is L or M, X₃ is N or H or P, X₄ is A or S or C, X₅ is V or Y or I or F or T or N, X₆ is A or S, X₇ is S or A or P, and X₈ is M or C or L or R or N or S or K or L. In some embodiments of any of the systems described herein, the sequence of SEQ ID NO: 216 is an N-terminal sequence. In some embodiments of any of the systems described herein, the sequence of SEQ ID NO: 219 is a C-terminal sequence. In some embodiments of any of the systems described herein, the sequence of SEQ ID NO: 220 is a C-terminal sequence. In some embodiments of any of the systems described herein, the sequence of SEQ ID NO: 221 is a C-terminal sequence. In some embodiments of any of the systems described herein, the sequence of SEQ ID NO: 222 is a C-terminal sequence. In some embodiments of any of the systems described herein, the sequence of SEQ ID NO: 223 is a C-terminal sequence.

In some embodiments of any of the systems described herein, the CRISPR-associated protein comprises one or more of the following sequences: (a) ECPITKDVINEYK (SEQ ID NO: 290); (b) NLTSITIG (SEQ ID NO: 231); (c) NYRTKIRTLN (SEQ ID NO: 232); (d) ISYIENVEN (SEQ ID NO: 233); (e) ELLSVEQLK (SEQ ID NO: 234); (f) HINSMTINIQDFKIE (SEQ ID NO: 235); (g) KENSLGFIL (SEQ ID NO: 236); (h) GNRQIKKG (SEQ ID NO: 237); (i) DVNFKHA (SEQ ID NO: 238); (j) GYINLYKYLLEH (SEQ ID NO: 239); (k) KEQVLSKLLY (SEQ ID NO: 240); (1) EYIYVSCVNKLRAKYVSYFILKEKYYEKQKEYDIEMGF (SEQ ID NO: 241); (m) DDSTESKESMDKRR (SEQ ID NO: 242); (n) NVQQDINGCLKNIINY (SEQ ID NO: 243); (o) ALENLENSNFEK (SEQ ID NO: 244); (p) QVLPTIKSLL (SEQ ID NO: 245); (q) YHKLENQN (SEQ ID NO: 246); (r) ASDKVKEYIE (SEQ ID NO: 247); (s) TNENNEIVDAKYT (SEQ ID NO: 248); (t) ANFFNLMMKSLHFAS (SEQ ID NO: 249); (u) LLSNNGKTQIALVPSE (SEQ ID NO: 250); (v) HINGLNADFNAANNIKYI (SEQ ID NO: 251), or a sequence having no more than 1, 2, or 3 sequence differences (e.g., substitutions) relative to any of the foregoing. In some embodiments, the CRISPR-associated protein has a sequence at least 70% identical to SEQ ID NO: 4. In some embodiments, the CRISPR-associated protein has a sequence at least 70% identical to SEQ ID NO: 10.

In some embodiments of any of the systems described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 57-90, SEQ ID NOs: 118-151, or SEQ ID NO: 213. In some embodiments of any of the systems described herein, the direct repeat sequence includes a nucleotide sequence that is at least 95% (e.g., 95%, 96%, 97%, 98%, 99% or 100%) identical to a nucleotide sequence set forth in any one of SEQ ID NOs: 57-90, SEQ ID NOs: 118-151, or SEQ ID NO: 213.

In some embodiments of any of the systems described herein, the direct repeat sequence comprises one or more of the following sequences: (a) X₁X₂TX₃X₄X₅X₆X₇X₈ (SEQ ID NO: 224), wherein X₁ is A or C or G, X₂ is T or C or A, X₃ is T or G or A, X₄ is T or G, X₅ is T or G or A, X₆ is G or T or A, X₇ is T or G or A, and X₈ is A or G or T (e.g., ATTGTTGDA (SEQ ID NO: 225)); (b) X₁X₂X₃X₄X₅X₆X₇X₈X₉ (SEQ ID NO: 226), wherein X₁ is T or C or A, X₂ is T or A or G, X₃ is T or C or A, X₄ is T or A, X₅ is T or A or G, X₆ is T or A, X₇ is A or T, X₈ is A or G or C or T, and X₉ is G or A or C (e.g., TTTTWTARG (SEQ ID NO: 227)); and (c) X₁X₂X₃AC (SEQ ID NO: 228), wherein X₁ is A or C or G, X₂ is C or A, and X₃ is A or C (e.g., ACAAC (SEQ ID NO: 229)). In some embodiments of any of the systems described herein, SEQ ID NO: 224 is proximal to the 5′ end of the direct repeat. In some embodiments of any of the systems described herein, SEQ ID NO: 228 is proximal to the 3′ end of the direct repeat.

In some embodiments of any of the systems described herein, the CRISPR-associated protein is capable of recognizing a protospacer adjacent motif (PAM), wherein the PAM includes a nucleic acid sequence, including a nucleic acid sequence set forth as 5′-NTTN-3′, 5′-NTTR-3′, 5′-RTTR-3′, 5′-TNNT-3′, 5′-TNRT-3′, 5′-TSRT-3′, 5′-TGRT-3′, 5′-TNRY-3′, 5′-TTNR-3′, 5′-TTYR-3′, 5′-TTTR-3′, 5′-TTCV-3′, 5′-DTYR-3′, 5′-WTTR-3′, 5′-NNR-3′, 5′-NYR-3′, 5′-YYR-3′, 5′-TYR-3′, 5′-TTN-3′, 5′-TTR-3′, 5′-CNT-3′, 5′-NGG-3′, 5′-BGG-3′, or 5′-R-3′, wherein “N” is any nucleotide, “B” is C or G or T, “D” is A or G or T, “R” is A or G, “S” is G or C, “V” is A or C or G, “W” is A or T, and “Y” is C or T.

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1, and wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 57. In some embodiments of any of the systems described herein, the CRISPR-associated protein is a protein having at least 95% (e.g., 95%, 96%, 97%, 98%, 99% or 100%) identity to an amino acid sequence set forth in SEQ ID NO: 1, and wherein the direct repeat sequence comprises a nucleotide sequence that is at least 95% (e.g., 95%, 96%, 97%, 98%, 99% or 100%) identical to a nucleotide sequence set forth in SEQ ID NO: 57. In some embodiments of any of the systems described herein, the CRISPR-associated protein is capable of recognizing a protospacer adjacent motif (PAM) sequence, wherein the PAM sequence comprises a nucleic acid sequence set forth as 5′-TNNT-3′ or 5′-TNRT-3′, wherein “N” is any nucleotide and “R” is A or G.

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 4, and wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 60. In some embodiments of any of the systems described herein, the CRISPR-associated protein is a protein having at least 95% (e.g., 95%, 96%, 97%, 98%, 99% or 100%) identity to an amino acid sequence set forth in SEQ ID NO: 4, and wherein the direct repeat sequence comprises a nucleotide sequence that is at least 95% (e.g., 95%, 96%, 97%, 98%, 99% or 100%) identical to a nucleotide sequence set forth in SEQ ID NO: 60. In some embodiments of any of the systems described herein, the CRISPR-associated protein is capable of recognizing a protospacer adjacent motif (PAM) sequence, wherein the PAM sequence comprises a nucleic acid sequence set forth as 5′-NTTN-3′, 5′-NTTR-3′ (e.g., 5 ‘-TTTG-3’), or 5′-NNR-3′, wherein “N” is any nucleotide and “R” is A or G.

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 10, and wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 62 or SEQ ID NO: 213. In some embodiments of any of the systems described herein, the CRISPR-associated protein is a protein having at least 95% (e.g., 95%, 96%, 97%, 98%, 99% or 100%) identity to an amino acid sequence set forth in SEQ ID NO: 10, and wherein the direct repeat sequence comprises a nucleotide sequence that is at least 95% (e.g., 95%, 96%, 97%, 98%, 99% or 100%) identical to a nucleotide sequence set forth in SEQ ID NO: 62 or SEQ ID NO: 213. In some embodiments of any of the systems described herein, the CRISPR-associated protein is capable of recognizing a protospacer adjacent motif (PAM) sequence, wherein the PAM sequence comprises a nucleic acid sequence set forth as 5′-NTTN-3′ or 5′-RTTR-3′ (e.g., 5 ‘-ATTG-3’ or 5′-GTTA-3′), wherein “N” is any nucleotide and “R” is A or G.

In some embodiments of any of the systems described herein, the spacer sequence of the RNA guide includes between about 15 nucleotides to about 55 nucleotides. In some embodiments of any of the systems described herein, the spacer sequence of the RNA guide includes between 20 and 45 nucleotides.

In some embodiments of any of the systems described herein, the CRISPR-associated protein comprises a catalytic residue (e.g., aspartic acid or glutamic acid). In some embodiments of any of the systems described herein, the CRISPR-associated protein cleaves the target nucleic acid. In some embodiments of any of the systems described herein, the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In some embodiments of any of the systems described herein, the nucleic acid encoding the CRISPR-associated protein is codon-optimized for expression in a cell, e.g., a eukaryotic cell, e.g., a mammalian cell, e.g., a human cell. In some embodiments of any of the systems described herein, the nucleic acid encoding the CRISPR-associated protein is operably linked to a promoter. In some embodiments of any of the systems described herein, the nucleic acid encoding the CRISPR-associated protein is in a vector. In some embodiments, the vector comprises a retroviral vector, a lentiviral vector, a phage vector, an adenoviral vector, an adeno-associated vector, or a herpes simplex vector.

In some embodiments of any of the systems described herein, the target nucleic acid is a DNA molecule. In some embodiments of any of the systems described herein, the target nucleic acid includes a PAM sequence.

In some embodiments of any of the systems described herein, the CRISPR-associated protein has non-specific nuclease activity.

In some embodiments of any of the systems described herein, recognition of the target nucleic acid by the CRISPR-associated protein and RNA guide results in a modification of the target nucleic acid. In some embodiments of any of the systems described herein, the modification of the target nucleic acid is a double-stranded cleavage event. In some embodiments of any of the systems described herein, the modification of the target nucleic acid is a single-stranded cleavage event. In some embodiments of any of the systems described herein, the modification of the target nucleic acid results in an insertion event. In some embodiments of any of the systems described herein, the modification of the target nucleic acid results in a deletion event. In some embodiments of any of the systems described herein, the modification of the target nucleic acid results in cell toxicity or cell death.

In some embodiments of any of the systems described herein, the system further includes a donor template nucleic acid. In some embodiments of any of the systems described herein, the donor template nucleic acid is a DNA molecule. In some embodiments of any of the systems described herein, wherein the donor template nucleic acid is an RNA molecule.

In some embodiments of any of the systems described herein, the RNA guide optionally includes a tracrRNA and/or a modulator RNA. In some embodiments of any of the systems described herein, the system further includes a tracrRNA. In some embodiments of any of the systems described herein, the system does not include a tracrRNA. In some embodiments of any of the systems described herein, the CRISPR-associated protein is self-processing. In some embodiments of any of the systems described herein, the system further includes a modulator RNA.

In some embodiments of any of the systems described herein, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 152, SEQ ID NO: 153, or SEQ ID NO: 154.

In some embodiments of any of the systems described herein, the system is present in a delivery composition comprising a nanoparticle, a liposome, an exosome, a microvesicle, or a gene-gun.

In some embodiments of any of the systems described herein, the systems are within a cell. In some embodiments, the cell is a eukaryotic cell. In some embodiments, the cell is a mammalian cell. In some embodiments, the cell is a human cell. In some embodiments, the cell is a prokaryotic cell.

In another aspect, the disclosure provides a cell, wherein the cell includes: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-56; and an RNA guide including a direct repeat sequence and a spacer sequence capable of hybridizing to a target nucleic acid. In another aspect, the disclosure provides a cell, wherein the cell includes: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-56; and an RNA guide including a direct repeat sequence and a spacer sequence capable of hybridizing to a target nucleic acid, or a nucleic acid encoding the RNA guide.

In some embodiments of any of the cells described herein, the CRISPR-associated protein includes at least one (e.g., one, two, or three) RuvC domain or at least one split RuvC domain.

In some embodiments of any of the cells described herein, the CRISPR-associated protein comprises one or more of the following sequences: (a) PX₁X₂X₃X₄F (SEQ ID NO: 216), wherein X₁ is L or M or I or C or F, X₂ is Y or W or F, X₃ is K or T or C or R or W or Y or H or V, and X₄ is I or L or M; (b) RX₁X₂X₃L (SEQ ID NO: 217), wherein X₁ is I or L or M or Y or T or F, X₂ is R or Q or K or E or S or T, and X₃ is L or I or T or C or M or K; (c) NX₁YX₂ (SEQ ID NO: 218), wherein X₁ is I or L or F and X₂ is K or R or V or E; (d) KX₁X₂X₃FAX₄X₅KD (SEQ ID NO: 219), wherein X₁ is T or I or N or A or S or F or V, X₂ is I or V or L or S, X₃ is H or S or G or R, X₄ is D or S or E, and X₅ is I or V or M or T or N; (e) LX₁NX₂ (SEQ ID NO: 220), wherein X₁ is G or S or C or T and X₂ is N or Y or K or S; (f) PX₁X₂X₃X₄SQX₅DS (SEQ ID NO: 221), wherein X₁ is S or P or A, X₂ is Y or S or A or P or E or Y or Q or N, X₃ is F or Y or H, X₄ is T or S, and X₅ is M or T or I; (g) KX₁X₂VRX₃X₄QEX₅H (SEQ ID NO: 222), wherein X₁ is N or K or W or R or E or T or Y, X₂ is M or R or L or S or K or V or E or T or I or D, X₃ is L or R or H or P or T or K or Q of P or S or A, X₄ is G or Q or N or R or K or E or I or T or S or C, and X₅ is R or W or Y or K or T or F or S or Q; and (h) X₁NGX₂X₃X₄DX₅NX₆X₇X₈N (SEQ ID NO: 223), wherein X₁ is I or K or V or L, X₂ is L or M, X₃ is N or H or P, X₄ is A or S or C, X₅ is V or Y or I or F or T or N, X₆ is A or S, X₇ is S or A or P, and X₈ is M or C or L or R or N or S or K or L. In some embodiments of any of the cells described herein, the sequence of SEQ ID NO: 216 is an N-terminal sequence. In some embodiments of any of the cells described herein, the sequence of SEQ ID NO: 219 is a C-terminal sequence. In some embodiments of any of the cells described herein, the sequence of SEQ ID NO: 220 is a C-terminal sequence. In some embodiments of any of the cells described herein, the sequence of SEQ ID NO: 221 is a C-terminal sequence. In some embodiments of any of the cells described herein, the sequence of SEQ ID NO: 222 is a C-terminal sequence. In some embodiments of any of the cells described herein, the sequence of SEQ ID NO: 223 is a C-terminal sequence.

In some embodiments of any of the cells described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 57-90, SEQ ID NOs: 118-151, or SEQ ID NO: 213. In some embodiments of any of the cells described herein, the direct repeat sequence includes a nucleotide sequence that is at least 95% (e.g., 95%, 96%, 97%, 98%, 99% or 100%) identical to a nucleotide sequence set forth in any one of SEQ ID NOs: 57-90, SEQ ID NOs: 118-151, or SEQ ID NO: 213.

In some embodiments of any of the cells described herein, the direct repeat sequence comprises one or more of the following sequences: (a) X₁X₂TX₃X₄X₅X₆X₇X₈ (SEQ ID NO: 224), wherein X₁ is A or C or G, X₂ is T or C or A, X₃ is T or G or A, X₄ is T or G, X₅ is T or G or A, X₆ is G or T or A, X₇ is T or G or A, and X₈ is A or G or T (e.g., ATTGTTGDA (SEQ ID NO: 225)); (b) X₁X₂X₃X₄X₅X₆X₇X₈X₉ (SEQ ID NO: 226), wherein X₁ is T or C or A, X₂ is T or A or G, X₃ is T or C or A, X₄ is T or A, X₅ is T or A or G, X₆ is T or A, X₇ is A or T, X₈ is A or G or C or T, and X₉ is G or A or C (e.g., TTTTWTARG (SEQ ID NO: 227)); and (c) X₁X₂X₃AC (SEQ ID NO: 228), wherein X₁ is A or C or G, X₂ is C or A, and X₃ is A or C (e.g., ACAAC (SEQ ID NO: 229)). In some embodiments of any of the cells described herein, SEQ ID NO: 224 is proximal to the 5′ end of the direct repeat. In some embodiments of any of the cells described herein, SEQ ID NO: 228 is proximal to the 3′ end of the direct repeat.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1, and wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 57. In some embodiments of any of the cells described herein, the CRISPR-associated protein is a protein having at least 95% (e.g., 95%, 96%, 97%, 98%, 99% or 100%) identity to an amino acid sequence set forth in SEQ ID NO: 1, and wherein the direct repeat sequence comprises a nucleotide sequence that is at least 95% (e.g., 95%, 96%, 97%, 98%, 99% or 100%) identical to a nucleotide sequence set forth in SEQ ID NO: 57. In some embodiments of any of the cells described herein, the CRISPR-associated protein is capable of recognizing a protospacer adjacent motif (PAM) sequence, wherein the PAM sequence comprises a nucleic acid sequence set forth as 5′-TNNT-3′ or 5′-TNRT-3′, wherein “N” is any nucleotide and “R” is A or G.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 4, and wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 60. In some embodiments of any of the cells described herein, the CRISPR-associated protein is a protein having at least 95% (e.g., 95%, 96%, 97%, 98%, 99% or 100%) identity to an amino acid sequence set forth in SEQ ID NO: 4, and wherein the direct repeat sequence comprises a nucleotide sequence that is at least 95% (e.g., 95%, 96%, 97%, 98%, 99% or 100%) identical to a nucleotide sequence set forth in SEQ ID NO: 60. In some embodiments of any of the cells described herein, the CRISPR-associated protein is capable of recognizing a protospacer adjacent motif (PAM) sequence, wherein the PAM sequence comprises a nucleic acid sequence set forth as 5′-NTTN-3′, 5′-NTTR-3′ (e.g., 5 ‘-TTTG-3’), or 5′-NNR-3′, wherein “N” is any nucleotide and “R” is A or G.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 10, and wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 62 or SEQ ID NO: 213. In some embodiments of any of the cells described herein, the CRISPR-associated protein is a protein having at least 95% (e.g., 95%, 96%, 97%, 98%, 99% or 100%) identity to an amino acid sequence set forth in SEQ ID NO: 10, and wherein the direct repeat sequence comprises a nucleotide sequence that is at least 95% (e.g., 95%, 96%, 97%, 98%, 99% or 100%) identical to a nucleotide sequence set forth in SEQ ID NO: 62 or SEQ ID NO: 213. In some embodiments of any of the cells described herein, the CRISPR-associated protein is capable of recognizing a protospacer adjacent motif (PAM) sequence, wherein the PAM sequence comprises a nucleic acid sequence set forth as 5′-NTTN-3′ or 5′-RTTR-3′ (e.g., 5′-ATTG-3′ or 5′-GTTA-3′), wherein “N” is any nucleotide and “R” is A or G.

In some embodiments of any of the cells described herein, the spacer sequence includes between about 15 nucleotides to about 55 nucleotides. In some embodiments of any of the cells described herein, the spacer sequence includes between 20 and 45 nucleotides.

In some embodiments of any of the cells described herein, the CRISPR-associated protein comprises a catalytic residue (e.g., aspartic acid or glutamic acid). In some embodiments of any of the cells described herein, the CRISPR-associated protein cleaves the target nucleic acid. In some embodiments of any of the cells described herein, the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In some embodiments of any of the cells described herein, the nucleic acid encoding the CRISPR-associated protein is codon-optimized for expression in a cell, e.g., a eukaryotic cell, e.g., a mammalian cell, e.g., a human cell. In some embodiments of any of the cells described herein, the nucleic acid encoding the CRISPR-associated protein is operably linked to a promoter. In some embodiments of any of the cells described herein, the nucleic acid encoding the CRISPR-associated protein is in a vector. In some embodiments, the vector comprises a retroviral vector, a lentiviral vector, a phage vector, an adenoviral vector, an adeno-associated vector, or a herpes simplex vector.

In some embodiments of any of the cells described herein, the RNA guide optionally includes a tracrRNA and/or a modulator RNA. In some embodiments of any of the cells described herein, the cell further includes a tracrRNA. In some embodiments of any of the cells described herein, the cell does not include a tracrRNA. In some embodiments of any of the cells described herein, the CRISPR-associated protein is self-processing. In some embodiments of any of the cells described herein, the cell further includes a modulator RNA.

In some embodiments of any of the cells described herein, the cell is a eukaryotic cell. In some embodiments of any of the cells described herein, the cell is a mammalian cell. In some embodiments of any of the cells described herein, the cell is a human cell. In some embodiments of any of the cells described herein, the cell is a prokaryotic cell.

In some embodiments of any of the cells described herein, the target nucleic acid is a DNA molecule. In some embodiments of any of the cells described herein, the target nucleic acid includes a PAM sequence.

In some embodiments of any of the cells described herein, the CRISPR-associated protein has non-specific nuclease activity.

In some embodiments of any of the cells described herein, recognition of the target nucleic acid by the CRISPR-associated protein and RNA guide results in a modification of the target nucleic acid. In some embodiments of any of the cells described herein, the modification of the target nucleic acid is a double-stranded cleavage event. In some embodiments of any of the cells described herein, the modification of the target nucleic acid is a single-stranded cleavage event. In some embodiments of any of the cells described herein, the modification of the target nucleic acid results in an insertion event. In some embodiments of any of the cells described herein, the modification of the target nucleic acid results in a deletion event. In some embodiments of any of the cells described herein, the modification of the target nucleic acid results in cell toxicity or cell death.

In another aspect, the disclosure provides a method of binding a system described herein to a target nucleic acid in a cell comprising: (a) providing the system; and (b) delivering the system to the cell, wherein the cell comprises the target nucleic acid, wherein the CRISPR-associated protein binds to the RNA guide, and wherein the spacer sequence binds to the target nucleic acid. In some embodiments, the cell is a eukaryotic cell, e.g., a mammalian cell, e.g., a human cell.

In another aspect, the disclosure provides methods of modifying a target nucleic acid, the method including delivering to the target nucleic acid an engineered, non-naturally occurring CRISPR-Cas system including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-56; and an RNA guide including a direct repeat sequence and a spacer sequence capable of hybridizing to the target nucleic acid; wherein the CRISPR-associated protein is capable of binding to the RNA guide; and wherein recognition of the target nucleic acid by the CRISPR-associated protein and RNA guide results in a modification of the target nucleic acid. In another aspect, the disclosure provides methods of modifying a target nucleic acid, the method including delivering to the target nucleic acid an engineered, non-naturally occurring CRISPR-Cas system including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-56; and an RNA guide including a direct repeat sequence and a spacer sequence capable of hybridizing to the target nucleic acid; wherein the CRISPR-associated protein is capable of binding to the RNA guide; and wherein recognition of the target nucleic acid by the CRISPR-associated protein and RNA guide results in a modification of the target nucleic acid.

In some embodiments of any of the methods described herein, the CRISPR-associated protein comprises one or more of the following sequences: (a) PX₁X₂X₃X₄F (SEQ ID NO: 216), wherein X₁ is L or M or I or C or F, X₂ is Y or W or F, X₃ is K or T or C or R or W or Y or H or V, and X₄ is I or L or M; (b) RX₁X₂X₃L (SEQ ID NO: 217), wherein X₁ is I or L or M or Y or T or F, X₂ is R or Q or K or E or S or T, and X₃ is L or I or T or C or M or K; (c) NX₁YX₂(SEQ ID NO: 218), wherein X₁ is I or L or F and X₂ is K or R or V or E; (d) KX₁X₂X₃FAX₄X₅KD (SEQ ID NO: 219), wherein X₁ is T or I or N or A or S or F or V, X₂ is I or V or L or S, X₃ is H or S or G or R, X₄ is D or S or E, and X₅ is I or V or M or T or N; (e) LX₁NX₂ (SEQ ID NO: 220), wherein X₁ is G or S or C or T and X₂ is N or Y or K or S; (f) PX₁X₂X₃X₄SQX₅DS (SEQ ID NO: 221), wherein X₁ is S or P or A, X₂ is Y or S or A or P or E or Y or Q or N, X₃ is F or Y or H, X₄ is T or S, and X₅ is M or T or I; (g) KX₁X₂VRX₃X₄QEX₅H (SEQ ID NO: 222), wherein X₁ is N or K or W or R or E or T or Y, X₂ is M or R or L or S or K or V or E or T or I or D, X₃ is L or R or H or P or T or K or Q of P or S or A, X₄ is G or Q or N or R or K or E or I or T or S or C, and X₅ is R or W or Y or K or T or F or S or Q; and (h) X₁NGX₂X₃X₄DX₅NX₆X₇X₈N (SEQ ID NO: 223), wherein X₁ is I or K or V or L, X₂ is L or M, X₃ is N or H or P, X₄ is A or S or C, X₅ is V or Y or I or F or T or N, X₆ is A or S, X₇ is S or A or P, and X₈ is M or C or L or R or N or S or K or L. In some embodiments of any of the methods described herein, the sequence of SEQ ID NO: 216 is an N-terminal sequence. In some embodiments of any of the methods described herein, the sequence of SEQ ID NO: 219 is a C-terminal sequence. In some embodiments of any of the methods described herein, the sequence of SEQ ID NO: 220 is a C-terminal sequence. In some embodiments of any of the methods described herein, the sequence of SEQ ID NO: 221 is a C-terminal sequence. In some embodiments of any of the methods described herein, the sequence of SEQ ID NO: 222 is a C-terminal sequence. In some embodiments of any of the methods described herein, the sequence of SEQ ID NO: 223 is a C-terminal sequence.

In some embodiments of any of the methods described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 57-90, SEQ ID NOs: 118-151, or SEQ ID NO: 213. In some embodiments of any of the methods described herein, the direct repeat sequence includes a nucleotide sequence that is at least 95% (e.g., 95%, 96%, 97%, 98%, 99% or 100%) identical to a nucleotide sequence set forth in any one of SEQ ID NOs: 57-90, SEQ ID NOs: 118-151, or SEQ ID NO: 213.

In some embodiments of any of the methods described herein, the direct repeat sequence comprises one or more of the following sequences: (a) X₁X₂TX₃X₄X₅X₆X₇X₈ (SEQ ID NO: 224), wherein X₁ is A or C or G, X₂ is T or C or A, X₃ is T or G or A, X₄ is T or G, X₅ is T or G or A, X₆ is G or T or A, X₇ is T or G or A, and X₈ is A or G or T (e.g., ATTGTTGDA (SEQ ID NO: 225)); (b) X₁X₂X₃X₄X₅X₆X₇X₈X₉ (SEQ ID NO: 226), wherein X₁ is T or C or A, X₂ is T or A or G, X₃ is T or C or A, X₄ is T or A, X₅ is T or A or G, X₆ is T or A, X₇ is A or T, X₈ is A or G or C or T, and X₉ is G or A or C (e.g., TTTTWTARG (SEQ ID NO: 227)); and (c) X₁X₂X₃AC (SEQ ID NO: 228), wherein X₁ is A or C or G, X₂ is C or A, and X₃ is A or C (e.g., ACAAC (SEQ ID NO: 229)). In some embodiments of any of the methods described herein, SEQ ID NO: 224 is proximal to the 5′ end of the direct repeat. In some embodiments of any of the methods described herein, SEQ ID NO: 228 is proximal to the 3′ end of the direct repeat.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1, and wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 57. In some embodiments of any of the methods described herein, the CRISPR-associated protein is a protein having at least 95% (e.g., 95%, 96%, 97%, 98%, 99% or 100%) identity to an amino acid sequence set forth in SEQ ID NO: 1, and wherein the direct repeat sequence comprises a nucleotide sequence that is at least 95% (e.g., 95%, 96%, 97%, 98%, 99% or 100%) identical to a nucleotide sequence set forth in SEQ ID NO: 57. In some embodiments of any of the methods described herein, the CRISPR-associated protein is capable of recognizing a protospacer adjacent motif (PAM) sequence, wherein the PAM sequence comprises a nucleic acid sequence set forth as 5′-TNNT-3′ or 5′-TNRT-3′, wherein “N” is any nucleotide and “R” is A or G.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 4, and wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 60. In some embodiments of any of the methods described herein, the CRISPR-associated protein is a protein having at least 95% (e.g., 95%, 96%, 97%, 98%, 99% or 100%) identity to an amino acid sequence set forth in SEQ ID NO: 4, and wherein the direct repeat sequence comprises a nucleotide sequence that is at least 95% (e.g., 95%, 96%, 97%, 98%, 99% or 100%) identical to a nucleotide sequence set forth in SEQ ID NO: 60. In some embodiments of any of the methods described herein, the CRISPR-associated protein is capable of recognizing a protospacer adjacent motif (PAM) sequence, wherein the PAM sequence comprises a nucleic acid sequence set forth as 5′-NTTN-3′, 5′-NTTR-3′ (e.g., 5 ‘-TTTG-3’), or 5′-NNR-3′, wherein “N” is any nucleotide and “R” is A or G.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 10, and wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 62 or SEQ ID NO: 213. In some embodiments of any of the methods described herein, the CRISPR-associated protein is a protein having at least 95% (e.g., 95%, 96%, 97%, 98%, 99% or 100%) identity to an amino acid sequence set forth in SEQ ID NO: 10, and wherein the direct repeat sequence comprises a nucleotide sequence that is at least 95% (e.g., 95%, 96%, 97%, 98%, 99% or 100%) identical to a nucleotide sequence set forth in SEQ ID NO: 62 or SEQ ID NO: 213. In some embodiments of any of the methods described herein, the CRISPR-associated protein is capable of recognizing a protospacer adjacent motif (PAM) sequence, wherein the PAM sequence comprises a nucleic acid sequence set forth as 5′-NTTN-3′ or 5′-RTTR-3′ (e.g., 5 ‘-ATTG-3’ or 5′-GTTA-3′), wherein “N” is any nucleotide and “R” is A or G.

In some embodiments of any of the methods described herein, the spacer sequence includes between about 15 nucleotides to about 55 nucleotides. In some embodiments of any of the methods described herein, the spacer sequence includes between 20 and 45 nucleotides.

In some embodiments of any of the methods described herein, the RNA guide optionally includes a tracrRNA and/or a modulator RNA. In some embodiments of any of the methods described herein, the system further includes a tracrRNA. In some embodiments of any of the methods described herein, the system does not include a tracrRNA. In some embodiments of any of the methods described herein, the CRISPR-associated protein is self-processing. In some embodiments of any of the methods described herein, the system further includes a modulator RNA.

In some embodiments of any of the methods described herein, the target nucleic acid is a DNA molecule. In some embodiments of any of the methods described herein, the target nucleic acid includes a PAM sequence.

In some embodiments of any of the methods described herein, the CRISPR-associated protein has non-specific nuclease activity.

In some embodiments of any of the methods described herein, the modification of the target nucleic acid is a double-stranded cleavage event. In some embodiments of any of the methods described herein, the modification of the target nucleic acid is a single-stranded cleavage event. In some embodiments of any of the methods described herein, the modification of the target nucleic acid results in an insertion event. In some embodiments of any of the methods described herein, the modification of the target nucleic acid results in a deletion event. In some embodiments of any of the methods described herein, the modification of the target nucleic acid results in cell toxicity or cell death.

In another aspect, the disclosure provides a method of editing a target nucleic acid, the method comprising contacting the target nucleic acid with a system described herein. In another aspect, the disclosure provides a method of modifying expression of a target nucleic acid, the method comprising contacting the target nucleic acid with a system described herein. In another aspect, the disclosure provides a method of targeting the insertion of a payload nucleic acid at a site of a target nucleic acid, the method comprising contacting the target nucleic acid with a system described herein. In another aspect, the disclosure provides a method of targeting the excision of a payload nucleic acid from a site at a target nucleic acid, the method comprising contacting the target nucleic acid with a system described herein. In another aspect, the disclosure provides a method of non-specifically degrading single-stranded DNA upon recognition of a DNA target nucleic acid, the method comprising contacting the target nucleic acid with a system described herein.

In some embodiments of any of the systems or methods provided herein, the contacting comprises directly contacting or indirectly contacting. In some embodiments of any of the systems or methods provided herein, contacting indirectly comprises administering one or more nucleic acids encoding an RNA guide or CRISPR-associated protein described herein under conditions that allow for production of the RNA guide and/or CRISPR-related protein. In some embodiments of any of the systems or methods provided herein, contacting includes contacting in vivo or contacting in vitro. In some embodiments of any of the systems or methods provided herein, contacting a target nucleic acid with the system comprises contacting a cell comprising the nucleic acid with the system under conditions that allow the CRISPR-related protein and guide RNA to reach the target nucleic acid. In some embodiments of any of the systems or methods provided herein, contacting a cell in vivo with the system comprises administering the system to the subject that comprises the cell, under conditions that allow the CRISPR-related protein and guide RNA to reach the cell or be produced in the cell.

In another aspect, the disclosure provides a system provided herein for use in an in vitro or ex vivo method of: (a) targeting and editing a target nucleic acid; (b) non-specifically degrading a single-stranded nucleic acid upon recognition of the nucleic acid; (c) targeting and nicking a non-spacer complementary strand of a double-stranded target upon recognition of a spacer complementary strand of the double-stranded target; (d) targeting and cleaving a double-stranded target nucleic acid; (e) detecting a target nucleic acid in a sample; (f) specifically editing a double-stranded nucleic acid; (g) base editing a double-stranded nucleic acid; (h) inducing genotype-specific or transcriptional-state-specific cell death or dormancy in a cell; (i) creating an indel in a double-stranded nucleic acid target; (j) inserting a sequence into a double-stranded nucleic acid target; or (k) deleting or inverting a sequence in a double-stranded nucleic acid target.

In another aspect, the disclosure provides method of introducing an insertion or deletion into a target nucleic acid in a mammalian cell, comprising a transfection of: (a) a nucleic acid sequence encoding a CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-56; and (b) an RNA guide (or a nucleic acid encoding the RNA guide) comprising a direct repeat sequence and a spacer sequence capable of hybridizing to the target nucleic acid; wherein the CRISPR-associated protein is capable of binding to the RNA guide; and wherein recognition of the target nucleic acid by the CRISPR-associated protein and RNA guide results in a modification of the target nucleic acid.

In some embodiments of any of the methods provided herein, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in SEQ ID NO: 4. In some embodiments of any of the methods provided herein, the CRISPR-associated protein comprises an amino acid sequence that is at least 95% (e.g., 95%, 96%, 97%, 98%, 99% or 100%) identical to an amino acid sequence set forth in SEQ ID NO: 4. In some embodiments of any of the methods provided herein, the direct repeat comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 60. In some embodiments of any of the methods provided herein, wherein the direct repeat comprises a nucleotide sequence that is at least 95% (e.g., 95%, 96%, 97%, 98%, 99% or 100%) identical to a nucleotide sequence set forth in SEQ ID NO: 60. In some embodiments of any of the methods provided herein, the target nucleic acid is adjacent to a PAM sequence, and the PAM sequence comprises a nucleic acid sequence set forth as 5′-NTTN-3′, 5′-NTTR-3′ (e.g., 5′-TTTG-3′), or 5′-NNR-3′, wherein “N” is any nucleotide and “R” is A or G.

In some embodiments of any of the methods provided herein, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in SEQ ID NO: 10. In some embodiments of any of the methods provided herein, the CRISPR-associated protein comprises an amino acid sequence that is at least 95% (e.g., 95%, 96%, 97%, 98%, 99% or 100%) identical to an amino acid sequence set forth in SEQ ID NO: 10. In some embodiments of any of the methods provided herein, the direct repeat comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in SEQ ID NO: 62 or SEQ ID NO: 213. In some embodiments of any of the methods provided herein, wherein the direct repeat comprises a nucleotide sequence that is at least 95% (e.g., 95%, 96%, 97%, 98%, 99% or 100%) identical to a nucleotide sequence set forth in SEQ ID NO: 62 or SEQ ID NO: 213. In some embodiments of any of the methods provided herein, the target nucleic acid is adjacent to a PAM sequence, and the PAM sequence comprises a nucleic acid sequence set forth as 5′-NTTN-3′ or 5′-RTTR-3′ (e.g., 5′-ATTG-3′ or 5′-GTTA-3′), wherein “N” is any nucleotide and “R” is A or G.

In some embodiments of any of the methods provided herein, the transfection is a transient transfection. In some embodiments of any of the methods provided herein, the cell is a human cell.

In another aspect, the disclosure provides a composition comprising: (a) a CRISPR-associated protein or a nucleic acid encoding the CRISPR-associated protein, and (b) an RNA guide comprising a direct repeat sequence and a spacer sequence; wherein the CRISPR-associated protein comprises one or more of the following amino acid sequences: (i) PX₁X₂X₃X₄F (SEQ ID NO: 216), wherein X₁ is L or M or I or C or F, X₂ is Y or W or F, X₃ is K or T or C or R or W or Y or H or V, and X₄ is I or L or M; (ii) RX₁X₂X₃L (SEQ ID NO: 217), wherein X₁ is I or L or M or Y or T or F, X₂ is R or Q or K or E or S or T, and X₃ is L or I or T or C or M or K; (iii) NX₁YX₂ (SEQ ID NO: 218), wherein X₁ is I or L or F and X₂ is K or R or V or E; (iv) KX₁X₂X₃FAX₄X₅KD (SEQ ID NO: 219), wherein X₁ is T or I or N or A or S or F or V, X₂ is I or V or L or S, X₃ is H or S or G or R, X₄ is D or S or E, and X₅ is I or V or M or T or N; (v) LX₁NX₂ (SEQ ID NO: 220), wherein X₁ is G or S or C or T and X₂ is N or Y or K or S; (vi) PX₁X₂X₃X₄SQX₅DS (SEQ ID NO: 221), wherein X₁ is S or P or A, X₂ is Y or S or A or P or E or Y or Q or N, X₃ is F or Y or H, X₄ is T or S, and X₅ is M or T or I; (vii) KX₁X₂VRX₃X₄QEX₅H (SEQ ID NO: 222), wherein X₁ is N or K or W or R or E or T or Y, X₂ is M or R or L or S or K or V or E or T or I or D, X₃ is L or R or H or P or T or K or Q of P or S or A, X₄ is G or Q or N or R or K or E or I or T or S or C, and X₅ is R or W or Y or K or T or F or S or Q; and (viii) X₁NGX₂X₃X₄DX₅NX₆X₇X₈N (SEQ ID NO: 223), wherein X₁ is I or K or V or L, X₂ is L or M, X₃ is N or H or P, X₄ is A or S or C, X₅ is V or Y or I or F or T or N, X₆ is A or S, X₇ is S or A or P, and X₈ is M or C or L or R or N or S or K or L; and wherein the CRISPR-associated protein is capable of binding to the RNA guide and of modifying the target nucleic acid sequence complementary to the spacer sequence.

In some embodiments of any of the compositions described herein, the direct repeat sequence comprises one or more of the following sequences: (a) X₁X₂TX₃X₄X₅X₆X₇X₈ (SEQ ID NO: 224), wherein X₁ is A or C or G, X₂ is T or C or A, X₃ is T or G or A, X₄ is T or G, X₅ is T or G or A, X₆ is G or T or A, X₇ is T or G or A, and X₈ is A or G or T (e.g., ATTGTTGDA (SEQ ID NO: 225)); (b) X₁X₂X₃X₄X₅X₆X₇X₈X₉ (SEQ ID NO: 226), wherein X₁ is T or C or A, X₂ is T or A or G, X₃ is T or C or A, X₄ is T or A, X₅ is T or A or G, X₆ is T or A, X₇ is A or T, X₈ is A or G or C or T, and X₉ is G or A or C (e.g., TTTTWTARG (SEQ ID NO: 227)); and (c) X₁X₂X₃AC (SEQ ID NO: 228), wherein X₁ is A or C or G, X₂ is C or A, and X₃ is A or C (e.g., ACAAC (SEQ ID NO: 229)). In some embodiments of any of the compositions described herein, SEQ ID NO: 224 is proximal to the 5′ end of the direct repeat. In some embodiments of any of the compositions described herein, SEQ ID NO: 228 is proximal to the 3′ end of the direct repeat.

In some embodiments of any of the compositions described herein, the CRISPR-associated protein includes at least one (e.g., one, two, or three) RuvC domain or at least one split RuvC domain.

In some embodiments of any of the compositions described herein, the spacer sequence of the RNA guide includes between about 15 nucleotides to about 55 nucleotides. In some embodiments of any of the compositions described herein, the spacer sequence of the RNA guide includes between 20 and 45 nucleotides.

In some embodiments of any of the compositions described herein, the CRISPR-associated protein comprises a catalytic residue (e.g., aspartic acid or glutamic acid). In some embodiments of any of the compositions described herein, the CRISPR-associated protein cleaves the target nucleic acid. In some embodiments of any of the compositions described herein, the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In some embodiments of any of the compositions described herein, the nucleic acid encoding the CRISPR-associated protein is codon-optimized for expression in a cell, e.g., a eukaryotic cell, e.g., a mammalian cell, e.g., a human cell. In some embodiments of any of the compositions described herein, the nucleic acid encoding the CRISPR-associated protein is operably linked to a promoter. In some embodiments of any of the compositions described herein, the nucleic acid encoding the CRISPR-associated protein is in a vector. In some embodiments, the vector comprises a retroviral vector, a lentiviral vector, a phage vector, an adenoviral vector, an adeno-associated vector, or a herpes simplex vector.

In some embodiments of any of the compositions described herein, the target nucleic acid is a DNA molecule. In some embodiments of any of the compositions described herein, the target nucleic acid includes a PAM sequence.

In some embodiments of any of the compositions described herein, the CRISPR-associated protein has non-specific nuclease activity.

In some embodiments of any of the compositions described herein, recognition of the target nucleic acid by the CRISPR-associated protein and RNA guide results in a modification of the target nucleic acid. In some embodiments of any of the compositions described herein, the modification of the target nucleic acid is a double-stranded cleavage event. In some embodiments of any of the compositions described herein, the modification of the target nucleic acid is a single-stranded cleavage event. In some embodiments of any of the compositions described herein, the modification of the target nucleic acid results in an insertion event. In some embodiments of any of the compositions described herein, the modification of the target nucleic acid results in a deletion event. In some embodiments of any of the compositions described herein, the modification of the target nucleic acid results in cell toxicity or cell death.

In some embodiments of any of the compositions described herein, the system further includes a donor template nucleic acid. In some embodiments of any of the compositions described herein, the donor template nucleic acid is a DNA molecule. In some embodiments of any of the compositions described herein, wherein the donor template nucleic acid is an RNA molecule.

In some embodiments of any of the compositions described herein, the RNA guide optionally includes a tracrRNA. In some embodiments of any of the compositions described herein, the system further includes a tracrRNA. In some embodiments of any of the compositions described herein, the system does not include a tracrRNA. In some embodiments of any of the compositions described herein, the CRISPR-associated protein is self-processing.

In some embodiments of any of the compositions described herein, the system is present in a delivery composition comprising a nanoparticle, a liposome, an exosome, a microvesicle, or a gene-gun.

In some embodiments of any of the compositions described herein, the compositions are within a cell. In some embodiments, the cell is a eukaryotic cell. In some embodiments, the cell is a mammalian cell. In some embodiments, the cell is a human cell. In some embodiments, the cell is a prokaryotic cell.

The effectors described herein provide additional features that include, but are not limited to, 1) novel nucleic acid editing properties and control mechanisms, 2) smaller size for greater versatility in delivery strategies, 3) genotype triggered cellular processes such as cell death, and 4) programmable RNA-guided DNA insertion, excision, and mobilization, and 5) differentiated profile of pre-existing immunity through a non-human commensal source. See, e.g., Examples 1, 4, and 5 and FIGS. 1-3 and 5-11D. Addition of the novel DNA-targeting systems described herein to the toolbox of techniques for genome and epigenome manipulation enables broad applications for specific, programmed perturbations.

Other features and advantages of the invention will be apparent from the following detailed description and from the claims.

### BRIEF FIGURE DESCRIPTION

The figures are a series of schematics that represent the results of analysis of a protein cluster referred to as CLUST.091979.

FIG. 1A, FIG. 1B, FIG. 1C, FIG. 1D, FIG. 1E, FIG. 1F, FIG. 1G, FIG. 1H, FIG. 1I, FIG. 1J, FIG. 1K, and FIG. 1L collectively show an alignment of the effectors of SEQ ID NOs: 1-4, 14, 15, 17-19, 21-25, 27-33, 35-49, 51-56.

FIG. 2 is a schematic showing the RuvC domains of CLUST.091979 effectors, which is based upon the consensus sequence of the sequences shown in Table 6.

FIG. 3 shows an alignment of the direct repeat sequences of SEQ ID NOs: 57, 58, 60, 62, 63, 70, 72-74, 76, 77, 80, 83, 84, 86-88, 90, 128, 130, 139, and 213. The consensus sequence (SEQ ID NO: 230) is shown at the top of the alignment.

FIG. 4A is a schematic representation of the components of the in vivo negative selection screening assay described in Example 4. CRISPR array libraries were designed including non-representative spacers uniformly sampled from both strands of the pACYC184 or E. coli essential genes flanked by two DRs and expressed by J23119.

FIG. 4B is a schematic representation of the in vivo negative selection screening workflow described in Example 4. CRISPR array libraries were cloned into the effector plasmid. The effector plasmid and the non-coding plasmid were transformed into E. coli followed by outgrowth for negative selection of CRISPR arrays conferring interference against transcripts from pACYC184 or E. coli essential genes. Targeted sequencing of the effector plasmid was used to identify depleted CRISPR arrays. Small RNAseq was further performed to identify mature crRNAs and potential tracrRNA requirements.

FIG. 5 is a graph for CLUST.091979 AUXO013988882 (effector set forth in SEQ ID NO: 1) showing the degree of depletion activity of the engineered compositions for spacers targeting pACYC184 and direct repeat transcriptional orientations, with a non-coding sequence. The degree of depletion with the direct repeat in the “forward” orientation (5′-ACTA . . . AACT-[spacer]-3′) and with the direct repeat in the “reverse” orientation (5′-AGTT . . . TAGT-[spacer]-3′) are depicted.

FIG. 6A is a graphical representation showing the density of depleted and non-depleted targets for CLUST.091979 AUXO013988882, with a non-coding sequence, by location on the pACYC184 plasmid. FIG. 6B is a graphic representation showing the density of depleted and non-depleted targets for CLUST.091979 AUXO013988882, with a non-coding sequence, by location on the E. coli strain, E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 7 is a WebLogo of the sequences flanking depleted targets in E. Cloni as a prediction of the PAM sequence for CLUST.091979 AUXO013988882 (with a non-coding sequence).

FIG. 8 is a graph for CLUST.091979 SRR3181151 (effector set forth in SEQ ID NO: 4) showing the degree of depletion activity of the engineered compositions for spacers targeting pACYC184 and direct repeat transcriptional orientations, with a non-coding sequence. The degree of depletion with the direct repeat in the “forward” orientation (5′-GTTG . . . CAGG-[spacer]-3′) and with the direct repeat in the “reverse” orientation (5′-CCTG . . . CAAC-[spacer]-3′) are depicted.

FIG. 9A is a graphical representation showing the density of depleted and non-depleted targets for CLUST.091979 SRR3181151, with a non-coding sequence, by location on the pACYC184 plasmid. FIG. 9B is a graphic representation showing the density of depleted and non-depleted targets for CLUST.091979 SRR3181151, with a non-coding sequence, by location on the E. coli strain, E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 10 is a WebLogo of the sequences flanking depleted targets in E. Cloni as a prediction of the PAM sequence for CLUST.091979 SRR3181151 (with a non-coding sequence).

FIG. 11A shows indels induced by the effector of SEQ ID NO: 4 at an AAV target locus of SEQ ID NO: 206 and a VEG-FA target locus of SEQ ID NO: 208 in HEK293 cells. FIG. 11B shows indels induced by the effector of SEQ ID NO: 4 at AAVS1 target loci of SEQ ID NOs: 253, 255, 257, 259, and 275, VEGFA target loci of SEQ NOs: 263, 265, 267, 269, 271, 273, and 277, and an EMX1 target locus of SEQ ID NO: 261 in HEK293 cells. FIG. 11C shows indels induced by the effector of SEQ ID NO: 10 at an AAVS1 target loci of SEQ ID NO: 210, an AAVS1 target locus of SEQ ID NO: 212, and a VEGFA target locus of SEQ ID NO: 215 in HEK293 cells. FIG. 11D shows indels induced by the effector of SEQ ID NO: 10 at AAVS1 target loci of SEQ ID NOs: 279, 281, 285, and 287, a VEGFA target locus of SEQ ID NO: 283, and an EMX1 target locus of SEQ ID NO: 289 in HEK293 cells.

### DETAILED DESCRIPTION

CRISPR-Cas systems, which are naturally diverse, comprise a wide range of activity mechanisms and functional elements that can be harnessed for programmable biotechnologies. In nature, these systems enable efficient defense against foreign DNA and viruses while providing self versus non-self discrimination to avoid self-targeting. In an engineered setting, these systems provide a diverse toolbox of molecular technologies and define the boundaries of the targeting space. The methods described herein have been used to discover additional mechanisms and parameters within single subunit Class 2 effector systems, which expand the capabilities of RNA-programmable nucleic acid manipulation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Applicant reserves the right to alternatively claim any disclosed invention using the transitional phrase “comprising,” “consisting essentially of,” or “consisting of,” according to standard practice in patent law.

As used herein, the singular forms “a,” “an,” and “the” include plural referents unless the context clearly dictates otherwise. For example, reference to “a nucleic acid” means one or more nucleic acids.

It is noted that terms like “preferably,” “suitably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention, it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The term “CRISPR-Cas system,” as used herein, refers to nucleic acids and/or proteins involved in the expression of, or directing the activity of, CRISPR effectors, including sequences encoding CRISPR effectors, RNA guides, and other sequences and transcripts from a CRISPR locus.

The terms “CRISPR-associated protein,” “CRISPR-Cas effector,” “CRISPR effector,” “effector,” “effector protein,” “CRISPR enzyme,” or the like, as used interchangeably herein, refer to a protein that carries out an enzymatic activity or that binds to a target site on a nucleic acid specified by an RNA guide. In some embodiments, a CRISPR effector has endonuclease activity, nickase activity, and/or exonuclease activity.

The terms “RNA guide,” “guide RNA,” “gRNA,” and “guide sequence,” as used herein, refer to any RNA molecule that facilitates the targeting of an effector described herein to a target nucleic acid, such as DNA and/or RNA. Exemplary “RNA guides” include, but are not limited to, crRNAs, as well as crRNAs hybridized to or fused to either tracrRNAs and/or modulator RNAs. In some embodiments, an RNA guide includes both a crRNA and a tracrRNA, either fused into a single RNA molecule or as separate RNA molecules. In some embodiments, an RNA guide includes a crRNA and a modulator RNA, either fused into a single RNA molecule or as separate RNA molecules. In some embodiments, an RNA guide includes a crRNA, a tracrRNA, and a modulator RNA, either fused into a single RNA molecule or as separate RNA molecules.

The terms “CRISPR effector complex,” “effector complex,” or “surveillance complex,” as used herein, refer to a complex containing a CRISPR effector and an RNA guide. A CRISPR effector complex may further comprise one or more accessory proteins. The one or more accessory proteins may be non-catalytic and/or non-target binding.

The terms “CRISPR RNA” and “crRNA,” as used herein, refer to an RNA molecule comprising a guide sequence used by a CRISPR effector specifically to recognize a nucleic acid sequence. A crRNA “spacer” sequence is complementary to and capable of partially or completely binding to a nucleic acid target sequence. A crRNA may comprise a sequence that hybridizes to a tracrRNA. In turn, the crRNA: tracrRNA duplex may bind to a CRISPR effector. As used herein, the term “pre-crRNA” refers to an unprocessed RNA molecule comprising a DR-spacer-DR sequence. As used herein, the term “mature crRNA” refers to a processed form of a pre-crRNA; a mature crRNA may comprise a DR-spacer sequence, wherein the DR is a truncated form of the DR of a pre-crRNA and/or the spacer is a truncated form of the spacer of a pre-crRNA.

The terms “trans-activating crRNA” or “tracrRNA,” as used herein, refer to an RNA molecule comprising a sequence that forms a structure and/or sequence motif required for a CRISPR effector to bind to a specified target nucleic acid.

The term “CRISPR array,” as used herein, refers to a nucleic acid (e.g., DNA) segment that comprises CRISPR repeats and spacers, starting with the first nucleotide of the first CRISPR repeat and ending with the last nucleotide of the final (terminal) CRISPR repeat. Typically, each spacer in a CRISPR array is located between two repeats. The terms “CRISPR repeat,” “CRISPR direct repeat,” and “direct repeat,” as used herein, refer to multiple short direct repeating sequences, which show very little or no sequence variation within a CRISPR array.

The term “modulator RNA” as described herein refers to any RNA molecule that modulates (e.g., increases or decreases) an activity of a CRISPR effector or a nucleoprotein complex that includes a CRISPR effector. In some embodiments, a modulator RNA modulates a nuclease activity of a CRISPR effector or a nucleoprotein complex that includes a CRISPR effector.

As used herein, the term “target nucleic acid” refers to a nucleic acid that comprises a nucleotide sequence complementary to the entirety or a part of the spacer in an RNA guide. In some embodiments, the target nucleic acid comprises a gene. In some embodiments, the target nucleic acid comprises a non-coding region (e.g., a promoter). In some embodiments, the target nucleic acid is single-stranded. In some embodiments, the target nucleic acid is double-stranded. A “transcriptionally-active site,” as used herein, refers to a site in a nucleic acid sequence being actively transcribed.

As used herein, the term “protospacer adjacent motif” or “PAM” refers to a DNA sequence adjacent to a target sequence to which a complex comprising an effector and an RNA guide binds. In some embodiments, a PAM is required for enzyme activity. As used herein, the term “adjacent” includes instances in which an RNA guide of the complex specifically binds, interacts, or associates with a target sequence that is immediately adjacent to a PAM. In such instances, there are no nucleotides between the target sequence and the PAM. The term “adjacent” also includes instances in which there are a small number (e.g., 1, 2, 3, 4, or 5) of nucleotides between the target sequence, to which the targeting moiety binds, and the PAM. As used herein, the term “recognizing a PAM sequence” refers to the binding of a complex comprising a CRISPR-associated protein and a crRNA to a target nucleic acid, wherein the target nucleic acid is adjacent to a PAM sequence.

The terms “activated CRISPR effector complex,” “activated CRISPR complex,” and “activated complex,” as used herein, refer to a CRISPR effector complex capable of modifying a target nucleic acid. In some embodiments, an activated CRISPR complex is capable of modifying a target nucleic acid following binding of the activated CRISPR complex to the target nucleic acid. In some embodiments, binding of an activated CRISPR complex to a target nucleic acid results in an additional cleavage event, such as collateral cleavage.

The term “cleavage event,” as used herein, refers to a break in a nucleic acid, such as DNA and/or RNA. In some embodiments, a cleavage event refers to a break in a target nucleic acid created by a nuclease of a CRISPR system described herein. In some embodiments, the cleavage event is a double-stranded DNA break. In some embodiments, the cleavage event is a single-stranded DNA break. In some embodiments, a cleavage event refers to a break in a collateral nucleic acid.

The term “collateral nucleic acid,” as used herein, refers to a nucleic acid substrate that is cleaved non-specifically by an activated CRISPR complex. The term “collateral DNase activity,” as used herein in reference to a CRISPR effector, refers to non-specific DNase activity of an activated CRISPR complex. The term “collateral RNase activity,” as used herein in reference to a CRISPR effector, refers to non-specific RNase activity of an activated CRISPR complex.

The term “donor template nucleic acid,” as used herein, refers to a nucleic acid molecule that can be used to make a templated change to a target sequence or target-proximal sequence after a CRISPR effector described herein has modified the target nucleic acid. In some embodiments, the donor template nucleic acid is a double-stranded nucleic acid. In some embodiments, the donor template nucleic acid is a single-stranded nucleic acid. In some embodiments, the donor template nucleic acid is linear. In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof. Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif)

The term “genetic modification” or “genetic engineering” broadly refers to manipulation of the genome or nucleic acids of a cell. Likewise, the terms “genetically engineered” and “engineered” refer to a cell comprising a manipulated genome or nucleic acids. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization.

The term “recombinant” indicates that a nucleic acid, protein, or cell is the product of genetic modification, engineering, or recombination. Generally, the term “recombinant” refers to a nucleic acid, protein, or cell that contains or is encoded by genetic material derived from multiple sources. As used herein, the term “recombinant” may also be used to describe a cell that comprises a mutated nucleic acid or protein, including a mutated form of an endogenous nucleic acid or protein. The terms “recombinant cell” and “recombinant host” can be used interchangeably. In some embodiments, a recombinant cell comprises a CRISPR effector disclosed herein. The CRISPR effector can be codon-optimized for expression in the recombinant cell. In some embodiments, a recombinant cell disclosed herein further comprises an RNA guide. In some embodiments, an RNA guide of a recombinant cell disclosed herein comprises a tracrRNA. In some embodiments, a recombinant cell disclosed herein comprises a modulator RNA. In some embodiments, the recombinant cell is a prokaryotic cell, such as an E. coli cell. In some embodiments, the recombinant cell is a eukaryotic cell, such as a mammalian cell, including a human cell.

### Identification of CLUST.091979

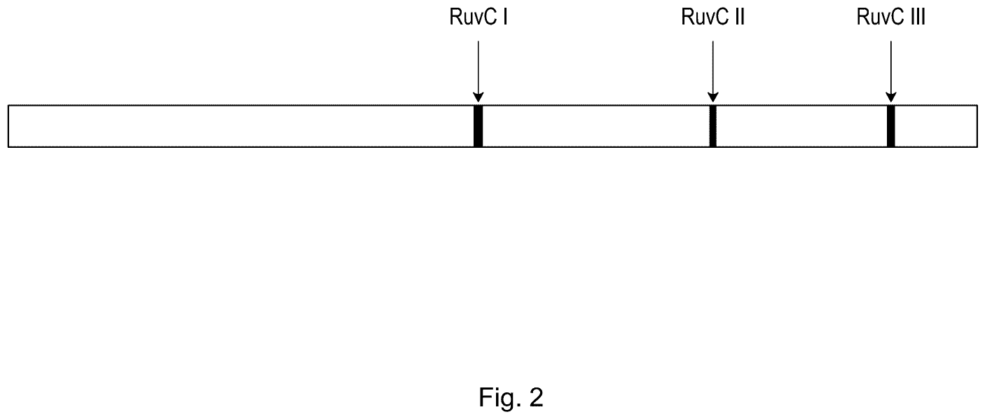

This application relates to the identification, engineering, and use of a novel protein family referred to herein as “CLUST.091979.” As shown in FIG. 2, the proteins of CLUST.091979 comprise a RuvC domain (denoted RuvC I, RuvC II, and RuvC III). As shown in TABLE 5, effectors of CLUST.091979 range in size from about 700 amino acids to about 800 amino acids. Therefore, the effectors of CLUST.091979 are smaller than effectors known in the art, as shown below. See, e.g., TABLE 1.

TABLE 1
Sizes of known CRISPR-Cas system effectors.

| Effector | Size (aa) |
|---|---|
| StCas9 | 1128 |
| SpCas9 | 1368 |
| SaCas9 | 1053 |
| FnCpf1 | 1300 |
| AsCpf1 | 1307 |
| LbCpf1 | 1246 |
| C2c1 | 1127 (average) |
| CasX | 982 (average) |
| CasY | 1189 (average) |
| C2c2 | 1232 (average) |

The effectors of CLUST.091979 were identified using computational methods and algorithms to search for and identify proteins exhibiting a strong co-occurrence pattern with certain other features. In certain embodiments, these computational methods were directed to identifying proteins that co-occurred in close proximity to CRISPR arrays. The methods disclosed herein are also useful in identifying proteins that naturally occur within close proximity to other features, both non-coding and protein-coding (e.g., fragments of phage sequences in non-coding areas of bacterial loci or CRISPR Cm′ proteins). It is understood that the methods and calculations described herein may be performed on one or more computing devices.

Sets of genomic sequences were obtained from genomic or metagenomic databases. The databases comprised short reads, or contig level data, or assembled scaffolds, or complete genomic sequences of organisms. Likewise, the databases may comprise genomic sequence data from prokaryotic organisms, or eukaryotic organisms, or may include data from metagenomic environmental samples. Examples of database repositories include the National Center for Biotechnology Information (NCBI) RefSeq, NCBI GenBank, NCBI Whole Genome Shotgun (WGS), and the Joint Genome Institute (JGI) Integrated Microbial Genomes (IMG).

In some embodiments, a minimum size requirement is imposed to select genome sequence data of a specified minimum length. In certain exemplary embodiments, the minimum contig length may be 100 nucleotides, 500 nt, 1 kb, 1.5 kb, 2 kb 3 kb, 4 kb, 5 kb, 10 kb, 20 kb, 40 kb, or 50 kb.

In some embodiments, known or predicted proteins are extracted from the complete or a selected set of genome sequence data. In some embodiments, known or predicted proteins are taken from extracting coding sequence (CDS) annotations provided by the source database. In some embodiments, predicted proteins are determined by applying a computational method to identify proteins from nucleotide sequences. In some embodiments, the GeneMark Suite is used to predict proteins from genome sequences. In some embodiments, Prodigal is used to predict proteins from genome sequences. In some embodiments, multiple protein prediction algorithms may be used over the same set of sequence data with the resulting set of proteins de-duplicated.

In some embodiments, CRISPR arrays are identified from the genome sequence data. In some embodiments, PILER-CR is used to identify CRISPR arrays. In some embodiments, CRISPR Recognition Tool (CRT) is used to identify CRISPR arrays. In some embodiments, CRISPR arrays are identified by a heuristic that identifies nucleotide motifs repeated a minimum number of times (e.g., 2, 3, or 4 times), where the spacing between consecutive occurrences of a repeated motif does not exceed a specified length (e.g., 50, 100, or 150 nucleotides). In some embodiments, multiple CRISPR array identification tools may be used over the same set of sequence data with the resulting set of CRISPR arrays de-duplicated.

In some embodiments, proteins in close proximity to CRISPR arrays (referred to herein as “CRISPR-proximal protein clusters”) are identified. In some embodiments, proximity is defined as a nucleotide distance, and may be within 20 kb, 15 kb, or 5 kb. In some embodiments, proximity is defined as the number of open reading frames (ORFs) between a protein and a CRISPR array, and certain exemplary distances may be 10, 5, 4, 3, 2, 1, or 0 ORFs. The proteins identified as being within close proximity to a CRISPR array are then grouped into clusters of homologous proteins. In some embodiments, blastclust is used to form CRISPR-proximal protein clusters. In certain other embodiments, mmseqs2 is used to form CRISPR-proximal protein clusters.

To establish a pattern of strong co-occurrence between the members of a CRISPR-proximal protein cluster, a BLAST search of each member of the protein cluster may be performed over the complete set of known and predicted proteins previously compiled. In some embodiments, UBLAST or mmseqs2 may be used to search for similar proteins. In some embodiments, a search may be performed only for a representative subset of proteins in the family.

In some embodiments, the CRISPR-proximal protein clusters are ranked or filtered by a metric to determine co-occurrence. One exemplary metric is the ratio of the number of elements in a protein cluster against the number of BLAST matches up to a certain E value threshold. In some embodiments, a constant E value threshold may be used. In other embodiments, the E value threshold may be determined by the most distant members of the protein cluster. In some embodiments, the global set of proteins is clustered and the co-occurrence metric is the ratio of the number of elements of the CRISPR-proximal protein cluster against the number of elements of the containing global cluster(s).

In some embodiments, a manual review process is used to evaluate the potential functionality and the minimal set of components of an engineered system based on the naturally occurring locus structure of the proteins in the cluster. In some embodiments, a graphical representation of the protein cluster may assist in the manual review and may contain information including pairwise sequence similarity, phylogenetic tree, source organisms/environments, predicted functional domains, and a graphical depiction of locus structures. In some embodiments, the graphical depiction of locus structures may filter for nearby protein families that have a high representation. In some embodiments, representation may be calculated by the ratio of the number of related nearby proteins against the size(s) of the containing global cluster(s). In certain exemplary embodiments, the graphical representation of the protein cluster may contain a depiction of the CRISPR array structures of the naturally occurring loci. In some embodiments, the graphical representation of the protein cluster may contain a depiction of the number of conserved direct repeats versus the length of the putative CRISPR array or the number of unique spacer sequences versus the length of the putative CRISPR array. In some embodiments, the graphical representation of the protein cluster may contain a depiction of various metrics of co-occurrence of the putative effector with CRISPR arrays predict new CRISPR-Cas systems and identify their components.

### Pooled-Screening of CLUST.091979

To efficiently validate the activity, mechanisms, and functional parameters of the engineered CLUST.091979 CRISPR-Cas systems identified herein, a pooled-screening approach in E. coli was used, as described in Example 4. First, from the computational identification of the conserved protein and noncoding elements of the CLUST.091979 CRISPR-Cas system, DNA synthesis and molecular cloning were used to assemble the separate components into a single artificial expression vector, which in one embodiment is based on a pET-28a+ backbone. In a second embodiment, the effectors and noncoding elements are transcribed on an mRNA transcript, and different ribosomal binding sites are used to translate individual effectors.

Second, the natural crRNA and targeting spacers were replaced with a library of unprocessed crRNAs containing non-natural spacers targeting a second plasmid, pACYC184. This crRNA library was cloned into the vector backbone comprising the effectors and noncoding elements (e.g., pET-28a+), and the library was subsequently transformed into E. coli along with the pACYC184 plasmid target. Consequently, each resulting E. coli cell contains no more than one targeting array. In an alternate embodiment, the library of unprocessed crRNAs containing non-natural spacers additionally target E. coli essential genes, drawn from resources such as those described in Baba et al. (2006) Mol. Syst. Biol. 2: 2006.0008; and Gerdes et al. (2003) J. Bacteriol. 185(19): 5673-84, the entire contents of each of which are incorporated herein by reference. In this embodiment, positive, targeted activity of the novel CRISPR-Cas systems that disrupts essential gene function results in cell death or growth arrest. In some embodiments, the essential gene targeting spacers can be combined with the pACYC184 targets.

Third, the E. coli were grown under antibiotic selection. In one embodiment, triple antibiotic selection is used: kanamycin for ensuring successful transformation of the pET-28a+ vector containing the engineered CRISPR effector system and chloramphenicol and tetracycline for ensuring successful co-transformation of the pACYC184 target vector. Since pACYC184 normally confers resistance to chloramphenicol and tetracycline, under antibiotic selection, positive activity of the novel CRISPR-Cas system targeting the plasmid will eliminate cells that actively express the effectors, noncoding elements, and specific active elements of the crRNA library. Typically, populations of surviving cells are analyzed 12-14 h post-transformation. In some embodiments, analysis of surviving cells is conducted 6-8 h post-transformation, 8-12 h post-transformation, up to 24 h post-transformation, or more than 24 h post-transformation. Examining the population of surviving cells at a later time point compared to an earlier time point results in a depleted signal compared to the inactive crRNAs.

In some embodiments, double antibiotic selection is used. Withdrawal of either chloramphenicol or tetracycline to remove selective pressure can provide novel information about the targeting substrate, sequence specificity, and potency. For example, cleavage of dsDNA in a selected or unselected gene can result in negative selection in E. coli, wherein depletion of both selected and unselected genes is observed. If the CRISPR-Cas system interferes with transcription or translation (e.g., by binding or by transcript cleavage), then selection will only be observed for targets in the selected resistance gene, rather than in the unselected resistance gene.

In some embodiments, only kanamycin is used to ensure successful transformation of the pET-28a+ vector comprising the engineered CRISPR-Cas system. This embodiment is suitable for libraries containing spacers targeting E. coli essential genes, as no additional selection beyond kanamycin is needed to observe growth alterations. In this embodiment, chloramphenicol and tetracycline dependence is removed, and their targets (if any) in the library provide an additional source of negative or positive information about the targeting substrate, sequence specificity, and potency.

Since the pACYC184 plasmid contains a diverse set of features and sequences that may affect the activity of a CRISPR-Cas system, mapping the active crRNAs from the pooled screen onto pACYC184 provides patterns of activity that can be suggestive of different activity mechanisms and functional parameters. In this way, the features required for reconstituting the novel CRISPR-Cas system in a heterologous prokaryotic species can be more comprehensively tested and studied.

The key advantages of the in vivo pooled-screen described herein include:
(1) Versatility—Plasmid design allows multiple effectors and/or noncoding elements to be expressed; library cloning strategy enables both transcriptional directions of the computationally predicted crRNA to be expressed;
(2) Comprehensive tests of activity mechanisms & functional parameters—Evaluates diverse interference mechanisms, including nucleic acid cleavage; examines co-occurrence of features such as transcription, plasmid DNA replication; and flanking sequences for crRNA library can be used to reliably determine PAMs with complexity equivalence of 4N's;
(3) Sensitivity—pACYC184 is a low copy plasmid, enabling high sensitivity for CRISPR-Cas activity since even modest interference rates can eliminate the antibiotic resistance encoded by the plasmid; and
(4) Efficiency—Optimized molecular biology steps to enable greater speed and throughput RNA-sequencing and protein expression samples can be directly harvested from the surviving cells in the screen.

The novel CLUST.091979 CRISPR-Cas family described herein was evaluated using this in vivo pooled-screen to evaluate is operational elements, mechanisms, and parameters, as well as its ability to be active and reprogrammed in an engineered system outside of its endogenous cellular environment.

### CRISPR Effector Activity and Modifications

In some embodiments, a CRISPR effector of CLUST.091979 and an RNA guide form a “binary” complex that may include other components. The binary complex is activated upon binding to a nucleic acid substrate that is complementary to a spacer sequence in the RNA guide (i.e., a sequence-specific substrate or target nucleic acid). In some embodiments, the sequence-specific substrate is a double-stranded DNA. In some embodiments, the sequence-specific substrate is a single-stranded DNA. In some embodiments, the sequence-specific substrate is a single-stranded RNA. In some embodiments, the sequence-specific substrate is a double-stranded RNA. In some embodiments, the sequence-specificity requires a complete match of the spacer sequence in the RNA guide (e.g., crRNA) to the target substrate. In other embodiments, the sequence specificity requires a partial (contiguous or non-contiguous) match of the spacer sequence in the RNA guide (e.g., crRNA) to the target substrate.

In some embodiments, a CRISPR effector of the present invention has enzymatic activity, e.g., nuclease activity, over a broad range of pH conditions. In some embodiments, the nuclease has enzymatic activity, e.g., nuclease activity, at a pH of from about 3.0 to about 12.0. In some embodiments, the CRISPR effector has enzymatic activity at a pH of from about 4.0 to about 10.5. In some embodiments, the CRISPR effector has enzymatic activity at a pH of from about 5.5 to about 8.5. In some embodiments, the CRISPR effector has enzymatic activity at a pH of from about 6.0 to about 8.0. In some embodiments, the CRISPR effector has enzymatic activity at a pH of about 7.0.

In some embodiments, a CRISPR effector of the present invention has enzymatic activity, e.g., nuclease activity, at a temperature range of from about 10° C. to about 100° C. In some embodiments, a CRISPR effector of the present invention has enzymatic activity at a temperature range from about 20° C. to about 90° C. In some embodiments, a CRISPR effector of the present invention has enzymatic activity at a temperature of about 20° C. to about 25° C. or at a temperature of about 37° C.

In some embodiments, the binary complex becomes activated upon binding to the target substrate. In some embodiments, the activated complex exhibits “multiple turnover” activity, whereby upon acting on (e.g., cleaving) the target substrate the activated complex remains in an activated state. In some embodiments, the activated binary complex exhibits “single turnover” activity, whereby upon acting on the target substrate the binary complex reverts to an inactive state. In some embodiments, the activated binary complex exhibits non-specific (i.e., “collateral”) cleavage activity whereby the complex cleaves non-target nucleic acids. In some embodiments, the non-target nucleic acid is a DNA molecule (e.g., a single-stranded or a double-stranded DNA). In some embodiments, the non-target nucleic acid is an RNA molecule (e.g., a single-stranded or a double-stranded RNA).

In some embodiments wherein a CRISPR effector of the present invention induces double-stranded breaks or single-stranded breaks in a target nucleic acid, (e.g. genomic DNA), the double-stranded break can stimulate cellular endogenous DNA-repair pathways, including Homology Directed Recombination (HDR), Non-Homologous End Joining (NHEJ), or Alternative Non-Homologues End-Joining (A-NHEJ). NHEJ can repair cleaved target nucleic acid without the need for a homologous template. This can result in deletion or insertion of one or more nucleotides at the target locus. HDR can occur with a homologous template, such as the donor DNA. The homologous template can comprise sequences that are homologous to sequences flanking the target nucleic acid cleavage site. In some cases, HDR can insert an exogenous polynucleotide sequence into the cleave target locus. The modifications of the target DNA due to NHEJ and/or HDR can lead to, for example, mutations, deletions, alterations, integrations, gene correction, gene replacement, gene tagging, transgene knock-in, gene disruption, and/or gene knock-outs.

In some embodiments, a CRISPR effector described herein can be fused to one or more peptide tags, including a His-tag, GST-tag, FLAG-tag, or myc-tag. In some embodiments, a CRISPR effector described herein can be fused to a detectable moiety such as a fluorescent protein (e.g., green fluorescent protein or yellow fluorescent protein). In some embodiments, a CRISPR effector and/or accessory protein of this disclosure is fused to a peptide or non-peptide moiety that allows the protein to enter or localize to a tissue, a cell, or a region of a cell. For instance, a CRISPR effector of this disclosure may comprise a nuclear localization sequence (NLS) such as an SV40 (simian virus 40) NLS, c-Myc NLS, or other suitable monopartite NLS. The NLS may be fused to the N-terminus and/or C-terminus of the CRISPR effector, and may be fused singly (i.e., a single NLS) or concatenated (e.g., a chain of 2, 3, 4, etc. NLS).

In some embodiments, at least one Nuclear Export Signal (NES) is attached to a nucleic acid sequences encoding the CRISPR effector. In some embodiments, a C-terminal and/or N-terminal NLS or NES is attached for optimal expression and nuclear targeting in eukaryotic cells, e.g., human cells.

In those embodiments where a tag is fused to a CRISPR effector, such tag may facilitate affinity-based or charge-based purification of the CRISPR effector, e.g., by liquid chromatography or bead separation utilizing an immobilized affinity or ion-exchange reagent. As a non-limiting example, a recombinant CRISPR effector of this disclosure comprises a polyhistidine (His) tag, and for purification is loaded onto a chromatography column comprising an immobilized metal ion (e.g. a Zn²⁺, Ni²⁺, Cu²⁺ ion chelated by a chelating ligand immobilized on the resin, which resin may be an individually prepared resin or a commercially available resin or ready to use column such as the HisTrap FF column commercialized by GE Healthcare Life Sciences, Marlborough, Massachusetts. Following the loading step, the column is optionally rinsed, e.g., using one or more suitable buffer solutions, and the His-tagged protein is then eluted using a suitable elution buffer. Alternatively, or additionally, if the recombinant CRISPR effector of this disclosure utilizes a FLAG-tag, such protein may be purified using immunoprecipitation methods known in the industry. Other suitable purification methods for tagged CRISPR effectors or accessory proteins of this disclosure will be evident to those of skill in the art.

The proteins described herein (e.g., CRISPR effectors or accessory proteins) can be delivered or used as either nucleic acid molecules or polypeptides. When nucleic acid molecules are used, the nucleic acid molecule encoding the CRISPR effector can be codon-optimized. The nucleic acid can be codon optimized for use in any organism of interest, in particular human cells or bacteria. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, PA).

In some instances, nucleic acids of this disclosure which encode CRISPR effectors for expression in eukaryotic (e.g., human, or other mammalian cells) cells include one or more introns, i.e., one or more non-coding sequences comprising, at a first end (e.g., a 5′ end), a splice-donor sequence and, at second end (e.g., the 3′ end) a splice acceptor sequence. Any suitable splice donor/splice acceptor can be used in the various embodiments of this disclosure, including without limitation simian virus 40 (SV40) intron, beta-globin intron, and synthetic introns. Alternatively, or additionally, nucleic acids of this disclosure encoding CRISPR effectors or accessory proteins may include, at a 3′ end of a DNA coding sequence, a transcription stop signal such as a polyadenylation (polyA) signal. In some instances, the polyA signal is located in close proximity to, or adjacent to, an intron such as the SV40 intron.

Deactivated/Inactivated CRISPR Effectors

The CRISPR effectors described herein can be modified to have diminished nuclease activity, e.g., nuclease inactivation of at least 50%, at least 60%, at least 70%, at least 80%, at least 90%, at least 95%, at least 97%, or 100% as compared with the wild type CRISPR effectors. The nuclease activity can be diminished by several methods known in the art, e.g., introducing mutations into the nuclease domains of the proteins. In some embodiments, catalytic residues for the nuclease activities are identified, and these amino acid residues can be substituted by different amino acid residues (e.g., glycine or alanine) to diminish the nuclease activity.

The inactivated CRISPR effectors can comprise or be associated with one or more functional domains (e.g., via fusion protein, linker peptides, “GS” linkers, etc.). These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, and switch activity (e.g., light inducible). In some embodiments, the functional domains are Krüppel associated box (KRAB), VP64, VP16, Fok1, P65, HSF1, MyoD1, and biotin-APEX.

The positioning of the one or more functional domains on the inactivated CRISPR effectors is one that allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., Fok1) is positioned to cleave or partially cleave the target. In some embodiments, the functional domain is positioned at the N-terminus of the CRISPR effector. In some embodiments, the functional domain is positioned at the C-terminus of the CRISPR effector. In some embodiments, the inactivated CRISPR effector is modified to comprise a first functional domain at the N-terminus and a second functional domain at the C-terminus.

Split Enzymes

The present disclosure also provides a split version of the CRISPR effectors described herein. The split version of the CRISPR effectors may be advantageous for delivery. In some embodiments, the CRISPR effectors are split to two parts of the enzymes, which together substantially comprises a functioning CRISPR effector.

The split can be done in a way that the catalytic domain(s) are unaffected. The CRISPR effectors may function as a nuclease or may be inactivated enzymes, which are essentially RNA-binding proteins with very little or no catalytic activity (e.g., due to mutation(s) in its catalytic domains).

In some embodiments, the nuclease lobe and a-helical lobe are expressed as separate polypeptides. Although the lobes do not interact on their own, the RNA guide recruits them into a ternary complex that recapitulates the activity of full-length CRISPR effectors and catalyzes site-specific DNA cleavage. The use of a modified RNA guide abrogates split-enzyme activity by preventing dimerization, allowing for the development of an inducible dimerization system. The split enzyme is described, e.g., in Wright et al. “Rational design of a split-Cas9 enzyme complex,” Proc. Natl. Acad. Sci., 112.10 (2015): 2984-2989, which is incorporated herein by reference in its entirety.

In some embodiments, the split enzyme can be fused to a dimerization partner, e.g., by employing rapamycin sensitive dimerization domains. This allows the generation of a chemically inducible CRISPR effector for temporal control of CRISPR effector activity. The CRISPR effector can thus be rendered chemically inducible by being split into two fragments, and rapamycin-sensitive dimerization domains can be used for controlled reassembly of the CRISPR effector.

The split point is typically designed in silico and cloned into the constructs. During this process, mutations can be introduced to the split enzyme and non-functional domains can be removed. In some embodiments, the two parts or fragments of the split CRISPR effector (i.e., the N-terminal and C-terminal fragments) can form a full CRISPR effector, comprising, e.g., at least 70%, at least 80%, at least 90%, at least 95%, or at least 99% of the sequence of the wild-type CRISPR effector.

Self-Activating or Inactivating Enzymes

The CRISPR effectors described herein can be designed to be self-activating or self-inactivating. In some embodiments, the CRISPR effectors are self-inactivating. For example, the target sequence can be introduced into the CRISPR effector coding constructs. Thus, the CRISPR effectors can cleave the target sequence, as well as the construct encoding the enzyme thereby self-inactivating their expression. Methods of constructing a self-inactivating CRISPR system is described, e.g., in Epstein et al., “Engineering a Self-Inactivating CRISPR System for AAV Vectors,” Mol. Ther., 24 (2016): S50, which is incorporated herein by reference in its entirety.

In some other embodiments, an additional RNA guide, expressed under the control of a weak promoter (e.g., 7SK promoter), can target the nucleic acid sequence encoding the CRISPR effector to prevent and/or block its expression (e.g., by preventing the transcription and/or translation of the nucleic acid). The transfection of cells with vectors expressing the CRISPR effector, RNA guides, and RNA guides that target the nucleic acid encoding the CRISPR effector can lead to efficient disruption of the nucleic acid encoding the CRISPR effector and decrease the levels of CRISPR effector, thereby limiting the genome editing activity.

In some embodiments, the genome editing activity of a CRISPR effector can be modulated through endogenous RNA signatures (e.g., miRNA) in mammalian cells. The CRISPR effector switch can be made by using a miRNA-complementary sequence in the 5′-UTR of mRNA encoding the CRISPR effector. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (Hirosawa et al. “Cell-type-specific genome editing with a microRNA-responsive CRISPR-Cas9 switch,” Nucl. Acids Res., 2017 Jul. 27; 45(13): e118).

Inducible CRISPR Effectors

The CRISPR effectors can be inducible, e.g., light inducible or chemically inducible. This mechanism allows for activation of the functional domain in a CRISPR effector. Light inducibility can be achieved by various methods known in the art, e.g., by designing a fusion complex wherein CRY2PHR/CIBN pairing is used in split CRISPR effectors (see, e.g., Konermann et al., “Optical control of mammalian endogenous transcription and epigenetic states,” Nature, 500.7463 (2013): 472). Chemical inducibility can be achieved, e.g., by designing a fusion complex wherein FKBP/FRB (FK506 binding protein/FKBP rapamycin binding domain) pairing is used in split CRISPR effectors. Rapamycin is required for forming the fusion complex, thereby activating the CRISPR effectors (see, e.g., Zetsche et al., “A split-Cas9 architecture for inducible genome editing and transcription modulation,” Nature Biotech., 33.2 (2015): 139-142).

Furthermore, expression of a CRISPR effector can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless et al., “Direct and specific chemical control of eukaryotic translation with a synthetic RNA—protein interaction,” Nucl. Acids Res., 40.9 (2012): e64-e64).

Various embodiments of inducible CRISPR effectors and inducible CRISPR systems are described, e.g., in U.S. Pat. No. 8,871,445, US 20160208243, and WO 2016205764, each of which is incorporated herein by reference in its entirety.

Functional Mutations

Various mutations or modifications can be introduced into a CRISPR effector as described herein to improve specificity and/or robustness. In some embodiments, the amino acid residues that recognize the Protospacer Adjacent Motif (PAM) are identified. The CRISPR effectors described herein can be modified further to recognize different PAMs, e.g., by substituting the amino acid residues that recognize PAM with other amino acid residues. In some embodiments, the CRISPR effectors can recognize, e.g., 5′-NTTN-3′, 5′-NTTR-3′, 5′-RTTR-3′, 5′-TNNT-3′, 5′-TNRT-3′, 5′-TSRT-3′, 5′-TGRT-3′, 5′-TNRY-3′, 5′-TTNR-3′, 5′-TTYR-3′, 5′-TTTR-3′, 5′-TTCV-3′, 5′-DTYR-3′, 5′-WTTR-3′, 5′-NNR-3′, 5′-NYR-3′, 5′-YYR-3′, 5′-TYR-3′, 5′-TTN-3′, 5′-TTR-3′, 5′-CNT-3′, 5′-NGG-3′, 5′-BGG-3′, or 5′-R-3′, wherein “N” is any nucleotide, “B” is C or G or T, “D” is A or G or T, “R” is A or G, “S” is G or C, “V” is A or C or G, “W” is A or T, and “Y” is C or T.

In some embodiments, the CRISPR effectors described herein can be mutated at one or more amino acid residue to modify one or more functional activities. For example, in some embodiments, the CRISPR effector is mutated at one or more amino acid residues to modify its helicase activity. In some embodiments, the CRISPR effector is mutated at one or more amino acid residues to modify its nuclease activity (e.g., endonuclease activity or exonuclease activity). In some embodiments, the CRISPR effector is mutated at one or more amino acid residues to modify its ability to functionally associate with an RNA guide. In some embodiments, the CRISPR effector is mutated at one or more amino acid residues to modify its ability to functionally associate with a target nucleic acid.

In some embodiments, the CRISPR effectors described herein are capable of cleaving a target nucleic acid molecule. In some embodiments, the CRISPR effector cleaves both strands of the target nucleic acid molecule. However, in some embodiments, the CRISPR effector is mutated at one or more amino acid residues to modify its cleaving activity. For example, in some embodiments, the CRISPR effector may comprise one or more mutations that increase the ability of the CRISPR effector to cleave a target nucleic acid. In another example, in some embodiments, the CRISPR effector may comprise one or more mutations that render the enzyme incapable of cleaving a target nucleic acid. In other embodiments, the CRISPR effector may comprise one or more mutations such that the enzyme is capable of cleaving a strand of the target nucleic acid (i.e., nickase activity). In some embodiments, the CRISPR effector is capable of cleaving the strand of the target nucleic acid that is complementary to the strand that the RNA guide hybridizes to. In some embodiments, the CRISPR effector is capable of cleaving the strand of the target nucleic acid that the RNA guide hybridizes to.

In some embodiments, one or more residues of a CRISPR effector disclosed herein are mutated to an arginine moiety. In some embodiments, one or more residues of a CRISPR effector disclosed herein are mutated to a glycine moiety. In some embodiments, one or more residues of a CRISPR effector disclosed herein are mutated based upon consensus residues of a phylogenetic alignment of CRISPR effectors disclosed herein.

In some embodiments, a CRISPR effector described herein may be engineered to comprise a deletion in one or more amino acid residues to reduce the size of the enzyme while retaining one or more desired functional activities (e.g., nuclease activity and the ability to interact functionally with an RNA guide). The truncated CRISPR effector may be used advantageously in combination with delivery systems having load limitations.

In one aspect, the present disclosure provides nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleic sequences described herein, while maintaining the domain architecture shown in FIG. 2. In another aspect, the present disclosure also provides amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequences described herein, while maintaining the domain architecture shown in FIG. 2.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

In some embodiments, a nuclease comprises a sequence set forth as PX₁X₂X₃X₄F (SEQ ID NO: 216), wherein X₁ is L or M or I or C or F, X₂ is Y or W or F, X₃ is K or T or C or R or W or Y or H or V, and X₄ is I or L or M. In some embodiments, the sequence set forth in SEQ ID NO: 216 is an N-terminal sequence. In some embodiments, a nuclease comprises a sequence set forth as RX₁X₂X₃L (SEQ ID NO: 217), wherein X₁ is I or L or M or Y or T or F, X₂ is R or Q or K or E or S or T, and X₃ is L or I or T or C or M or K. In some embodiments, a nuclease comprises a sequence set forth as NX₁YX₂(SEQ ID NO: 218), wherein X₁ is I or L or F and X₂ is K or R or V or E. In some embodiments, a nuclease comprises a sequence set forth as KX₁X₂X₃FAX₄X₅KD (SEQ ID NO: 219), wherein X₁ is T or I or N or A or S or F or V, X₂ is I or V or L or S, X₃ is H or S or G or R, X₄ is D or S or E, and X₅ is I or V or M or T or N. In some embodiments of any of the systems described herein, the sequence of SEQ ID NO: 219 is a C-terminal sequence. In some embodiments, a nuclease comprises a sequence set forth as LX₁NX₂(SEQ ID NO: 220), wherein X₁ is G or S or C or T and X₂ is N or Y or K or S. In some embodiments of any of the systems described herein, the sequence of SEQ ID NO: 220 is a C-terminal sequence. In some embodiments, a nuclease comprises a sequence set forth as PX₁X₂X₃X₄SQX₅DS (SEQ ID NO: 221), wherein X₁ is S or P or A, X₂ is Y or S or A or P or E or Y or Q or N, X₃ is F or Y or H, X₄ is T or S, and X₅ is M or T or I. In some embodiments of any of the systems described herein, the sequence of SEQ ID NO: 221 is a C-terminal sequence. In some embodiments, a nuclease comprises a sequence set forth as KX₁X₂VRX₃X₄QEX₅H (SEQ ID NO: 222), wherein X₁ is N or K or W or R or E or T or Y, X₂ is M or R or L or S or K or V or E or T or I or D, X₃ is L or R or H or P or T or K or Q of P or S or A, X₄ is G or Q or N or R or K or E or I or T or S or C, and X₅ is R or W or Y or K or T or F or S or Q. In some embodiments of any of the systems described herein, the sequence of SEQ ID NO: 222 is a C-terminal sequence. In some embodiments, a nuclease comprises a sequence set forth as X₁NGX₂X₃X₄DX₅NX₆X₇X₈N (SEQ ID NO: 223), wherein X₁ is I or K or V or L, X₂ is L or M, X₃ is N or H or P, X₄ is A or S or C, X₅ is V or Y or I or F or T or N, X₆ is A or S, X₇ is S or A or P, and X₈ is M or C or L or R or N or S or K or L. In some embodiments of any of the systems described herein, the sequence of SEQ ID NO: 223 is a C-terminal sequence.

### RNA and RNA Guide Modifications

In some embodiments, an RNA guide described herein comprises a uracil (U). In some embodiments, an RNA guide described herein comprises a thymine (T). In some embodiments, a direct repeat sequence of an RNA guide described herein comprises a uracil (U). In some embodiments, a direct repeat sequence of an RNA guide described herein comprises a thymine (T). In some embodiments, a direct repeat sequence according to TABLE 2 or TABLE 8 comprises a sequence comprising a uracil, in one or more places indicated as thymine in the corresponding sequences in TABLE 2 or TABLE 8.

In some embodiments, the direct repeat comprises only one copy of a sequence that is repeated in an endogenous CRISPR array. In some embodiments, the direct repeat is a full-length sequence adjacent to (e.g., flanking) one or more spacer sequences found in an endogenous CRISPR array. In some embodiments, the direct repeat is a portion (e.g., processed portion) of a full-length sequence adjacent to (e.g., flanking) one or more spacer sequences found in an endogenous CRISPR array.

Spacer and Direct Repeat

The spacer length of RNA guides can range from about 15 to 55 nucleotides. The spacer length of RNA guides can range from about 20 to 45 nucleotides. In some embodiments, the spacer length of an RNA guide is at least 15 nucleotides, at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, or longer.

In some embodiments, the direct repeat length of the RNA guide is at least 16 nucleotides, or is from 16 to 20 nucleotides (e.g., 16, 17, 18, 19, or 20 nucleotides). In some embodiments, the direct repeat length of the RNA guide is about 19 to about 40 nucleotides.

Exemplary direct repeat sequences (e.g., direct repeat sequences of pre-crRNAs (e.g., unprocessed crRNAs) or mature crRNAs (e.g., direct repeat sequences of processed crRNAs)) are shown in TABLE 2. See also TABLE 8.

TABLE 2
Exemplary direct repeat sequences of crRNA sequences.

| Effector | Direct Repeat Sequence |
|---|---|
| SEQ ID NO: 1 | ACTATGTTGGAATACATTTTTATAGGTATTTACAACT (SEQ ID NO: 57) |
| SEQ ID NO: 2 | ATTGTTGGAATATCACTTTTGTAGGGTATTCACAAC (SEQ ID NO: 58) |
| SEQ ID NO: 3 | AATGTTGTTCACCCTTTTT (SEQ ID NO: 59) |
| SEQ ID NO: 4 | CCTGTTGTGAATACTCTTTTATAGGTATCAAACAAC (SEQ ID NO: 60) |
| SEQ ID NO: 10 | ATTGTTGTAGACACCTTTTTATAAGGATTGAACAAC (SEQ ID NO: 62) |
| | CTTGTTGTATATGTCCTTTTATAGGTATTAAACAAC (SEQ ID NO: 213) |
| SEQ ID NO: 14 | GTTGTTTAATACCTATAAAAGAATATATACAACAAG (SEQ ID NO: |
| | 128) |
| SEQ ID NO: 15 | CTTGTTGTATATACTCTTTTATAGGTATTAAACAAC (SEQ ID NO: 63) |
| SEQ ID NO: 17 | GTTGTATCCACCGTATAAAACATAGTGTCCAACATC (SEQ ID NO: |
| | 130) |
| SEQ ID NO: 18 | GATGTTGTTATGCTGTTTTTGTAAGTAATAAACAAC (SEQ ID NO: 70) |
| SEQ ID NO: 21 | ATTGTTGTACGAACCATTTTATATGGTAATAACAAC (SEQ ID NO: 72) |
| SEQ ID NO: 22 | ACTGTAAAACCCCTGCAGATGAAAGGAAAGTACAACAGT (SEQ ID |
| | NO: 73) |
| SEQ ID NO: 23 | ATCATGTTGTACATACTATTTTTTAAGTATTAAACAACTA (SEQ ID |
| | NO: 74) |
| SEQ ID NO: 24 | CTTGTTGTATATACTCTTTTATAGGTATTAAACAAC (SEQ ID NO: 63) |
| SEQ ID NO: 27 | ATTGTTGGGGTACTTCTTTTATAGGGTACTCACAAC (SEQ ID NO: 76) |
| SEQ ID NO: 28 | ATTGTTGTAGACCTTGTGTTTTAGGGGTCTAACAACG (SEQ ID NO: |
| | 77) |
| SEQ ID NO: 29 | GTTGTAAATACATCTCATATTGTATTCCAACACAGT (SEQ ID NO: 139) |
| SEQ ID NO: 31 | ATTGTTGGAATATCACTTTTGTAGGGTATTCACAAC (SEQ ID NO: 58) |
| SEQ ID NO: 32 | AATTGTTGAGATACCGTTTTTTATGGTATTGGCAAC (SEQ ID NO: 80) |
| SEQ ID NO: 35 | ATTGTTGTAGACCTTGTGTTTTAGGGGTCTAACAACG (SEQ ID NO: |
| | 77) |
| SEQ ID NO: 36 | GTTGTAAATACATCTCATATTGTATTCCAACACAGT (SEQ ID NO: 139) |
| SEQ ID NO: 38 | AATTGTTGAGATACCGTTTTTTATGGTATTGGCAAC (SEQ ID NO: 80) |
| SEQ ID NO: 39 | ATTGTTGGAATATCACTTTTGTAGGGTATTCACAAC (SEQ ID NO: 58) |
| SEQ ID NO: 41 | ATTGTGTTGGGATACACTTTTATAGGTATTTACAAC (SEQ ID NO: 83) |
| SEQ ID NO: 42 | TATTGTTGAATACCTTTCTTATAAAGGTAATTACAAC (SEQ ID NO: 84) |
| SEQ ID NO: 44 | ATTGTTGAATGTATTCTTTTTTAGGACAGATACAAC (SEQ ID NO: 86) |
| SEQ ID NO: 45 | GTTGTATCCACCGTATAAAACATAGTGTCCAACATC (SEQ ID NO: |
| | 130) |
| SEQ ID NO: 46 | TATTGTTGAATACCTTTCTTATAAAGGTAATTACAAC (SEQ ID NO: 84) |
| SEQ ID NO: 47 | ATTGTTGAATGGTATCTTTTATAGACTGATTACAACT (SEQ ID NO: 87) |
| SEQ ID NO: 48 | ATTGTTGGATAATAGGTTTTTTATCTTAATTACAAC (SEQ ID NO: 88) |
| SEQ ID NO: 51 | TATTGTTGAATACCTTTCTTATAAAGGTAATTACAAC (SEQ ID NO: 84) |
| SEQ ID NO: 53 | TATTGTTGAATACCTTTCTTATAAAGGTAATTACAAC (SEQ ID NO: 84) |
| SEQ ID NO: 55 | ATTGTTGGATAATAGGTTTTTTATCTTAATTACAAC (SEQ ID NO: 88) |
| SEQ ID NO: 56 | ATTGTTGTAGATACCTTTTTGTAAGGATTGAACAAC (SEQ ID NO: 90) |

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 57. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 58. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 3, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 59. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 4, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 60. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 10, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 62 or SEQ ID NO: 213. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 14, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 128. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 15, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 63. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 17, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 130. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 18, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 70. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 21, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 72. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 22, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 73. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 23, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 74. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 24, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 63. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 27, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 76. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 28, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 77. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 29, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 139. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 31, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 58. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 32, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 80. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 35, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 77. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 36, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 139. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 38, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 80. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 39, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 58. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 41, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 83. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 42, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 84. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 44, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 86. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 45, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 130. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 46, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 84. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 47, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 87. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 48, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 88. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 51, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 84. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 53, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 84. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 55, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 88. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 56, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 90.

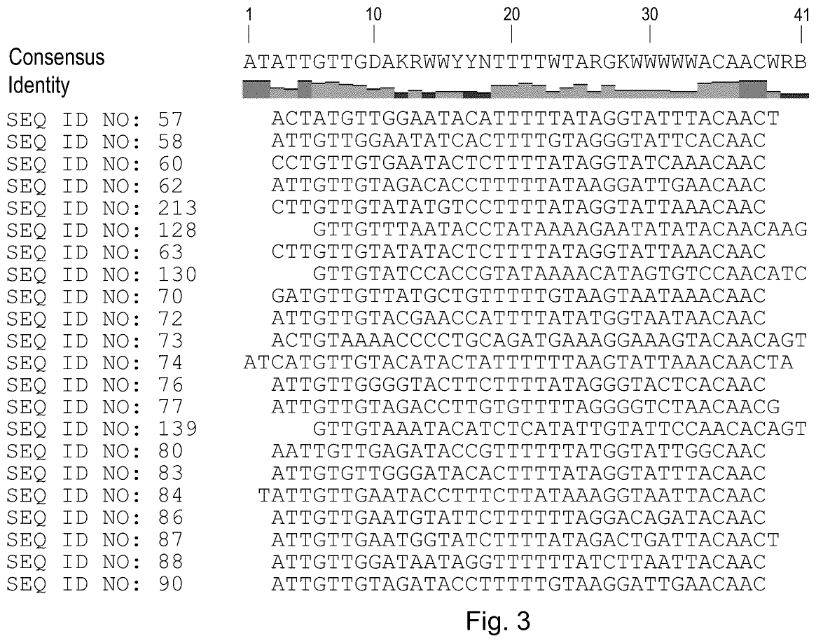

In some embodiments, an RNA guide comprises a direct repeat sequence set forth in FIG. 3. For example, in some embodiments, the RNA guide comprises a direct repeat of the consensus sequence shown in FIG. 3 or a portion of the consensus sequence shown in FIG. 3. In some embodiments, an RNA guide comprises a direct repeat having a sequence set forth as X₁X₂TX₃X₄X₅X₆X₇X₈ (SEQ ID NO: 224), wherein X₁ is A or C or G, X₂ is T or C or A X₃ is T or G or A X₄ is T or G, X₅ is T or G or A, X₆ is G or T or A, X₇ is T or G or A, and X₈ is A or G or T. For example, in some embodiments, an RNA guide comprises a direct repeat having a sequence set forth as ATTGTTGDA (SEQ ID NO: 225). In some embodiments, SEQ ID NO: 224 is proximal to the 5′ end of the direct repeat. In some embodiments, SEQ ID NO: 225 is proximal to the 5′ end of the direct repeat. In some embodiments, an RNA guide comprises a direct repeat having a sequence set forth as X₁X₂X₃X₄X₅X₆X₇X₈X₉ (SEQ ID NO: 226), wherein X₁ is T or C or A, X₂ is T or A or G, X₃ is T or C or A, X₄ is T or A, X₅ is T or A or G, X₆ is T or A, X₇ is A or T, X₈ is A or G or C or T, and X₉ is G or A or C. For example, in some embodiments, an RNA guide comprises a direct repeat having a sequence set forth as TTTTWTARG (SEQ ID NO: 227). In some embodiments, an RNA guide comprises a direct repeat having a sequence set forth as X₁X₂X₃AC (SEQ ID NO: 228), wherein X₁ is A or C or G, X₂ is C or A, and X₃ is A or C. For example, in some embodiments, an RNA guide comprises a direct repeat having a sequence set forth as ACAAC (SEQ ID NO: 229). In some embodiments, SEQ ID NO: 228 is proximal to the 3′ end of the direct repeat. In some embodiments, SEQ ID NO: 229 is proximal to the 3′ end of the direct repeat.

In some embodiments, the spacer of an RNA guide binds to a target nucleic acid adjacent to a PAM sequence of TABLE 3. For example, in some embodiments, a complex of an effector and an RNA guide disclosed herein binds to a target nucleic acid adjacent to a PAM sequence as indicated in TABLE 3.

TABLE 3
PAM sequences corresponding to CLUST.091979
effectors.

| Effector | PAM Sequence |
|---|---|
| SEQ ID NO: 1 | 5′-TTNT-3′ |
| | 5′-TNRT-3′ |
| SEQ ID NO: 2 | 5′-TTR-3′ |
| | 5′-WTTR-3′ |
| SEQ ID NO: 4 | 5′-NNR-3′ |
| | 5′-NTTN-3′ |
| | 5′-NTTR-3′ |
| | 5′-TTTN-3′ |
| | 5′-TTTG-3′ |
| SEQ ID NO: 10 | 5′-NTTN-3′ |
| | 5′-RTTR-3′ |
| | 5′-ATTR-3′ |
| | 5′-RTTG-3′ |
| | 5′-ATTG-3′ |
| | 5′-GTTA-3′ |
| SEQ ID NO: 14 | 5′-TIN-3′ |
| | 5′-TTY-3′ |
| | 5′-YYR-3′ |
| SEQ ID NO: 15 | 5′-CNT-3′ |
| SEQ ID NO: 21 | 5′-TTCV-3′ |
| | 5′-TTYR-3′ |
| SEQ ID NO 23 | 5′-GTA-3′ |
| SEQ ID NO 24 | 5′-CNT-3′ |
| SEQ ID NO 27 | 5′-TTR-3′ |
| | 5′-YYR-3′ |
| | 5′-TYR-3′ |
| SEQ ID NO: 28 | 5′-NGG-3′ |
| | 5′-BGG-3′ |
| | 5′-CGG-3′ |
| | 5′-GG-3′ |
| SEQ ID NO 31 | 5′-TTR-3′ |
| SEQ ID NO 32 | 5′-TYR-3′ |
| SEQ ID NO 35 | 5′-NGG-3′ |
| | 5′-BGG-3′ |
| | 5′-CGG-3′ |
| | 5′-GG-3′ |
| SEQ ID NO 38 | 5′-TYR-3′ |
| SEQ ID NO 39 | 5′-TTR-3′ |
| SEQ ID NO 41 | 5′-TYR-3′ |
| SEQ ID NO 42 | 5′-TYR-3′ |
| | 5′-TTYR-3′ |
| | 5′-DTYR-3′ |
| SEQ ID NO: 44 | 5′-TTNR-3′ |
| | 5′-TTTR-3′ |
| SEQ ID NO: 46 | 5′-TYR-3′ |
| | 5′-TTYR-3′ |
| | 5′-DTYR-3′ |
| SEQ ID NO: 48 | 5′-YYR-3′ |
| | 5′-TTR-3′ |
| | 5′-TTG-3′ |
| SEQ ID NO: 51 | 5′-TYR-3′ |
| | 5′-TTYR-3′ |
| | 5′-DTYR-3′ |
| SEQ ID NO: 53 | 5′-TYR-3′ |
| | 5′-TTYR-3′ |
| | 5′-DTYR-3′ |
| SEQ ID NO: 55 | 5′-YYR-3′ |
| | 5′-TTR-3′ |
| | 5′-TTG-3′ |
| SEQ ID NO: 56 | 5′-TTG-3′ |
| | 5′-NYR-3′ |
| | 5′-TYR-3′ |

In some embodiments, an RNA guide further comprises a tracrRNA. In some embodiments, the tracrRNA is not required (e.g., the tracrRNA is optional). In some embodiments, the tracrRNA is a portion of the non-coding sequences shown in TABLE 9. For example, in some embodiments, the tracrRNA is a sequence of TABLE 4.

TABLE 4
Exemplary tracrRNA sequences.

| Effector | tracrRNA Sequence |
|---|---|
| SEQ ID NO: 1 | ATTGGGACTTCCGGAAGTAAAATATCCACCTGAGGATTTTAGGACAT |
| | ATAATTTCTAATAAAAATGAACGGAAAAATTTCCGTTCATTTTTTTTT |
| | TGTTTATT (SEQ ID NO: 152) |
| | TATTGGGACTTCCGGAAGTAAAATATCCACCTGAGGATTTTAGGACA |
| | TATAATTTCTAATAAAAATGAACGGAAAAATTTCCGTTCATTTTTTTT |
| | TTGTTTATTG (SEQ ID NO: 153) |
| | GACGAGAACGGAGTGTGGCTCCTGAGGAAAAACGACAAACATCCAA |
| | CATATTTTATCTACCAGAACGGAACACTCTATCAATATGAGGAAGAT |
| | TGATTAGTTGATGTTTTCATAATAATTTTATCTGGAATTTGAAAAGAT |
| | TCCAGATTTTTTTTTTATTTCG (SEQ ID NO: 154) |
| SEQ ID NO: 2 | GCAATCAACAAGACTTTCATTTTCAAGGCAAAATGCGATAAGAACG |
| | ATGTCATATCGTTATGGGAA (SEQ ID NO: 155) |
| | GATGCTCCGAAAACGTGGTTGTTCGGACAACAAAAAAATGAATGTT |
| | TCTAATGTATTAA (SEQ ID NO: 156) |
| | GACGGAAAAATAAATGAGGATGGTATGTTTGTTGAAAACTTGGAAT |
| | AATTCTGTATATACCAATTAGAAT (SEQ ID NO: 157) |
| | TGTTGATTGCTGATTCTTCGTTGTTTGATTTGTGTTGTGCCATAATCT |
| | TAAAATT (SEQ ID NO: 158) |
| SEQ ID NO: 3 | CGCAAGATATAAGGCAATCGGAAACGGATGGACAGTTGATGTAATT |
| | TCACATATTTTTAAGAATTTGAAAAATTAATTTGGTA (SEQ ID NO: |
| | 159) |
| | GGACATTTCGTAAATCATATGGAGATACGGAGTTCAAGTCAATTGA |
| | AGAGCTTCCTGAATTTAGAGATAACATACTTATACAACTAGATTGAT |
| | TG (SEQ ID NO: 160) |
| | ATCAATACATAGATGATGAGAAATGGAGAAAAAAATTTGTTCGCCC |
| | AACAAACACTAAT (SEQ ID NO: 161) |
| SEQ ID NO: 14 | CTGGTAATACTGTAAAATCTCCGTGTATAGGGCAAGTAATTGTAACT |
| | GGGGTAATTCTATCTACTATTATAGTTTTAGAA (SEQ ID NO: 162) |
| SEQ ID NO: 17 | CAGAAGTCGTTCAAGTTCAAGGTCAAAACGGACAAGGAGACGGTCG |
| | AATTATTCAG (SEQ ID NO: 163) |
| | GGGAGGGTGACATTCAGAAGTCGTTCAAGTTCAAGGTCAAAACGGA |
| | CAAGGAGACGGTCGAATTAT (SEQ ID NO: 164) |
| | AAGTGTCTTCAACACATTGAAGAAAACTCTCGGTGCAATATATGGA |
| | AAGCTCGATGAAAACGGAAATTTTATTGAGAATGAATGTAATAAGT |
| | AACTGGAATA (SEQ ID NO: 165) |
| | CCGTGGGAGGATTTGGATTTGGTTGAAGACATCAGAAAAATTTTCGA |
| | AATGGAATAGAGGGAACCGGAATTTTTTCCGGTTTTTCTTTGTCCTTT |
| | CGA (SEQ ID NO: 166) |
| SEQ ID NO: 18 | CAGAGTAACCTTTCCTGATATGTTGTTACACATTTTTGTAAGTGTTAA |
| | ACAACTGACGCATTGATATTGCCTTGTCTATTAA (SEQ ID NO: 167) |
| | CAATCGCGAGTTTATACTGAAATGTTGTTACACTGTTTTTGTAAGTGT |
| | TAAACAACCTTGCACAAATGTCATCTACCAGTAC (SEQ ID NO: 168) |
| SEQ ID NO: 21 | CCGAGCGACCCACAAACCTATTGTCGTACGCATCATTTCACATGATA |
| | ATAACAACGAATATTCCTGCAAGCATGATTT (SEQ ID NO: 169) |
| | TATGACATTATGATATTGTTGTATGCATCATTTCACATGGTAATAAC |
| | AACGAAGAGAAACACCGAGCGACCCACAAA (SEQ ID NO: 170) |
| | ACATCTTTTATGACATTATGATATTGTTGTATGCATCATTTCACATGG |
| | TAATAACAACGAAGAGAAACACCGAGCGACCCACAAA (SEQ ID NO: |
| | 171) |
| SEQ ID NO: 22 | GCTAAAATATAGTCCTGTGGATGTTGAATACATTTCTTTTAAGTGTA |
| | CTTACAACCAACGCTGTACACATTGCTAATGGATG (SEQ ID NO: 172) |
| | TGCTAAAATATAGTCCTGTGGATGTTGAATACATTTCTTTTAAGTGT |
| | ACTTACAACCAACGCTGTACACATTGCTAATGGATG (SEQ ID NO: |
| | 173) |
| | CAACACCAAGGCTGAGGCAAAGAAGAGGGCTGATGATATGAACAA |
| | ACAGAATAGGGTCATACACCAGCTGTCTGTTTATTTGTGTCC (SEQ ID |
| | NO: 174) |
| | AATTAGACTGATAAACAAAGAATAATGAGAACTATAATAGGGAGGT |
| | GTACCCCCGAATTTAAGCCAGTGGAGAACCATACAAACCTATCATAT |
| | AG (SEQ ID NO: 175) |
| SEQ ID NO: 23 | TGGGTATGCGTTGTTTAATACTTAAAAAAATGTATGTACAACATGTC |
| | TGTGGAAAGTCTTTCTATTGTATAT (SEQ ID NO: 176) |
| | CGITGTTTAATACTTAAAAAAATGTATGTACAACATGTCTGTGGAAA |
| | GTCTTTCTATTGTATATAGGA (SEQ ID NO: 177) |
| | TGGGTATGCGTTGTTTAATACTTAAAAAAATGTATGTACAACATGTC |
| | TGTGGAAAGTCTTTCTATTGTATATAGGAATTTTATATAATTATTTAA |
| | TTATCAATGAATTATATTAGTAT (SEQ ID NO: 178) |
| | GGTGGGTATGCGTTGTTTAATACTTAAAAAAATGTATGTACAACATG |
| | TCTGTGGAAAG (SEQ ID NO: 179) |
| SEQ ID NO: 27 | AATGAACGAGATTGTTGGGATATACCTTTTATAGGATTTTCACAACA |
| | TCTGAGTTGTTTGATGTTAAAAACTT (SEQ ID NO: 180) |
| | GATAAAAATGAACGAGATTGTTGGGATATACCTTTTATAGGATTTTC |
| | ACAACATCTGAGTTGTTTGATGTTAAAAACTTT (SEQ ID NO: 181) |
| SEQ ID NO: 29 | GCTAATATAAAGATTGTACTGTGTTGAGATACACTTTTAGAGGTATT |
| | TACAACAAAATGCGTGATATGGAAATGA (SEQ ID NO: 182) |
| | ATACCAACATAAATACAGGTCTTGCTGTTTCTGGTCGGTCGTAAACA |
| | CCTCTAAAAGGATTGTTTCGACATAGGTTACTGACGCTTCAAG (SEQ |
| | ID NO: 183) |
| | AATGAAGAAATAACTGTGTTGAGATACACTTTTAGAGGTATTTACAA |
| | CACCATATAAACCTGACCATCTCCT (SEQ ID NO: 184) |
| SEQ ID NO: 31 | AGGAAGATGTCAGACGTTTTTATTGTTGGAATACTCGTTTTTTACGG |
| | TATTTACAACTGCCCCGTAGCGGAATCAAAATACCAC (SEQ ID NO: |
| | 185) |
| | ATGTCAGACGTTTTTATTGTTGGAATACTCGTTTTTTACGGTATTTAC |
| | AACTGCCCCGTAGCGGAATCAAAATACC (SEQ ID NO: 186) |
| | AAATAACAAAAATTCTGGACGGGAAAGGAAGATGTCAGACGTTTTT |
| | ATTGTTGGAATACTCGTTTTTTACGGTATTTACAACTGCCCCGTAGCG |
| | GAATC (SEQ ID NO: 187) |
| | ATAACAAAAATTCTGGACGGGAAAGGAAGATGTCAGACGTTTTTAT |
| | TGTTGGAATACTCGTTTTTTACGGTATTTACAACTGCCCCGTAGCGG |
| | AAT (SEQ ID NO: 188) |
| SEQ ID NO: 32 | TATTGCAACTATTACAACAAACTTAGCGAATGGATTGGCAAAGATAT |
| | GTATAACACGCCG (SEQ ID NO: 189) |
| | ATTGCAACTATTACAACAAACTTAGCGAATGGATTGGCAAAGATAT |
| | GTATAACACGCCG (SEQ ID NO: 190) |
| SEQ ID NO: 36 | GCTAATATAAAGATTGTACTGTGTTGAGATACACTTTTAGAGGTATT |
| | TACAACAAAATGCGTGATATGGAAATGA (SEQ ID NO: 182) |
| | ATACCAACATAAATACAGGTCTTGCTGTTTCTGGTCGGTCGTAAACA |
| | CCTCTAAAAGGATTGTTTCGACATAGGTTACTGACGCTTCAAG (SEQ |
| | ID NO: 183) |
| | AATGAAGAAATAACTGTGTTGAGATACACTTTTAGAGGTATTTACAA |
| | CACCATATAAACCTGACCATCTCCT (SEQ ID NO: 184) |
| SEQ ID NO: 38 | TATTGCAACTATTACAACAAACTTAGCGAATGGATTGGCAAAGATAT |
| | GTATAACACGCCG (SEQ ID NO: 189) |
| | ATTGCAACTATTACAACAAACTTAGCGAATGGATTGGCAAAGATAT |
| | GTATAACACGCCG (SEQ ID NO: 190) |
| SEQ ID NO: 39 | AGGAAGATGTCAGACGTTTTTATTGTTGGAATACTCGTTTTTTACGG |
| | TATTTACAACTGCCCCGTAGCGGAATCAAAATACCAC (SEQ ID NO: |
| | 185) |
| | ATGTCAGACGTTTTTATTGTTGGAATACTCGTTTTTTACGGTATTTAC |
| | AACTGCCCCGTAGCGGAATCAAAATACC (SEQ ID NO: 186) |
| | AAATAACAAAAATTCTGGACGGGAAAGGAAGATGTCAGACGTTTTT |
| | ATTGTTGGAATACTCGTTTTTTACGGTATTTACAACTGCCCCGTAGCG |
| | GAATC (SEQ ID NO: 187) |
| | ATAACAAAAATTCTGGACGGGAAAGGAAGATGTCAGACGTTTTTAT |
| | TGTTGGAATACTCGTTTTTTACGGTATTTACAACTGCCCCGTAGCGG |
| | AAT (SEQ ID NO: 188) |
| SEQ ID NO: 41 | GTATGATGACAGAAGAAACACGGAAGACAATAGAGAGCGTCATAGT |
| | GGTTCTCGGCATAGCAATCATGCTG (SEQ ID NO: 191) |
| | ATGATGACAGAAGAAACACGGAAGACAATAGAGAGCGTCATAGTG |
| | GTTCTCGGCATAGCAATCATGCTGGCAGCCGCCGTCCGAATAATGAC |
| | GCAGAACAAAGCAATTGTGAAATATG (SEQ ID NO: 192) |
| | AGAAGGTACTGCCGCCTTATGACCGACGAGAACGGAGTGTGGCTCC |
| | TGAGGAAAAAC (SEQ ID NO: 193) |
| | GACGAGAACGGAGTGTGGCTCCTGAGGAAAAACGACAAACATCCAA |
| | CATATTTTATCTACCAGAACGGAACACTCTATCAATATGAGGAAGAT |
| | TGATTAGTTGATGTTTTCATAATAATTTTATCTGGAATTTGAAAAGAT |
| | TCCAGATTTTTTTTTTATTTCG (SEQ ID NO: 194) |
| SEQ ID NO: 43 | TCGTTGAATACGATATCGCCGAAACAATTGATTGGAGAAGTACGCTT |
| | TGTTTCAAGACATGGAATACGTATGGTTCTCCTCAATGGGACTCGAA |
| | GATCAAGAA (SEQ ID NO: 197) |
| | ATCGTTGAATACGATATCGCCGAAACAATTGATTGGAGAAGTACGC |
| | TTTGTTTCAAGACATGGAATACGTATGGTTCTCCTCAATGGGACTCG |
| | AAGATCAAGAACCAG (SEQ ID NO: 198) |
| | GAGCTTTTCTGGCAATGTAGACATTAAAGCTGGTATCGTTGAATACG |
| | ATATCGCCGAAACAATTGATTGGAGA (SEQ ID NO: 199) |
| SEQ ID NO: 44 | TTTTTGTTATATATTTGTCCTGTTAGGTTAAATCACCGCGCCTGATGA |
| | CGAAGTCGGTGGTAGAATTAGACTAATATTAAATATGTCTCATG |
| | (SEQ ID NO: 195) |
| | CCTATTAGATATTCCGTATTTCTTTAAGACTGTTATAATACAAATATA |
| | CTACAAATCATGCAATTTTTGATTTTTAACAAAA (SEQ ID NO: 196) |
| SEQ ID NO: 45 | CAGAAGTCGTTCAAGTTCAAGGTCAAAACGGACAAGGAGACGGTCG |
| | AATTATTCAG (SEQ ID NO: 163) |
| | GGGAGGGTGACATTCAGAAGTCGTTCAAGTTCAAGGTCAAAACGGA |
| | CAAGGAGACGGTCGAATTAT (SEQ ID NO: 164) |
| | AAGTGTCTTCAACACATTGAAGAAAACTCTCGGTGCAATATATGGA |
| | AAGCTCGATGAAAACGGAAATTTTATTGAGAATGAATGTAATAAGT |
| | AACTGGAATA (SEQ ID NO: 165) |
| | CCGTGGGAGGATTTGGATTTGGTTGAAGACATCAGAAAAATTTTCGA |
| | AATGGAATAGAGGGAACCGGAATTTTTTCCGGTTTTTCTTTGTCCTTT |
| | CGA (SEQ ID NO: 166) |
| SEQ ID NO: 48 | TTTTTCATTGTTCTCAAATTGTTGGATAATGTTTTGTGTGTTTCATTTT |
| | TGTCATTGTGTCACCTTAACTGACAAGGTGGCACATTTTTTATGTCA |
| | AT (SEQ ID NO: 200) |
| | TTTTCATTGTTCTCAAATTGTTGGATAATGTTTTGTGTGTTTCATTTTT |
| | GTCATTGTGTCACCTTAACTGACAAGGTGGCACATTTTTTATGTCAA |
| | TA (SEQ ID NO: 201) |
| | AATATATCTGCTAAGGTCATATTTTTCATTGTTCTCAAATTGTTGGAT |
| | AATGTTTTGTGTGTTTCATTTTTGTCATTGTGTCACCTTAACTGACAA |
| | GGTGGCACATTTTTTATGTCAATATG (SEQ ID NO: 202) |
| SEQ ID NO: 52 | TCGTTGAATACGATATCGCCGAAACAATTGATTGGAGAAGTACGCTT |
| | TGTTTCAAGACATGGAATACGTATGGTTCTCCTCAATGGGACTCGAA |
| | GATCAAGAA (SEQ ID NO: 197) |
| | ATCGTTGAATACGATATCGCCGAAACAATTGATTGGAGAAGTACGC |
| | TTTGTTTCAAGACATGGAATACGTATGGTTCTCCTCAATGGGACTCG |
| | AAGATCAAGAACCAG (SEQ ID NO: 198) |
| | GAGCTTTTCTGGCAATGTAGACATTAAAGCTGGTATCGTTGAATACG |
| | ATATCGCCGAAACAATTGATTGGAGA (SEQ ID NO: 199) |
| SEQ ID NO: 55 | TTTTTCATTGTTCTCAAATTGTTGGATAATGTTTTGTGTGTTTCATTTT |
| | TGTCATTGTGTCACCTTAACTGACAAGGTGGCACATTTTTTATGTCA |
| | AT (SEQ ID NO: 200) |
| | TTTTCATTGTTCTCAAATTGTTGGATAATGTTTTGTGTGTTTCATTTTT |
| | GTCATTGTGTCACCTTAACTGACAAGGTGGCACATTTTTTATGTCAA |
| | TA (SEQ ID NO: 201) |
| | AATATATCTGCTAAGGTCATATTTTTCATTGTTCTCAAATTGTTGGAT |
| | AATGTTTTGTGTGTTTCATTTTTGTCATTGTGTCACCTTAACTGACAA |
| | GGTGGCACATTTTTTATGTCAATATG (SEQ ID NO: 202) |
| SEQ ID NO: 56 | ACAAATTTTTGATTATGGCACACAAAAAGAACATAGGAGCAGAGAT |
| | AGTAAAAACTTACTCTTTTAAGGTGAAGA (SEQ ID NO: 203) |
| | TTATTTTATAGGATAATAGAGCTAACAAGCATTAACAATTATTAAAA |
| | CGATTTATATTGAAAATAAATTTTGTGGGAATATTTATTTTTACTACC |
| | TTTGCATCGTAATACAATTAAACAAATTTTTGATTATGGCA (SEQ ID |
| | NO: 204) |

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 152, SEQ ID NO: 153, or SEQ ID NO: 154. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 155, SEQ ID NO: 156, SEQ ID NO: 157, or SEQ ID NO: 158. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 3, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO:159 SEQ ID NO: 160, or SEQ ID NO: 161. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 14, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 162. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 17, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 163, SEQ ID NO: 164, SEQ ID NO: 165, or SEQ ID NO: 166. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 18, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 167 or SEQ ID NO: 168. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 21, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO:169, SEQ ID NO: 170, or SEQ ID NO: 171. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 22, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 172, SEQ ID NO: 173, SEQ ID NO: 174, or SEQ ID NO: 175. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 23, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 176, SEQ ID NO: 177, SEQ ID NO: 178, or SEQ ID NO: 179. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 27, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 180 or SEQ ID NO: 181. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 29, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 182, SEQ ID NO: 183, or SEQ ID NO: 184. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 31, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 185, SEQ ID NO: 186, SEQ ID NO: 187, or SEQ ID NO: 188. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 32, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 189 or SEQ ID NO: 190. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 36, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 182, SEQ ID NO: 183, or SEQ ID NO: 184. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 38, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 189 or SEQ ID NO: 190. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 39, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 185, SEQ ID NO: 186, SEQ ID NO:187, or SEQ ID NO: 188. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 41, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 191, SEQ ID NO: 192, SEQ ID NO: 193, or SEQ ID NO: 194. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 43, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 197, SEQ ID NO: 198, or SEQ ID NO: 199. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 44, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 195 or SEQ ID NO: 196. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 45, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 163, SEQ ID NO: 164, SEQ ID NO: 165, or SEQ ID NO: 166. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 48, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 200, SEQ ID NO: 201, or SEQ ID NO: 202. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 52, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 197, SEQ ID NO: 198, or SEQ ID NO: 199. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 55, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 200, SEQ ID NO: 201, or SEQ ID NO: 202. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 56, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 203 or SEQ ID NO: 204.

The RNA guide sequences can be modified in a manner that allows for formation of the CRISPR complex and successful binding to the target, while at the same time not allowing for successful nuclease activity (i.e., without nuclease activity/without causing indels). These modified guide sequences are referred to as “dead guides” or “dead guide sequences.” These dead guides or dead guide sequences may be catalytically inactive or conformationally inactive with regard to nuclease activity. Dead guide sequences are typically shorter than respective guide sequences that result in active RNA cleavage. In some embodiments, dead guides are 5%, 10%, 20%, 30%, 40%, or 50% shorter than respective RNA guides that have nuclease activity. Dead guide sequences of RNA guides can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR systems including a functional CLUST.091979 CRISPR effector as described herein, and an RNA guide wherein the RNA guide comprises a dead guide sequence, whereby the RNA guide is capable of hybridizing to a target sequence such that the CRISPR system is directed to a genomic locus of interest in a cell without detectable cleavage activity. A detailed description of dead guides is described, e.g., in WO 2016094872, which is incorporated herein by reference in its entirety.

Inducible RNA Guides

RNA guides can be generated as components of inducible systems. The inducible nature of the systems allows for spatiotemporal control of gene editing or gene expression. In some embodiments, the stimuli for the inducible systems include, e.g., electromagnetic radiation, sound energy, chemical energy, and/or thermal energy.

In some embodiments, the transcription of RNA guide can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, each of which is incorporated herein by reference in its entirety.

Chemical Modifications

Chemical modifications can be applied to the phosphate backbone, sugar, and/or base of the RNA guide. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3:154). Additionally, RNA is amenable to both 5′ and 3′ end conjugations with a variety of functional moieties including fluorescent dyes, polyethylene glycol, or proteins.

A wide variety of modifications can be applied to chemically synthesized RNA guide molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the RNA guide includes one or more phosphorothioate modifications. In some embodiments, the RNA guide includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965, each which is incorporated by reference in its entirety.

Sequence Modifications

The sequences and the lengths of the RNA guides, tracrRNAs, and crRNAs described herein can be optimized. In some embodiments, the optimized length of RNA guide can be determined by identifying the processed form of tracrRNA and/or crRNA, or by empirical length studies for RNA guides, tracrRNAs, crRNAs, and the tracrRNA tetraloops.

The RNA guides can also include one or more aptamer sequences. Aptamers are oligonucleotide or peptide molecules that can bind to a specific target molecule. The aptamers can be specific to gene effectors, gene activators, or gene repressors. In some embodiments, the aptamers can be specific to a protein, which in turn is specific to and recruits/binds to specific gene effectors, gene activators, or gene repressors. The effectors, activators, or repressors can be present in the form of fusion proteins. In some embodiments, the RNA guide has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX₁, TW18, VK, SP, FI, ID2, NL95, TW19, AP205, ϕCb5, ϕCb8r, ϕCb12r, ϕCb23r, 7s, and PRR1. Accordingly, in some embodiments, the aptamer is selected from binding proteins specifically binding any one of the adaptor proteins as described herein. In some embodiments, the aptamer sequence is a MS2 loop. A detailed description of aptamers can be found, e.g., in Nowak et al., “Guide RNA engineering for versatile Cas9 functionality,” Nucl. Acid. Res., 2016 Nov. 16; 44(20):9555-9564; and WO 2016205764, each of which is incorporated herein by reference in its entirety.

Guide: Target Sequence Matching Requirements

In CRISPR systems, the degree of complementarity between a guide sequence and its corresponding target sequence can be about 50%, 60%, 75%, 80%, 85%, 90%, 95%, 97.5%, 99%, or 100%. To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required provided that there is sufficient complementarity to be functional. Modulations of cleavage efficiency can be exploited by introduction of mismatches, e.g., one or more mismatches, such as 1 or 2 mismatches between spacer sequence and target sequence, including the position of the mismatch along the spacer/target. The more central (i.e., not at the 3′ or 5′ ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

### Methods of Using CRISPR Systems

The CRISPR systems described herein have a wide variety of utilities including modifying (e.g., deleting, inserting, translocating, inactivating, or activating) a target polynucleotide in a multiplicity of cell types. The CRISPR systems have a broad spectrum of applications in, e.g., DNA/RNA detection (e.g., specific high sensitivity enzymatic reporter unlocking (SHERLOCK)), tracking and labeling of nucleic acids, enrichment assays (extracting desired sequence from background), detecting circulating tumor DNA, preparing next generation library, drug screening, disease diagnosis and prognosis, and treating various genetic disorders.

DNA/RNA Detection

In one aspect, the CRISPR systems described herein can be used in DNA/RNA detection. Single effector RNA-guided DNases can be reprogrammed with CRISPR RNAs (crRNAs) to provide a platform for specific single-stranded DNA (ssDNA) sensing. Upon recognition of its DNA target, activated Type V single effector DNA-guided DNases engage in “collateral” cleavage of nearby non-targeted ssDNAs. This crRNA-programmed collateral cleavage activity allows the CRISPR systems to detect the presence of a specific DNA by nonspecific degradation of labeled ssDNA.

The collateral ssDNA activity can be combined with a reporter in DNA detection applications such as a method called the DNA Endonuclease-Targeted CRISPR trans reporter (DETECTR) method, which achieves attomolar sensitivity for DNA detection (see, e.g., Chen et al., Science, 360(6387):436-439, 2018), which is incorporated herein by reference in its entirety. One application of using the enzymes described herein is to degrade non-specific ssDNA in an in vitro environment. A “reporter” ssDNA molecule linking a fluorophore and a quencher can also be added to the in vitro system, along with an unknown sample of DNA (either single-stranded or double-stranded). Upon recognizing the target sequence in the unknown piece of DNA, the effector complex cleaves the reporter ssDNA resulting in a fluorescent readout.

In other embodiments, the SHERLOCK method (Specific High Sensitivity Enzymatic Reporter UnLOCKing) also provides an in vitro nucleic acid detection platform with attomolar (or single-molecule) sensitivity based on nucleic acid amplification and collateral cleavage of a reporter ssDNA, allowing for real-time detection of the target. Methods of using CRISPR in SHERLOCK are described in detail, e.g., in Gootenberg, et al. “Nucleic acid detection with CRISPR-Cas13a/C2c2,” Science, 356(6336):438-442 (2017), which is incorporated herein by reference in its entirety.

In some embodiments, the CRISPR systems described herein can be used in multiplexed error-robust fluorescence in situ hybridization (MERFISH). These methods are described in, e.g., Chen et al., “Spatially resolved, highly multiplexed RNA profiling in single cells,” Science, 2015 Apr. 24; 348(6233):aaa6090, which is incorporated herein by reference in its entirety.

Tracking and Labeling of Nucleic Acids

Cellular processes depend on a network of molecular interactions among proteins, RNAs, and DNAs. Accurate detection of protein-DNA and protein-RNA interactions is key to understanding such processes. In vitro proximity labeling techniques employ an affinity tag combined with, a reporter group, e.g., a photoactivatable group, to label polypeptides and RNAs in the vicinity of a protein or RNA of interest in vitro. After UV irradiation, the photoactivatable groups react with proteins and other molecules that are in close proximity to the tagged molecules, thereby labelling them. Labelled interacting molecules can subsequently be recovered and identified. The RNA targeting effector proteins can for instance be used to target probes to selected RNA sequences. These applications can also be applied in animal models for in vivo imaging of diseases or difficult-to culture cell types. The methods of tracking and labeling of nucleic acids are described, e.g., in U.S. Pat. No. 8,795,965; WO 2016205764; and WO 2017070605, each of which is incorporated herein by reference in its entirety.

High-Throughput Screening

The CRISPR systems described herein can be used for preparing next generation sequencing (NGS) libraries. For example, to create a cost-effective NGS library, the CRISPR systems can be used to disrupt the coding sequence of a target gene, and the CRISPR effector transfected clones can be screened simultaneously by next-generation sequencing (e.g., on the Ion Torrent PGM system). A detailed description regarding how to prepare NGS libraries can be found, e.g., in Bell et al., “A high-throughput screening strategy for detecting CRISPR-Cas9 induced mutations using next-generation sequencing,” BMC Genomics, 15.1 (2014): 1002, which is incorporated herein by reference in its entirety.

Engineered Cells

Microorganisms (e.g., E. coli, yeast, and microalgae) are widely used for synthetic biology. The development of synthetic biology has a wide utility, including various clinical applications. For example, the programmable CRISPR systems can be used to split proteins of toxic domains for targeted cell death, e.g., using cancer-linked RNA as target transcript. Further, pathways involving protein-protein interactions can be influenced in synthetic biological systems with e.g., fusion complexes with the appropriate effectors such as kinases or enzymes.

In some embodiments, RNA guide sequences that target phage sequences can be introduced into the microorganism. Thus, the disclosure also provides methods of “vaccinating” a microorganism (e.g., a production strain) against phage infection.

In some embodiments, the CRISPR systems provided herein can be used to engineer microorganisms, e.g., to improve yield or improve fermentation efficiency. For example, the CRISPR systems described herein can be used to engineer microorganisms, such as yeast, to generate biofuel or biopolymers from fermentable sugars, or to degrade plant-derived lignocellulose derived from agricultural waste as a source of fermentable sugars. More particularly, the methods described herein can be used to modify the expression of endogenous genes required for biofuel production and/or to modify endogenous genes, which may interfere with the biofuel synthesis. These methods of engineering microorganisms are described e.g., in Verwaal et al., “CRISPR/Cpf1 enables fast and simple genome editing of Saccharomyces cerevisiae,” Yeast, 2017 Sep. 8. doi: 10.1002/yea.3278; and Hlavova et al., “Improving microalgae for biotechnology—from genetics to synthetic biology,” Biotechnol. Adv., 2015 Nov. 1; 33:1194-203, each of which is incorporated herein by reference in its entirety.

In some embodiments, the CRISPR systems provided herein can be used to engineer eukaryotic cells or eukaryotic organisms. For example, the CRISPR systems described herein can be used to engineer eukaryotic cells not limited to a plant cell, a fungal cell, a mammalian cell, a reptile cell, an insect cell, an avian cell, a fish cell, a parasite cell, an arthropod cell, an invertebrate cell, a vertebrate cell, a rodent cell, a mouse cell, a rat cell, a primate cell, a non-human primate cell, or a human cell. In some embodiments, eukaryotic cell is in an in vitro culture. In some embodiments, the eukaryotic cell is in vivo. In some embodiments, the eukaryotic cell is ex vivo.

In some embodiments, the cell is derived from a cell line. A wide variety of cell lines for tissue culture are known in the art. Examples of cell lines include, but are not limited to, 293T, MF7, K562, HeLa, and transgenic varieties thereof. Cell lines are available from a variety of sources known to those with skill in the art (see, e.g., the American Type Culture Collection (ATCC) (Manassas, Va.)). In some embodiments, a cell transfected with one or more nucleic acids (such as nuclease polypeptide encoding vector and RNA guide) is used to establish a new cell line comprising one or more vector-derived sequences to establish a new cell line comprising modification to the target nucleic acid or target locus. In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a mammalian cell, e.g., a human cell or a murine cell. In some embodiments, the murine cell is derived from a wild-type mouse, an immunosuppressed mouse, or a disease-specific mouse model.

Gene Drives

Gene drive is the phenomenon in which the inheritance of a particular gene or set of genes is favorably biased. The CRISPR systems described herein can be used to build gene drives. For example, the CRISPR systems can be designed to target and disrupt a particular allele of a gene, causing the cell to copy the second allele to fix the sequence. Because of the copying, the first allele will be converted to the second allele, increasing the chance of the second allele being transmitted to the offspring. A detailed method regarding how to use the CRISPR systems described herein to build gene drives is described, e.g., in Hammond et al., “A CRISPR-Cas9 gene drive system targeting female reproduction in the malaria mosquito vector Anopheles gambiae,” Nat. Biotechnol., 2016 January; 34(1):78-83, which is incorporated herein by reference in its entirety.

Pooled-Screening

As described herein, pooled CRISPR screening is a powerful tool for identifying genes involved in biological mechanisms such as cell proliferation, drug resistance, and viral infection. Cells are transduced in bulk with a library of RNA guide-encoding vectors described herein, and the distribution of gRNAs is measured before and after applying a selective challenge. Pooled CRISPR screens work well for mechanisms that affect cell survival and proliferation, and they can be extended to measure the activity of individual genes (e.g., by using engineered reporter cell lines). Arrayed CRISPR screens, in which only one gene is targeted at a time, make it possible to use RNA-seq as the readout. In some embodiments, the CRISPR systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods., 2017 March; 14(3):297-301, which is incorporated herein by reference in its entirety.

Saturation Mutagenesis (“Bashing”)

The CRISPR systems described herein can be used for in situ saturating mutagenesis. In some embodiments, a pooled RNA guide library can be used to perform in situ saturating mutagenesis for particular genes or regulatory elements. Such methods can reveal critical minimal features and discrete vulnerabilities of these genes or regulatory elements (e.g., enhancers). These methods are described, e.g., in Canver et al., “BCL11A enhancer dissection by Cas9-mediated in situ saturating mutagenesis,” Nature, 2015 Nov. 12; 527(7577):192-7, which is incorporated herein by reference in its entirety.

Therapeutic Applications

In some embodiments, the CRISPR systems described herein can be used to edit a target nucleic acid to modify the target nucleic acid (e.g., by inserting, deleting, or mutating one or more amino acid residues). For example, in some embodiments the CRISPR systems described herein comprise an exogenous donor template nucleic acid (e.g., a DNA molecule or an RNA molecule), which comprises a desirable nucleic acid sequence. Upon resolution of a cleavage event induced with the CRISPR system described herein, the molecular machinery of the cell can utilize the exogenous donor template nucleic acid in repairing and/or resolving the cleavage event. Alternatively, the molecular machinery of the cell can utilize an endogenous template in repairing and/or resolving the cleavage event. In some embodiments, the CRISPR systems described herein may be used to modify a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double-stranded or single-stranded nucleic acid molecules (e.g., DNA or RNA). Methods of designing exogenous donor template nucleic acids are described, for example, in WO 2016094874, the entire contents of which is expressly incorporated herein by reference.

In another aspect, the disclosure provides the use of a system described herein in a method selected from the group consisting of RNA sequence specific interference; RNA sequence-specific gene regulation; screening of RNA, RNA products, lncRNA, non-coding RNA, nuclear RNA, or mRNA; mutagenesis; inhibition of RNA splicing; fluorescence in situ hybridization; breeding; induction of cell dormancy; induction of cell cycle arrest; reduction of cell growth and/or cell proliferation; induction of cell anergy; induction of cell apoptosis; induction of cell necrosis; induction of cell death; or induction of programmed cell death.

The CRISPR systems described herein can have various therapeutic applications. In some embodiments, the new CRISPR systems can be used to treat various diseases and disorders, e.g., genetic disorders (e.g., monogenetic diseases) or diseases that can be treated by nuclease activity (e.g., Pcsk9 targeting or BCL11a targeting). In some embodiments, the methods described here are used to treat a subject, e.g., a mammal, such as a human patient. The mammalian subject can also be a domesticated mammal, such as a dog, cat, horse, monkey, rabbit, rat, mouse, cow, goat, or sheep.

The methods can include the condition or disease being infectious, and wherein the infectious agent is selected from the group consisting of human immunodeficiency virus (HIV), herpes simplex virus-1 (HSV1), and herpes simplex virus-2 (HSV2).

In one aspect, the CRISPR systems described herein can be used for treating a disease caused by overexpression of RNAs, toxic RNAs and/or mutated RNAs (e.g., splicing defects or truncations). For example, expression of the toxic RNAs may be associated with the formation of nuclear inclusions and late-onset degenerative changes in brain, heart, or skeletal muscle. In some embodiments, the disorder is myotonic dystrophy. In myotonic dystrophy, the main pathogenic effect of the toxic RNAs is to sequester binding proteins and compromise the regulation of alternative splicing (see, e.g., Osborne et al., “RNA-dominant diseases,” Hum. Mol. Genet., 2009 Apr. 15; 18(8):1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR systems as described herein can target overexpressed RNA or toxic RNA, e.g., the DMPK gene or any of the mis-regulated alternative splicing in DM1 skeletal muscle, heart, or brain.

The CRISPR systems described herein can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR systems described herein is summarized in Cooper et al., “RNA and disease,” Cell, 136.4 (2009): 777-793, and WO 2016205764, each of which is incorporated herein by reference in its entirety.

The CRISPR systems described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR systems described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis.

The CRISPR systems described herein can further be used for antiviral activity, in particular, against RNA viruses. The effector proteins can target the viral RNAs using suitable RNA guides selected to target viral RNA sequences.

Furthermore, in vitro RNA sensing assays can be used to detect specific RNA substrates. The RNA targeting effector proteins can be used for RNA-based sensing in living cells. Examples of applications are diagnostics by sensing of, for examples, disease-specific RNAs.

A detailed description of therapeutic applications of the CRISPR systems described herein can be found, e.g., in U.S. Pat. No. 8,795,965, EP 3009511, WO 2016205764, and WO 2017070605, each of which is incorporated herein by reference in its entirety.

Applications in Plants

The CRISPR systems described herein have a wide variety of utility in plants. In some embodiments, the CRISPR systems can be used to engineer genomes of plants (e.g., improving production, making products with desired post-translational modifications, or introducing genes for producing industrial products). In some embodiments, the CRISPR systems can be used to introduce a desired trait to a plant (e.g., with or without heritable modifications to the genome) or regulate expression of endogenous genes in plant cells or whole plants.

In some embodiments, the CRISPR systems can be used to identify, edit, and/or silence genes encoding specific proteins, e.g., allergenic proteins (e.g., allergenic proteins in peanuts, soybeans, lentils, peas, green beans, and mung beans). A detailed description regarding how to identify, edit, and/or silence genes encoding proteins is described, e.g., in Nicolaou et al., “Molecular diagnosis of peanut and legume allergy,” Curr. Opin. Allergy Clin. Immunol., 11(3):222-8 (2011) and WO 2016205764, each of which is incorporated herein by reference in its entirety.

Delivery of CRISPR Systems

Through this disclosure and knowledge in the art, the CRISPR systems described herein, components thereof, nucleic acid molecules thereof, or nucleic acid molecules encoding or providing components thereof can be delivered by various delivery systems such as vectors, e.g., plasmids or viral delivery vectors. The CRISPR effectors and/or any of the RNAs (e.g., RNA guides) disclosed herein can be delivered using suitable vectors, e.g., plasmids or viral vectors, such as adeno-associated viruses (AAV), lentiviruses, adenoviruses, and other viral vectors, or combinations thereof. An effector and one or more RNA guides can be packaged into one or more vectors, e.g., plasmids or viral vectors.

In some embodiments, vectors, e.g., plasmids or viral vectors, are delivered to the tissue of interest by, e.g., intramuscular injection, intravenous administration, transdermal administration, intranasal administration, oral administration, or mucosal administration. Such delivery may be either via one dose or multiple doses. One skilled in the art understands that the actual dosage to be delivered herein may vary greatly depending upon a variety of factors, including, but not limited to, the vector choices, the target cells, organisms, tissues, the general conditions of the subject to be treated, the degrees of transformation/modification sought, the administration routes, the administration modes, and the types of transformation/modification sought.

In certain embodiments, delivery is via adenoviruses, which can be one dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 and U.S. Pat. No. 8,454,972, each of which is incorporated herein by reference in its entirety.

In some embodiments, delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR effector, operably linked to the promoter; (iii) a selectable marker; (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, delivery is via liposomes or lipofectin formulations or the like and can be prepared by methods known to those skilled in the art. Such methods are described, for example, in WO 2016205764, U.S. Pat. Nos. 5,593,972, 5,589,466, and 5,580,859, each of which is incorporated herein by reference in its entirety.

In some embodiments, delivery is via nanoparticles or exosomes. For example, exosomes have been shown to be particularly useful in delivery RNA.

Further means of introducing one or more components of the CRISPR systems described herein to a cell is by using cell-penetrating peptides (CPP). In some embodiments, a cell penetrating peptide is linked to a CRISPR effector. In some embodiments, a CRISPR effector and/or RNA guide is coupled to one or more CPPs for transportation into a cell (e.g., plant protoplasts). In some embodiments, the CRISPR effector and/or RNA guide(s) are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HW type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hallbrink et al., “Prediction of cell-penetrating peptides,”Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764, each of which is incorporated herein by reference in its entirety.

Various delivery methods for the CRISPR systems described herein are also described, e.g., in U.S. Pat. No. 8,795,965, EP 3009511, WO 2016205764, and WO 2017070605, each of which is incorporated herein by reference in its entirety.

### EXAMPLES

The invention is further described in the following examples, which do not limit the scope of the invention described in the claims.

### Example 1—Identification of Components of CLUST.091979 CRISPR-Cas System

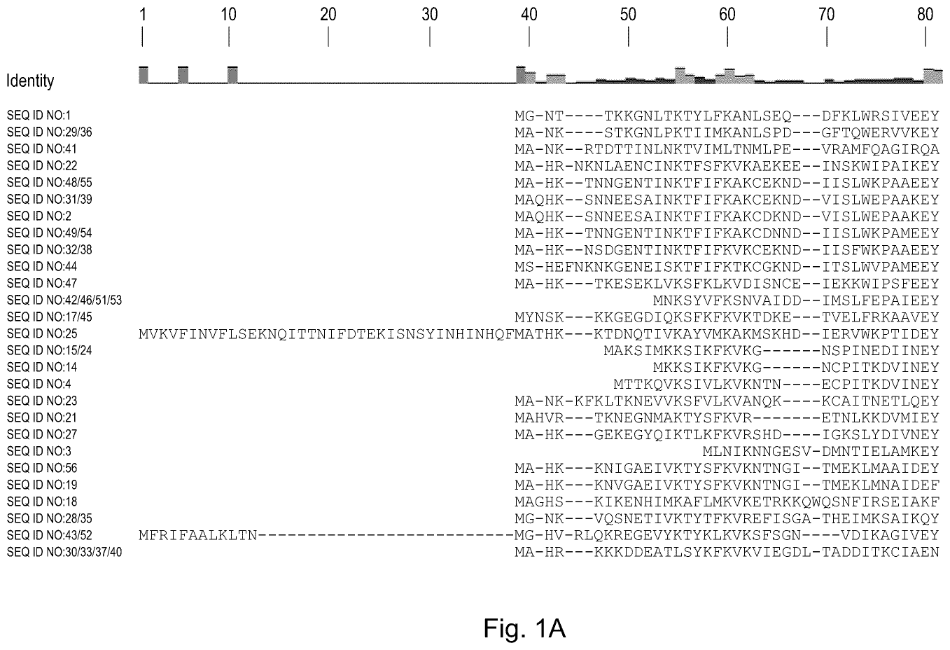

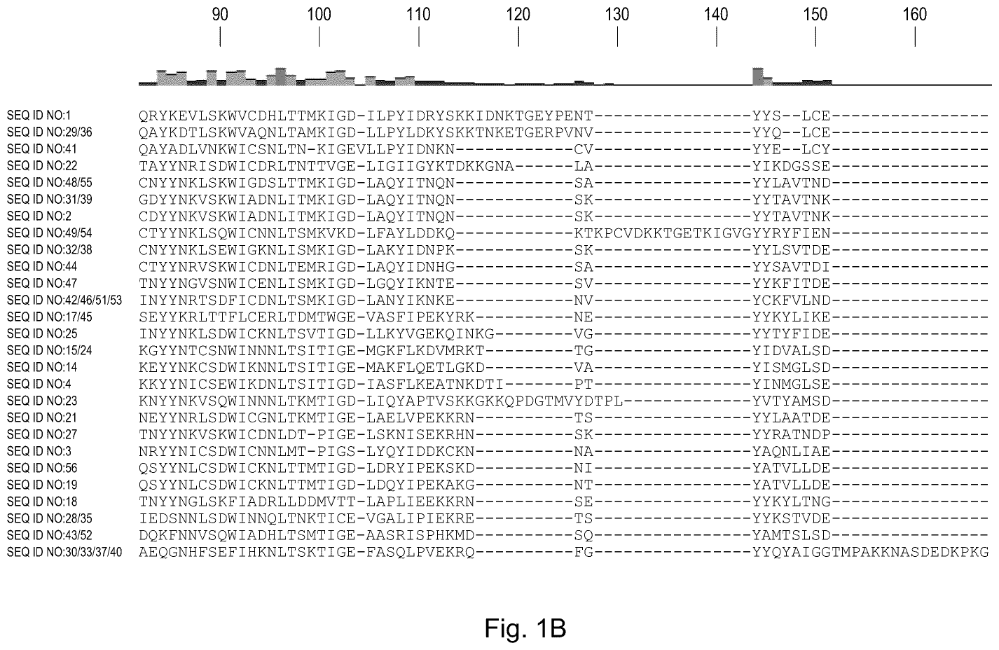

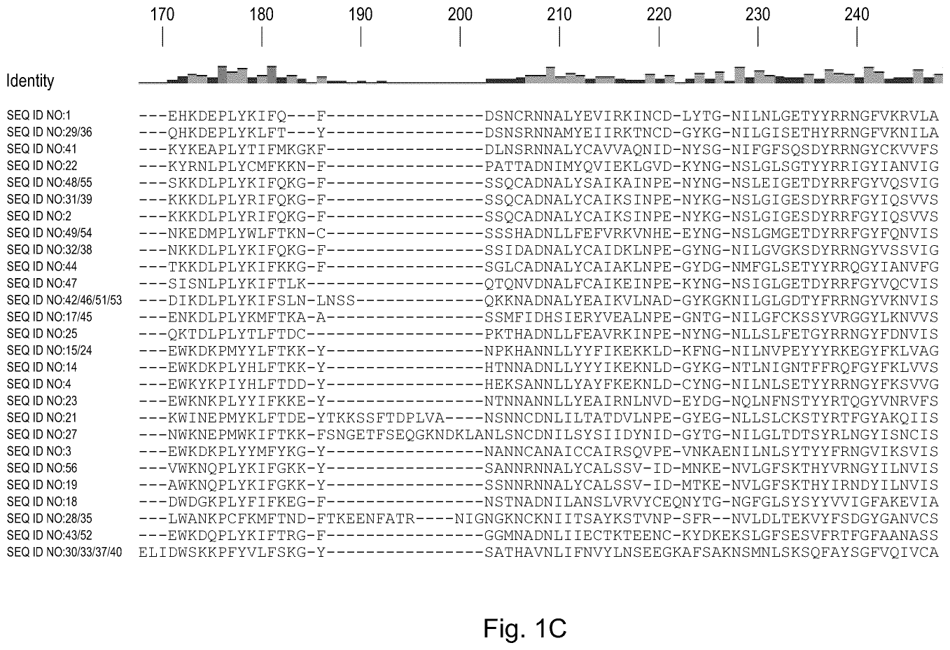

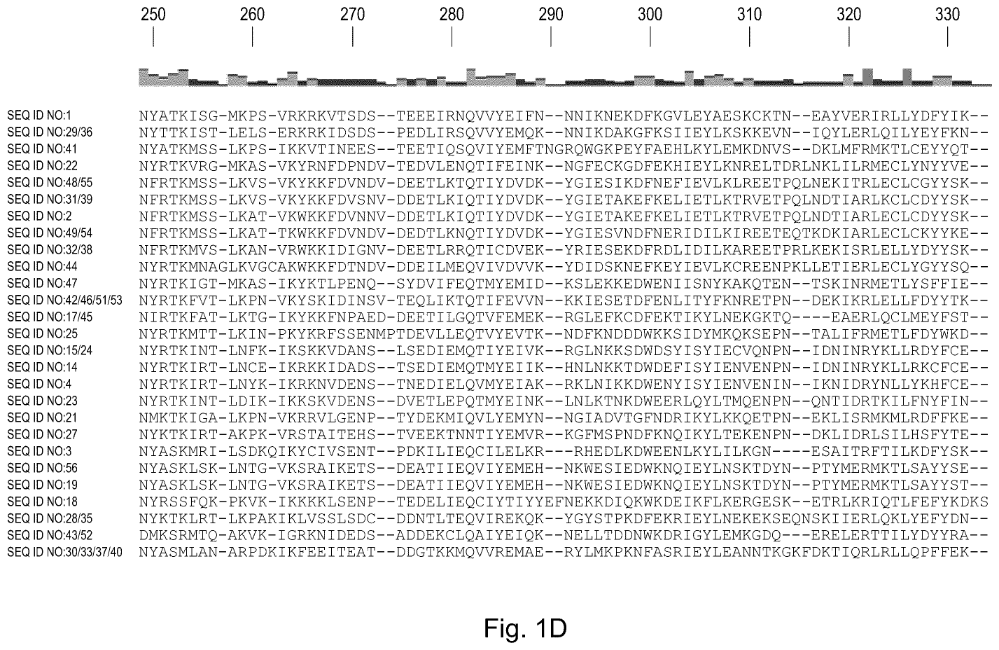

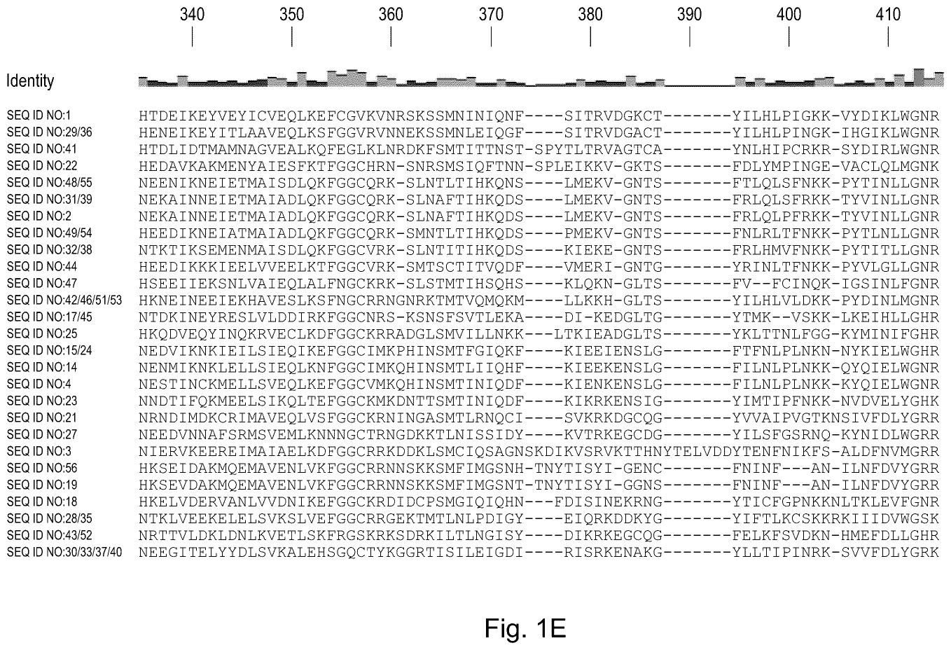

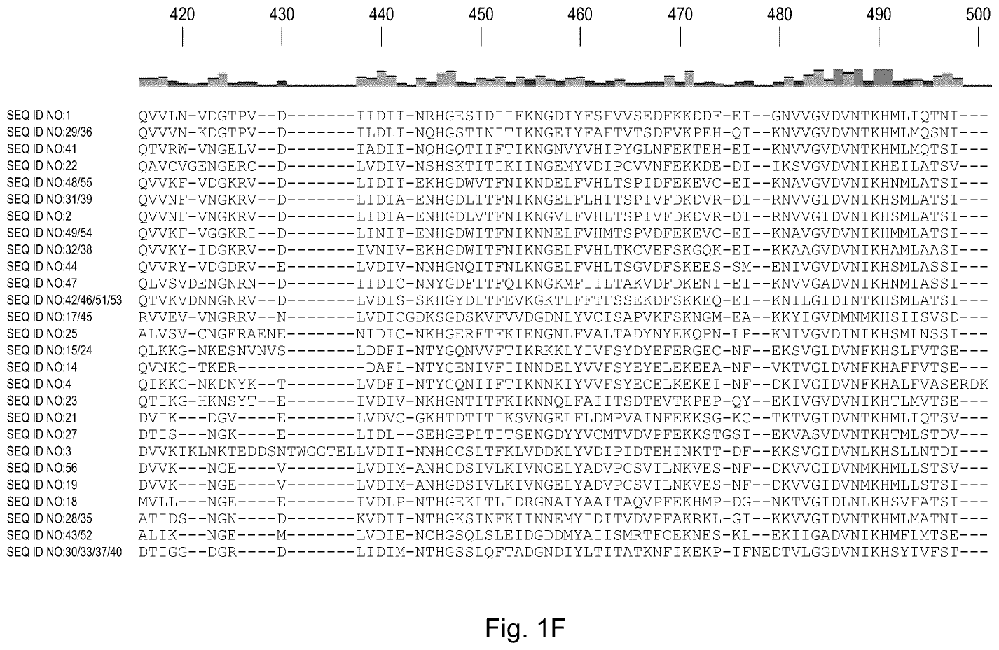

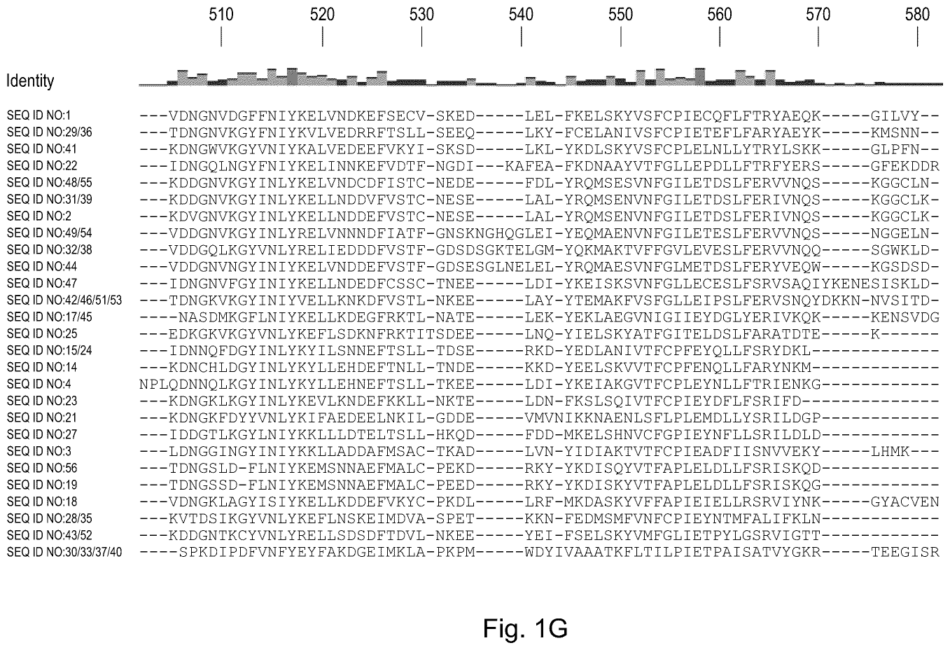

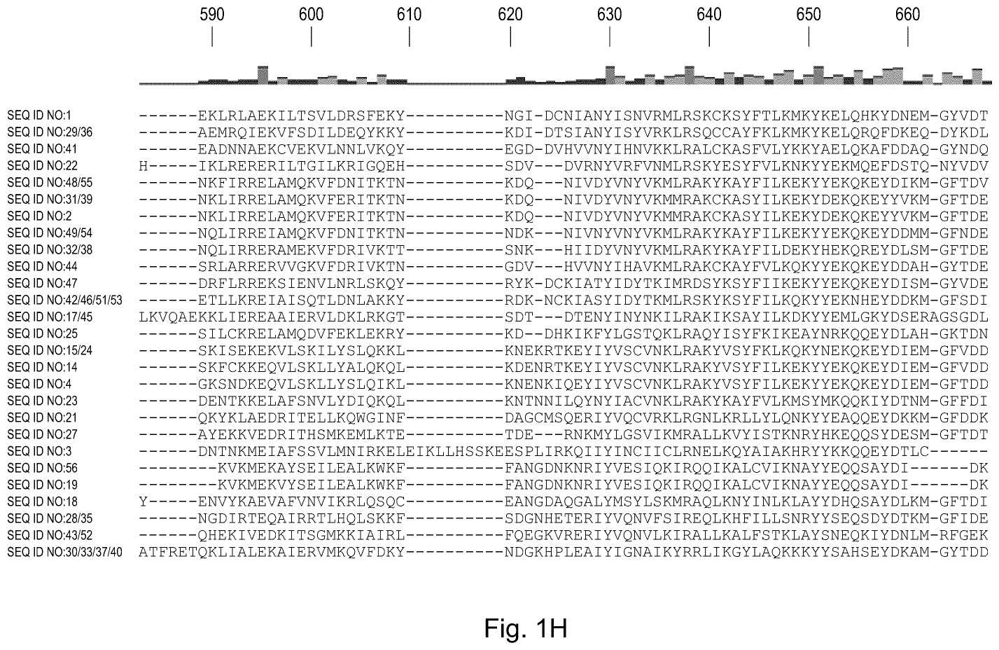

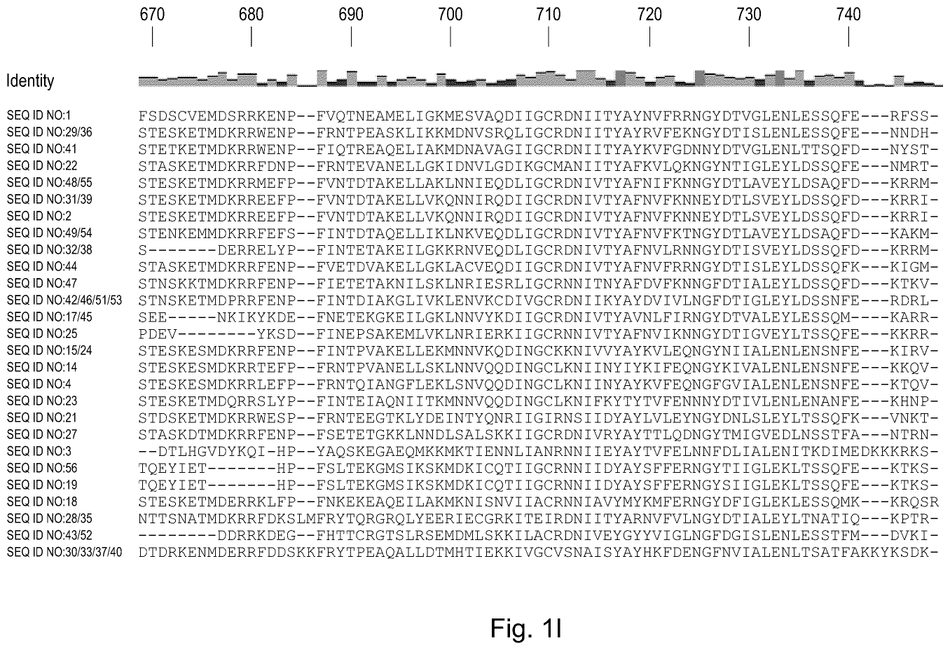

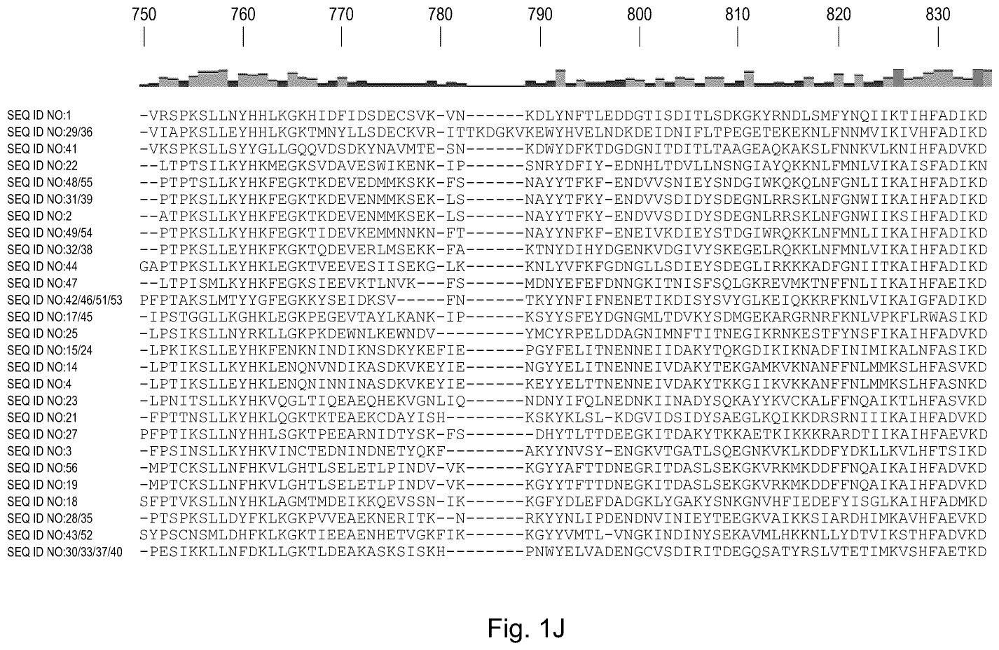

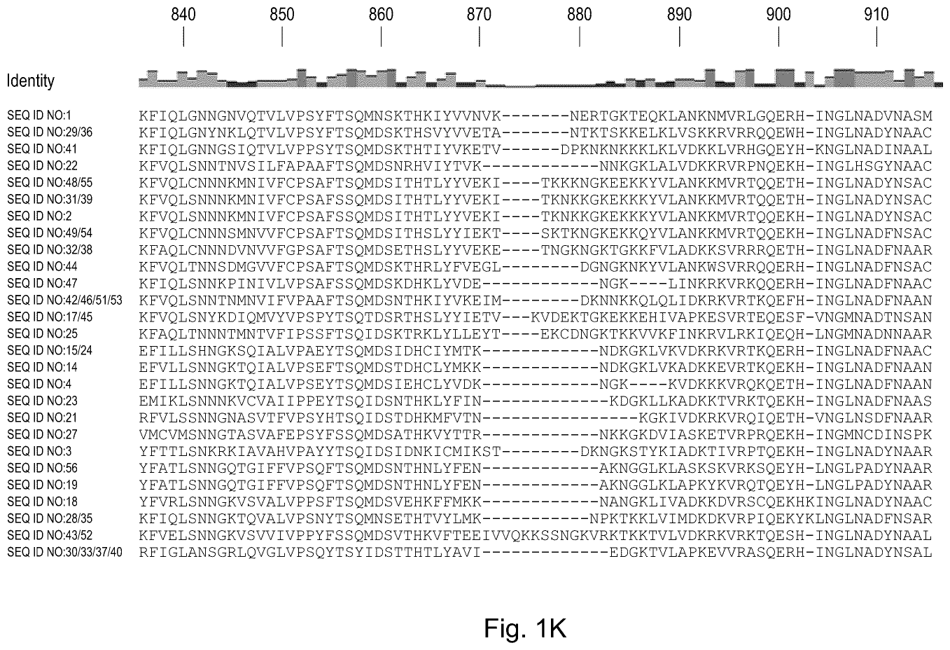

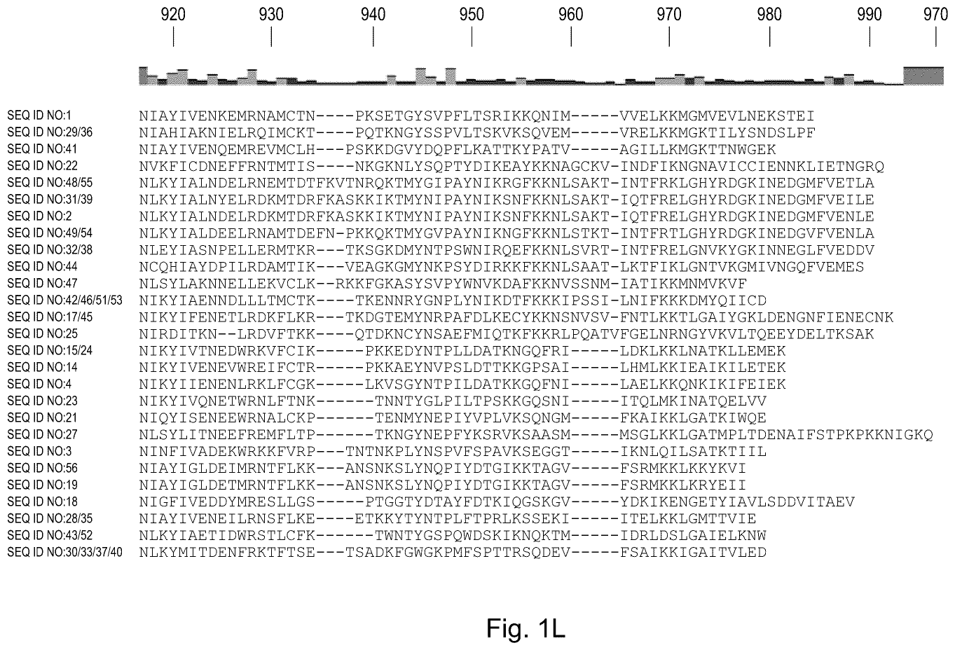

This protein family was identified using the computational methods described above. The CLUST.091979 system comprises single effectors associated with CRISPR systems found in uncultured metagenomic sequences collected from environments not limited to gut, bovine gut, human gut, sheep gut, terrestrial, feces, and mammalian digestive system environments (TABLE 5). Exemplary CLUST.091979 effectors include those shown in TABLE 5 and TABLE 6, below. The effector sequences set forth in SEQ ID NOs: 1-4, 14, 15, 17-19, 21-25, 27-33, 35-49, 51-56 were aligned to identify regions of sequence similarity, as shown in FIGS. 1A-1L. A bar graph depicts sequence similarity, with the tallest bars indicating the residues with the highest sequence similarity. Non-limiting regions of sequence similarity are shown in TABLE 7. The regions of sequence similarity indicate that the effectors disclosed herein are a family with a conserved C-terminal RuvC domain representative of nucleases.

TABLE 5
Representative CLUST.091979 Effector Proteins

| | | | | SEQ |
|---|---|---|---|---|
| | | # | effector | ID |
| source | effector accession | spacers | size | NO |
| gut metagenome | AUXO013988882_8\|P | 4 | 775 | 1 |
| bovine gut | SRR094437_845781_4\|M | 11 | 786 | 2 |
| metagenome | | | | |
| gut metagenome | SRR1221442_316828_61\|P | 2 | 774 | 3 |
| bovine gut | SRR3181151_741875_3\|M | 8 | 756 | 4 |
| metagenome | | | | |
| bovine gut | SRR5371369_1764679_7\|P | 7 | 746 | 5 |
| metagenome | | | | |
| bovine gut | SRR5371371_1138852_2\|M | 3 | 733 | 6 |
| metagenome | | | | |
| bovine gut | SRR5371379_2478682_1\|M | 9 | 744 | 7 |
| metagenome | | | | |
| bovine gut | SRR5371385_201181_1\|P | 4 | 754 | 8 |
| metagenome | | | | |
| bovine gut | SRR5371385_201181_1\|M | 4 | 746 | 9 |
| metagenome | | | | |
| bovine gut | SRR5371401_ | 15 | 745 | 10 |
| metagenome | 1055766_58\|M | | | |
| bovine gut | SRR5371439_988701_11\|M | 5 | 744 | 11 |
| metagenome | | | | |
| bovine gut | SRR5371497_203858_6\|M | 5 | 745 | 12 |
| metagenome | | | | |
| bovine gut | SRR5371501_2762794_1\|M | 2 | 712 | 13 |
| metagenome | | | | |
| terrestrial | SRR5678926_1309611_3\|P | 6 | 741 | 14 |
| metagenome | | | | |
| feces metagenome | SRR6059713_382107_4\|P | 4 | 752 | 15 |
| feces metagenome | SRR6060192_2608084_13\|P | 16 | 766 | 16 |
| sheep gut | SRR7634052_ | 8 | 784 | 17 |
| metagenome | 1662339_24\|M | | | |
| gut metagenome | AUXO017332817_2\|M | 5 | 782 | 18 |
| human gut | OQVL01000914_15\|P | 6 | 735 | 19 |
| metagenome | | | | |
| mammals-digestive | 3300001598\| | 2 | 774 | 20 |
| system-asian | EMG_10017415_6\|P | | | |
| elephant fecal- | | | | |
| elephas maximus | | | | |
| mammals-digestive | 3300021254\| | 3 | 755 | 21 |
| system-cattle | Ga0223824_10022219_2\|P | | | |
| and sheep rumen | | | | |
| mammals-digestive | 3300021431\| | 11 | 789 | 22 |
| system-cattle | Ga0224423_10015012_2\|P | | | |
| and sheep rumen | | | | |
| mammals-digestive | 3300012973\| | 6 | 766 | 23 |
| system-fecal | Ga0123351_1009859_3\|P | | | |
| mammals-digestive | 3300012979\| | 4 | 752 | 24 |
| system-fecal | Ga0123348_10005323_4\|M | | | |
| mammals-digestive | 3300028797\| | 26 | 814 | 25 |
| system-rumen- | Ga0265301_10000251_12\|M | | | |
| bos taurus | | | | |
| mammals-digestive | 3300028797\| | 26 | 776 | 26 |
| system-rumen- | Ga0265301_10000251_10\|P | | | |
| bos taurus | | | | |
| mammals-digestive | 3300028797\| | 2 | 778 | 27 |
| system-rumen- | Ga0265301_10009039_3\|M | | | |
| bos taurus | | | | |
| mammals-digestive | 3300028887\| | 8 | 772 | 28 |
| system-rumen- | Ga0265299_ | | | |
| bos taurus | 10000013_320\|P | | | |
| mammals-digestive | 3300028887\| | 2 | 781 | 29 |
| system-rumen- | Ga0265299_10000026_77\|P | | | |
| bos taurus | | | | |
| mammals-digestive | 3300028887\| | 11 | 798 | 30 |
| system-rumen- | Ga0265299_10000133_30\|M | | | |
| bos taurus | | | | |
| mammals-digestive | 3300028887\| | 15 | 786 | 31 |
| system-rumen- | Ga0265299_10011526_3\|M | | | |
| bos taurus | | | | |
| mammals-digestive | 3300028887\| | 10 | 781 | 32 |
| system-rumen- | Ga0265299_10012919_3\|P | | | |
| bos taurus | | | | |
| mammals-digestive | 3300028914\| | 2 | 798 | 33 |
| system-rumen- | Ga0265300_10009460_3\|M | | | |
| bos taurus | | | | |
| mammals-digestive | 3300031853\| | 4 | 724 | 34 |
| system-rumen- | Ga0326514_10013355_6\|M | | | |
| bos taurus | | | | |
| mammals-digestive | 3300031993\| | 8 | 772 | 35 |
| system-rumen- | Ga0310696_ | | | |
| bos taurus | 10000014_323\|P | | | |
| mammals-digestive | 3300031993\| | 2 | 781 | 36 |
| system-rumen- | Ga0310696_10000226_76\|P | | | |
| bos taurus | | | | |
| mammals-digestive | 3300031993\| | 11 | 798 | 37 |
| system-rumen- | Ga0310696_10000447_27\|M | | | |
| bos taurus | | | | |
| mammals-digestive | 3300031993\| | 2 | 781 | 38 |
| system-rumen- | Ga0310696_10026614_2\|M | | | |
| bos taurus | | | | |
| mammals-digestive | 3300031993\| | 14 | 786 | 39 |
| system-rumen- | Ga0310696_10030100_3\|M | | | |
| bos taurus | | | | |
| mammals-digestive | 3300031998\| | 9 | 798 | 40 |
| system-rumen- | Ga0310786_ | | | |
| bos taurus | 10000003_467\|M | | | |
| mammals-digestive | AUXO013988882\| | 6 | 771 | 41 |
| system-rumen- | Ga0247611_10000101_23\|P | | | |
| ovis aries | | | | |
| mammals-digestive | 3300028805\| | 7 | 764 | 42 |
| system-rumen- | Ga0247608_10000186_37\|P | | | |
| ovis aries | | | | |
| mammals-digestive | 3300028805\| | 8 | 768 | 43 |
| system-rumen- | Ga0247608_10000895_42\|M | | | |
| ovis aries | | | | |
| mammals-digestive | 3300028805\| | 10 | 789 | 44 |
| system-rumen- | Ga0247608_10006074_1\|M | | | |
| ovis aries | | | | |
| mammals-digestive | 3300028833\| | 8 | 784 | 45 |
| system-rumen- | Ga0247610_ | | | |
| ovis aries | 10000007_379\|M | | | |
| mammals-digestive | 3300028833\| | 7 | 764 | 46 |
| system-rumen- | Ga0247610_10004486_2\|M | | | |
| ovis aries | | | | |
| mammals-digestive | 3300028888\| | 11 | 758 | 47 |
| system-rumen- | Ga0247609_10000668_74\|M | | | |
| ovis aries | | | | |
| mammals-digestive | 3300028888\| | 8 | 785 | 48 |
| system-rumen- | Ga0247609_10003329_9\|M | | | |
| ovis aries | | | | |
| mammals-digestive | 3300028888\| | 2 | 805 | 49 |
| system-rumen- | Ga0247609_10016480_8\|M | | | |
| ovis aries | | | | |
| mammals-digestive | 3300031992\| | 8 | 784 | 50 |
| system-rumen- | Ga0310694_ | | | |
| ovis aries | 10000010_351\|M | | | |
| mammals-digestive | 3300031992\| | 7 | 764 | 51 |
| system-rumen- | Ga0310694_10022272_2\|M | | | |
| ovis aries | | | | |
| mammals-digestive | 3300031994\| | 8 | 768 | 52 |
| system-rumen- | Ga0310691_ | | | |
| ovis aries | 10000084_157\|M | | | |
| mammals-digestive | 3300031994\| | 7 | 764 | 53 |
| system-rumen- | Ga0310691_10000270_20\|M | | | |
| ovis aries | | | | |
| mammals-digestive | 3300032030\| | 2 | 805 | 54 |
| system-rumen- | Ga0310697_10001273_44\|P | | | |
| ovis aries | | | | |
| mammals-digestive | 3300032030\| | 8 | 785 | 55 |
| system-rumen- | Ga0310697_10005481_13\|P | | | |
| ovis aries | | | | |
| pig gut metagenome | OBLI01003123_14\|M | 4 | 735 | 56 |

TABLE 6
Amino Acid Sequences of Representative CLUST.091979 Effector Proteins

>AUXO013988882_8|P
[gut metagenome]
MGNTTKKGNLTKTYLFKANLSEQDFKLWRSIVEEYQRYKEVLSKWVCDHLTTMKIGDILPYIDRYSKKIDNKTGEYPENTYYSL
CEEHKDEPLYKIFQFDSNCRNNALYEVIRKINCDLYTGNILNLGETYYRRNGFVKRVLANYATKISGMKPSVRKRKVTSDSTEE
EIRNQVVYEIFNNNIKNEKDFKGVLEYAESKCKTNEAYVERIRLLYDFYIKHTDEIKEYVEYICVEQLKEFCGVKVNRSKSSMN
INIQNFSITRVDGKCTYILHLPIGKKVYDIKLWGNRQVVLNVDGTPVDIIDIINRHGESIDIIFKNGDIYFSFVVSEDFKKDDF
EIGNVVGVDVNTKHMLIQTNIVDNGNVDGFFNIYKELVNDKEFSECVSKEDLELFKELSKYVSFCPIECQFLFTRYAEQKGILV
YEKLRLAEKILTSVLDRSFEKYNGIDCNIANYISNVRMLRSKCKSYFTLKMKYKELQHKYDNEMGYVDTFSDSCVEMDSRRKEN
PFVQTNEAMELIGKMESVAQDIIGCRDNIITYAYNVFRRNGYDTVGLENLESSQFERFSSVRSPKSLLNYHHLKGKHIDFIDSD
ECSVKVNKDLYNFTLEDDGTISDITLSDKGKYRNDLSMFYNQIIKTIHFADIKDKFIQLGNNGNVQTVLVPSYFTSQMNSKTHK
IYVVNVKNERTGKTEQKLANKNMVRLGQERHINGLNADVNASMNIAYIVENKEMRNAMCTNPKSETGYSVPFLTSRIKKQNIMV
VELKKMGMVEVLNEKSTEI (SEQ ID NO: 1)
>SRR094437_845781_4|M
[bovine gut metagenome]
MAQHKSNNEESAINKTFIFKAKCDKNDVISLWEPAAKEYCDYYNKVSKWIADNLITMKIGDLAQYITNQNSKYYTAVTNKKKKD
LPLYRIFQKGFSSQCADNALYCAIKSINPENYKGNSLGIGESDYRRFGYIQSVVSNFRTKMSSLKATVKWKKFDVNNVDDETLK
IQTIYDVDKYGIETAKEFKELIETLKTRVETPQLNDTIARLECLCDYYSKNEKAINNEIETMAIADLQKFGGCQRKSLNAFTIH
KQDSLMEKVGNTSFRLQLPFRKKTYVINLLGNRQVVNFVNGKRVDLIDIAENHGDLVTFNIKNGVLFVHLTSPIVFDKDVRDIR
NVVGIDVNIKHSMLATSIKDVGNVKGYINLYKELLNDDEFVSTCNESELALYRQMSENVNFGILETDSLFERIVNQSKGGCLKN
KLIRRELAMQKVFERITKTNKDQNIVDYVNYVKMMRAKCKASYILKEKYDEKQKEYYVKMGFTDESTESKETMDKRREEFPFVN
TDTAKELLVKQNNIRQDIIGCRDNIVTYAFNVFKNNEYDTLSVEYLDSSQFDKRRIATPKSLLKYHKFEGKTKDEVENMMKSEK
LSNAYYTFKYENDVVSDIDYSDEGNLRRSKLNFGNWIIKSIHFADIKDKFVQLSNNNKMNIVFCPSAFSSQMDSITHTLYYVEK
ITKNKKGKEKKKYVLANKKMVRTQQEKHINGLNADYNSACNLKYIALNDELRDKMTDRFKASKKIKTMYNIPAYNIKSNFKKNL
SAKTIQTFRELGHYRDGKINEDGMFVENLE (SEQ ID NO: 2)
>SRR1221442_316828_61|P
[gut metagenome]
MLNIKNNGESVDMNTIELAMKEYNRYYNICSDWICNNLMTPIGSLYQYIDDKCKNNAYAQNLIAEEWKDKPLYYMFYKGYNANN
CANAICCAIRSQVPEVNKAENILNLSYTYYFRNGVIKSVISNYASKMRILSDKQIKYCIVSENTPDKILIEQCILELKRRHEDL
KDWEENLKYLILKGNESAITRFTILKDFYSKNIERVKEEREIMAIAELKDFGGCRRKDDKLSMCIQSAGNSKDIKVSRVKTTHN
YTELVDDYTENFNIKFSALDFNVMGRRDVVKTKLNKTEDDSNTWGGTELLVDIINNHGCSLTFKLVDDKLYVDIPIDTEHINKT
TDFKKSVGIDVNLKHSLLNTDILDNGGINGYINIYKKLLADDAFMSACTKADLVNYIDIAKTVTFCPIEADFIISNVVEKYLHM
KDNTNKMEIAFSSVLMNIRKELEIKLLHSSKEESPLIRKQIIYINCIICLRNELKQYAIAKHRYYKKQQEYDTLCDTLHGVDYK
QIHPYAQSKEGAEQMKKMKTIENNLIANRNNIlEYAYTVFELNNFDLIALENITKDIMEDKKKRKSFPSINSLLKYHKVINCTE
DNINDNETYQKFAKYYNVSYENGKVTGATLSQEGNKVKLKDDFYDKLLKVLHFTSIKDYFTTLSNKRKIAVAHVPAYYTSQIDS
IDNKICMIKSTDKNGKSTYKIADKTIVRPTQEKHINGLNADYNAARNINFIVADEKWRKKFVRPTNTNKPLYNSPVFSPAVKSE
GGTIKNLQILSATKTIIL (SEQ ID NO: 3)
>SRR3181151_741875_3|M
[bovine gut metagenome]
MTTKQVKSIVLKVKNTNECPITKDVINEYKKYYNICSEWIKDNLTSITIGDIASFLKEATNKDTIPTYINMGLSEEWKYKPIYH
LFTDDYHEKSANNLLYAYFKEKNLDCYNGNILNLSETYYRRNGYFKSVVGNYRTKIRTLNYKIKRKNVDENSTNEDIELQVMYE
lAKRKLNIKKDWENYISYIENVENINIKNIDRYNLLYKHFCENESTINCKMELLSVEQLKEFGGCVMKQHINSMTINIQDFKIE
NKENSLGFILNLPLNKKKYQIELWGNRQIKKGNKDNYKTLVDFINTYGQNIIFTIKNNKIYVVFSYECELKEKEINFDKIVGID
VNFKHALFVASERDKNPLQDNNQLKGYINLYKYLLEHNEFTSLLTKEELDIYKEIAKGVTFCPLEYNLLFTRIENKGGKSNDKE
QVLSKLLYSLQIKLKNENKIQEYIYVSCVNKLRAKYVSYFILKEKYYEKQKEYDIEMGFTDDSTESKESMDKRRLEFPFRNTQI
ANGFLEKLSNVQQDINGCLKNIINYAYKVFEQNGFGVIALENLENSNFEKTQVLPTIKSLLEYHKLENQNINNINASDKVKEYI
EKEYYELTTNENNEIVDAKYTKKGIIKVKKANFFNLMMKSLHFASNKDEFILLSNNGKTQIALVPSEYTSQMDSIEHCLYVDKN
GKKVDKKKVRQKQETHINGLNADFNAANNIKYIIENENLRKLFCGKLKVSGYNTPILDATKKGQFNILAELKKQNKIKIFEIEK
(SEQ ID NO: 4)
>SRR5371369_1764679_7|P
[bovine gut metagenome]
MASHKKTESNQIIKTFPFKLKNANGLSLDVLNDAITEYQNYYNICSDWIKDHLTMKISELYKYIPDEKKNSGYALTLISDEWKD
KPMYMMFKKGYPANNRDNAIYETLNTCNTEHYTGNILNFPDTYYRRFGYVASTISNYVTKISKMSTGSRSKNISNDSDVDTIME
QVIYEMEHNGWTSVKDWENQMEYLESKTDSNPNFVYRMTTLYEFYKSHIDEVNSKMETMSIDLLIKFGGCRRKDSKKSMYIMGG
SNTPFDITQIGDNSLNIKFSKNLNVDVFGRYDVIKDNTLLVDIINGHGASFVLKIINDEIYIDINVSVPFDKKIATTNKVVGID
VNIKHMLLATNILDDGNVKGYVNIYKEVINDSDFKKVCNSTVMKYFTDFSKFVTFCPLEFDFLFSRVCNQKGIYNDNSVMEKSF
SDVLNKLKWNFIETGDNTKRIYIENVMKLRTQMKAYAIVKNAYYKQQSEYDFGKSEEFIQEHPFSNTDKGIEILHKLDNISKKI
LGCRNNIIQYSYNLFEINGYDMISLEKLTSSQFKKKSFPTVNSLLKYHKILGCTQEEMEKKDIYSVIKKGYYDIIFDNDVVTDA
KLSTKGELSKFKDDFFNLMIKSIHFADIKDYFITLSNNGTAGVSLVPSFFTSQMDSIDHKIYFVQDNKSGKLKLANKHKVRSSQ
EKHINGLNADYNAARNIAYIMENTECRNMFMKQSRTDKSLYNKPSYETFIKTQGSAVAKLKKEGFMKILDEASV (SEQ ID
NO: 5)
>SRR5371371_1138852_2|M
[bovine gut metagenome]
MAHKKNIGAEIVKTYSFKVKNTNGITMEKLMNAIDEYQSYYNLCSDWICKNLTTMTIGDLDRYIPEKAKDNIYATVLLDEVWKN
QPLYKIFGKKYSSNNRNNALYCALSSVIDMTKENVLGFSKTHYIRNGYILNVISNYASKLSKLNTGVKSRAIKETSDEATIIEQ
VIYEMEHNKWESIEDWKNQIEYLNSKTDYNPTYMERMKTLSAYYSTHKSEVDAKMQEMAVENLVKFGGCRRNNSKKSMFIMGSN
TTNYTISYIGDNCFNINFANILNFDVYGRRDVVKNGEVLVDIMANHGDSIVLKIVNGELYADVPCSVTLNKVESNFDKVVGIDV
NMKHMLLSTSVTDNGSSDFVNIYKEMSNNAEFMALCPEKDRKYYKDISQYVTFAPLELDLLFSRISKQGEVKMEKAYSEILESL
KWKFFANGDNKNRIYVESIQKIRQQIKALCVIKNAYYEQQSAYDIDKTQEYIETHPFSLTEKGMSIKSKMDKICQTIIGCRNNI
IDLAYSFFERNGYSIIGLEKLTSSQFKNTKSMPTCKSLLNLHKVLGHTLSELETLPINDIVKYYTFTTDNEGRITDASLSEKGK
IRKMKDRFLNQAIKAIHFADVKDYFATLSNNGQTGIFFVPSQFTSQMDSNTHNLYFEVDKNGGLKMASKDKTRPKQEYHRNGLP
ADYNAARNIAYIGLDETMRNTFLKKVNSNKSLYNQPIYDTGIKKTAGVFSRMKKLKRYEII (SEQ ID NO: 6)
>SRR5371379_2478682_1|M
[bovine gut metagenome]
MIKSIKLKVKGDCPITKDVINEYKEYYNRCSDWIKNNLTSITIGEIGKFLQDVTGKTTGYIEVALSDKWKDKPMYYLFTDQYDT
NHANNLLYSFIQENNLDGYDGNSLNISGTYYRKQGYFKLVSSNYRTKIRTLNCKIKRKKVDVDSTSEDIESQVMYEIINRSLNK
KSDWDSFISYIENVENPNIDSINRYTLLRDYFCDNEDVIKNKIELLSIEQLKDFGGCIMKQHINTMSLNIQHFKIEEKENSLGF
ILYLPLNKKQYQIELWGHRQIKKGSKESCETLVDFINTYGENIVFTINNDELYVVFSYESEFGKEETNFEKSVGLDINFKHALF
VTSELDNDQFDGYINLYKYILSHSEFTNLLTEDERKDYEELSKVVTFCPFENQLLFARYDKMSKFCKKEQVLSKLLYSLQKKLK
NENRTKEYIYVSCVNKLRAKYISYFILREKYDEKNKEYDIEMGFVDDSTESKESMDKRRFENPFRNTLVANELLAKMSKVQQDI
NGCMSNIINYVYKVFEQNGYNIIALENLENSNFEKRQVLPTIKSLLKYHKLENQNINDIKASDKIKEYIENGYYSFTTNENNEI
VDAKYTAKGDIKVKNAKFFNLMMKILHFASIKDEFVLLSNNGKSQIALVPPEYTSQMDSIDHCIYMTENDKGKIVKVDKRKVRT
KQERHINGLNADFNAANNIKYIVSNEKWRNVFCTPKKAKYNTPALDATKKGQFRILDDMKKLNATKLLEIEK (SEQ ID NO:
7)
>SRR5371385_201181_1|P
[bovine gut metagenome]
MYQLNQYIMASHKKTESNQIIKTFSFKIKNANGLSLDVLNDAITEYQNYYNICSDWIKDHLTMKISELYKYIPDEKKNSGYALT
LISDEWKDKPMYMMFKKGYPANNRDNAIYETLNTCNTEHYTGNILNFSDTYYRRFGYVASAISNYVTKISKMSTGSRYKNISND
SDVDTIMEQVIYEMEHNGWTSVKDWENQMEYLESKTDSNPNFVYRMTTLYEFYKSHIDEVNSKMETMSIDSLIKFGGCRRKDSK
KSMYIMGGSNTPFDITQIGGNSLNIKFSKNLNVDVFGRYDVIKDNTLLVDIINGHGASFVLKIINDEIYIDINVSVPFDKKIAT
TNKVVGIDVNIKHMLLATNILDDGNVKGYVNIYKEVINDSDFKKVCNSTVMKYFTDFSKFVTFCPLEFDFLFSRVCNQKGIYND
NSAMEKSFSDVLNKLKWNFIETGDNTKRIYIENVMKLRSQMKAYAIVKNAYYKQQSEYDFGKSEEFIQEHPFSNTDKGIEILHK
LDNISKKILGCRNNIIQYSYNLFEINGYDMISLEKLTSSQFKKKPFPTVNSLLKYHKILGCTQEEMEKKDIYSVIKKGYYDIIF
DNGVVIDAKLSAKGELSKFKDDFFNLMIKSIHFADIKDYFITLSNNGTAGVSLVPSYFTSQMDSIDHKIYFVQDNKSGKLKLAN
KHKVRSSQEKHINGLNADYNAARNIAYIMENTECRNMFMKQSRTDKSLYNKPSYETFIKTQGSAVSKLKKDGFVKILDEASV
(SEQ ID NO: 8)
>SRR5371385_201181_1|M
[bovine gut metagenome]
MASHKKTESNQIIKTFSFKIKNANGLSLDVLNDAITEYQNYYNICSDWIKDHLTMKISELYKYIPDEKKNSGYALTLISDEWKD
KPMYMMFKKGYPANNRDNAIYETLNTCNTEHYTGNILNFSDTYYRRFGYVASAISNYVTKISKMSTGSRYKNISNDSDVDTIME
QVIYEMEHNGWTSVKDWENQMEYLESKTDSNPNFVYRMTTLYEFYKSHIDEVNSKMETMSIDSLIKFGGCRRKDSKKSMYIMGG
SNTPFDITQIGGNSLNIKFSKNLNVDVFGRYDVIKDNTLLVDIINGHGASFVLKIINDEIYIDINVSVPFDKKIATTNKVVGID
VNIKHMLLATNILDDGNVKGYVNIYKEVINDSDFKKVCNSTVMKYFTDFSKFVTFCPLEFDFLFSRVCNQKGIYNDNSAMEKSF
SDVLNKLKWNFIETGDNTKRIYIENVMKLRSQMKAYAIVKNAYYKQQSEYDFGKSEEFIQEHPFSNTDKGIEILHKLDNISKKI
LGCRNNIIQYSYNLFEINGYDMISLEKLTSSQFKKKPFPTVNSLLKYHKILGCTQEEMEKKDIYSVIKKGYYDIIFDNGVVIDA
KLSAKGELSKFKDDFFNLMIKSIHFADIKDYFITLSNNGTAGVSLVPSYFTSQMDSIDHKIYFVQDNKSGKLKLANKHKVRSSQ
EKHINGLNADYNAARNIAYIMENTECRNMFMKQSRTDKSLYNKPSYETFIKTQGSAVSKLKKDGFVKILDEASV (SEQ ID
NO: 9)
>SRR5371401_1055766_58|M
[bovine gut metagenome]
MIKSIQLKVKGECPITKDVINEYKEYYNNCSDWIKNNLTSITIGEMAKFLQSLSDKEVAYISMGLSDEWKDKPLYHLFTKKYHT
KNADNLLYYYIKEKNLDGYKGNTLNISNTSFRQFGYFKLVVSNYRTKIRTLNCKIKRKKIDADSTSEDIEMQVMYEIIKYSLNK
KSDWDNFISYIENVENPNIDNINRYKLLRECFCENENMIKNKLELLSVEQLKKFGGCIMKPHINSMTINIQDFKIEEKENSLGF
ILHLPLNKKQYQIELLGNRQIKKGTKEIHETLVDITNTHGENIVFTIKNDNLYIVFSYESEFEKEEVNFAKTVGLDVNFKHAFF
VTSEKDNCHLDGYINLYKYLLEHDEFTNLLTEDERKDYEELSKVVTFCPFENQLLFARYNKMSKFCKKEQVLSKLLYALQKKLK
DENRTKEYIYVSCVNKLRAKYVSYFILKEKYYEKQKEYDIEMGFVDDSTESKESMDKRRTEYPFRNTPVANELLSKLNNVQQDI
NGCLKNIINYIYKIFEQNGYKVVALENLENSNFEKKQVLPTIKSLLKYHKLENQNVNDIKASDKVKEYIENGYYELMTNENNEI
VDAKYTEKGAMKVKNANFFNLMMKSLHFASVKDEFVLLSNNGKTQIALVPSEFTSQMDSTDHCLYMKKNDKGKLVKADKKEVRT
KQERHINGLNADFNAANNIKYIVENEVWRGIFCTRPKKTEYNVPSLDTTKKGPSAILNMLKKIEAIKVLETEK (SEQ ID
NO: 10)
>SRR5371439_988701_11|M
[bovine gut metagenome]
MIKSIVFKVKGDCPITKDVIKEYKEYYNRCSEWIKNNLTSITIGEIGKFLQDTMGKTHGYIKVALSDEWKDKPMYYLFTEKYDT
KHANNLLYYFIQENNLDRYEGNSLNIPSYYYKREGYFKLVTSNYRTKIRTLNCKIKRKKIDVDSTCVDIENQVIYEIIKKGLNK
KSDWDNYISYIENIEMPNIDSINRYKLLRDYFCENENVIKNKIELLSIEQLKNFGGCIMKQHINTMILNIKRLKIEEKENSLGF
ILHLPLNKKQYQIELWGNRQIKKGTKESNETLVDFINTYGEDVVFTIKKNELYAKFSYECEFEKEETNFEKSVGLDINFKHALF
VTSELDDDQFYGYINLYKYILSHSEFTNLLTEDEKKDYEDLSNAITFCPFENQLLFTRYDKKSKLYKKEQVLSKILYSLQKKLK
DENRKQEYIYVSCVNKLRAKYVSYFILKEKYNEKQKEYDIEMGFVDDSTESKESMDKRRYEYPFRNTPVANELLEKMNNVQQDI
SGCLKNIINYAYKVFEQNGYNIVALENLENSNFEKRNVLPTIKSLLKYHKLENQNITDIKASDKIKEYIENGYYELITNENNEI
IDAKYTENGDIKVKNARFFNLMMKSLHFASIKDEFVLLSNNGKSQIALVPSEYTSQMDSTDHCIYMTENDKGKLVKVDKRKVRT
KQERHINGLNADFNAANNIKYIVENEKWRKVFCAPQKAKYNTPTLDATKKGQFRILEDLKKLKATKLLEIGK (SEQ ID NO:
11)
>SRR5371497_203858_6|M
[bovine gut metagenome]
MIKSIQLKVKGECPITKDVINEYKEYYNNCSDWIKNNLTSITIGEMAKFLQSLSDKEVAYISMGLSDEWKDKPLYHLFTKKYHT
KNADNLLYYYIKEKNLDGYKGNTLNISNTSFRQFGYFKLVVSNYRTKIRTLNCKIKRKKIDADSTSEDIEMQVMYEIIKYSLNK
KSDWDNFISYIENVENPNIDNINRYKLLRECFCENENMIKNKLELLSVEQLKKFGGCIMKPHINSMTINIQDFKIEEKENSLGF
ILHLPLNKKQYQIELLGNRQIKKGTKESHETLVDITNTHGENIVFTIKNDNLYIVFSYESEFEKEEVNFAKTVGLDVNFKHAFF
VTSEKDNCHLDGYINLYKYLLEHDEFTNLLTEDERKDYEELSKVVTFCPFENQLLFARYNKMSKFCKKEQVLSKLLYALQKKLK
DENRTKEYIYVSCVNKLRAKYVSYFILKEKYYEKQKEYDIEMGFVDDSTESKESMDKRRTEYPFRNTPVANELLSKLNNVQQDI
NGCLKNIINYIYKIFEQNGYKVVALENLENSNFEKKQVLPTIKSLLKYHKLENQNVNDIKASDKVKEYIENGYYELMTNENNEI
VDAKYTEKGAMKVKNANFFNLMMKSLHFASVKDEFVLLSNNGKTQIALVPSEFTSQMDSTDHCLYMKKNDKGKLVKADKKEVRT
KQERHINGLNADFNAANNIKYIVENEVWRGIFCTRPKKTEYNVPSLDTTKKGPSAILNMLKKIEAVKILETEK (SEQ ID
NO: 12)
>SRR5371501_2762794_1|M
[bovine gut metagenome]
MKNNLTTVTIGEMAKFLQETTGKNVTYITMGLSEEWKDKPLYHLFYGKYHTKNADNLLYYFIKAKKLDEYDGNMLNLGDTYYRQ
FGYFKLVVSNYRTKIRTLNLNVKRKRVDVDSTSEDIESQVMYEIVKRNLNTISDWENYISYIEDVETPNIDNINRYKFLQNYFC
ENEEDIKNKIEFLSIEQLKDFGGCIMKPHINSMTINIQDFKIEEIENSLGFVLQLPLNKKYHQIELYGNRQVKKGTKENYKTLV
DIINTHGENIVFTIENNELYVVFSYEYELKKKDINFEKMAGIDVNFKHALFVTSETDNNQLNHYINLYKHILEHNEFTTLLTDS
ERKDYEEIAKTVTFCPFEYQLLFTRFDKNSNANVKEQALSKILYDLQKKLKSQNKIKEYIYVSCVNKLRAKYVSYFILKEKYYE
KQKEYDIQMGFVDDSTESKSSMVKRRVEYPFRNTPVANALLAIVNNVQQDINGCLKNIINYAYKVFELNDYNVVALENLENANF
EKKQVIPTIKSLLKYHKLEMQNINDIKANDTIKKYIENEYYQLITNENNEIVNAIYTPKGITKLKYANFFNLLMKSLHFASIKD
EFILLSNNGNTNIALVPHEYTSQMDSIDHCIYMVQNDKGNLVKAHKTKVRTKQEKHINGLNADFNAANNIKYIVENEKWRNIFC
KIPKKIEYNTPVLDVTKKGQSNIIKTLKNLNATKILEIKK (SEQ ID NO: 13)
>SRR5678926_1309611_3|P
[terrestrial metagenome]
MKKS1KFKVKGNCPITKDVINEYKEYYNKCSDWIKNNLTSITIGEMAKFLQETLGKDVAYISMGLSDEWKDKPLYHLFTKKYHT
NNADNLLYYYIKEKNLDGYKGNTLNIGNTFFRQFGYFKLVVSNYRTKIRTLNCEIKRKKIDADSTSEDIEMQTMYEIIKHNLNK
KTDWDEFISYIENVENPNIDNINRYKLLRKCFCENENMIKNKLELLSIEQLKNFGGCIMKQHINSMTLIIQHFKIEEKENSLGF
ILNLPLNKKQYQIELWGNRQVNKGTKERDAFLNTYGENIVFIINNDELYVVFSYEYELEKEEANFVKTVGLDVNFKHAFFVTSE
KDNCHLDGYINLYKYLLEHDEFTNLLTNDEKKDYEELSKVVTFCPFENQLLFARYNKMSKFCKKEQVLSKLLYALQKQLKDENR
TKEYIYVSCVNKLRAKYVSYFILKEKYYEKQKEYDIEMGFVDDSTESKESMDKRRTEFPFRNTPVANELLSKLNNVQQDINGCL
KNIINYIYKIFEQNGYKIVALENLENSNFEKKQVLPTIKSLLKYHKLENQNVNDIKASDKVKEYIENGYYELITNENNEIVDAK
YTEKGAMKVKNANFFNLMMKSLHFASVKDEFVLLSNNGKTQIALVPSEFTSQMDSTDHCLYMKKNDKGKLVKADKKEVRTKQEK
HINGLNADFNAANNIKYIVENEVWREIFCTRPKKAEYNVPSLDTTKKGPSAILHMLKKIEAIKILETEK (SEQ ID NO:
14)
>SRR6059713_382107_4|P
[feces metagenome]
MAKSIMKKSIKFKVKGNSPINEDIINEYKGYYNTCSNWINNNLTSITIGEMGKFLKDVMRKTTGYIDVALSDEWKDKPMYYLFT
KKYNPKHANNLLYYFIKEKKLDKFNGNILNVPEYYYRKEGYFKLVAGNYRTKINTLNFKIKSKKVDANSLSEDIEMQTIYEIVK
RGLNKKSDWDSYISYIECVQNPNIDNINRYKLLRDYFCENEDVIKNKIEILSIEQIKEFGGCIMKPHINSMTFGIQKFKIEEIE
NSLGFTFNLPLNKNNYKIELWGHRQLKKGNKESNVNVSLDDFINTYGQNVVFTIKRKKLYIVFSYDYEFERGECNFEKSVGLDV
NFKHSLFVTSEIDNNQFDGYINLYKYILSNNEFTSLLTDSERKDYEDLANIVTFCPFEYQLLFSRYDKLSKISEKEKVLSKILY
SLQKKLKNEKRTKEYIYVSCVNKLRAKYVSYFKLKQKYNEKQKEYDIEMGFVDDSTESKESMDKRRFENPFINTPVAKELLEKM
NNVKQDINGCKKNIVVYAYKVLEQNGYNIIALENLENSNFEKIRVLPKIKSLLEYHKFENKNINDIKNSDKYKEFIEPGYFELI
TNENNEIIDAKYTQKGDIKIKNADFINIMIKALNFASIKDEFILLSHNGKSQIALVPAEYTSQMDSIDHCIYMTKNDKGKLVKV
DKRKVRTKQERHINGLNADFNAACNIKYIVTNEDWRKVFCIKPKKEDYNTPLLDATKNGQFRILDKLKKLNATKLLEMEK
(SEQ ID NO: 15)
>SRR6060192_2608084_13|P
[feces metagenome]
MANKKFKLTKNEVVKSFVLKVANQKKCAITNETLQEYKNYYNKVSQWINNNLTKMTIGDLIQYAPTVSKKGKKQPDGTMVYDTP
LYVTYAMSDEWKNKPLYYIFKKEYNTNNANNLLYEAIRNLNVDEYDGNQLNFNSTYYRTQGYVNRVFSNYRTKINTLDIKIKKS
KVDENSDVETLELQTMYEINKLNLKTNKDWEERLQYLTMQENPNQNTIDRTKILFNYFINNNDTIFQKMEELSIKQLTEFGGCK
MKDNTTSMTINIQDFKIKRKENSIGYIMTIPFNKKNVDVELYGHKQTIKGHKNSYTEIVDIVNKHGNTITFKIKNNQLFAIITS
DTEVTKPEPQYEKIVGVDVNIKHTLMVTSEKDNGKLKGYINLYKEVLKNDEFKKLLNKTELDNFKSLSQIVTFCPIEYDFLFSR
IFDDENTKKELAFSNVLYDIQKQLKNTNNILQYNYIACVNKLRAKYKAYFVLKMSYMKQQKIYDTNMGFFDISTESKETMDQRR
SLYPFINTEIAQNIITKMNNVQQDINGCLKNIFKYTYTVFENNNYDTIVLENLENANFEKHNPLPNITSLLKYHKVQGLTIQEA
EQHEKVGNLIQNDNYIFQLNEDNKIINADYSQKAYYKVCKALFFNQAIKTLHFASVKDEMIKLSNNNKVCVAIIPPEYTSQIDS
NTHKLYFINKDGKLLKADKKTVRKTQEKHINGLNADFNAASNIKYIVQNETWRNLFTNKTNNTYGLPILTPSKKGQSNIITQLM
KINATQELW (SEQ ID NO: 16)
>SRR7634052_1662339_24|M
[sheep gut metagenome]
MYNSKKKGEGDIQKSFKFKVKTDKETVELFRKAAVEYSEYYKRLTTFLCERLTDMTWGEVASFIPEKYRKNEYYKYLIKEENKD
LPLYKMFTKAASSMFIDHSIERYVEALNPEGNTGNILGFCKSSYVRGGYLKNVVSNIRTKFATLKTGIKYKKFNPAEDDEETIL
GQTVFEMEKRGLEFKCDFEKTIKYLNEKGKTQEAERLQCLMEYFSTNTDKINEYRESLVLDDIRKFGGCNRSKSNSFSVTLEKA
DIKEDGLTGYTMKVSKKLKEIHLLGHRRVVEVVNGRRVNLVDICGDKSGDSKVFVVDGDNLYVCISAPVKFSKNGMEAKKYIGV
DMNMKHSIISVSDNASDMKGFLNIYKELLKDEGFRKTLNATELEKYEKLAEGVNIGIIEYDGLYERIVKQKKENSVDGLKVQAE
KKLIEREAAIERVLDKLRKGTSDTDTENYINYNKILRAKIKSAYILKDKYYEMLGKYDSERAGSGDLSEENKIKYKDEFNETEK
GKEILGKLNNVYKDIIGCRDNIVTYAVNLFIRNGYDTVALEYLESSQMKARRIPSTGGLLKGHKLEGKPEGEVTAYLKANKIPK
SYYSFEYDGNGMLTDVKYSDMGEKARGRNRFKNLVPKFLRWASIKDKFVQLSNYKDIQMVYVPSPYTSQTDSRTHSLYYIETVK
VDEKTGKEKKEHIVAPKESVRTEQESFVNGMNADTNSANNIKYIFENETLRDKFLKRTKDGTEMYNRPAFDLKECYKKNSNVSV
FNTLKKTLGAIYGKLDENGNFIENECNK (SEQ ID NO: 17)
>AUXO017332817_2|M
[gut metagenome]
MAGHSKIKENHIMKAFLMKVKETRKKQWQSNFIRSEIAKFTNYYNGLSKFIADRLLDDMVTTLAPLIEEKKRNSEYYKYLTNGD
WDGKPLYFIFKEGFNSTNADNILANSLVRVYCEQNYTGNGFGLSYSYYVVIGFAKEVIANYRSSFQKPKVKIKKKKLSENPTED
ELIEQCIYTIYYEFNEKKDIQKWKDEIKFLKERGESKETRLKRIQTLFEFYKDKSHKELVDERVANLVVDNIKEFGGCKRDIDC
PSMGIQIQHNFDISINEKRNGYTICFGPNKKNLTKLEVFGNRMVLLNGEEIVDLPNTHGEKLTLIDRGNAIYAAITAQVPFEKH
MPDGNKTVGIDLNLKHSVFATSIVDNGKLAGYISIYKELLKDDEFVKYCPKDLLRFMKDASKYVFFAPIEIELLRSRVIYNKGY
ACVENYENVYKAEVAFVNVIKRLQSQCEANGDAQGALYMSYLSKMRAQLKNYINLKLAYYDHQSAYDLKMGFTDISTESKETMD
ERRKLFPFNKEKEAQEILAKMKNISNVIIACRNNIAVYMYKMFERNGYDFIGLEKLESSQMKKRQSRSFPTVKSLLNYHKLAGM
TMDEIKKQEVSSNIKKGFYDLEFDADGKLYGAKYSNKGNVHFIEDEFYISGLKAIHFADMKDYFVRLSNNGKVSVALVPPSFTS
QMDSVEHKFFMKKNANGKLIVADKKDVRSCQEKHKINGLNADYNAACNIGFIVEDDYMRESLLGSPTGGTYDTAYFDTKIQGSK
GVYDKIKENGETYIAVLSDDVITAEV (SEQ ID NO: 18)
>OQVL01000914_15|P
[human gut metagenome]
MAHKKNVGAEIVKTYSFKVKNTNGITMEKLMNAIDEFQSYYNLCSDWICKNLTTMTIGDLDQYIPEKAKGNTYATVLLDEAWKN
QPLYKIFGKKYSSNNRNNALYCALSSVIDMTKENVLGFSKTHYIRNDYILNVISNYASKLSKLNTGVKSRAIKETSDEATIIEQ
VIYEMEHNKWESIEDWKNQIEYLNSKTDYNPTYMERMKTLSAYYSTHKSEVDAKMQEMAVENLVKFGGCRRNNSKKSMFIMGSN
TTNYTISYIGGNSFNINFANILNFDVYGRRDVVKNGEVLVDIMANHGDSIVLKIVNGELYADVPCSVTLNKVESNFDKVVGIDV
NMKHMLLSTSITDNGSSDFLNIYKEMSNNAEFMALCPEEDRKYYKDISKYVTFAPLELDLLFSRISKQGKVKMEKVYSEILEAL
KWKFFANGDNKNRIYVESIQKIRQQIKALCVIKNAYYEQQSAYDIDKTQEYIETHPFSLTEKGMSIKSKMDKICQTIIGCRNNI
IDYAYSFFERNGYSIIGLEKLTSSQFEKTKSMPTCKSLLNFHKVLGHTLSELETLPINDVVKKGYYTFTTDNEGKITDASLSEK
GKVRKMKDDFFNQAIKAIHFADVKDYFATLSNNGQTGIFFVPSQFTSQMDSNTHNLYFENAKNGGLKLAPKYKVRQTQEYHLNG
LPADYNAARNIAYIGLDETMRNTFLKKANSNKSLYNQPIYDTGIKKTAGVFSRMKKLKRYEII (SEQ ID NO: 19)
>3300001598|EMG_10017415_6|P
[mammals-digestive system-asian elephant fecal-elephas maximus]
MLNIKNNGESVDMNTIELAMKEYNRYYNICSDWICNNLMTPIGSLYQYIDDKCKNNAYAQNLIAEEWKDKPLYYMFYKGYNANN
CANAICCAIRSQVPEVNKAENILNLSYTYYFRNGVIKSVISNYASKMRILSDKQIKYCIVSENTPDKILIEQCILELKRRHEDL
KDWEENLKYLILKGNESAITRFTILKDFYSKNIERVKEEREIMAIAELKDFGGCRRKDDKLSMCIQSAGNSKDIKVSRVKTTHN
YTELVDDYTENFNIKFSALDFNVMGRRDVVKTKLNKTEDDSNTWGGTELLVDIINNHGCSLTFKLVDDKLYVDIPIDTEHINKT
TDFKKSVGIDVNLKHSLLNTDILDNGGINGYINIYKKLLADDAFMSACTKADLVNYIDIAKTVTFCPIEADFIISNVVEKYLHM
KDNTNKMEIAFSSVLMNIRKELEIKLLHSSKEESPLIRKQIIYINCIICLRNELKQYAIAKHRYYKKQQEYDTLCDTLHGVDYK
QIHPYAQSKEGAEQMKKMKTIENNLIANRNNIlEYAYTVFELNNFDLIALENITKDIMEDKKKRKSFPSINSLLKYHKVINCTE
DNINDNETYQKFAKYYNVSYENGKVTGATLSQEGNKVKLKDDFYDKLLKVLHFTSIKDYFTTLSNKRKIAVAHVPAYYTSQIDS
IDNKICMIKSTDKNGKSTYKIADKTIVRPTQEKHINGLNADYNAARNINFIVADEKWRKKFVRPTNTNKPLYNSPVFSPAVKSE
GGTIKNLQILSATKTIIL (SEQ ID NO: 20)
>3300021254|Ga0223824_10022219_2|P
[mammals-digestive system-cattle and sheep rumen]
MAHVRTKNEGNMAKTYSFKVRETNLKKDVMIEYNEYYNRLSDWICGNLTKMTIGELAELVPEKKRNTSYYLAATDEKWINEPMY
KLFTDEYTKKSSFTDPLVANSNNCDNLILTATDVLNPEGYEGNLLSLCKSTYRTFGYAKQIISNMKTKIGALKPNVKRRVLGEN
PTYDEKMIQVLYEMYNNGIADVTGFNDRIKYLKKQETPNEKLISRMKMLRDFFKENRNDIMDKCRIMAVEQLVSFGGCKRNING
ASMTLRNQCISVKRKDGCQGYVVAIPVGTKNSIVFDLYGRRDVIKDGVELVDVCGKHTDTITIKSVNGELFLDMPVAINFEKKS
GKCTKTVGIDVNTKHMLIQTSVKDNGKFDYYVNLYKIFAEDEELNKILGDDEVMVNIKKNAENLSFLPLEMDLLYSRILDGPQK
YKLAEDRITELLKQWGINFDAGCMSQERIYVQCVRKLRGNLKRLLYLQNKYYEAQQEYDKKMGFDDKSTDSKETMDKRRWESPF
RNTEEGTKLYDEINTYQNRIIGIRNSIIDYAYLVLEYNGYDNLSLEYLTSSQFKVNKTFPTTNSLLKYHKLQGKTKTEAEKCDA
YISHKSKYKLSLKDGVIDSIDYSAEGLKQIKKDRSRNIIIKAIHFADVKDRFVLSSNNGNASVTFVPSYHTSQIDSTDHKMFVT
NKGKIVDKRKVRQIQETHVNGLNSDFNAARNIQYISENEEWRNALCKPTENMYNEPIYVPLVKSQNGMFKAIKKLGATKIWQE
(SEQ ID NO: 21)
>3300021431|Ga0224423_10015012_2|P
[mammals-digestive system-cattle and sheep rumen]
MAHRNKNLAENCINKTFSFKVKAEKEEINSKWIPAIKEYTAYYNRISDWICDRLTNTTVGELIGIIGYKTDKKGNALAYIKDGS
SEKYRNLPLYCMFKKNFPATTADNIMYQVIEKLGVDKYNGNSLGLSGTYYRRIGYIANVIGNYRTKVRGMKASVKYRNFDPNDV
TEDVLENQTIFEINKNGFECKGDFEKHIEYLKNRELTDRLNKLILRMECLYNYYVEHEDAVKAKMENYAIESFKTFGGCHRNSN
RSMSIQFTNNSPLEIKKVGKTSFDLYMPINGEVACLQLMGNKQAVCVGENGERCDLVDIVNSHSKTITIKIINGEMYVDIPCVV
NFEKKDEDTIKSVGVDVNIKHEILATSVIDNGQLNGYFNIYKELINNKEFVDTFNGDIKAFEAFKDNAAYVTFGLLEPDLLFTR
FYERSGFEKDDRHIKLRERERILTGILKRIGQEHSDVDVRNYVRFVNMLRSKYESYFVLKNKYYEKMQEFDSTQNYVDVSTASK
ETMDKRRFDNPFRNTEVANELLGKIDNVLGDIKGCMANIITYAFKVLQKNGYNTIGLEYLDSSQFENMRTLTPTSILKYHKMEG
KSVDAVESWIKENKIPSNRYDFIYEDNHLTDVLLNSNGIAYQKKNLFMNLVIKAISFADIKNKFVQLSNNTNVSILFAPAAFTS
QMDSNRHVIYTVKNNKGKLALVDKKRVRPNQEKHINGLHSGYNAACNVKFICDNEFFRNTMTISNKGKNLYSQPTYDIKEAYKK
NAGCKVINDFIKNGNAVICCIENNKLIETNGRQ (SEQ ID NO: 22)
>3300012973|Ga0123351_1009859_3|P
[mammals-digestive system-fecal]
MANKKFKLTKNEVVKSFVLKVANQKKCAITNETLQEYKNYYNKVSQWINNNLTKMTIGDLIQYAPTVSKKGKKQPDGTMVYDTP
LYVTYAMSDEWKNKPLYYIFKKEYNTNNANNLLYEAIRNLNVDEYDGNQLNFNSTYYRTQGYVNRVFSNYRTKINTLDIKIKKS
KVDENSDVETLEPQTMYEINKLNLKTNKDWEERLQYLTMQENPNQNTIDRTKILFNYFINNNDTIFQKMEELSIKQLTEFGGCK
MKDNTTSMTINIQDFKIKRKENSIGYIMTIPFNKKNVDVELYGHKQTIKGHKNSYTEIVDIVNKHGNTITFKIKNNQLFAIITS
DTEVTKPEPQYEKIVGVDVNIKHTLMVTSEKDNGKLKGYINLYKEVLKNDEFKKLLNKTELDNFKSLSQIVTFCPIEYDFLFSR
IFDDENTKKELAFSNVLYDIQKQLKNTNNILQYNYIACVNKLRAKYKAYFVLKMSYMKQQKIYDTNMGFFDISTESKETMDQRR
SLYPFINTEIAQNIITKMNNVQQDINGCLKNIFKYTYTVFENNNYDTIVLENLENANFEKHNPLPNITSLLKYHKVQGLTIQEA
EQHEKVGNLIQNDNYIFQLNEDNKIINADYSQKAYYKVCKALFFNQAIKTLHFASVKDEMIKLSNNNKVCVAIIPPEYTSQIDS
NTHKLYFINKDGKLLKADKKTVRKTQEKHINGLNADFNAASNIKYIVQNETWRNLFTNKTNNTYGLPILTPSKKGQSNIITQLM
KINATQELW (SEQ ID NO: 23)
>3300012979|Ga0123348_10005323_4|M
[mammals-digestive system-fecal]
MAKSIMKKSIKFKVKGNSPINEDIINEYKGYYNTCSNWINNNLTSITIGEMGKFLKDVMRKTTGYIDVALSDEWKDKPMYYLFT
KKYNPKHANNLLYYFIKEKKLDKFNGNILNVPEYYYRKEGYFKLVAGNYRTKINTLNFKIKSKKVDANSLSEDIEMQTIYEIVK
RGLNKKSDWDSYISYIECVQNPNIDNINRYKLLRDYFCENEDVIKNKIEILSIEQIKEFGGCIMKPHINSMTFGIQKFKIEEIE
NSLGFTFNLPLNKNNYKIELWGHRQLKKGNKESNVNVSLDDFINTYGQNVVFTIKRKKLYIVFSYDYEFERGECNFEKSVGLDV
NFKHSLFVTSEIDNNQFDGYINLYKYILSNNEFTSLLTDSERKDYEDLANIVTFCPFEYQLLFSRYDKLSKISEKEKVLSKILY
SLQKKLKNEKRTKEYIYVSCVNKLRAKYVSYFKLKQKYNEKQKEYDIEMGFVDDSTESKESMDKRRFENPFINTPVAKELLEKM
NNVKQDINGCKKNIVVYAYKVLEQNGYNIIALENLENSNFEKIRVLPKIKSLLEYHKFENKNINDIKNSDKYKEFIEPGYFELI
TNENNEIIDAKYTQKGDIKIKNADEINIMIKALNFASIKDEFILLSHNGKSQIALVPAEYTSQMDSIDHCIYMTKNDKGKLVKV
DKRKVRTKQERHINGLNADFNAACNIKYIVTNEDWRKVFCIKPKKEDYNTPLLDATKNGQFRILDKLKKLNATKLLEMEK
(SEQ ID NO: 24)
>3300028797|Ga0265301_10000251_12|M
[mammals-digestive system-rumen-bos taurus]
MVKVFINVFLSEKNQITTNIFDTEKISNSYINHINHQFMATHKKTDNQTIVKAYVMKAKMSKHDIERVWKPTIDEYINYYNKLS
DWICKNLTSVTIGDLLKYVGEKQINKGVGYYTYFIDEQKTDLPLYTLFTDCPKTHADNLLFEAVRKINPENYNGNLLSLFETGY
RRNGYFDNVISNYRTKMTTLKINPKYKRFSSENMPTDEVLLEQTVYEVTKNDFKNDDDWKKSIDYMKQKSEPNTALIFRMETLF
DYWKDHKQDVEQYINQKRVECLKDFGGCKRRADGLSMVILLNKKLTKIEADGLTSYKLTTNLFGGKYMINIFGHRALVSVCNGE
RAENENIDICNKHGERFTFKIENGNLFVALTADYNYEKQPNLPKNIVGVDINIKHSMLNSSIEDKGKVKGYVNLYKEFLSDKNF
RKTITSDEELNQYIELSKYATFGITELDSLFARATDTEKSILCKRELAMQDVFEKLEKRYKDDHKIKFYLGSTQKLRAQYISYF
KIKEAYNRKQQEYDLAHGKTDNPDEVYKSDFINEPSAKEMLVKLNRIERKIIGCRNNIVTYAFNVIKNNGYDTIGVEYLTSSQF
EKKRRLPSIKSLLNYRKLLGKPKDEWNLKEWNDVYMCYRPELDDAGNIMNFTITNEGIKRNKESTFYNSFIKAIHFADVKDKFA
QLTNNNTMNTVFIPSSFTSQIDSKTRKLYLLEYTEKCDNGKTKKVVKFINKRVLRKIQEQHLNGMNADNNAARNIRDITKNLRD
VFTKKQTDKNCYNSAEFMIQTKFKKRLPQATVFGELNRNGYVKVLTQEEYDELTKSAK (SEQ ID NO: 25)
>3300028797|Ga0265301_10000251_10|P
[mammals-digestive system-rumen-bos taurus]
MATHKKTDNQTIVKAYVMKAKMSKHDIERVWKPTIDEYINYYNKLSDWICKNLTSVTIGDLLKYVGEKQINKGVGYYTYFIDEQ
KTDLPLYTLFTDCPKTHADNLLFEAVRKINPENYNGNLLSLFETGYRRNGYFDNVISNYRTKMTTLKINPKYKRFSSENMPTDE
VLLEQTVYEVTKNDFKNDDDWKKSIDYMKQKSEPNTALIFRMETLFDYWKDHKQDVEQYINQKRVECLKDFGGCKRRADGLSMV
ILLNKKLTKIEADGLTSYKLTTNLFGGKYMINIFGHRALVSVCNGERAENENIDICNKHGERFTFKIENGNLFVALTADYNYEK
QPNLPKNIVGVDINIKHSMLNSSIEDKGKVKGYVNLYKEFLSDKNFRKTITSDEELNQYIELSKYATFGITELDSLFARATDTE
KSILCKRELAMQDVFEKLEKRYKDDHKIKFYLGSTQKLRAQYISYFKIKEAYNRKQQEYDLAHGKTDNPDEVYKSDFINEPSAK
EMLVKLNRIERKIIGCRNNIVTYAFNVIKNNGYDTIGVEYLTSSQFEKKRRLPSIKSLLNYRKLLGKPKDEWNLKEWNDVYMCY
RPELDDAGNIMNFTITNEGIKRNKESTFYNSFIKAIHFADVKDKFAQLTNNNTMNTVFIPSSFTSQIDSKTRKLYLLEYTEKCD
NGKTKKVVKFINKRVLRKIQEQHLNGMNADNNAARNIRDITKNLRDVFTKKQTDKNCYNSAEFMIQTKFKKRLPQATVFGELNR
NGYVKVLTQEEYDELTKSAK (SEQ ID NO: 26)
>3300028797|Ga0265301_10009039_3|M
[mammals-digestive system-rumen-bos taurus]
MAHKGEKEGYQIKTLKFKVRSHDIGKSLYDIVNEYTNYYNKVSKWICDNLDTPIGELSKNISEKRHNSKYYRATNDPNWKNEPM
WKIFTKKFSNGETFSEQGKNDKLANLSNCDNILSYSIIDYNIDGYTGNILGLTDTSYRLNGYISNCISNYKTKIRTAKPKVRST
AITEHSTVEEKTNNTIYEMVRKGFMSPNDFKNQIKYLTEKENPNDKLIDRLSILHSFYTENEEDVNNAFSRMSVEMLKNNNGCT
RNGDKKTLNISSIDYKVTRKEGCDGYILSFGSRNQKYNIDLWGRRDTISNGKELIDLSEHGEPLTITSENGDYYVCMTVDVPFE
KKSTGSTEKVASVDVNTKHTMLSTDVIDDGTLKGYLNIYKKLLLDTELTSLLHKQDFDDMKELSHNVCFGPIEYNFLLSRILDL
DAYEKKVEDRITHSMKEMLKTETDERNKMYLGSVIKMRALLKVYISTKNRYHKEQQSYDESMGFTDTSTASKDTMDKRRFENPF
SETETGKKLNNDLSALSKKIIGCRDNIVRYAYTTLQDNGYTMIGVEDLNSSTFANTRNPFPTIKSLLNYHHLSGKTPEEARNID
TYSKFSDHYTLTTDEEGKITDAKYTKKAETKIKKKRARDTIIKAIHFAEVKDVMCVMSNNGTASVAFEPSYFSSQMDSATHKVY
TTRNKKGKDVIASKETVRPRQEKHINGMNCDINSPKNLSYLITNEEFREMFLTPTKNGYNEPFYKSRVKSAASMMSGLKKLGAT
MPLTDENAIFSTPKPKKNIGKQ (SEQ ID NO: 27)
>3300028887|Ga0265299_10000013_320|P
[mammals-digestive system-rumen-bos taurus]
MGNKVQSNETIVKTYTFKVREFISGATHEIMKSAIKQYIEDSNNLSDWINNQLTNKTICEVGALIPIEKRETSYYKSTVDELWA
NKPCFKMFTNDFTKEENFATRNIGNGKNCKNIITSAYKSTVNPSFRNVLDLTEKVYFSDGYGANVCSNYKTKLRTLKPAKIKLV
SSLSDCDDNTLTEQVIREKQKYGYSTPKDFEKRIEYLNEKEKSEQNSKIIERLQKLYEFYDNNTKLVEEKELELSVKSLVEFGG
CRRGEKTMTLNLPDIGYEIQRKDDKYGYIFTLKCSKKRKIIIDVWGSKATIDSNGNDKVDIINTHGKSINFKIINNEMYIDITV
DVPFAKRKLGIKKVVGIDVNTKHMLMATNIKVTDSIKGYVNLYKEFLNSKEIMDVASPETKKNFEDMSMFVNFCPIEYNTMFAL
IFKLNNGDIRTEQAIRRTLHQLSKKFSDGNHETERIYVQNVFSIREQLKHFILLSNRYYSEQSDYDTKMGFIDENTTSNATMDK
RRFDKSLMFRYTQRGRQLYEERIECGRKITEIRDNIITYARNVFVLNGYDTIALEYLTNATIQKPTRPTSPKSLLDYFKLKGKP
VVEAEKNERITKNRKYYNLIPDENDNVINIEYTEEGKVAIKKSIARDHIMKAVHFAEVKDKFIQLSNNGKTQVALVPSNYTSQM
NSETHTVYLMKNPKTKKLVIMDKDKVRPIQEKYKLNGLNADFNSARNIAYIVENEILRNSFLKEETKKYTYNTPLFTPRLKSSE
KIITELKKLGMTTVIE (SEQ ID NO: 28)
>3300028887|Ga0265299_10000026_77|P
[mammals-digestive system-rumen-bos taurus]
MANKSTKGNLPKTIIMKANLSPDGFTQWERVVKEYQAYKDTLSKWVAQNLTAMKIGDLLPYLDKYSKKTNKETGERPVNVYYQL
CEQHKDEPLYKLFTYDSNSRNNAMYEIIRKTNCDGYKGNILGISETHYRRNGFVKNILANYTTKISTLELSERKRKIDSDSPED
LIRSQVVYEMQKNNIKDAKGFKSIIEYLKSKKEVNIQYLERLQILYEYFKNHENEIKEYITLAAVEQLKSFGGVRVNNEKSSMN
LEIQGFSITRVDGACTYILHLPINGKIHGIKLWGNRQVVVNKDGTPVDILDLTNQHGSTINITIKNGEIYFAFTVTSDFVKPEH
QIKNVVGVDVNTKHMLMQSNITDNGNVKGYFNIYKVLVEDRRFTSLLSEEQLKYFCELANIVSFCPIETEFLFARYAEYKKMSN
NAEMRQIEKVFSDILDEQYKKYKDIDTSIANYISYVRKLRSQCCAYFKLKMKYKELQRQFDKEQDYKDLSTESKETMDKRRWEN
PFRNTPEASKLIKKMDNVSRQLIGCRDNIITYAYRVFEKNGYDTISLENLESSQFENNDHVIAPKSLLEYHHLKGKTMNYLLSD
ECKVRITTKDGKVKEWYHVELNDKDEIDNIFLTPEGETEKEKNLFNNMVIKIVHFADIKDKFIQLGNYNKLQTVLVPSYFTSQM
DSKTHSVYVVETANTKTSKKELKLVSKKRVRRQQEWHINGLNADYNAACNIAHIAKNIELRQIMCKTPQTKNGYSSPVLTSKVK
SQVEMVRELKKMGKTILYSNDSLPF (SEQ ID NO: 29)
>3300028887|Ga0265299_10000133_30|M
[mammals-digestive system-rumen-bos taurus]
MAHRKKKDDEATLSYKFKVKVIEGDLTADDITKCIAENAEQGNHFSEFIHKNLTSKTIGEFASQLPVEKRQFGYYQYAIGGTMP
AKKNASDEDKPKGELIDWSKKPFYVLFSKGYSATHAVNLIFNVYLNSEEGKAFSAKNSMNLSKSQFAYSGFVQIVCANYASMLA
NARPDKIKFEEITEATDDGTKKMQVVREMAERYLMKPKNFASRIEYLEANNTKGKFDKTIQRLRLLQPFFEKNEEGITELYYDL
SVKALEHSGQCTYKGGRTISILEIGDIRISRKENAKGYLLTIPINRKSVVFDLYGRKDTIGGDGRDLIDIMNTHGSSLQFTADG
NDIYLTITATKNFIKEKPTFNEDTVLGGDVNIKHSYTVFSTSPKDIPDFVNFYEYFAKDGEIMKLAPKPMWDYIVAAATKFLTI
LPIETPAISATVYGKRTEEGISRATFRETQKLIALEKAIERVMKQVFDKYNDGKHPLEAIYIGNAIKYRRLIKGYLAQKKKYYS
AHSEYDKAMGYTDDDTDRKENMDERRFDDSKKFRYTPEAQALLDTMHTIEKKIVGCVSNAISYAYHKFDENGFNVIALENLTSA
TFAKKYKSDKPESIKKLLNFDKLLGKTLDEAKASKSISKHPNWYELVADENGCVSDIRITDEGQSATYRSLVTETIMKVSHFAE
TKDRFIGLANSGRLQVGLVPSQYTSYIDSTTHTLYAVIEDGKTVLAPKEVVRASQERHINGLNADYNSALNLKYMITDENFRKT
FTSETSADKFGWGKPMFSPTTRSQDEVFSAIKKIGAITVLED (SEQ ID NO: 30)
>3300028887|Ga0265299_10011526_3|M
[mammals-digestive system-rumen-bos taurus]
MAQHKSNNEESAINKTFIFKAKCEKNDVISLWEPAAKEYGDYYNKVSKWIADNLITMKIGDLAQYITNQNSKYYTAVTNKKKKD
LPLYRIFQKGFSSQCADNALYCAIKSINPENYKGNSLGIGESDYRRFGYIQSVVSNFRTKMSSLKVSVKYKKFDVSNVDDETLK
IQTIYDVDKYGIETAKEFKELIETLKTRVETPQLNDTIARLKCLCDYYSKNEKAINNEIETMAIADLQKFGGCQRKSLNAFTIH
KQDSLMEKVGNTSFRLQLSFRKKTYVINLLGNRQVVNFVNGKRVDLIDIAENHGDLITFNIKNGELFLHITSPIVFDKDVRDIR
NVVGIDVNIKHSMLATSIKDDGNVKGYINLYKELLNDDVFVSTCNESELALYRQMSENVNFGILETDSLFERIVNQSKGGCLKN
KLIRRELAMQKVFERITKTNKDQNIVDYVNYVKMMRAKCKASYILKEKYDEKQKEYYVKMGFTDESTESKETMDKRREEFPFVN
TDTAKELLVKQNNIRQDIIGCRDNIVTYAFNVFKNNEYDTLSVEYLDSSQFDKRRIPTPKSLLKYHKFEGKTKDEVENMMKSEK
LSNAYYTFKYENDVVSDIDYSDEGNLRRSKLNFGNWIIKAIHFADIKDKFVQLSNNNKMNIVFCPSAFSSQMDSITHTLYYVEK
ITKNKKGKEKKKYVLANKKMVRTQQETHINGLNADYNSACNLKYIALNYELRDKMTDRFKASKKIKTMYNIPAYNIKSNFKKNL
SAKTIQTFRELGHYRDGKINEDGMFVEILE (SEQ ID NO: 31)
>3300028887|Ga0265299_10012919_3|P
[mammals-digestive system-rumen-bos taurus]
MAHKNSDGENTINKTFIFKVKCEKNDIISFWKPAAEEYCNYYNKLSEWIGKNLISMKIGDLAKYIDNPKSKYYLSVTDENKKDL
PLYKIFQKGFSSIDADNALYCAIDKLNPEGYNGNILGVGKSDYRRNGYVSSVIGNFRTKMVSLKANVRWKKIDIGNVDEETLRR
QTICDVEKYRIESEKDFRDLIDILKAREETPRLKEKISRLELLYDYYSKNTKTIKSEMENMAISDLQKFGGCVRKSLNTITIHK
QDSKIEKEGNTSFRLHMVFNKKPYTITLLGNRQVVKYIDGKRVDIVNIVEKHGDWITFNIKNGELFVHLTKCVEFSKGQKEIKK
AAGVDVNIKHAMLAASIVDDGQLKGYVNLYRELIEDDDFVSTFGDSDSGKTELGMYQKMAKTVFFGVLEVESLFERVVNQQSGW
KLDNQLIRRERAMEKVFDRIVKTTSNKHIIDYVNYVKMLRAKYKAYFILDEKYHEKQREYDLSMGFTDESDERRELYPFINTET
AKEILGKKRNVEQDLIGCRDNIVTYAFNVLRNNGYDTISVEYLDSSQFDKRRMPTPKSLLEYHKFKGKTQDEVERLMSEKKFAK
TNYDIHYDGENKVDGIVYSKEGELRQKKLNFMNLVIKAIHFADIKDKFAQLCNNNDVNVVFGPSAFTSQMDSETHSLYYVEKET
NGKNGKTGKKFVLADKKSVRRRQETHINGLNADFNAARNLEYIASNPELLERMTKRTKSGKDMYNTPSWNIRQEFKKNLSVRTI
NTFRELGNVKYGKINNEGLFVEDDV (SEQ ID NO: 32)
>3300028914|Ga0265300_10009460_3|M
[mammals-digestive system-rumen-bos taurus]
MAHRKKKDDEATLSYKFKVKVIEGDLTADDITKCIAENAEQGNHFSEFIHKNLTSKTIGEFASQLPAEKRQFGYYQYAIGGTMP
AKKNASDEDKPKGELIDWSKKPFYVLFSKGYSATHAVNLIFNVYLNSEEGKAFSAKNSMNLSKSQFAYSGFVQIVCANYASMLA
NARPDKIKFEEITEATDDGTKKMQVVREMAERYLMKPKNFASRIEYLEANNTKGKFDKTIQRLRLLQPFFEKNEESITELYYDL
SVKALEHSGQCTYKGGRTISILEIGDIRISRKENAKGYLLTIPINRKSVVFDLYGRKDTIGGDGRDLIDIMNTHGSSLQFTADE
NDIYLTITATKNFIKEKPTFNEDTVLGGDVNIKHSYTVFSASPKDIPDFVNFYEYFAKDGEIMKLAPKPMWDYIVAAATKFLTI
LPIETPAISATVYGKRTEEGISRATFRETQKLIALEKAIERVMKQVFDKYNDGKHPLEAIYIGNAIKYRRLIKGYLAQKKKYYS
AHSEYDKAMGYTDDDTDRKENMDERRFDDSKKFRYTPEAQALLDTMHTIEKKIVGCVSNAISYAYHKFDENGFNVIALENLTSA
TFAKKYKSDKPESIKKLLNFDKLLGKTLDEAKASKSISKHPNWYELVADENGCVSDIRITDEGQSATYRSLVTETIMKVSHFAE
TKDRFIGLANSGRLQVGLVPSQYTSYIDSTTHTLYAVIEDGKTVLAPKEVVRASQERHINGLNADYNSALNLKYMITDENFRKT
FTSETSADKFGWGKPMFSPTTRSQDEVFSAIKKIGAITVLED (SEQ ID NO: 33)
>3300031853|Ga0326514_10013355_6|M
[mammals-digestive system-rumen-bos taurus]
MVTTLAPLIEEKKRDSEYYKYLTNGDWDGKPLYFIFKEGFNSTNADNILANSLVRVYCEQNYTGNGFGLSYSYYVVIGFAKEVI
ANYRSSFQKPKVKIKKKKLSENPTEDELIEQCIYTIYYEFNEKKDIKKWKDEIKFLKERGESKETRLKRIQTLFEFYKDKNHKE
LVDERVANLVVDNIKEFGGCKRDIGCPSMGIQIQHNFDISINEKRNGYTICFGPNKKNLTKLEVFGNRMVLLNGEEIVDLPNTH
GEKLTLIDRGNAIYAALTAQVPFEKHMPDGNKTVGIDLNLKHSVFATSIVDNGKLAGYISIYKELLKDDEFVKYCPKDLLRFMK
DASKYVFFAPIEIELLRSRVIYNKGYACVENYENVYKAEVAFVNVIKRLQSQCEANGDAQGALYMSYLSKMRAQLKNYINLKLA
YYDHQSAYDLKMGFNDISAESKETIDERRKLFPFSKEKEAQEILAKMKNISNVIIACRNNIAVYMYKMFERNGYDFIGLEKLES
SQMKKRQSRSFPTVKSLLNYHKLAGMTMDEIKKQEVSSNIKKGFYDLEFDADGKLYGAKYSNKGNVHFIEDEFYISGLKAIHFA
DMKDYFVRLSNNGKVSVALVPPSFTSQMDSVEHKFFMKKNANGKLIVADKKDVRSCQEKHKINGLNADYNAACNIGFIVEDDYM
RESLLGSPTGGTYDTAYFDTKIQGSKGVYDKIKENGETYIAVLSDDVITAEE (SEQ ID NO: 34)
>3300031993|Ga0310696_10000014_323|P
[mammals-digestive system-rumen-bos taurus]
MGNKVQSNETIVKTYTFKVREFISGATHEIMKSAIKQYIEDSNNLSDWINNQLTNKTICEVGALIPIEKRETSYYKSTVDELWA
NKPCFKMFTNDFTKEENFATRNIGNGKNCKNIITSAYKSTVNPSFRNVLDLTEKVYFSDGYGANVCSNYKTKLRTLKPAKIKLV
SSLSDCDDNTLTEQVIREKQKYGYSTPKDFEKRIEYLNEKEKSEQNSKIIERLQKLYEFYDNNTKLVEEKELELSVKSLVEFGG
CRRGEKTMTLNLPDIGYEIQRKDDKYGYIFTLKCSKKRKIIIDVWGSKATIDSNGNDKVDIINTHGKSINFKIINNEMYIDITV
DVPFAKRKLGIKKVVGIDVNTKHMLMATNIKVTDSIKGYVNLYKEFLNSKEIMDVASPETKKNFEDMSMFVNFCPIEYNTMFAL
IFKLNNGDIRTEQAIRRTLHQLSKKFSDGNHETERIYVQNVFSIREQLKHFILLSNRYYSEQSDYDTKMGFIDENTTSNATMDK
RRFDKSLMFRYTQRGRQLYEERIECGRKITEIRDNIITYARNVFVLNGYDTIALEYLTNATIQKPTRPTSPKSLLDYFKLKGKP
VVEAEKNERITKNRKYYNLIPDENDNVINIEYTEEGKVAIKKSIARDHIMKAVHFAEVKDKFIQLSNNGKTQVALVPSNYTSQM
NSETHTVYLMKNPKTKKLVIMDKDKVRPIQEKYKLNGLNADFNSARNIAYIVENEILRNSFLKEETKKYTYNTPLFTPRLKSSE
KIITELKKLGMTTVIE (SEQ ID NO: 35)
>3300031993|Ga0310696_10000226_76|P
[mammals-digestive system-rumen-bos taurus]
MANKSTKGNLPKTIIMKANLSPDGFTQWERVVKEYQAYKDTLSKWVAQNLTAMKIGDLLPYLDKYSKKTNKETGERPVNVYYQL
CEQHKDEPLYKLFTYDSNSRNNAMYEIIRKTNCDGYKGNILGISETHYRRNGFVKNILANYTTKISTLELSERKRKIDSDSPED
LIRSQVVYEMQKNNIKDAKGFKSIIEYLKSKKEVNIQYLERLQILYEYFKNHENEIKEYITLAAVEQLKSFGGVRVNNEKSSMN
LEIQGFSITRVDGACTYILHLPINGKIHGIKLWGNRQVVVNKDGTPVDILDLTNQHGSTINITIKNGEIYFAFTVTSDFVKPEH
QIKNVVGVDVNTKHMLMQSNITDNGNVKGYFNIYKVLVEDRRFTSLLSEEQLKYFCELANIVSFCPIETEFLFARYAEYKKMSN
NAEMRQIEKVFSDILDEQYKKYKDIDTSIANYISYVRKLRSQCCAYFKLKMKYKELQRQFDKEQDYKDLSTESKETMDKRRWEN
PFRNTPEASKLIKKMDNVSRQLIGCRDNIITYAYRVFEKNGYDTISLENLESSQFENNDHVIAPKSLLEYHHLKGKTMNYLLSD
ECKVRITTKDGKVKEWYHVELNDKDEIDNIFLTPEGETEKEKNLFNNMVIKIVHFADIKDKFIQLGNYNKLQTVLVPSYFTSQM
DSKTHSVYVVETANTKTSKKELKLVSKKRVRRQQEWHINGLNADYNAACNIAHIAKNIELRQIMCKTPQTKNGYSSPVLTSKVK
SQVEMVRELKKMGKTILYSNDSLPF (SEQ ID NO: 36)
>3300031993|Ga0310696_10000447_27|M
[mammals-digestive system-rumen-bos taurus]
MAHRKKKDDEATLSYKFKVKVIEGDLTADDITKCIAENAEQGNHFSEFIHKNLTSKTIGEFASQLPVEKRQFGYYQYAIGGTMP
AKKNASDEDKPKGELIDWSKKPFYVLFSKGYSATHAVNLIFNVYLNSEEGKAFSAKNSMNLSKSQFAYSGFVQIVCANYASMLA
NARPDKIKFEEITEATDDGTKKMQVVREMAERYLMKPKNFASRIEYLEANNTKGKFDKTIQRLRLLQPFFEKNEEGITELYYDL
SVKALEHSGQCTYKGGRTISILEIGDIRISRKENAKGYLLTIPINRKSVVFDLYGRKDTIGGDGRDLIDIMNTHGSSLQFTADG
NDIYLTITATKNFIKEKPTFNEDTVLGGDVNIKHSYTVFSTSPKDIPDFVNFYEYFAKDGEIMKLAPKPMWDYIVAAATKFLTI
LPIETPAISATVYGKRTEEGISRATFRETQKLIALEKAIERVMKQVFDKYNDGKHPLEAIYIGNAIKYRRLIKGYLAQKKKYYS
AHSEYDKAMGYTDDDTDRKENMDERRFDDSKKFRYTPEAQALLDTMHTIEKKIVGCVSNAISYAYHKFDENGFNVIALENLTSA
TFAKKYKSDKPESIKKLLNFDKLLGKTLDEAKASKSISKHPNWYELVADENGCVSDIRITDEGQSATYRSLVTETIMKVSHFAE
TKDRFIGLANSGRLQVGLVPSQYTSYIDSTTHTLYAVIEDGKTVLAPKEVVRASQERHINGLNADYNSALNLKYMITDENFRKT
FTSETSADKFGWGKPMFSPTTRSQDEVFSAIKKIGAITVLED (SEQ ID NO: 37)
>3300031993|Ga0310696_10026614_2|M
[mammals-digestive system-rumen-bos taurus]
MAHKNSDGENTINKTFIFKVKCEKNDIISFWKPAAEEYCNYYNKLSEWIGKNLISMKIGDLAKYIDNPKSKYYLSVTDENKKDL
PLYKIFQKGFSSIDADNALYCAIDKLNPEGYNGNILGVGKSDYRRNGYVSSVIGNFRTKMVSLKANVRWKKIDIGNVDEETLRR
QTICDVEKYRIESEKDFRDLIDILKAREETPRLKEKISRLELLYDYYSKNTKTIKSEMENMAISDLQKFGGCVRKSLNTITIHK
QDSKIEKEGNTSFRLHMVFNKKPYTITLLGNRQVVKYIDGKRVDIVNIVEKHGDWITFNIKNGELFVHLTKCVEFSKGQKEIKK
AAGVDVNIKHAMLAASIVDDGQLKGYVNLYRELIEDDDFVSTFGDSDSGKTELGMYQKMAKTVFFGVLEVESLFERVVNQQSGW
KLDNQLIRRERAMEKVFDRIVKTTSNKHIIDYVNYVKMLRAKYKAYFILDEKYHEKQREYDLSMGFTDESDERRELYPFINTET
AKEILGKKRNVEQDLIGCRDNIVTYAFNVLRNNGYDTISVEYLDSSQFDKRRMPTPKSLLEYHKFKGKTQDEVERLMSEKKFAK
TNYDIHYDGENKVDGIVYSKEGELRQKKLNEMNLVIKAIHFADIKDKFAQLCNNNDVNVVFGPSAFTSQMDSETHSLYYVEKET
NGKNGKTGKKFVLADKKSVRRRQETHINGLNADFNAARNLEYIASNPELLERMTKRTKSGKDMYNTPSWNIRQEFKKNLSVRTI
NTFRELGNVKYGKINNEGLFVEDDV (SEQ ID NO: 38)
>3300031993|Ga0310696_10030100_3|M
[mammals-digestive system-rumen-bos taurus]
MAQHKSNNEESAINKTFIFKAKCEKNDVISLWEPAAKEYGDYYNKVSKWIADNLITMKIGDLAQYITNQNSKYYTAVTNKKKKD
LPLYRIFQKGFSSQCADNALYCAIKSINPENYKGNSLGIGESDYRRFGYIQSVVSNFRTKMSSLKVSVKYKKFDVSNVDDETLK
IQTIYDVDKYGIETAKEFKELIETLKTRVETPQLNDTIARLKCLCDYYSKNEKAINNEIETMAIADLQKFGGCQRKSLNAFTIH
KQDSLMEKVGNTSFRLQLSFRKKTYVINLLGNRQVVNFVNGKRVDLIDIAENHGDLITFNIKNGELFLHITSPIVFDKDVRDIR
NVVGIDVNIKHSMLATSIKDDGNVKGYINLYKELLNDDVFVSTCNESELALYRQMSENVNFGILETDSLFERIVNQSKGGCLKN
KLIRRELAMQKVFERITKTNKDQNIVDYVNYVKMMRAKCKASYILKEKYDEKQKEYYVKMGFTDESTESKETMDKRREEFPFVN
TDTAKELLVKQNNIRQDIIGCRDNIVTYAFNVFKNNEYDTLSVEYLDSSQFDKRRIPTPKSLLKYHKFEGKTKDEVENMMKSEK
LSNAYYTFKYENDVVSDIDYSDEGNLRRSKLNFGNWIIKAIHFADIKDKFVQLSNNNKMNIVFCPSAFSSQMDSITHTLYYVEK
ITKNKKGKEKKKYVLANKKMVRTQQETHINGLNADYNSACNLKYIALNYELRDKMTDRFKASKKIKTMYNIPAYNIKSNFKKNL
SAKTIQTFRELGHYRDGKINEDGMFVEILE (SEQ ID NO: 39)
>3300031998|Ga0310786_10000003 467 M
[mammals-digestive system-rumen-bos taurus]
MAHRKKKDDEATLSYKFKVKVIEGDLTADDITKCIAENAEQGNHFSEFIHKNLTSKTIGEFASQLPAEKRQFGYYQYAIGGTMP
AKKNASDEDKPKGELIDWSKKPFYVLFSKGYSATHAVNLIFNVYLNSEEGKAFSAKNSMNLSKSQFAYSGFVQIVCANYASMLA
NARPDKIKFEEITEATDDGTKKMQVVREMAERYLMKPKNFASRIEYLEANNTKGKFDKTIQRLRLLQPFFEKNEESITELYYDL
SVKALEHSGQCTYKGGRTISILEIGDIRISRKENAKGYLLTIPINRKSVVFDLYGRKDTIGGDGRDLIDIMNTHGSSLQFTADE
NDIYLTITATKNFIKEKPTFNEDTVLGGDVNIKHSYTVFSASPKDIPDFVNFYEYFAKDGEIMKLAPKPMWDYIVAAATKFLTI
LPIETPAISATVYGKRTEEGISRATFRETQKLIALEKAIERVMKQVFDKYNDGKHPLEAIYIGNAIKYRRLIKGYLAQKKKYYS
AHSEYDKAMGYTDDDTDRKENMDERRFDDSKKFRYTPEAQALLDTMHTIEKKIVGCVSNAISYAYHKFDENGFNVIALENLTSA
TFAKKYKSDKPESIKKLLNFDKLLGKTLDEAKASKSISKHPNWYELVADENGCVSDIRITDEGQSATYRSLVTETIMKVSHFAE
TKDRFIGLANSGRLQVGLVPSQYTSYIDSTTHTLYAVIEDGKTVLAPKEVVRASQERHINGLNADYNSALNLKYMITDENFRKT
FTSETSADKFGWGKPMFSPTTRSQDEVFSAIKKIGAITVLED (SEQ ID NO: 40)
>AUXO013988882|Ga0247611_10000101_23|P
[mammals-digestive system-rumen-ovis aries]
MANKRTDTTINLNKTVIMLTNMLPEVRAMFQAGIRQAQAYADLVNKWICSNLTNKIGEVLLPYIDNKNCVYYELCYKYKEAPLY
TIFMKGKFDLNSRNNALYCAVVAQNIDNYSGNIFGFSQSDYRRNGYCKVVFSNYATKMSSLKPSIKKVTINEESTEETIQSQVI
YEMFTNGRQVVGKPEYFAEHLKYLEMKDNVSDKLMFRMKTLCEYYQTHTDLIDTMMNAGVEALKQFEGLKLNRDKFSMTITTNS
TSPYTLTRVAGTCAYNLHIPCRKRSYDIRLWGNRQTVRWVNGELVDIADIINQHGQTIIFTIKNGNVYVHIPYGLNFEKTEHEI
KNVVGVDVNTKHMLMQTSIKDNGWVKGYVNIYKALVEDEEFVKYISKSDLKLYKDLSKYVSFCPLELNLLYTRYLSKKGLPFNE
ADNNAEKCVEKVLNNLVKQYEGDDVHVVNYIHNVKKLRALCKASFVLYKKYAELQKAFDDAQGYNDQSTETKETMDKRRWENPF
IQTREAQELIAKMDNAVAGIIGCRDNIITYAYKVFGDNNYDTVGLENLTTSQFDNYSTVKSPKSLLSYYGLLGQQVDSDKYNAV
MTESNKDWYDFKTDGDGNITDITLTAAGEAQKAKSLFNNKVLKNIHFADVKDKFIQLGNNGSIQTVLVPPSYTSQMDSKTHTIY
VKETVDPKNKNKKKLKLVDKKLVRHGQEYHKNGLNADINAALNIAYIVENQEMREVMCLHPSKKDGVYDQPFLKATTKYPATVA
GILLKMGKTTNWGEK (SEQ ID NO: 41)
>3300028805|Ga0247608_10000186_37|P
[mammals-digestive system-rumen-ovis aries]
MNKSYVFKSNVAIDDIMSLFEPAIEEYINYYNRTSDFICDNLTSMKIGDLANYIKNKENVYCKFVLNDDIKDLPLYKIFSLNLN
SSQKKNADNALYEAIKVLNADGYKGKNILGLGDTYFRRNGYVKNVISNYRTKFVTLKPNVKYSKIDINSVTEQLIKTQTIFEVV
NKKIESETDFENLITYFKNRETPNDEKIKRLELLFDYYTKHKNEINEEIEKHAVESLKSFNGCRRNGNRKTMTVQMQKMLLKKH
GLTSYILHLVLDKKPYDINLMGNRQTVKVDNNGNRVDLVDISSKHGYDLTFEVKGKTLFFTFSSEKDFSKKEQEIKNILGIDIN
TKHSMLATSITDNGKVKGYINIYVELLKNKDFVSTLNKEELAYYTEMAKFVSFGLLEIPSLFERVSNQYDKKNNVSITDETLLK
REIAISQTLDNLAKKYRDKNCKIASYIDYTKMLRSKYKSYFILKQKYYEKNHEYDDKMGFSDISTNSKETMDPRRFENPFINTD
IAKGLIVKLENVKCDIVGCRDNIIKYAYDVIVLNGFDTIGLEYLDSSNFERDRLPFPTAKSLMTYYGFEGKKYSEIDKSVFNTK
YYNFIFNENETIKDISYSVYGLKEIQKKRFKNLVIKAIGFADIKDKFVQLSNNTNMNVIFVPAAFTSQMDSNTHKIYVKEIMDK
NNKKQLQLIDKRKVRTKQEFHINGLNADFNAANNIKYIAENNDLLLTMCTKTKENNRYGNPLYNIKDTFKKKIPSSILNIFKKK
DMYQIICD (SEQ ID NO: 42)
>3300028805|Ga0247608_10000895_42|M
[mammals-digestive system-rumen-ovis aries]
MERIFAALKLTNMGHVRLQKREGEVYKTYKLKVKSFSGNVDIKAGIVEYDQKFNNVSQWIADHLTSMTIGEAASRISPHKMDSQ
YAMTSLSDEWKDQPLYKIFTRGFGGMNADNLIIECTKTEENCKYDKEKSLGFSESVFRTFGFAANASSDMKSRMTQAKVKIGRK
NIDEDSADDEKCLQAIYEIQKNELLTDDNWKDRIGYLEMKGDQERELERTTILYDYYRANRTTVLDKLDNLKVETLSKFRGSKR
KSDRKILTLNGISYDIKRKEGCQGFELKFSVDKNHMEFDLLGHRALIKNGEMLVDIENCHGSQLSLEIDGDDMYAIISMRTFCE
KNESKLEKIIGADVNIKHMFLMTSEKDDGNTKCYVNLYRELLSDSDFTDVLNKEEYEIFSELSKYVMFGLIETPYLGSRVIGTT
QHEKIVEDKITSGMKKIAIRLFQEGKVRERIYVQNVLKIRALLKALFSTKLAYSNEQKIYDNLMRFGEKDDRRKDEGFHTTCRG
TSLRSEMDMLSKKILACRDNIVEYGYYVIGLNGFDGISLENLESSTFMDVKISYPSCNSMLDHFKLKGKTIEEAENHETVGKFI
KKGYYVMTLVNGKINDINYSEKAVMLHKKNLLYDTVIKSTHFADVKDKFVELSNNGKVSVVIVPPYFSSQMDSVTHKVFTEEIV
VQKKSSNGKVRKTKKTVLVDKRKVRKTQESHINGLNADYNAALNLKYIAETIDWRSTLCFKTWNTYGSPQWDSKIKNQKTMIDR
LDSLGAIELKNW (SEQ ID NO: 43)
>3300028805|Ga0247608_10006074_1|M
[mammals-digestive system-rumen-ovis aries]
MSHEFNKNKGENEISKTFIFKTKCGKNDITSLWVPAMEEYCTYYNRVSKWICDNLTEMRIGDLAQYIDNHGSAYYSAVTDITKK
DLPLYKIEKKGFSGLCADNALYCAIAKLNPEGYDGNMFGLSETYYRRQGYIANVFGNYRTKMNAGLKVGCAKWKKFDTNDVDDE
ILMEQVIVDVVKYDIDSKNEFKEYIEVLKCREENPKLLETIERLECLYGYYSQHEEDIKKKIEELVVEELKTFGGCVRKSMTSC
TITVQDFVMERIGNTGYRINLTFNKKPYVLGLLGNRQVVRYVDGDRVELVDIVNNHGNQITFNLKNGELFVHLTSGVDFSKEES
SMENIVGVDVNIKHSMLASSIVDDGNVNGYINIYKELVNDDEFVSTFGDSESGLNELELYRQMAESVNFGLMETDSLFERYVEQ
WKGSDSDSRLARRERVVGKVFDRIVKTNGDVHVVNYIHAVKMLRAKCKAYFVLKQKYYEKQKEYDDAHGYTDESTASKETMDKR
RFENPFVETDVAKELLGKLACVEQDIIGCRDNIVTYAFNVFRRNGYDTISLEYLDSSQFKKIGMGAPTPKSLLKYHKLEGKTVE
EVESIISEKGLKKNLYVFKFGDNGLLSDIEYSDEGLIRKKKADFGNIITKAIHFADIKDKFVQLTNNSDMGVVFCPSAFTSQMD
SKTHRLYFVEGLDGNGKNKYVLANKWSVRRQQERHINGLNADFNSACNCQHIAYDPILRDAMTIKVEAGKGMYNKPSYDIRKKF
KKNLSAATLKTFIKLGNTVKGMIVNGQFVEMES (SEQ ID NO: 44)
>3300028833|Ga0247610_10000007_379|M
[mammals-digestive system-rumen-ovis aries]
MYNSKKKGEGDIQKSFKFKVKTDKETVELFRKAAVEYSEYYKRLTTFLCERLTDMTWGEVASFIPEKYRKNEYYKYLIKEENKD
LPLYKMFTKAASSMFIDHSIERYVEALNPEGNTGNILGFCKSSYVRGGYLKNVVSNIRTKFATLKTGIKYKKFNPAEDDEETIL
GQTVFEMEKRGLEFKCDFEKTIKYLNEKGKTQEAERLQCLMEYFSTNTDKINEYRESLVLDDIRKFGGCNRSKSNSFSVTLEKA
DIKEDGLTGYTMKVSKKLKEIHLLGHRRVVEVVNGRRVNLVDICGDKSGDSKVFVVDGDNLYVCISAPVKFSKNGMEAKKYIGV
DMNMKHSIISVSDNASDMKGFLNIYKELLKDEGFRKTLNATELEKYEKLAEGVNIGIIEYDGLYERIVKQKKENSVDGLKVQAE
KKLIEREAAIERVLDKLRKGTSDTDTENYINYNKILRAKIKSAYILKDKYYEMLGKYDSERAGSGDLSEENKIKYKDEFNETEK
GKEILGKLNNVYKDIIGCRDNIVTYAVNLFIRNGYDTVALEYLESSQMKARRIPSTGGLLKGHKLEGKPEGEVTAYLKANKIPK
SYYSFEYDGNGMLTDVKYSDMGEKARGRNRFKNLVPKFLRWASIKDKFVQLSNYKDIQMVYVPSPYTSQTDSRTHSLYYIETVK
VDEKTGKEKKEHIVAPKESVRTEQESFVNGMNADTNSANNIKYIFENETLRDKFLKRTKDGTEMYNRPAFDLKECYKKNSNVSV
FNTLKKTLGAIYGKLDENGNFIENECNK (SEQ ID NO: 45)
>3300028833|Ga0247610_10004486_2|M
[mammals-digestive system-rumen-ovis aries]
MNKSYVFKSNVAIDDIMSLFEPAIEEYINYYNRTSDFICDNLTSMKIGDLANYIKNKENVYCKFVLNDDIKDLPLYKIFSLNLN
SSQKKNADNALYEAIKVLNADGYKGKNILGLGDTYFRRNGYVKNVISNYRTKFVTLKPNVKYSKIDINSVTEQLIKTQTIFEVV
NKKIESETDFENLITYFKNRETPNDEKIKRLELLFDYYTKHKNEINEEIEKHAVESLKSFNGCRRNGNRKTMTVQMQKMLLKKH
GLTSYILHLVLDKKPYDINLMGNRQTVKVDNNGNRVDLVDISSKHGYDLTFEVKGKTLFFTFSSEKDFSKKEQEIKNILGIDIN
TKHSMLATSITDNGKVKGYINIYVELLKNKDFVSTLNKEELAYYTEMAKFVSFGLLEIPSLFERVSNQYDKKNNVSITDETLLK
REIAISQTLDNLAKKYRDKNCKIASYIDYTKMLRSKYKSYFILKQKYYEKNHEYDDKMGFSDISTNSKETMDPRRFENPFINTD
IAKGLIVKLENVKCDIVGCRDNIIKYAYDVIVLNGFDTIGLEYLDSSNFERDRLPFPTAKSLMTYYGFEGKKYSEIDKSVFNTK
YYNFIFNENETIKDISYSVYGLKEIQKKRFKNLVIKAIGFADIKDKFVQLSNNTNMNVIFVPAAFTSQMDSNTHKIYVKEIMDK
NNKKQLQLIDKRKVRTKQEFHINGLNADFNAANNIKYIAENNDLLLTMCTKTKENNRYGNPLYNIKDTFKKKIPSSILNIFKKK
DMYQIICD (SEQ ID NO: 46)
>3300028888|Ga0247609_10000668_74|M
[mammals-digestive system-rumen-ovis aries]
MAHKTKESEKLVKSFKLKVDISNCEIEKKWIPSFEEYTNYYNGVSNWICENLISMKIGDLGQYIKNTESVYYKFITDESISNLP
LYKIFTLKQTQNVDNALFCAIKEINPEKYNGNSIGLGETDYRRFGYVQCVISNYRTKIGTMKASIKYKTLPENQSYDVIFEQTM
YEMIDKSLEKKEDWENIISNYKAKQTENTSKINRMETLYSFFIEHSEEIIEKSNLVAIEQLALFNGCKRKSLSTMTIHSQHSKL
QKNGLTSFVFCINQKIGSINLFGNRQLVSVDENGNRNDIIDICNNYGDFITFQIKNGKMFIILTAKVDFDKENIEIKNVVGADV
NIKHNMIASSIIDNGNVFGYINIYKELLNDEDFCSSCTNEELDIYKEISKSVNFGLLECESLFSRVSAQIYKENESISKLDDRF
LRREKSIENVLNRLSKQYRYKDCKIATYIDYTKIMRDSYKSYFIIKEKYYEKQKEYDISMGYVDESTNSKKTMDKRRFENPFIE
TETAKNILSKLNRIESRLIGCRNNITNYAFDVFKNNGFDTIALEYLDSSQFDKTKVLTPISMLKYHKFEGKSIEEVKTLNVKFS
MDNYEFEFDNNGKITNISFSQLGKREVMKTNFFNLIIKAIHFAEIKDKFIQLSNNKPINIVLVPSAFSSQMDSKDHKLYVDENG
KLINKRKVRKQQERHINGLNADFNAACNLSYLAKNNELLEKVCLKRKKFGKASYSVPYWNVKDAFKKNVSSNMIATIKKMNMVK
VF (SEQ ID NO: 47)
>3300028888|Ga0247609_10003329_9|M
[mammals-digestive system-rumen-ovis aries]
MAHKTNNGENTINKTFIFKAKCEKNDIISLWKPAAEEYCNYYNKLSKWIGDSLTTMKIGDLAQYITNQNSAYYLAVTNDSKKDL
PLYKIFQKGFSSQCADNALYSAIKAINPENYNGNSLEIGETDYRRFGYVQSVIGNFRTKMSSLKVSVKYKKFDVNDVDEETLKT
QTIYDVDKYGIESIKDFNEFIEVLKLREETPQLNEKITRLECLCGYYSKNEENIKNEIETMAISDLQKFGGCQRKSLNTLTIHK
QNSLMEKVGNTSFTLQLSFNKKPYTINLLGNRQVVKFVDGKRVDLIDITEKHGDWVTFNIKNDELFVHLTSPIDFEKEVCEIKN
AVGVDVNIKHNMLATSIKDDGNVKGYINLYKELVNDCDFISTCNEDEFDLYRQMSESVNFGILETDSLFERVVNQSKGGCLNNK
FIRRELAMQKVFDNITKTNKDQNIVDYVNYVKMLRAKYKAYFILKEKYYEKQKEYDIKMGFTDVSTESKETMDKRRMEFPFVNT
DTAKELLAKLNNIEQDLIGCRDNIVTYAFNIFKNNGYDTLAVEYLDSAQFDKRRMPTPTSLLKYHKFEGKTKDEVEDMMKSKKF
SNAYYTFKFENDVVSNIEYSNDGIWKQKQLNFGNLIIKAIHFADIKDKFVQLCNNNKMNIVFCPSAFTSQMDSITHTLYYVEKI
TKKKNGKEEKKYVLANKKMVRTQQETHINGLNADYNSACNLKYIALNDELRNEMTDTFKVTNRQKTMYGIPAYNIKRGFKKNLS
AKTINTFRKLGHYRDGKINEDGMFVETLA (SEQ ID NO: 48)
>3300028888|Ga0247609_10016480_8|M
[mammals-digestive system-rumen-ovis aries]
MAHKTNNGENTINKTFIFKAKCDNNDIISLWKPAMEEYCTYYNKLSQWICNNLTSMKVKDLFAYLDDKQKTKPCVDKKTGETKI
GVGYYRYFIENNKEDMPLYWLFTKNCSSSHADNLLFEFVRKVNHEEYNGNSLGMGETDYRRFGYFQNVISNFRTKMSSLKATTK
WKKFDVNDVDEDTLKNQTIYDVDKYGIESVNDFNERIDILKIREETEQTKDKIARLECLCKYYKEHEEDIKNEIATMAIADLQK
FGGCQRKSMNTLTIHKQDSPMEKVGNTSFNLRLTFNKKPYTLNLLGNRQVVKFVGGKRIDLINITENHGDWITFNIKNNELFVH
MTSPVDFEKEVCEIKNAVGVDVNIKHMMLATSIVDDGNVKGYINLYRELVNNNDFIATFGNSKNGHQGLEIYEQMAENVNFGIL
ETESLFERVVNQSNGGELNNQLIRREIAMQKVEDNITKTNNDKNIVNYVNYVKMLRAKYKAYFILKEKYYEKQKEYDDMMGFND
ESTENKEMMDKRRFEFSFINTDTAQELLIKLNKVEQDLIGCRDNIVTYAFNVFKTNGYDTLAVEYLDSAQFDKAKMPTPKSLLK
YHKFEGKTIDEVKEMMNNKNFTNAYYNFKFENEIVKDIEYSTDGIWRQKKLNFMNLIIKAIHFADIKDKFVQLCNNNSMNVVFC
PSAFTSQMDSITHSLYYIEKTSKTKNGKEKKQYVLANKKMVRTQQEKHINGLNADFNSACNLKYIALDEELRNAMTDEFNPKKQ
KTMYGVPAYNIKNGFKKNLSTKTINTFRTLGHYRDGKINEDGVFVENLA (SEQ ID NO: 49)
>3300031992|Ga0310694_10000010_351|M
[mammals-digestive system-rumen-ovis aries]
MYNSKKKGEGDIQKSFKFKVKTDKETVELFRKAAVEYSEYYKRLTTFLCERLTDMTWGEVASFIPEKYRKNEYYKYLIKEENKD
LPLYKMFTKAASSMFIDHSIERYVEALNPEGNTGNILGFCKSSYVRGGYLKNVVSNIRTKFATLKTGIKYKKFNPAEDDEETIL
GQTVFEMEKRGLEFKCDFEKTIKYLNEKGKTQEAERLQCLMEYFSTNTDKINEYRESLVLDDIRKFGGCNRSKSNSFSVTLEKA
DIKEDGLTGYTMKVSKKLKEIHLLGHRRVVEVVNGRRVNLVDICGDKSGDSKVFVVDGDNLYVCISAPVKFSKNGMEAKKYIGV
DMNMKHSIISVSDNASDMKGFLNIYKELLKDEGFRKTLNATELEKYEKLAEGVNIGIIEYDGLYERIVKQKKENSVDGLKVQAE
KKLIEREAAIERVLDKLRKGTSDTDTENYINYNKILRAKIKSAYILKDKYYEMLGKYDSERAGSGDLSEENKIKYKDEFNETEK
GKEILGKLNNVYKDIIGCRDNIVTYAVNLFIRNGYDTVALEYLESSQMKARRIPSTGGLLKGHKLEGKPEGEVTAYLKANKIPK
SYYSFEYDGNGMLTDVKYSDMGEKARGRNRFKNLVPKFLRWASIKDKFVQLSNYKDIQMVYVPSPYTSQTDSRTHSLYYIETVK
VDEKTGKEKKEHIVAPKESVRTEQESFVNGMNADTNSANNIKYIFENETLRDKFLKRTKDGTEMYNRPAFDLKECYKKNSNVSV
FNTLKKTLGAIYGKLDENGNFIENECNK (SEQ ID NO: 50)
>3300031992|Ga0310694_10022272_2|M
[mammals-digestive system-rumen-ovis aries]
MNKSYVFKSNVAIDDIMSLFEPAIEEYINYYNRTSDFICDNLTSMKIGDLANYIKNKENVYCKFVLNDDIKDLPLYKIFSLNLN
SSQKKNADNALYEAIKVLNADGYKGKNILGLGDTYFRRNGYVKNVISNYRTKFVTLKPNVKYSKIDINSVTEQLIKTQTIFEVV
NKKIESETDFENLITYFKNRETPNDEKIKRLELLFDYYTKHKNEINEEIEKHAVESLKSFNGCRRNGNRKTMTVQMQKMLLKKH
GLTSYILHLVLDKKPYDINLMGNRQTVKVDNNGNRVDLVDISSKHGYDLTFEVKGKTLFFTFSSEKDFSKKEQEIKNILGIDIN
TKHSMLATSITDNGKVKGYINIYVELLKNKDFVSTLNKEELAYYTEMAKFVSFGLLEIPSLFERVSNQYDKKNNVSITDETLLK
REIAISQTLDNLAKKYRDKNCKIASYIDYTKMLRSKYKSYFILKQKYYEKNHEYDDKMGFSDISTNSKETMDPRRFENPFINTD
IAKGLIVKLENVKCDIVGCRDNIIKYAYDVIVLNGFDTIGLEYLDSSNFERDRLPFPTAKSLMTYYGFEGKKYSEIDKSVFNTK
YYNFIFNENETIKDISYSVYGLKEIQKKRFKNLVIKAIGFADIKDKFVQLSNNTNMNVIFVPAAFTSQMDSNTHKIYVKEIMDK
NNKKQLQLIDKRKVRTKQEFHINGLNADFNAANNIKYIAENNDLLLTMCTKTKENNRYGNPLYNIKDTFKKKIPSSILNIFKKK
DMYQIICD (SEQ ID NO: 51)
>3300031994|Ga031069l_10000084_157|M
[mammals-digestive system-rumen-ovis aries]
MERIFAALKLTNMGHVRLQKREGEVYKTYKLKVKSFSGNVDIKAGIVEYDQKFNNVSQWIADHLTSMTIGEAASRISPHKMDSQ
YAMTSLSDEWKDQPLYKIFTRGFGGMNADNLIIECTKTEENCKYDKEKSLGFSESVFRTFGFAANASSDMKSRMTQAKVKIGRK
NIDEDSADDEKCLQAIYEIQKNELLTDDNWKDRIGYLEMKGDQERELERTTILYDYYRANRTTVLDKLDNLKVETLSKFRGSKR
KSDRKILTLNGISYDIKRKEGCQGEELKFSVDKNHMEFDLLGHRALIKNGEMLVDIENCHGSQLSLEIDGDDMYAIISMRTFCE
KNESKLEKIIGADVNIKHMFLMTSEKDDGNTKCYVNLYRELLSDSDFTDVLNKEEYEIFSELSKYVMFGLIETPYLGSRVIGTT
QHEKIVEDKITSGMKKIAIRLFQEGKVRERIYVQNVLKIRALLKALFSTKLAYSNEQKIYDNLMRFGEKDDRRKDEGFHTTCRG
TSLRSEMDMLSKKILACRDNIVEYGYYVIGLNGFDGISLENLESSTFMDVKISYPSCNSMLDHFKLKGKTIEEAENHETVGKFI
KKGYYVMTLVNGKINDINYSEKAVMLHKKNLLYDTVIKSTHFADVKDKFVELSNNGKVSVVIVPPYFSSQMDSVTHKVFTEEIV
VQKKSSNGKVRKTKKTVLVDKRKVRKTQESHINGLNADYNAALNLKYIAETIDWRSTLCFKTWNTYGSPQWDSKIKNQKTMIDR
LDSLGAIELKNW (SEQ ID NO: 52)
>3300031994|Ga0310691_10000270_20|M
[mammals-digestive system-rumen-ovis aries]
MNKSYVFKSNVAIDDIMSLFEPAIEEYINYYNRTSDFICDNLTSMKIGDLANYIKNKENVYCKFVLNDDIKDLPLYKIFSLNLN
SSQKKNADNALYEAIKVLNADGYKGKNILGLGDTYFRRNGYVKNVISNYRTKFVTLKPNVKYSKIDINSVTEQLIKTQTIFEVV
NKKIESETDFENLITYFKNRETPNDEKIKRLELLFDYYTKHKNEINEEIEKHAVESLKSFNGCRRNGNRKTMTVQMQKMLLKKH
GLTSYILHLVLDKKPYDINLMGNRQTVKVDNNGNRVDLVDISSKHGYDLTFEVKGKTLFFTFSSEKDFSKKEQEIKNILGIDIN
TKHSMLATSITDNGKVKGYINIYVELLKNKDFVSTLNKEELAYYTEMAKFVSFGLLEIPSLFERVSNQYDKKNNVSITDETLLK
REIAISQTLDNLAKKYRDKNCKIASYIDYTKMLRSKYKSYFILKQKYYEKNHEYDDKMGFSDISTNSKETMDPRRFENPFINTD
IAKGLIVKLENVKCDIVGCRDNIIKYAYDVIVLNGFDTIGLEYLDSSNFERDRLPFPTAKSLMTYYGFEGKKYSEIDKSVFNTK
YYNFIFNENETIKDISYSVYGLKEIQKKRFKNLVIKAIGFADIKDKFVQLSNNTNMNVIFVPAAFTSQMDSNTHKIYVKEIMDK
NNKKQLQLIDKRKVRTKQEFHINGLNADFNAANNIKYIAENNDLLLTMCTKTKENNRYGNPLYNIKDTFKKKIPSSILNIFKKK
DMYQIICD (SEQ ID NO: 53)
>3300032030|Ga0310697_10001273_44|P
[mammals-digestive system-rumen-ovis aries]
MAHKTNNGENTINKTFIFKAKCDNNDIISLWKPAMEEYCTYYNKLSQWICNNLTSMKVKDLFAYLDDKQKTKPCVDKKTGETKI
GVGYYRYFIENNKEDMPLYWLFTKNCSSSHADNLLFEFVRKVNHEEYNGNSLGMGETDYRRFGYFQNVISNFRTKMSSLKATTK
WKKFDVNDVDEDTLKNQTIYDVDKYGIESVNDFNERIDILKIREETEQTKDKIARLECLCKYYKEHEEDIKNEIATMAIADLQK
FGGCQRKSMNTLTIHKQDSPMEKVGNTSFNLRLTFNKKPYTLNLLGNRQVVKFVGGKRIDLINITENHGDWITFNIKNNELFVH
MTSPVDFEKEVCEIKNAVGVDVNIKHMMLATSIVDDGNVKGYINLYRELVNNNDFIATFGNSKNGHQGLEIYEQMAENVNFGIL
ETESLFERVVNQSNGGELNNQLIRREIAMQKVFDNITKTNNDKNIVNYVNYVKMLRAKYKAYFILKEKYYEKQKEYDDMMGFND
ESTENKEMMDKRRFEFSFINTDTAQELLIKLNKVEQDLIGCRDNIVTYAFNVFKTNGYDTLAVEYLDSAQFDKAKMPTPKSLLK
YHKFEGKTIDEVKEMMNNKNFTNAYYNFKFENEIVKDIEYSTDGIWRQKKLNFMNLIIKAIHFADIKDKFVQLCNNNSMNVVFC
PSAFTSQMDSITHSLYYIEKTSKTKNGKEKKQYVLANKKMVRTQQEKHINGLNADFNSACNLKYIALDEELRNAMTDEFNPKKQ
KTMYGVPAYNIKNGFKKNLSTKTINTFRTLGHYRDGKINEDGVFVENLA (SEQ ID NO: 54)
>3300032030|Ga0310697_10005481_13|P
[mammals-digestive system-rumen-ovis aries]
MAHKTNNGENTINKTFIFKAKCEKNDIISLWKPAAEEYCNYYNKLSKWIGDSLTTMKIGDLAQYITNQNSAYYLAVTNDSKKDL
PLYKIFQKGFSSQCADNALYSAIKAINPENYNGNSLEIGETDYRRFGYVQSVIGNFRTKMSSLKVSVKYKKFDVNDVDEETLKT
QTIYDVDKYGIESIKDFNEFIEVLKLREETPQLNEKITRLECLCGYYSKNEENIKNEIETMAISDLQKFGGCQRKSLNTLTIHK
QNSLMEKVGNTSFTLQLSFNKKPYTINLLGNRQVVKFVDGKRVDLIDITEKHGDWVTFNIKNDELFVHLTSPIDFEKEVCEIKN
AVGVDVNIKHNMLATSIKDDGNVKGYINLYKELVNDCDFISTCNEDEFDLYRQMSESVNFGILETDSLFERVVNQSKGGCLNNK
FIRRELAMQKVFDNITKTNKDQNIVDYVNYVKMLRAKYKAYFILKEKYYEKQKEYDIKMGFTDVSTESKETMDKRRMEFPFVNT
DTAKELLAKLNNIEQDLIGCRDNIVTYAFNIFKNNGYDTLAVEYLDSAQFDKRRMPTPTSLLKYHKFEGKTKDEVEDMMKSKKF
SNAYYTFKFENDVVSNIEYSNDGIWKQKQLNFGNLIIKAIHFADIKDKFVQLCNNNKMNIVFCPSAFTSQMDSITHTLYYVEKI
TKKKNGKEEKKYVLANKKMVRTQQETHINGLNADYNSACNLKYIALNDELRNEMTDTFKVTNRQKTMYGIPAYNIKRGFKKNLS
AKTINTFRKLGHYRDGKINEDGMFVETLA (SEQ ID NO: 55)
>OBLI01003123_14|M
[pig gut metagenome]
MAHKKNIGAEIVKTYSFKVKNTNGITMEKLMAAIDEYQSYYNLCSDWICKNLTTMTIGDLDRYIPEKSKDNIYATVLLDEVWKN
QPLYKIFGKKYSANNRNNALYCALSSVIDMNKENVLGFSKTHYVRNGYILNVISNYASKLSKLNTGVKSRAIKETSDEATIIEQ
VIYEMEHNKWESIEDWKNQIEYLNSKTDYNPTYMERMKTLSAYYSEHKSEIDAKMQEMAVENLVKFGGCRRNNSKKSMFIMGSN
HTNYTISYIGENCFNINFANILNFDVYGRRDVVKNGEVLVDIMANHGDSIVLKIVNGELYADVPCSVTLNKVESNFDKVVGIDV
NMKHMLLSTSVTDNGSLDFLNIYKEMSNNAEFMALCPEKDRKYYKDISQYVTFAPLELDLLFSRISKQDKVKMEKAYSEILEAL
KWKFFANGDNKNRIYVESIQKIRQQIKALCVIKNAYYEQQSAYDIDKTQEYIETHPFSLTEKGMSIKSKMDKICQTIIGCRNNI
IDYAYSFFERNGYTIIGLEKLTSSQFEKTKSMPTCKSLLNFHKVLGHTLSELETLPINDVVKKGYYAFTTDNEGRITDASLSEK
GKVRKMKDDFFNQAIKAIHFADVKDYFATLSNNGQTGIFFVPSQFTSQMDSNTHNLYFENAKNGGLKLASKSKVRKSQEYHLNG
LPADYN7\ARNIAYIGLDEIMRNTFLKKANSNKSLYNQPIYDTGIKKTAGVFSRMKKLKKYKVI (SEQ ID NO: 56)

TABLE 73
Conserved Sequences of CLUST.091979 Effectors.

| Sequence | Residues | Position |
|---|---|---|
| PX₁X₂X₃X₄F | X₁ is L or M or I or C or F | N- |
| (SEQ ID NO: 216) | X₂ is Y or W or F | terminal |
| | X₃ is K or T or C or R or W or | |
| | Y or H or V | |
| | X₄ is I or L or M | |
| RX₁X₂X₃L | X₁ is I or L or M or Y or T or F | Mid |
| (SEQ ID NO: 217) | X₂ is R or Q or K or E or S or T | sequence |
| | X₃ is L or I or T or C or M or K | |
| NX₁YX₂ | X₁ is I or L or F | Mid |
| (SEQ ID NO: 218) | X₂ is K or R or V or E | sequence |
| KX₁X₂X₃FAX₄X₅KD | X₁ is T or I or N or A or S or F | C- |
| (SEQ ID NO: 219) | or V | terminal |
| | X₂ is I or V or L or S | |
| | X₃ is H or S or G or R | |
| | X₄ is D or S or E | |
| | X₅ is I or V or M or T or N | |
| LX₁NX₂ | X₁ is G or S or C or T | C- |
| (SEQ ID NO: 220) | X₂ is N or Y or K or S | terminal |
| PX₁X₂X₃X₄SQX₅DS | X₁ is S or P or A | C- |
| (SEQ ID NO: 221) | X₂ is Y or S or A or P or E or Y | terminal |
| | or Q or N | |
| | X₃ is F or Y or H | |
| | X₄ is T or S | |
| | X₅ is M or T or I | |
| KX₁X₂VRX₃X₄QEX₅H | X₁ is N or K or W or R or E or | C- |
| (SEQ ID NO: 222) | T or Y | terminal |
| | X₂ is M or R or L or S or K or | |
| | V or E or T or I or D | |
| | X₃ is L or R or H or P or T or K | |
| | or Q of P or S or A | |
| | X₄ is G or Q or N or R or K or | |
| | E or I or T or S or C | |
| | X₅ is R or W or Y or K or T or | |
| | F or S or Q | |
| X₁NGX₂X₃X₄DX₅NX₆X₇X₈N | X₁ is I or K or V or L | C- |
| (SEQ ID NO: 223) | X₂ is L or M | terminal |
| | X₃ is N or H or P | |
| | X₄ is A or S or C | |
| | X₅ is V or Y or I or For T or N | |
| | X₆ is A or S | |
| | X₇ is S or A or P | |
| | X₈ is M or C or L or R or N or S | |
| | or K or L | |

Examples of direct repeat sequences and spacer lengths for these systems are shown in TABLE 8.

TABLE 8
Nucleotide Sequences of Representative CLUST.091979 Direct Repeats and Spacer Lengths

| CLUST.091979 | | Spacer |
|---|---|---|
| Effector Protein Accession | Direct Repeat Nucleotide Sequence | Length(s) |
| AUXO013988882_8\|P | ACTATGTTGGAATACATTTTTATAGGTATTTACAACT (SEQ | 28-29 |
| (SEQ ID NO: 1) | ID NO: 57) | |
| | AGTTGTAAATACCTATAAAAATGTATTCCAACATAGT (SEQ | |
| | ID NO: 118) | |
| SRR094437_845781_4\|M | ATTGTTGGAATATCACTTTTGTAGGGTATTCACAAC (SEQ | 30-31 |
| (SEQ ID NO: 2) | ID NO: 58) | |
| | GTTGTGAATACCCTACAAAAGTGATATTCCAACAAT (SEQ | |
| | ID NO: 119) | |
| SRR1221442_316828_61\|P | AATGTTGTTCACCCTTTTT (SEQ ID NO: 59) | 47 |
| (SEQ ID NO: 3) | AAAAAGGGTGAACAACATT (SEQ ID NO: 120) | |
| SRR3181151_741875_3\|M | CCTGTTGTGAATACTCTTTTATAGGTATCAAACAAC (SEQ | 26-30 |
| (SEQ ID NO: 4) | ID NO: 60) | |
| | GTTGTTTGATACCTATAAAAGAGTATTCACAACAGG (SEQ | |
| | ID NO: 121) | |
| SRR5371369_1764679_7\|P | ATTGTTGTAACTCTTATTTTGTATGGAGTAAACAAC (SEQ | 30 |
| (SEQ ID NO: 5) | ID NO: 61) | |
| | GTTGTTTACTCCATACAAAATAAGAGTTACAACAAT (SEQ | |
| | ID NO: 122) | |
| SRR5371371_1138852_2\|M | ATTGTTGTAGACACCTTTTTATAAGGATTGAACAAC (SEQ | 29-43 |
| (SEQ ID NO: 6) | ID NO: 62) | |
| | GTTGTTCAATCCTTATAAAAAGGTGTCTACAACAAT (SEQ | |
| | ID NO: 123) | |
| SRR5371379_2478682_1\|M | CTTGTTGTATATACTCTTTTATAGGTATTAAACAAC (SEQ | 29-38 |
| (SEQ ID NO: 7) | ID NO: 63) | |
| | GTTGTTTAATACCTATAAAAGAGTATATACAACAAG (SEQ | |
| | ID NO: 124) | |
| SRR5371385_201181_1\|P | ATTGTTGTAACTCTTATTTTGTATGGAGTAAACAAC (SEQ | 25-30 |
| (SEQ ID NO: 8) | ID NO: 61) | |
| | GTTGTTTACTCCATACAAAATAAGAGTTACAACAAT (SEQ | |
| | ID NO: 122) | |
| SRR5371385_201181_1\|M | ATTGTTGTAACTCTTATTTTGTATGGAGTAAACAAC (SEQ | 25-30 |
| (SEQ ID NO: 9) | ID NO: 61) | |
| | GTTGTTTACTCCATACAAAATAAGAGTTACAACAAT (SEQ | |
| | ID NO: 122) | |
| SRR5371401_1055766_58\|M | CTTGTTGTATATGTCCTTTTATAGGTATT (SEQ ID NO: | 30-51 |
| 10)(SEQ ID NO: | 64) | |
| | AATACCTATAAAAGGACATATACAACAAG (SEQ ID NO: | |
| | 125) | |
| SRR5371439_988701_11\|M | CTTGTTGTATATACTCTTTTATAGGTATTAAACAAC (SEQ | 29-30 |
| (SEQ ID NO: 11) | ID NO: 63) | |
| | GTTGTTTAATACCTATAAAAGAGTATATACAACAAG (SEQ | |
| | ID NO: 124) | |
| SRR5371497_203858_6\|M | CTTGTTGTATATGTCTTTTTATAGGTATTGAACAAC (SEQ | 30 |
| (SEQ ID NO: 12) | ID NO: 65) | |
| | GTTGTTCAATACCTATAAAAAGACATATACAACAAG (SEQ | |
| | ID NO: 126) | |
| SRR5371501_2762794_1\|M | TACTCTTTTTTAGGTAATGAACAAC (SEQ ID NO: 66) | 41 |
| (SEQ ID NO: 13) | GTTGTTCATTACCTAAAAAAGAGTA (SEQ ID NO: 127) | |
| SRR5678926_1309611_3\|P | CTTGTTGTATATATTCTTTTATAGGTATTAAACAAC (SEQ | 24-37 |
| (SEQ ID NO: 14) | ID NO: 67) | |
| | GTTGTTTAATACCTATAAAAGAATATATACAACAAG (SEQ | |
| | ID NO: 128) | |
| SRR6059713_382107_4\|P | CTTGTTGTATATACTCTTTTATAGGTATTAAACAAC (SEQ | 28-31 |
| (SEQ ID NO: 15) | ID NO: 63) | |
| | GTTGTTTAATACCTATAAAAGAGTATATACAACAAG (SEQ | |
| | ID NO: 124) | |
| SRR6060192_2608084_13\|P | CATGTTGTACATACTATTTTTTAAGTATTAAACAAC (SEQ | 27-42 |
| (SEQ ID NO: 16) | ID NO: 68) | |
| | GTTGTTTAATACTTAAAAAATAGTATGTACAACATG (SEQ | |
| | ID NO: 129) | |
| SRR7634052_1662339_24\|M | GATGTTGGACACTATGTTTTATACGGTGGATACAAC (SEQ | 30 |
| (SEQ ID NO: 17) | ID NO: 69) | |
| | GTTGTATCCACCGTATAAAACATAGTGTCCAACATC (SEQ | |
| | ID NO: 130) | |
| AUXO017332817_2\|M | GATGTTGTTATGCTGTTTTTGTAAGTAATAAACAAC (SEQ | 29-30 |
| (SEQ ID NO: 18) | ID NO: 70) | |
| | GTTGTTTATTACTTACAAAAACAGCATAACAACATC (SEQ | |
| | ID NO: 131) | |
| OQVL01000914_15\|P | ATTGTTGTAGACCTCTTTTTATAAGGATTGAACAAC (SEQ | 30 |
| (SEQ ID NO: 19) | ID NO: 71) | |
| | GTTGTTCAATCCTTATAAAAAGAGGTCTACAACAAT (SEQ | |
| | ID NO: 132) | |
| 33000015981 EMG_10017415_6\|P | AATGTTGTTCACCCTTTTT (SEQ ID NO: 59) | 47 |
| (SEQ ID NO: 20) | AAAAAGGGTGAACAACATT (SEQ ID NO: 120) | |
| 3300021254\|Ga0223824_10022219_ | ATTGTTGTACGAACCATTTTATATGGTAATAACAAC (SEQ | 29-30 |
| 2\|P (SEQ ID NO: 21) | ID NO: 72) | |
| | GTTGTTATTACCATATAAAATGGTTCGTACAACAAT (SEQ | |
| | ID NO: 133) | |
| 3300021431\|Ga0224423_10015012_ | ACTGTAAAACCCCTGCAGATGAAAGGAAAGTACAACAGT | 27-42 |
| 2\|P (SEQ ID NO: 22) | (SEQ ID NO: 73) | |
| | ACTGTTGTACTTTCCTTTCATCTGCAGGGGTTTTACAGT | |
| | (SEQ ID NO: 134) | |
| 3300012973\|Ga0123351_1009859_ | ATCATGTTGTACATACTATTTTTTAAGTATTAAACAACTA | 26-29 |
| 3\|P (SEQ ID NO: 23) | (SEQ ID NO: 74) | |
| | TAGTTGTTTAATACTTAAAAAATAGTATGTACAACATGAT | |
| | (SEQ ID NO: 135) | |
| 3300012979\|Ga0123348_10005323_ | CTTGTTGTATATACTCTTTTATAGGTATTAAACAAC (SEQ | 28-31 |
| 4\|M (SEQ ID NO: 24) | ID NO: 63) | |
| | GTTGTTTAATACCTATAAAAGAGTATATACAACAAG (SEQ | |
| | ID NO: 124) | |
| 3300028797\|Ga0265301_10000251_ | ATTGTTGAATGGCTATGTTTGTATGCTATTTACAAC (SEQ | 28-30 |
| 12\|M (SEQ ID NO: 25) | ID NO: 75) | |
| | GTTGTAAATAGCATACAAACATAGCCATTCAACAAT (SEQ | |
| | ID NO: 136) | |
| 3300028797\|Ga0265301_10000251_ | ATTGTTGAATGGCTATGTTTGTATGCTATTTACAAC (SEQ | 28-30 |
| 10\|P (SEQ ID NO: 26) | ID NO: 75) | |
| | GTTGTAAATAGCATACAAACATAGCCATTCAACAAT (SEQ | |
| | ID NO: 136) | |
| 3300028797\|Ga0265301_10009039_ | ATTGTTGGGGTACTTCTTTTATAGGGTACTCACAAC (SEQ | 29-30 |
| 3\|M (SEQ ID NO: 27) | ID NO: 76) | |
| | GTTGTGAGTACCCTATAAAAGAAGTACCCCAACAAT (SEQ | |
| | ID NO: 137) | |
| 3300028887\|Ga0265299_10000013_ | ATTGTTGTAGACCTTGTGTTTTAGGGGTCTAACAACG (SEQ | 29-30 |
| 320\|P (SEQ ID NO: 28) | ID NO: 77) | |
| | CGTTGTTAGACCCCTAAAACACAAGGTCTACAACAAT (SEQ | |
| | ID NO: 138) | |
| 3300028887\|Ga0265299_10000026_ | ACTGTGTTGGAATACAATATGAGATGTATTTACAAC (SEQ | 30 |
| 77\|P (SEQ ID NO: 29) | ID NO: 78) | |
| | GTTGTAAATACATCTCATATTGTATTCCAACACAGT (SEQ | |
| | ID NO: 139) | |
| 3300028887\|Ga0265299_10000133_ | ATTGTTGTGGCATACCGCAAGGCGGATGCTGACAAC (SEQ | 26-34 |
| 30\|M (SEQ ID NO: 30) | ID NO: 79) | |
| | GTTGTCAGCATCCGCCTTGCGGTATGCCACAACAAT (SEQ | |
| | ID NO: 140) | |
| 3300028887\|Ga0265299_10011526_ | ATTGTTGGAATATCACTTTTGTAGGGTATTCACAAC (SEQ | 30-31 |
| 3\|M (SEQ ID NO: 31) | ID NO: 58) | |
| | GTTGTGAATACCCTACAAAAGTGATATTCCAACAAT (SEQ | |
| | ID NO: 119) | |
| 3300028887\|Ga0265299_10012919_ | AATTGTTGAGATACCGTTTTTTATGGTATTGGCAAC (SEQ | 28-43 |
| 3\|P (SEQ ID NO: 32) | ID NO: 80) | |
| | GTTGCCAATACCATAAAAAACGGTATCTCAACAATT (SEQ | |
| | ID NO: 141) | |
| 3300028914\|Ga0265300_10009460_ | ATTGTTGTGGCATACCGTATTACGGGTGCTGACAA (SEQ ID | 31 |
| 3\|M (SEQ ID NO: 33) | NO: 81) | |
| | TTGTCAGCACCCGTAATACGGTATGCCACAACAAT (SEQ ID | |
| | NO: 142) | |
| 3300031853\|Ga0326514_10013355_ | GATGTTGTTATGCTGTTTTTGTAAGTAATAAACAAC (SEQ | 28-30 |
| 6\|M (SEQ ID NO: 34) | ID NO: 70) | |
| | GTTGTTTATTACTTACAAAAACAGCATAACAACATC (SEQ | |
| | ID NO: 131) | |
| 3300031993\|Ga0310696_10000014_ | ATTGTTGTAGACCTTGTGTTTTAGGGGTCTAACAACG (SEQ | 29-30 |
| 323\|P (SEQ ID NO: 35) | ID NO: 77) | |
| | CGTTGTTAGACCCCTAAAACACAAGGTCTACAACAAT (SEQ | |
| | ID NO: 138) | |
| 3300031993\|Ga0310696_10000226_ | ACTGTGTTGGAATACAATATGAGATGTATTTACAAC (SEQ | 30 |
| 76\|P (SEQ ID NO: 36) | ID NO: 78) | |
| | GTTGTAAATACATCTCATATTGTATTCCAACACAGT (SEQ | |
| | ID NO: 139) | |
| 3300031993\|Ga0310696_10000447_ | ATTGTTGTGGCATACCGCAAGGCGGATGCTGACAAC (SEQ | 26-34 |
| 27\|M (SEQ ID NO: 37) | ID NO: 79) | |
| | GTTGTCAGCATCCGCCTTGCGGTATGCCACAACAAT (SEQ | |
| | ID NO: 140) | |
| 3300031993\|Ga0310696_10026614_ | AATTGTTGAGATACCGTTTTTTATGGTATTGGCAAC (SEQ | 30 |
| 2\|M (SEQ ID NO: 38) | ID NO: 80) | |
| | GTTGCCAATACCATAAAAAACGGTATCTCAACAATT (SEQ | |
| | ID NO: 141) | |
| 3300031993\|Ga0310696_10030100_ | ATTGTTGGAATATCACTTTTGTAGGGTATTCACAAC (SEQ | 30-31 |
| 3\|M (SEQ ID NO: 39) | ID NO: 58) | |
| | GTTGTGAATACCCTACAAAAGTGATATTCCAACAAT (SEQ | |
| | ID NO: 119) | |
| 3300031998\|Ga0310786_10000003_ | ATTGTTGTGGCATACCGTATTACGGGTGCTGACAAC (SEQ | 25-31 |
| 467\|M (SEQ ID NO: 40) | ID NO: 82) | |
| | GTTGTCAGCACCCGTAATACGGTATGCCACAACAAT (SEQ | |
| | ID NO: 143) | |
| AUXO013988882\|Ga0247611_ | ATTGTGTTGGGATACACTTTTATAGGTATTTACAAC (SEQ | 29-31 |
| 10000101_23\|P (SEQ ID NO: 41) | ID NO: 83) | |
| | GTTGTAAATACCTATAAAAGTGTATCCCAACACAAT (SEQ | |
| | ID NO: 144) | |
| 3300028805\|Ga0247608_10000186_ | TATTGTTGAATACCTTTCTTATAAAGGTAATTACAAC (SEQ | 29-46 |
| 37\|P (SEQ ID NO: 42) | ID NO: 84) | |
| | GTTGTAATTACCTTTATAAGAAAGGTATTCAACAATA (SEQ | |
| | ID NO: 145) | |
| 3300028805\|Ga0247608_10000895_ | TGTTGTAAATGGCTTTTTATGGGCAACGAACAACTC (SEQ | 28-45 |
| 42\|M (SEQ ID NO: 43) | ID NO: 85) | |
| | GAGTTGTTCGTTGCCCATAAAAAGCCATTTACAACA (SEQ | |
| | ID NO: 146) | |
| 3300028805\|Ga0247608_10006074_ | ATTGTTGAATGTATTCTTTTTTAGGACAGATACAAC (SEQ | 28-30 |
| 1\|M (SEQ ID NO: 44) | ID NO: 86) | |
| | GTTGTATCTGTCCTAAAAAAGAATACATTCAACAAT (SEQ | |
| | ID NO: 147) | |
| 3300028833\|Ga0247610_10000007_ | GATGTTGGACACTATGTTTTATACGGTGGATACAAC (SEQ | 30 |
| 379\|M (SEQ ID NO: 45) | ID NO: 69) | |
| | GTTGTATCCACCGTATAAAACATAGTGTCCAACATC (SEQ | |
| | ID NO: 130) | |
| 3300028833\|Ga0247610_10004486_ | TATTGTTGAATACCTTTCTTATAAAGGTAATTACAAC (SEQ | 29-46 |
| 2\|M (SEQ ID NO: 46) | ID NO: 84) | |
| | GTTGTAATTACCTTTATAAGAAAGGTATTCAACAATA (SEQ | |
| | ID NO: 145) | |
| 3300028888\|Ga0247609_10000668_ | ATTGTTGAATGGTATCTTTTATAGACTGATTACAACT (SEQ | 29-41 |
| 74\|M (SEQ ID NO: 47) | ID NO: 87) | |
| | AGTTGTAATCAGTCTATAAAAGATACCATTCAACAAT (SEQ | |
| | ID NO: 148) | |
| 3300028888\|Ga0247609_10003329_ | ATTGTTGGATAATAGGTTTTTTATCTTAATTACAAC (SEQ | 29-30 |
| 9\|M (SEQ ID NO: 48) | ID NO: 88) | |
| | GTTGTAATTAAGATAAAAAACCTATTATCCAACAAT (SEQ | |
| | ID NO: 149) | |
| 3300028888\|Ga0247609_10016480_ | ACTGTTGAATAGTTGATTTTATATCCTATTTACAAC (SEQ | 29-30 |
| 8\|M (SEQ ID NO: 49) | ID NO: 89) | |
| | GTTGTAAATAGGATATAAAATCAACTATTCAACAGT (SEQ | |
| | ID NO: 150) | |
| 3300031992\|Ga0310694_10000010_ | GATGTTGGACACTATGTTTTATACGGTGGATACAAC (SEQ | 30 |
| 351\|M (SEQ ID NO: 50) | ID NO: 69) | |
| | GTTGTATCCACCGTATAAAACATAGTGTCCAACATC (SEQ | |
| | ID NO: 130) | |
| 3300031992\|Ga0310694_10022272_ | TATTGTTGAATACCTTTCTTATAAAGGTAATTACAAC (SEQ | 29-46 |
| 2\|M (SEQ ID NO: 51) | ID NO: 84) | |
| | GTTGTAATTACCTTTATAAGAAAGGTATTCAACAATA (SEQ | |
| | ID NO: 145) | |
| 3300031994\|Ga0310691_10000084_ | TGTTGTAAATGGCTTTTTATGGGCAACGAACAACTC (SEQ | 28-45 |
| 157\|M (SEQ ID NO: 52) | ID NO: 85) | |
| | GAGTTGTTCGTTGCCCATAAAAAGCCATTTACAACA (SEQ | |
| | ID NO: 146) | |
| 3300031994\|Ga0310691_10000270_ | TATTGTTGAATACCTTTCTTATAAAGGTAATTACAAC (SEQ | 29-46 |
| 20\|M (SEQ ID NO: 53) | ID NO: 84) | |
| | GTTGTAATTACCTTTATAAGAAAGGTATTCAACAATA (SEQ | |
| | ID NO: 145) | |
| 3300032030\|Ga0310697_10001273_ | ACTGTTGAATAGTTGATTTTATATCCTATTTACAAC (SEQ | 29-30 |
| 44\|P (SEQ ID NO: 54) | ID NO: 89) | |
| | GTTGTAAATAGGATATAAAATCAACTATTCAACAGT (SEQ | |
| | ID NO: 150) | |
| 3300032030\|Ga0310697_10005481_ | ATTGTTGGATAATAGGTTTTTTATCTTAATTACAAC (SEQ | 29-30 |
| 13\|P (SEQ ID NO: 55) | ID NO: 88) | |
| | GTTGTAATTAAGATAAAAAACCTATTATCCAACAAT (SEQ | |
| | ID NO: 149) | |
| OBLI01003123_14\|M | ATTGTTGTAGATACCTTTTTGTAAGGATTGAACAAC (SEQ | 30 |
| (SEQ ID NO: 56) | ID NO: 90) | |
| | GTTGTTCAATCCTTACAAAAAGGTATCTACAACAAT (SEQ | |
| | ID NO: 151) | |

### Example 2—Identification of Transactivating RNA Elements

In addition to an effector protein and a crRNA, some CRISPR systems described herein may also include an additional small RNA that activates robust enzymatic activity referred to as a transactivating RNA (tracrRNA). Such tracrRNAs typically include a complementary region that hybridizes to the crRNA. The crRNA-tracrRNA hybrid forms a complex with an effector resulting in the activation of programmable enzymatic activity.
tracrRNA sequences can be identified by searching genomic sequences flanking CRISPR arrays for short sequence motifs that are homologous to the direct repeat portion of the crRNA. Search methods include exact or degenerate sequence matching for the complete direct repeat (DR) or DR subsequences. For example, a DR of length n nucleotides can be decomposed into a set of overlapping 6-10 nt kmers. These kmers can be aligned to sequences flanking a CRISPR locus, and regions of homology with 1 or more kmer alignments can be identified as DR homology regions for experimental validation as tracrRNAs. Alternatively, RNA cofold free energy can be calculated for the complete DR or DR subsequences and short kmer sequences from the genomic sequence flanking the elements of a CRISPR system. Flanking sequence elements with low minimum free energy structures can be identified as DR homology regions for experimental validation as tracrRNAs.
tracrRNA elements frequently occur within close proximity to CRISPR associated genes or a CRISPR array. As an alternative to searching for DR homology regions to identify tracrRNA elements, non-coding sequences flanking CRISPR effectors or the CRISPR array can be isolated by cloning or gene synthesis for direct experimental validation of tracrRNAs.
Experimental validation of tracrRNA elements can be performed using small RNA sequencing of the host organism for a CRISPR system or synthetic sequences expressed heterologously in non-native species. Alignment of small RNA sequences from the originating genomic locus can be used to identify expressed RNA products containing DR homology regions and sterotyped processing typical of complete tracrRNA elements.
Complete tracrRNA candidates identified by RNA sequencing can be validated in vitro or in vivo by expressing the crRNA and effector in combination with or without the tracrRNA candidate and monitoring the activation of effector enzymatic activity.
In engineered constructs, the expression of tracrRNAs can be driven by promoters including, but not limited to U6, U1, and H1 promoters for expression in mammalian cells or J23119 promoter for expression in bacteria.
In some instances, a tracrRNA can be fused with a crRNA and expressed as a single RNA guide.
The system can include a tracrRNA that is contained within a non-coding sequence listed in TABLE 9. For example, in some embodiments, the system includes a tracrRNA set forth in any one of SEQ ID NOs: 152-204.

TABLE 9
Non-coding Sequences of Representative CLUST.091979 Systems

>3300028887|Ga0265299_10012919_3|P
TATATCGTGGCCGAATATGTTAACGCGGACGACGTCCGTCTTGTGAAGTTTCAGGACGAGGATTTCGACAGGCTTCTTGACAAG
GTTAGAGAATGGAACAAGAAACATCTTGTTGTTGGAAATCGGAACTTCGAAGAAAAATTTGCGTAATCCAAAAATTTTCCGTAT
ATTTGCGGCGTGAAATTAAAAATATGTTTAACTAAAAACAAAGATTATGGCACACAAGAATCCTGATGGGGAGAACACCATCAA
CAAAACTTTTATTTTCAAAGTGAAATGCGAGAAGAATGATATTATATCGTTCTGGAAACCCGCAGCTGAAGAGTATTGCAACTA
TTACAACAAACTTAGCGAATGGATTGGCAAAGATATGTATAACACGCCGTCATGGAACATCCGGCAAGAGTTCAAGAAGAATTT
AAGTGTTAGAACCATAAACACGTTTCGTGAGCTTGGCAATGTGAAATACGGCAAAATCAACAATGAAGGGCTTTTTGTCGAAGA
CGATGTGTAAACATTAAGATTTCCATACGACAGGATTCAAAAAAACGTTCTTTGAAATATTGGATTGGTGGCAAGAGGCTGTTT
TTTTTAGGCTAAAAAGTTGTGTAAATAGCAGAAACACAGAACATAACATAAAATCT (SEQ ID NO: 91)
>3300028797|Ga0265301_10000251_12|M
AACTGCTACAATTCTGCCGAGTTTATGATTCAGACAAAATTCAAAAAAAGACTTCCGCAAGCAACCGTTTTTGGTGAATTGAAC
AGAAACGGGTATGTTAAAGTATTGACCCAAGAAGAATATGACGAACTCACAAAATCAGCAAAATAATTTATTACTGATTGAAAA
ATAAAGCGTTCTTTGACATATTGTATAACAAACAAGCATTTTTGTAAGAGATAACCCATTTCATTTTATTGATATACAATGAAA
TGAAAAGAATAT (SEQ ID NO: 92)
>SRR094437_845781_4|M
GATAAATTTGCCCGTAATGTTATCGGGTTCAAGTCATATCACGAACTGCTTGATAATGCTATCATAAAAGAAAAATTACAACGG
GAATTTGGTTATGAAGATGCTCCGAAAACGTGGTTGTTCGGACAACAAAAAAATGAATGTTTCTAATGTATTAAAACAATAATT
CAATTACAATTTTAAGATTATGGCACAACACAAATCAAACAACGAAGAATCAGCAATCAACAAGACTTTCATTTTCAAGGCAAA
ATGCGATAAGAACGATGTCATATCGTTATGGGAACCAGCGGCAAAGGAATACTGCGACTATTATAACAAAGTGAGCAAGTGGAT
TAAAACTATGTATAACATACCCGCATATAACATTAAGTCCAATTTCAAGAAAAATTTGAGCGCCAAAACAATTCAAACTTTTAG
AGAACTTGGACACTACCGTGACGGAAAAATAAATGAGGATGGTATGTTTGTTGAAAACTTGGAATAATTCTGTATATACCAATT
AGAATTGAAAAAAAAACGCTCTTTGACATATTGTTTTCTACATAAAAACAAGATTTTACACAACGCAATACATCATAAAGTGTT
GCGTTATAACAAATAACAAAAATTCT (SEQ ID NO: 93)
>3300021254|Ga0223824_10022219_2|P
TTTATTCAATGCGAACCAGAGGTCTTGACGCATGAATCTGGCTATACATATCGTTATGCGACCGACGAAGAGAAAATATTGATT
AAAAGATGCAAATATTGAATAGGCAATTTTAAATTGTGAAAAAAAAAATGATTGAATATAAGTTTACGTTTGAACTGGATGGAC
ATCTATCGGCGTACGATTTTGTTACGTTGCAAGAACGGTTTGAAAGGGAATTGAATCCTTATTTTGATGATGGGAGCATATCTG
GTACTCTTTCTTATGCAAATGATGATTAATATGCAAATAATATGGCACATGTAAGAACAAAAAATGAAGGAAACATGGCAAAAA
CATATTCTTTTAAGGTCAGAGAAACAAACCTTAAAAAGGATGTGATGATTGAATATAACGAATATTATAACAGGTTATCCGATT
GGATATGTGGCAATTTAACCAAAATCTCGGAAAATGAAGAATGGAGGAATGCCTTATGCAAACCAACAGAAAACATGTACAACG
AACCGATTTACGTTCCCTTGGTTAAATCACAGAACGGAATGTTCAAGGCAATTAAAAAATTGGGCGCAACGAAGATATGGCAAG
AATAGAAAGACCGATTTTTAAATCTGAAATCACTTCTAACGAATTGTATACTAAAGAAATATAAAGAATATACATCTTTTATGA
CATTATGATATTGTTGTATGCATCATTTCACATGGTAATAACAACGAAGAGAAACACCGAGCGACCCACAAACCTATTGTCGTA
CGCATCATTTCACATGATAATAACAACGAATATTCCTGCAAGCATGATTTAACAATTTTTAAGAACCTGGTGGTTTCTCCGTTG
GGTTCTTTTTAGTATCTTTGCCTTGTTGAAACAAATAAAACAAATTGAATTATGATTTATAAAGGCAAAGAAATAGACGAAAGT
TACCACATCAATAAATGGGAAGATGAAGAGATTTACTCTGGTCCAACCCATTATGAATCATTCGAAGCCGATGAAATAAAAGAG
TTCTACCTCAAGGCACTTGCAAAGGAAAAGGAA (SEQ ID NO: 94)
>AUXO017332817_2|M
GTGCGCATATACACTCAATTCGCCGATGACCGTGTGTACGCGAAGGATTGTATCGACGGATTCTTTAGTATAAGACAAGATACC
GAAATGCGCCTCGTGTATAAAAATGAGATAGCACGCGGGCTTGAGTGTATCAATATTGTAAGATAGTAGTTTTCTGTTATTTTA
CATATTGATGTGTTTTGGCATGGTTTTTGTTAAAATATAATCTAGCAGTATTGAGACTGCGGAGTAACGTGTCTAACTGTTTCA
TTATAAGCAGTAAAGACTAATATTTTTATATCTTAAACTTATTTTTATTATGGCTGGTCACAGCAAAATCAAAGAAAATCACAT
TATGAAGGCGTTTCTTATGAAAGTAAAAGAAACGCGAAAAAAACAGTGGCAATCAAATTTTATTAGAAGTGAGATTGCTAAGTT
TACAAATTATTACAATGGGCTGTCAAAGTTCCTTCTTGGAAGCCCGACTGGAGGGACATATGACACTGCATATTTTGATACAAA
GATTCAAGGCTCCAAGGGGGTATATGATAAGATTAAAGAAAACGGAGAAACTTATATTGCAGTATTAAGTGATGACGTTATTAC
GGCAGAGGTGTAAAATCCTCTGCCAACATCGCAAGTAACTCATTGAAAATTAGTTAAATGCGAATGCCAACAAAAGTGAACGAA
CTGACTTGTAAAGCAGGATGTTGTTATATCTTTTTGTAGATAATAAGCAACAAGATACAATCAATCGCGAGTTTATACTGAAAT
GTTGTTACACTGTTTTTGTAAGTGTTAAACAACCTTGCACAAATGTCATCTACCAGTACAATAGATGTTGTTATACTGTTTTGT
AGGTATTAAACAACCATTGCGCAGACTGACAGAGTAACCTTTCCTGATATGTTGTTACACATTTTTGTAAGTGTTAAACAACTG
ACGCATTGATATTGCCTTGTCTATTAAGAATGTTGTTATGCTCTTTTTATTGGTATAAACAACCGAGCAACTGGTACTCAAATT
TTAAATACTGTCGCGCTATGTTATGTACATCGAACAGCTACCACTCAATGGCTTTGTTTGCAACCGTGATTAATTCAATCGCGG
TTGCATTTGTTTTATGATGTGTTTTTGTATATATTATGTATATATGGAAAAGGAAAACAGGGTATCGGAGTTATGGAGCAAGTT
CTCTGATATTGACTTGCGCCGAAGCCAAATGACATATATGCCAATAAGAGGTAGTAAAAGATACGGCAGAAGAATAAAACGTAG
TGACATCGAGTACGAGTACAGATATCTGTATAGAGCAAACAAACATTGGTAATATGACCGTAGCTAAATTATCAAGTAATCATA
AGCCAGCGTGCCTTGGACGAATCTCAGCTTTAAACACCCCGATTAGATTTGAGTGTCGGGCTGGTAATAGTATAAGGCCTGGCA
ACATAGAGTATAGCTATAAAAGATGGAAAACGTCGTAATTTCAACTATGCACAACCCGCATACGCTGGCTTATTACCAAGGTAA
GCTGGCTCCTATGCATTTCAGACAAGATACAGG (SEQ ID NO: 95)
>3300021431|Ga0224423_10015012_2|P
AGCCTGTATACAGGGACAAGGTTAAGTACAACACCAAGGCTGAGGCAAAGAAGAGGGCTGATGATATGAACAAACAGAATAGGG
TCATACACCAGCTGTCTGTTTATTTGTGTCCTAAATGTCATAAGTGGCATATAGGTAGGAGCAGTGTGGAGAGTGTGCGCAGGG
AAGGGTACTTTAGTCAGATTTGAAATTAATTGTTATATGGCGCATAGAAATAAAAACCTAGCAGAAAACTGCATTAACAAAACA
TTCAGTTTTAAAGTCAAAGCCGAAAAAGAGGAGATAAATTCAAAATGGATTCCAGCCATTAAAGAATATACTGCTTATTATAAC
AGGATAAGTGACTGGATAAACCTGTATTCACAGCCTACTTATGATATTAAGGAAGTTTATAAGAAAAACGCTGGTTGCAAAGTG
ATAAACGACTTCATTAAAAACGGTAACGCCGTTATATGTTGTATCGAAAATAACAAACTAATTGAGACAAATGGAAGACAATAG
TTCAAATTTTAAATGTAAAACAGTCATTAATGTATTAATATATAATACATAGCAAAAATCCAGATGTTGAATACATTTCTTTTA
AGTGTACTTACAACGCGGTGGCATTGCTAAAATATAGTCCTGTGGATGTTGAATACATTTCTTTTAAGTGTACTTACAACCAAC
GCTGTACACATTGCTAATGGATGATGACGATATAGAGGTGTTGAACTACCTTAATGAAAACTACACCAATGAAAACATTGAGTA
TATACGCGGTTGGTGGATGGATGACGACGATAAACTCCAGACACTTGACAGGTTTTTGAAAAATTTTTCAATATAGACCTGTCA
CTGTTGCGGCTATAAGAAGACCGATTTGACACTGAAAGACCGATACTGGGTTTGCCCCGAATGCGGTGCAAAACTAGACCGCGA
TACCAATGCAGGAATAAACATTAAGAATGAGACAATTAGACTGATAAACAAAGAATAATGAGAACTATAATAGGGAGGTGTACC
CCCGAATTTAAGCCAGTGGAGAACCATACAAACCTATCATATAGGGGTTCAATGAATCTGGAATTTCTGACAAAAACAGGGTTT
AACAGCCAGTGTACCAATGACTAACACAGGACATATAAAGACAAATCTAACAATAAAAAAAAATATTGACCAATTCTGCAGAAA
AAACAGGTTGGTTTCGGTTATGTTGGTGAATAAAGACAGTTAGATTAATTTTATATGGAAATGAAAATAGAGACAAAAGACGAG
AACATCTACGTATTCATCTATGCCAAGTCCGCCTACTTCGGCAATACATTTGAATATGGCGGCACATTTTCCGTCGGCAAGGAC
GACAACTGGAACGATGTGAGAGGCCACGTTACCGAA (SEQ ID NO: 96)
>AUXO013988882|Ga0247611_10000101_23|P
GACAACATCCTGGTCAAGACCGAGGTTAACAGAAGGTACTGCCGCCTTATGACCGACGAGAACGGAGTGTGGCTCCTGAGGAAA
AACGACAAACATCCAACATATTTTATCTACCAGAACGGAACACTCTATCAATATGAGGAAGATTGATTAGTTGATGTTTTCATA
ATAATTTTATCTGGAATTTGAAAAGATTCCAGATTTTTTTTTTATTTCGACTGTACAAAAAACAGGTTCCGTTGCGTTATATAG
GTGTAAATTAAAAATTCAGTCAAACAAAAATTGGAATAAAATATGGCTAACAAGAGAACAGACACAACAATCAACCTTAACAAA
ACCGTTATAATGTTAACGAACATGCTGCCAGAAGTACGGGCAATGTTTCAGGCGGGAATACGCCAGGCTCAAGTTTATGCAGAC
TTGGTGAACAAGTGGATATGTTCACAGGAAATGAGAGAGGTTATGTGTCTCCATCCGTCAAAAAAGGACGGGGTGTACGACCAA
CCGTTCCTGAAAGCTACAACCAAATACCCAGCCACGGTAGCTGGTATCCTGCTTAAGATGGGAAAAACAACCAATTGGGGTGAG
AAATAATACCCACCCGCCCCATTTTTTTACACTGATTAGTTCTTTGACTTATTGATTTATATTGGTTTACACAAATTATCGACA
CAATAAATAAAAAAAATTGTATATTAGTAGTATGATGACAGAAGAAACACGGAAGACAATAGAGAGCGTCATAGTGGTTCTCGG
CATAGCAATCATGCTGGCAGCCGCCGTCCGAATAATGACGCAGAACAAAGCAATTGTGAAATATGATGAACAGGTTGAAACCAT
GCAAACTTGCATA (SEQ ID NO: 97)
>AUXO013988882_8|P
ATGGAAGTTGTACGTGGTGGAAATCAATGGGAGGTTTATGACAATTACGATGAGACTATGAAAGCATCAAAAAATGTAAGGTCT
GTATTGGGACTTCCGGAAGTAAAATATCCACCTGAGGATTTTAGGACATATAATTTCTAATAAAAATGAACGGAAAAATTTCCG
TTCATTTTTTTTTTGTTTATTGGTGAAAAAATAGTATCTTTGTAAAAAATAAATGTTAAAATATTTTTTATGGGAAATACTACA
AAAAAAGGAAATTTGACGAAGACTTATTTATTCAAAGCCAATCTTTCAGAACAAGACTTTAAATTATGGAGGTCTATTGTTGAA
GAGTATCAAAGATATAAGGAAGTGTTGAGTAAATGGGTATGTGACCATCTTAGAAATGCAATGTGTACGAACCCGAAAAGTGAG
ACTGGATATTCTGTACCGTTCTTGACTTCAAGAATCAAGAAACAGAACATTATGGTTGTAGAATTGAAAAAAATGGGCATGGTT
GAAGTCTTGAATGAAAAATCAACAGAAATTTAAGAAAAAAATATTTATATAATGTACTGAAAATAAGTAAATAATAAATATTGT
GTAAAAAACTTGATATTTTTTTTTTGTTATCTTTATAATATAAAATAAAATGTAAATATGAAAAATCTGTTAAAACTCAAAGAA
CAAATCAAGGATTACAAACATCTTCAGTTTGTGTTGGAGAAAGAAGATGAATCTGAACTCCATTATAGATGTATGACTGAAGAT
TTTTCGTTCAAGGTATCTGAAGAAAAAGACGGAACACTT (SEQ ID NO: 98)
>SRR3181151_741875_3|M
TTATAAACATCTAAAAAGAAAGACTTATGACAACAAAACAAGTTAAATCAATCGTTTTAAAAGTAAAAAACACTAATGAATGCC
CTATTACAAAAGATGTAATAAATGAATATAAAAAATATTATAATATATGTAGTGAATGGATTAAAGATAATCTAACAAGTATTA
CTATTGGAAACGAAAATTTACGAAAATTATTTTGTGGTAAACTTAAAGTAAGTGGATATAATACACCAATATTAGACGCAACAA
AAAAAGGTCAATTTAATATATTGGCAGAATTAAAAAAACAGAATAAAATTAAAATATTTGAAATAGAAAAATAAGTCTTATGAT
TACAAAAATAATAGATTTCAAACATTTTTTTTAATTCTATTTTATTGACTAATTCATTGAAATATAAATAATTACAAATAACCC
(SEQ ID NO: 99)
>3300028805|Ga0247608_10000186_37|P
GATAGATATAGTATTGCAGCATTTCTGGCTTGCGAATCATCAGCAATGCAAAAATGTGACTATTGGAACAATGATGATGCCCAA
GATTACATAAGAAACTACAAAGAGGCTTATAGTAATGCAGTAAGACTTGCGTTTTTTAATGATTAAGCAACACGCTTAACATTG
TCAAATGTAACGACATTAAGTGCGTGTTTCATAAGGGCAGCGAACCTTTCGCCGCCCTTCTTTTTTTGTTGCTGTAACGGAATT
ATGTTTACTTTTGTGCCATCAAGTATATAGTTCCCTTAATAAATTGTATATTAATTAAAAGTTTGGCACAATATTTGATGCGTA
CAAATTAAAATAAAAACATTTTGAATTTTAAAATTTAATTTGTAATTTTAAATAAGAAAGTTTTATTTAACTAAAATAAAAAAA
ATGAATAAATCTTATGTTTTTAAGTCGAATGTGGCTATTGATGACATTATGTCTTTATTTGAACCGGCAATTGAAGAGTACATA
AACTATTACAATAGAACCAGCGATTTCATTTGTGATAATCTTACATCAATGAAAATCGGAGATTTGTTGCTTCTAACAATGTGT
ACTAAGACAAAAGAAAATAATAGATACGGTAACCCCCTCTATAATATCAAAGATACTTTTAAAAAGAAAATACCATCTTCAATA
CTTAATATATTCAAAAAAAAGGATATGTATCAAATAATATGTGATTAATTATGCCTTTTTTTAATAAAAAATTGTTAAATAATA
CTTTGTTTATTAATAAATTATAAATATCACAGTAAACTATTAGGGATTTGTAAAATTTATGGAAATTATATACATGATGGCACT
AAGATTTGGTTATTAAGAAATTTTTCTGTATAAGTATAATAACCTATTTATAATTATAATTGAATAAAATGTATAATATGGAAA
ACACAGGCTTTTATACAGTTTCAAATATTGAAACTTCTCATAAGCCAACCGAAAATTCTAATGACGAAATTCTTAGGATTTTCA
ATAAAAGAAGGCCTTATTGCCCTTCAGACTTTAAGAAGCAACATTTTATT (SEQ ID NO: 100)
>3300028833|Ga0247610_10000007_379|M
AGGCTCAACCTCCTCAACCCGATTTATCTTGAGATCGCCAAGTACGGACACTTCGGGAGGAAGAGCTATGTGAAGGACGGCATC
AAGTACTTCCCGTGGGAGGATTTGGATTTGGTTGAAGACATCAGAAAAATTTTCGAAATGGAATAGAGGGAACCGGAATTTTTT
CCGGTTTTTCTTTGTCCTTTCGAAAATAAATAGTATCTTTGTAAAAAAACAACAGATTATGTACAATAGTAAGAAGAAGGGGGA
GGGTGACATTCAGAAGTCGTTCAAGTTCAAGGTCAAAACGGACAAGGAGACGGTCGAATTATTCAGAAAGGCCGCAGTCGAATA
CTCGGAATACTACAAGAGGCTGACAACATTCCTCTGTGAGATGTATAACAGACCAGCGTTTGACTTGAAGGAGTGCTACAAGAA
AAATTCCAATGTAAGTGTCTTCAACACATTGAAGAAAACTCTCGGTGCAATATATGGAAAGCTCGATGAAAACGGAAATTTTAT
TGAGAATGAATGTAATAAGTAACTGGAATAAAAGAAATTAGACAGAGTAA (SEQ ID NO: 101)
>3300028887|Ga0265299_10011526_3|M
TTGTATTGGTTGCTGTATGGCGACGGAAGTGACATATATGATGACGGGTGGTTTGACTGTGTTCATAATTTTGCCCGTAATGTT
ATCGGGTTTCAGTCATATCACGAACTGCTTGATAATGCTATTATAAAAGAAAAATTACAACGGTAATTTGGTTATGAAGATGCT
CCGAAAACGTGGTTGTTCGGACAACAAAAAAATGAATGTTTCTAATGTATTAAAACAATAATTCAATTACAATTTTAAGATTAT
GGCACAACACAAATCAAACAACGAAGAATCAGCAATCAACAAGACTTTCATTTTCAAGGCAAAATGCGAGAAGAACGATGTCAT
ATCGTTATGGGAACCAGCAGCAAAGGAATACGGCGACTATTATAACAAAGTGAGCAAGTGGATTAAAACTATGTATAACATACC
CGCATATAACATTAAGTCCAATTTCAAGAAAAATTTGAGCGCCAAAACAATTCAAACTTTTAGAGAACTTGGACACTACCGTGA
CGGAAAAATAAATGAGGATGGTATGTTTGTTGAAATTTTGGAATAATTCTGTATATACCAATTAGAATTGAAAAAAAAACGCTC
TTTGACATATTGTTTTCTACATAAAAACAAGATTTTACACAACGCAATACATCATAAAGTGTTGCGTTATAACAAATAACAAAA
ATTCTGGACGGGAAAGGAAGATGTCAGACGTTTTTATTGTTGGAATACTCGTTTTTTACGGTATTTACAACTGCCCCGTAGCGG
AATCAAAATACCACCGCATTGTTGGAGTACAAGTTTTACACGGTATTCACAGTACGAACACCGAATGAACTGAAAAAAATAAAC
CCGACCTTGCAACCGTAGATATAAATAAAGCAATACAAAATTTGAAACTATGGCACACATTAAAAAAATTGACGAAATGGCAAG
TCAAACTGTTTCACTCCGTTCTGACGCATTGTTCAAAAAAGCGTTTGAGGAATTTGAAAAGGAGTTGAAAGAAGTTCTCAAATC
GCACAACAATATCATTTATTGTGGAGGTGAT (SEQ ID NO: 102)
>3300028797|Ga0265301_10009039_3|M
CTCATCAAATTGTACAAGTCGTTGACGGACACTGAATTTGACAAGAAGAAAATCATCAATGATGTCTACGACGGCACTTTTGAG
ATAATCCTCAAATACCCAAAGAAGAAGAACGGGACATTCGTGTTCTGGAAACATTACAAGAAGTAACACAATGATACACAGTAT
GTTGTAAGAAATAAGATTTAGGCTTTAATTTTAATATATGAAAATATGGCACACAAAGGAGAAAAGGAAGGCTACCAAATCAAG
ACACTGAAGTTCAAGGTACGCTCGCATGACATCGGGAAATCACTTTATGATATTGTCAACGAATACACCAACTACTATAACAAA
GTAAGCAAATGGATATGTGACAACCTTGGTTACAACGAGCCATTCTACAAGTCAAGGGTGAAAAGCGCCGCCTCCATGATGTCA
GGATTGAAAAAACTGGGCGCCACCATGCCATTGACGGATGAAAATGCCATTTTTTCAACACCAAAACCGAAGAAAAACATTGGA
AAACAATAATTTACACAAAGTCTACGGCGGGAATCGTGATAAAAATGAACGAGATTGTTGGGATATACCTTTTATAGGATTTTC
ACAACATCTGAGTTGTTTGATGTTAAAAACTTTAACTAATAAGGCAAGAAGTCCCATTCCTTCAGGTGGGGGTAGTTCATTTGT
TGGGATACTCGTTTCACACGGTATTCACAACTTCCAACCAACCATTAAAAAACCTTCAAATATTGTTGGAGTACCCGTTTTATA
CGGTGCAAAGCCTCCCCGACGATTTCAAGTTCCTGTACGAAGATGTCAATTTTGGATAGCAACTGTTACCAATAAACATATTCA
AAAGTAATCAAATATATTCAAAAACAACTCGTATAAATATATAAAGTTCGTGATATTTATTATAAAGAAGCCGAAGGAGAGAGC
GGTTTCCGAACAATAAAGATATACAGAGGTTTTATTCTTGACGGCACTCTCTCCTTTAGCCGCAAGTTTAATTCCTCTTTTTTA
TTGCACTATGGTCATCGACAGCAAATATACCAAGACATTCAAGTCAAACGGACTGACCCATCAGAAATATGACGAGTTGCTCTC
GTTTGCTTCTATGCTGCGTGACCATAAGAACACCATCTCCGAATATGTCAATGCCAACCTTGAACACTACCTCGAATACTCAAA
ACTCGACTTCCTTAAGGAAATGCGTGCGAGGTACAAGGATGTCGTTCCGAGTTCGTTTGACGCTCAACTCTACACG (SEQ ID
NO: 103)
>OBLI01003123_14|M
AGAATCTGTCCTATATGTGGGAAACATTGCGAATATGAGGAAATGGAGGGCGACCACATTGTTCCATGGTCAAAGGGCGGTAAA
ACCGATATAGGCAACCTCCAAATGCTATGCAAGAAGTGCAATCACGAAAAGTCCAATAGATATTAGTGGCGTAATCAAAAATTT
GTTTGTGTTGAGGAAAAGCAGTGAAAAAAAACATTGTTTTTCCTCAATTTTTATTTGCATAATTCAAATAATTTTTTATTTTAT
AGGATAATAGAGCTAACAAGCATTAACAATTATTAAAACGATTTATATTGAAAATAAATTTTGTGGGAATATTTATTTTTACTA
CCTTTGCATCGTAATACAATTAAACAAATTTTTGATTATGGCACACAAAAAGAACATAGGAGCAGAGATAGTAAAAACTTACTC
TTTTAAGGTGAAGAATACCAATGGTATCACAATGGAAAAATTAATGGCCGCCATTGATGAGTATCAGTCGTACTATAACCTTTG
CAGTGATTGGATATGCAAGGGTCTTGACGAAATAATGAGGAATACTTTTCTGAAAAAAGCAAATAGCAATAAATCATTGTATAA
TCAGCCAATCTACGATACGGGTATCAAGAAAACCGCAGGTGTGTTTCCTAGAATGAAAAAATTAAAGAAATATAAAGTTATCTG
AAATAAAATATGTATTTTTCTTTGTGGAAATACCTATTAATAGACTGATTTCTAATAAGTTATAAGAAATACTGTATGTAGTAA
ATAAGATATCATATTTTTGCGGAGAGGCACATGGAGTATGCTATAGGGTTTTTGCTACCGAGCAGAAAGCAAAAGAAAAAATGC
AGGGATGATATCATTTCATTCTTGCATTTTGCTTATACATATTCAATCAAGTATCATTTTCTGTTTTTACTATTATCCTATAAA
ATAAAATTTTCCTCAACATTTCCAAATTTAATTTGCAATAATTTTTTTTGATAAAAAGTGCAAATAAATTTTATAGATTCAAAA
CTTTTGATTAACTTTGTAACAAGAAAAACATTAAGGATTATGGGTTACACATATTTTAGGGTTACTGATGAAAGGGCAAGGGAT
GTTATGCCAAAGGCGGCTGAAATCATAAAGGATATTTTC (SEQ ID NO: 104)
>3300028887|Ga0265299_10000133_30|M
CTTCACCTCGTACAGCCGACAATAAGTTTCGCTTGGACTGAACTTATGTGCGCCTGCGCATTCATAGCGGGTGGCGTATCAGGC
TATCTCATCAAGGGCAAGATGCCAAACGACGGGAACAAGTACCAGTCGGTAGAGGGAAAGGAATAGGACAAAAAAAAACACATC
ACCCCCAGCGCATCGGGCGCGGAGGTCGGGTGTGCATATAACGGTGTCTGTGGCGCAACTGGTAGCGCAGTGGATTGTGGTTCC
AAAGGTTGCGAGTTCGAGCCTCGCCAGACACCCATTATCACACGGAAGCATTGGATGGAAGTGCAAGTACCTACTGGGAACTTC
CTGAAAGCGCAAGCAAAGTCGAGGTCTAACGGTACTTATGACCGAGGTAATGGCGGGGCGTTGGTTCGAGTCCAACACAATGTT
TCCATTTACACGGAGAGTTGCAGGAGTGGTAACTGGTCAGATTGCTAATCTGAAGCCCACCTCGTTGTGGCAGGGGTCCGAATC
CCTTACTCTCCGCCAAGCAACATACCCGCAGAGTAGTCGCGTATATTCTGTCGGTGTGGTCAGAAAGAAGTGAATGTGATGCGA
ACGCGCGAAACCATCGCATTTAGAGTCCGAATCTCCTCTGCGGTAGCCAGTCCGCATAGTTTAATCAGGTTAAAACATTCTGAC
GCTTTTTTAAATCGCGGGAGTAGTTCAGTGGTAGAACATCGGCTTCCCAAGCCGAGGGTCGCGGGTTCGAGTCCCGTTTCCCGC
TCAACACATAGGCTGTGGACAAGGTGGGCGAAAGTATTTTTTCCATAGTTTTACACCAACGCCCGCCTTTTCCTAAACGCATTG
GAGAGATAGAGGACTTGCCTTCTAAACAAGCAGTACGGGGGAACTTGCATCCGACCTCCGTTTCAATGCGGTAGAACTCCGCTC
CCGTGACAGCGACGAATGATGCAATAGCGGTTCACGAGATACCTCAAGAAACTTCATTTTTCAAAAGCCACAATAGTTCAACTG
GTAGAACGGCGGTATCGTAAACCGCAGGTTGCTGGTTCAATTCCTGCTTGTGGCTCAACAATTTCGGGGGCTTGCAACGCTGCC
ACTGCGGGTGGAAGCCAGCGACAAGAACTTGTGTGAAGCCGAAACGCAGTCCTTCGGGAGAGGGGCGAAGGGGCAAGCGAGATG
TGTCCCACTTTTTTAAAGTAACAGGCTTTAATAAATATTTATCATTCCCGAAAGGCTGTGCGGAACAGCCTCTCGGCTTTTACG
GGGATTTAGTTCAGTTGGTAGAACATCTGGTTCGCAATCAGAAGGTCGCGGGTTCGACTCCCGCAATCTCCACAAATATAAATA
TAGTATTGCCCTGTGGTGCAATCGGTAACACACCAGATTCTGAATCTGGAATTTCGAGTTCGAGCCTCGGTGGGGCAACACAAT
AGGCAGCCGTACTGCCGAATACAAGCCTGTGGAGAACCCAACCGTGGATGACCGTTGCCTATGCAACCTAAAAAGCGGTGGTTC
TGTGAAGCAGGAAGCGGAAATACAATATTCCGCATACGGTGGTGGTGTAATCGGTAACATAACAATATCCGAAAAGTTTAAACC
ATACACCCGACGATTATTTTTATTCATTGTTAGCGACCGCCGTGAGGCGGACGCAGGCTGGCGGTCGGATAATGACGCATAATG
GCGGTTGTGAAAGCCGACGGAAAGCACTACATCGTTAAGTGCCAGCCACCATAATAGGCAGCCGTACTGCCGAATTTAAGCCTG
TGGAGAACCCAACCGTGGATGACCGTTGCGTAAGCAACCTAAAAAGCGATGGTTCTGCGAAGCAGGAAGGAAATGCCCAATTTA
TTAGGTTTTTCCATACGGTATGACAGCCTCTAACTGTAGCGCATTACAAAACAAACGCTACCATTACATAAATGGTCAGAGGCA
TAACGCCGAGCGCAGGTATGGTATGCGTTCAAGTCGCAGTCACGGAAGCCCCAGATAAAAATGGGAGGTGCTTGCGGTCAAGCG
AGTGGTCAGCGGGCTTGCACTCGGTGTGGCAACAATGGTCGTTTCCGAACTTACGACCATTCAAAAAGATAAGGTAGTGGCTTG
TGAGTGAAAAGAAACTCTCGATACGCTCCTTTCGTCTAACGGTCAGGACGCGAGATTCTCAATCTCGTAATGCGGGTTCGATTC
CCGCAGGGAGTACAATGGCGAACACACGACAATCCAAACTGAAGGGGAACTGGAAAACCCTCGCTCCGAGATAACATCAGCGCA
GAGAGGTTGGTGAGGCAACCGTAAAAGTAATCCTGTGTGCAAGCAAGAAGGAAGTTCGGGTTCAAGTCCCGATGAGGATTATTG
TTGAAGAGGGATATGATTCAACCATAGCACTTATGGTGCTGTGCAAGGGTTATAGGCAGCCGTACTGCCGAATACAAGCCTGTG
GAGAACCCAACAGTGGATGACCGTTGCCTATGCAACCTAAAAAGCGGTGGTTCTGCGAAGCAGGAAGGAAATGCCCAATTTATT
AGGTTTTTCCATACGGTATCACTACTCGCGGTGGATGTGGAAATAACCGCGATTTGGTCAGTTGGTGAAGTTGGTTATCATACC
TGCCTGTCACGCAGGTGTTCACGAGTTCGAGCCTCGTACTGACCGCAGACAAAGACAAAGAACGAGAGGACTTGTATGACTTGC
AAATGTCACGGACTCAAACAAGAAAAGTTTATAGGCTATTAGAGGATGACTGTTTCTTTAATTTGTTTTCTTGTACTGAAGGTC
ATCACTGCCGTGCCACCAAGCCGTGCAAGTCCAAATGGTGCGTTAGTTCAGTTGGTTAGAATGCCAGCCTGTCACGCTGGAGGT
CGCGGGTTCGATTCCCGCACGCACCGCAATAATCTGGATATAGGCAAATTACACATATCATATGTCGCCCCGCGTAATCATAGA
CGACACTGCGGACGACAGCGGCGAGAATGTCGAAAGGCTCGACAGCATAATGACATTCGACATCACCGACACCCCGATATACGA
AGGCGGGGAGGAACTTGAGATAAACGCAAAATTCAACAGATAGAAATAATTAAAACAAACGGCAATGGCACACAGAAAAAAGAA
AGATGACGAAGCAACGCTATCGTACAAGTTCAAGGTAAAGGTCATAGAGGGCGACCTGACGGCAGACGACATAACGAAGTGTAT
CGCGGAAAACGCGGAGCAGGGCAACCATTTCTCCGAGTTCATACACGATGAGAATTTCAGGAAGACCTTCACATCCGAGATCAG
CGCGGACAAGTTCGGATGGGGCAAGCCGATGTTCAGCCCGACCACCAGAAGTCAGGACGAAGTGTTCTCCGCGATAAAGAAAAT
CGGGGCGATAACCGTGCTGGAAGATTAGCGCATATTATTCTCATATCTAAAATTGGAAGGACACCTGCGGACGCGGGTGTCCTT
TTTTCTTAAAATGCCAATTTATAAATAATATATAACTTATATTTATTGTACTTTTTTTGTTTAACTAAAACACATAGACAAATA
TGGAAATTCAACAGATTAGGTTTATAAACCCAGTTGATTTTGAAGAAACAATCGTTAATGTACCCACGGAGAAGGGCGAAAGAT
TCCTGAGAACAAAAATCTATACGGACGAGTATTCACCCGAAACATTCATAAAACTCTGCGAGAAG (SEQ ID NO: 105)
>3300028888|Ga0247609_10000668_74|M
TGGCGATTATTCTTACGGCAAAGGCCTTATCCATGCATACATAAATCGAGACATCAAAAGTTTTTGCTTGCCAAACACTTTAAT
ATGTGAATGCCATATACCAAAACATACCAGATATATTACTGATTACTCAGGTACAAATATAGCCGCAAAGAAAATCATCATCGA
CAAAGTTGTCTGGGAGAAGGTATGTATAAAAACATAATGGTATTAGGGGAGAAATTTTCTTGGACGGAATGAATATAATTTCAT
ACCAACACCGTGCATTGATTAAACTAAATTAAATTATCAAGCATAAAAAGTTTGGCACGGTTTTTGATATAGTAAATTTGTATT
TAAAATTTTTAATATGGCACACAAAACTAAAGAATCAGAAAAATTAGTAAAGTCTTTCAAATTAAAAGTAGACATTAGCAATTG
CGAAATTGAAAAGAAATGGATTCCTTCTTTTGAAGAATACACAAATTATTATAATGGAGTAAGTAATTGGATTTGTGAACTATT
AGAAAAAGTTTGCCTGAAAAGAAAAAAATTTGGAAAGGCTTCTTATTCAGTACCATATTGGAACGTTAAAGACGCATTTAAGAA
AAACGTTAGCTCAAACATGATTGCTACAATTAAAAAAATGAATATGGTAAAGGTTTTTTAATGCGTGATTATGGCGTTTTTTAA
ACATAAAATCATTTATAATATATTGAAAAACATTTTATTATATAAAATATGCATCTTAGTGAAACCGTGTTTTCGTATAGATTG
CTGGATTATACTTTTTTATAGGATAATTACAGCTCGAACTTCTTTGATGGCATTAATAAGATATTGTTGGATTAT (SEQ ID
NO: 106)
>3300028805|Ga0247608_10000895_42|M
ATCATGGCTGAAAGCGTCCGCCTGATTGCAGAGCAAACCGCAAGCCCGAAGGTTGTCATCAAGAGCCGTTACGCTCTGGTCGAC
GCAGGTTTCTATCCTGAGTTGAACTATGTGACCTTCTTCGTGAACACTCCAGATCAACTGGTTTAATCACTGCGGGTAGCAAGC
GATTGACTACGGAAGGCCGATTCGATAGAGTCGGTCTTCTTTTTTTTTTGTATATTTTCTTTTTTTGGTTTGGAAATGTTCCGT
ATATTTGCAGCACTAAAACTAACCAATATGGGACATGTACGTTTGCAAAAAAGAGAGGGAGAGGTTTATAAGACCTACAAACTT
AAAGTAAAGAGCTTTTCTGGCAATGTAGACATTAAAGCTGGTATCGTTGAATACGATATCGCCGAAACAATTGATTGGAGAAGT
ACGCTTTGTTTCAAGACATGGAATACGTATGGTTCTCCTCAATGGGACTCGAAGATCAAGAACCAGAAAACGATGATCGATCGA
CTGGATTCGTTGGGTGCAATAGAATTGAAAAACTGGTGATTTTGATCATGGTTTTGAAACAAAATATTGATTTTTCGTTCTTTG
ACATGCTTGTTAAAAATTGAGTATCAGTTTAATATAAAGAATATAT (SEQ ID NO: 107)
>OQVL01000914_15|P
GGAAACAATTATAACGATGCCTACAAAACGTTAATTCAAATGAGAGACAAAGGAATTTTAACGCAGGAAGTTGTAAATGTATTT
ACCCTATTGAAAGGGCGGTATATTAAAGAAAAAGAATACGGAACACAATATAATACTATCAATTAAATTTTTTGGTAGTTTCAT
TTGGAATTGCCAATTATTTTTTTATTTTATAGAATAATAGAGCCAACAAGCATTAGCAATTATTAAATCGATTTATATTGAAAA
TAAATTTTGTGGGAATATTTATTTTTACTATCTTTGCATCGTAAGATAATTACAAAACATTAACAACATTTATTAAACAATTAA
ACAAATTTTAATTATGGCGCACAAAAAGAACGTAGGAGCAGAGATAGTAAAAACTTACTCTTTTAAGGTAAAGAATACCAATGG
TATCACAATGGAAAAATTGATGAACGCCATTGACGAGTTTCAGTCATACTATAACCTTTGTAGCGATTGGATATGCAAGGGTCT
TGACGAAACAATGAGGAACACTTTTCTGAAAAAAGCAAATAGCAATAAATCATTGTATAATCAGCCAATCTACGATACGGGTAT
CAAGAAGACCGCAGGTGTGTTTTCCAGAATGAAAAAATTAAAGAGATATGAAATTATCTAAAATAAAATATGAATTTTTCTTTG
CGGAAATACCTTTTAATAGATTGATTTCTAATAAGTTATAAGAAATACAATAGATACTGAAGGAAAATCAAAGTGTAATCAAAA
ATTTGTTTGTGTTGAGGAAGCAGTGAAGAAATTTCATTGTTTCCTCAATTTTTATTTGCATAATCCAAAAAGTTTTTTATTTTA
TAGGATAATAAGACTAACAAATCTCAACGACTATTAAAACGATTTATATAAAAAAAGTTTTGCAGTTCCAATCTTTTTTGCTAT
CTTTGCAGTGTTGAAAGACAACAAAGATTTAAGTTTAACAAACAAATACTTTTTATTACATATTTTAATTTTTTTGTATTATGA
CAATAGAAGAAAAAGCAAGGGAAGAATACCCTTATATAACCCCATCTGATGGGTATGAATGCCATGATTATAATGAAGCCGCTA
AAGACGGTTTTATTGAGGGGGCAAAATGGATGCTTGAAAAAGCCGCTGAATGGTTTAAGAAT (SEQ ID NO: 108)
>3300028888|Ga0247609_10003329_9|M
ATATGGGCAAAGCGTGATAAAATTGAAAACAAATATGTCAAAGAACCATTAAAACGAGTCAATGAAGATATGTGGTGGATGTAC
TATGTTTATGAATGGAATGTGTTTTATGTGCTTGAAGAAAATGTCCATCCATATATGAAAAAATAAATTTTACCACACATATTA
TTATTCGTGTCATGCCGATGAGGTTTGGCACGATTTTTGTTTATATGGAGAGACATAATGTCAGTCAATACATGACAACTTGTC
ACAATAACTGACATTAAAAGTTTGGCACAATATTTGCTTATAAGAAAAACGAACAAGTAAAATTAAAATTTTATAGATTATGGC
ACACAAAACAAACAACGGAGAAAACACCATCAACAAAACTTTCATCTTCAAAGCAAAATGCGAGAAGAACGATATTATATCGTT
ATGGAAACCCGCAGCAGAAGAGTATTGCAACTATTATAACAAATTGAGCAAATGGATTGGTAAAACAATGTACGGCATTCCTGC
ATATAACATCAAAAGAGGTTTTAAGAAGAATTTAAGTGCCAAAACTATAAACACATTTAGAAAACTTGGACACTATCGTGATGG
AAAAATAAATGAGGATGGCATGTTTGTTGAAACTTTGGCATAGAATTTGCATATACCAATTAGAATTGAAAAAATCGCTCTTTG
ACACACTGAAACATACAAAAACACCACAATTTTTTAATCCTTTTCTATTTGTATTTTATTGAAATAAAATGTATTATAGTAATA
TATCTGCTAAGGTCATATTTTTCATTGTTCTCAAATTGTTGGATAATGTTTTGTGTGTTTCATTTTTGTCATTGTGTCACCTTA
ACTGACAAGGTGGCACATTTTTTATGTCAATATGTCAGTTGAGGTTTTGGCATAATTTTTGTATAATGGTAAATGGATAAGAAT
TGAAATTACAATGACAACAAAACAAAGGTTAATAAAGAGAATAAACAAGGCATTCGGATTTGAATTAACGGATGCAACACCTTG
TTTCCACCATCAAGGTAGAAGATGGGGAAGCGGTGGTTTC (SEQ ID NO: 109)
>3300028805|Ga0247608_10006074_1|M
GAAGGCGGCGCGTTTGAAATCGCTAACGTAATTGAAAATGCCAAGAAGCAGAATCTCGGGGAGGGTGGATACAAGGAATTGTGC
AATGATTTCCTGAAACATGCGAGGGAAACGTTTTTCAGTGGGAAATACGAACACCATTCTTGGTAGTGGATTTGTTATTTTGGT
AAATATAATTAACGCGGCATTGTCGTCAGTGAATATAATATTGCATTTCGACAGTATTTTATAAGTATTTTGACTTATAAACAG
TATTTATAAGTTATTCGGCTTATAGGTTAATTAGCCTATAGATGTTGTTTATAGGTTGGATGACCTATAGTGCCAAGTTTTGAA
GAAATCGTTATAGTCATCGTTCTGCCCTATTAGATATTCCGTATTTCTTTAAGACTGTTATAATACAAATATACTACAAATCAT
GCAATTTTTGATTTTTAACAAAAATTAAGAAATAGGGTATTATTGTGTATTGTTTTTTGTTATATATTTGTCCTGTTAGGTTAA
ATCACCGCGCCTGATGACGAAGTCGGTGGTAGAATTAGACTAATATTAAATATGTCTCATGAATTTAACAAGAATAAAGGTGAG
AATGAGATTAGCAAGACCTTTATTTTCAAAACAAAATGCGGGAAGAATGATATTACATCATTATGGGTTCCCGCGATGGAGGAG
TATTGCACGTATTACAACAGGGTAAGCAAATGGGGGAAAGGTATGTACAACAAGCCGTCATATGACATACGGAAGAAATTCAAG
AAGAACTTGAGTGCGGCTACTTTGAAAACTTTCATTAAGTTGGGAAACACGGTGAAAGGGATGATTGTCAACGGACAGTTTGTT
GAAATGGAATCATAGGTTGACAGAAACGGAAAATCGGTTTGTTTGTTAGAAGAATATTTGTTGAAATTCATTTTTCTTTTGCTA
ACGTATATACAAATAACTGTAATAGAATATCTTATATAAGATAT (SEQ ID NO: 110)
>3300012973|Ga0123351_1009859_3|P
ACAAATGAAATTATGGGACAAGTAAAACTTAATAAACCTCTTCTGTATATCAAAATATTGACTATCTTTAGACATAACCTTGTC
AAATAATAAATCTAAATTACTCTTTTCCTTTTCTTTTTTAAATAATTTCATATTAAATATTCCCATAATTTATTAATATATTTT
TTTTTCATTACTTATTTCTCTGTTATATAAATAGTTACATAAAAAAATTAAAACTATTTTTTAAAAAGTCTTGTGTATATAAAA
AAAATATAGTACCTTTGCACCCGAAATCAAGATTTAATCCTGTTTTCATATTATATTTATCAATTTTATACTAATTAATAAACT
TATGGCAAATAAAAAATTTAAACTTACAAAAAATGAAGTCGTGAAATCATTCGTACTCAAAGTTGCTAACCAAAAAAAATGTGC
TATCACTAACGAAACACTTCAAGAATATAAAAACTATTATAATAAGGTAAGTCAGTGGATTAATAACATCGTACAAAATGAAAC
GTGGAGAAATCTATTTACTAACAAAACCAATAATACATATGGATTACCTATACTAACACCTTCAAAAAAAGGACAATCTAATAT
CATTACACAATTAATGAAAATTAATGCAACACAAGAACTTGTTGTATAATATAATCTATTTTTAAATTTATAATACTAATATAA
TTCATTGATAATTAAATAATTATATAAAATTCCTATATACAATAGAAAGACTTTCCACAGACATGTTGTACATACATTTTTTTA
AGTATTAAACAACGCATACCCACCAATGGTACACGAAAATTTTCATGTTGTACATACTATTTTTAGGTATTAAACAACTCACTG
TTTTGACGATTAATATAGGCATGTTGTACATACTCTTTTTAGATATTAACAACCTGTAAACAATAACAATATTTACAACAATAA
TCCATTTTTGAAATAATGAAAAATTTTCTGGAAAAATTTTTTAACAAGTCTGTTTTTGAAATAATGAAAAAATTTCTGGAAAAA
TTTTTTTAACAAACCCATTTTTGATTGGTTCATTTTTTATTGGAAAATTAGTGTGTGGAACTACCCACCCGTATATGAGCAAGT
GTTATGGGGTGTAACGTGGGGAGGGTTACATAGGGGGGTCTTTGGTAGGGGGTACATAGGTAGGGTAATAATGGGGTCTTTGGT
AGGGGGTACATAGGTAGTCCCCATATATTATTATAAAAAGTAAAATAAATGATATATGCAAGAGTTTTTGAAAATTTATTTTTA
TTTTGCTACTTAGACTTTACAAAAAGTAGATATATAGTATTTTCTTTTCAAAATATTTTGTAGTTTGGAAAAAAAGCAGTACCT
TTGCACACGGAAACGAAAAACAAGTTTAACCTATTAAATTTTTAGTTTATGGCAATAAACATTTTGACTTATTCTGCTATGGCA
GAAAAATCTTGGGAAAATTTTATGCGTGAAAATTGCGGTTACGAGCGCATTAGTACATTTTATAGTGATTTCACTATTGCAGAC
CATTGTGGTGGTGTAAACGCAATAAAAGAC (SEQ ID NO: 111)
>3300012979|Ga0123348_10005323_4|M
GATGTGAATGAAGAATTTCTTGGTGGCTTGCGAAGCACTATGACATATCTTGGAGCAAAGAGATTGAAAGATATTCCGAAATGT
TGCGTTTTCTATCGTGTAAATCATCAGTTGAATACAATTTATGAGAATACAACGATAGGAAAATAATATAAATTTTATATTATT
TTGAGAAAAAGAGTCTAAATTTGGGCTCTTTTTTCGTTTTTTATGAAAAAATATGAAAAAAGTTTGTAAAAAATTTGTAATATT
GAAAAAATAGTATTATATTTGTATCAAATTTAAAAATAAAATATAAATATGGCAAAATCAATAATGAAAAAATCAATTAAATTC
AAAGTAAAAGGAAATAGTCCAATAAACGAAGATATTATAAATGAGTATAAAGGTTATTATAATACCTGTAGTAATTGGATTAAT
AATAATTTAACAAGCATAACTATTGGTGAAAATGAAGACTGGAGAAAAGTGTTTTGTATCAAACCAAAAAAAGAAGATTACAAT
ACACCTTTATTGGATGCTACGAAAAATGGTCAATTTAGAATACTTGACAAGTTGAAAAAATTAAATGCTACTAAATTATTAGAA
ATGGAAAAATAATAAATATATACAATAAATTTATATAATTTTGTCTATTTTTAATTTTAGTTCATTAGATAATATGTTCATAAA
TTCATTGACATATAATTATAAATAAATATATATGCAATAAAATTCGAGAGACATTTCATCAGAGATGTCTCTTTTTTATTTTTT
GTTATATTTATATTATGAATATTAGATTGGAACTCATAAAGACAAAGGATAAACAGAACATTGCAAAGCGTATAGTGGAAAGCA
ATCACTCATATGTTCCAACCTGGCGTAGTGTAGGACGAAGGATAGATTATCTTATTTATTTGGATAATGATGTTGTCGGA
(SEQ ID NO: 112)
>3300028888|Ga0247609_10016480_8|M
GTGAACTATATCTACGAATCAATCGAAGGAATATTGACAAAAACAATGAATCCAACCACTTTACAGGATATCATCCTTAACGGA
ATCACATATACACCAGTGGAAGACAACACAACAACATGCGACGGATGTGAATTTAAAGACACATAAGGCCAATGTATGCTAACA
CACCTATTCGATAACGACATGGTCCAAAACTGCCTCAAGGAAAAAAACGGCGTTGCAGATATCATATATGTCAAAAAAGAAAAT
TAATCGGAATCTTGATTTGGATTTTAATATTATTTGTTGTATAATTACAATAGAAAGAAAATTTTGTATATTTTAAAATTTGTA
AATTAAAATTTAGAAAAATGGCACACAAAACAAACAACGGAGAAAATACAATCAATAAAACTTTTATTTTCAAAGCAAAGTGCG
ATAATAACGATATTATATCGTTATGGAAACCCGCAATGGAAGAGTATTGTACTTATTACAATAAATTAAGCCAATGGATTTGCA
AGACAATGTATGGAGTACCAGCTTACAACATTAAAAACGGTTTCAAAAAAAATCTGAGCACAAAGACAATCAATACGTTTAGAA
CGCTTGGCCACTATCGTGACGGAAAAATAAACGAAGACGGCGTATTCGTTGAAAACCTGGCATAATAAGGAGTAAAAAAATGTT
CTTTGATATTCTGACACAAATGAAAAAACAATCAAAAATTTATTTCTGTTTTGCTTGTAATTTATTGAAATAAAATGTATTATA
TAGAAATATGTCGGTGGATAATAGTCAAATAGTCTGTTGACTGTTGAATAGTAAGTTTTTTACTCTATTGACAACAGGTGATGT
GGATGGAACATACAAAGTTTATTGTTGAGTAATAGGTTTTACACTTTTACCACAACTTTAGTGATTTTATGTATAAAATAATTA
AAATCATATATAAAAATTTTTCCAGAAAGTAGTACTTATTGAATTAAAATTATATTGTGAAAAATGGTTTTTGATTTTAATTTT
ATTTGTTGTATAATTGAAATGTAATTTAATTTAGAATTGTATAAATAAAAAACGTAAAAATGAGACTGCCAACAGAAATTTATG
AGTCAGGCACAATGGTTAGTAAGATATCGGAAAAACCATTTAAATCAGGTTTAAGGGTTAATACTGTAAAGTCTGTAGTTGAAC
ATCCACATAAGATTGACCCGAATACTAATAAGGGTGTTCCA (SEQ ID NO: 113)
>3300028887|Ga0265299_10000013_320|P
GACTACGACTGGTTCTCAAATGTGTACGGCGCCATCAGGGAGGAACGTGAGAAAATGAGAAGGGAAGAGGAGGAACGCAGGAAG
AACGAACCCAAGACGGTGAAAACCAAAGAGGTTGACTTGTTCGGGGATGATGACCTGCCGTTCTAATAAAAAAAAAAACAAACC
TCTCCGAAATTGAACGTATCAACTTCGGAGAGGTTATATAGGGTGATGGAAATGTTAAATAAAAAGTTTAAAAATAACTATGGG
AAACAAAGTACAAAGTAATGAAACAATAGTTAAGACTTATACATTTAAAGTGCGTGGATTCATAAGTGGTGCTACCCACGAAAT
AATGAAATCAGCCATAAAACAATATATAGAAGATTCTAACAATCTATCAGATTGGATTAATGTAGAGAATGAAATACTTAGGAA
CTCTTTCCTTAAAGAAGAGACTAAAAAATACACTTATAATACACCATTATTCACTCCCAGACTTAAGTCATCGGAAAAAATAAT
AACAGAATTGAAAAAATTGGGTATGACTACGGTTATAGAATAACCATTACACATTTTTTTCATAACAAACGTTCTTTAACATAT
TGGAAAATAAGAAAATACGATATTCATATAAAAATCCGTCCCACACAAAATTAATGTAATATCTTAGTTTTGTTACATCAACAC
TATATAATTAAAAAAATAAAAAAATATTTTGTGGATTCAAAAAATCATTATATATTTGCGTCCGAAAATTAACACTTATGTCAA
ACAAATTTAAAATGTAAAAGAACTATGCAAACAGAAACACAGAATTTCACAGGCGAGTTGAGAGCAATCAACACAACAATGGGT
TCAAGCAAGAGCTACAAGACAATCTGCCGTTGCGCACTTGACATCCTCAAGGGATATATCGTTACGCACGACATTAGGGACAAC
TTCTCA (SEQ ID NO: 114)
>3300028887|Ga0265299_10000026_77|P
ACAGAGGGTGTATGGATAGGCATGAACCACCAAGGCAAAATACTGATGGCTTGCAGGGAGGCTTTGTGTAACAACTGTGAACCC
CCGATTGATTACAAGGCACTGAACGATGCCGAGATATATTTTTATGGAAAAGAAGTTAAATTTTAAAAATTAAAAGATATGGCG
AACAAAAGCACAAAAGGAAACCTGCCCAAGACAATCATAATGAAGGCAAACCTTAGCCCCGATGGTTTCACTCAATGGGAAAGG
GTTGTAAAAGAATACCAAGCCTACAAAGACACGTTGAGTAAATGGGTAGCCCAAAATCTCAGACAAATAATGTGCAAGACACCG
CAGACAAAGAACGGCTACTCATCACCTGTGCTCACCTCAAAGGTTAAAAGCCAAGTGGAAATGGTAAGAGAATTGAAAAAAATG
GGAAAAACCATTCTTTATTCCAATGATTCACTTCCTTTTTGAAACTAAAATGTCTTATGTGTATTTGAATTATAGGCTAATATA
AAGATTGTACTGTGTTGAGATACACTTTTAGAGGTATTTACAACAAAATGCGTGATATGGAAATGAAGAAATAACTGTGTTGAG
ATACACTTTTAGAGGTATTTACAACACCATATAAACCTGACCATCTCCTGAATCTCGCCCGACACGGATAATGTTAGATATGTT
CACAATACAACTGCATGTGCTATTCAAGAAAAAATAGTATATTTACAATATGTTGGTGCATAATATTAGATGTGCTTACACAAC
GCAGACCTGAAAAGCCAGGATAAAAGTATGCGGGATTGTGTTTTTAGAACACTGTTCAATCCGCTGTATGTCGCTTGAAGCGTC
AGTAACCTATGTCGAAACAATCCTTTTAGAGGTGTTTACGACCGACCAGAAACAGCAAGACCTGTATTTATGTTGGTATACGGT
TCTTTTTAGGGGATTAGTAGTTGAATCCCTTTTCACCCTTGGTGTTCACGGGTTGTGAGACATTCTTCATACCCATGCGTGTCT
TCTCAGCCATCTTACCGAAAGTTATAGGCACAATATGTTCAATGCCTGCCTGCTGAGCATTGTAGCATATATCAGACAG (SEQ
ID NO: 115)
>SRR1221442_316828_61|P
AGAATGCTTTCCCCAATTGAATGTGAAAGACTACAGACACTGCCAGATAACTATACCGAAGGTGTTAGCAAATGCGCAAGATAT
AAGGCAATCGGAAACGGATGGACAGTTGATGTAATTTCACATATTTTTAAGAATTTGAAAAATTAATTTGGTATTTTGAAATAT
TTGACTTATTTTTGCAACATAAAATTTAAAACAAATTTATATGGCACACGCGAAAAAAAAATTTTGACAAAGGAAAGCAAATAA
CAAAAACGTTCTCTTTCAAGGTGTTAAATATTAAGAACAATGGCGAATCAGTTGATATGAATACTATAGAATTAGCCATGAAAG
AGTACAATAGGTATTATAACATTTGTAGTGATTGGATTTGCAACAATCTAATGACGCCAATTGGTTCCCTATATCAATACATAG
ATGATGAGAAATGGAGAAAAAAATTTGTTCGCCCAACAAACACTAATAAACCGTTGTATAACTCTCCAGTTTTCTCCCCTGCTG
TAAAATCTGAAGGTGGTACTATTAAAAATCTCCAAATTTTAAGCGCAACAAAGACCATAATTCTTTGATTTAATTATTAATACA
TATATCGTTCGTAAATTTAATACAACCACAACCAAATATGATAATTTGCATAATTAAAAAAATTCACATATCTTTGTAGCATAA
AAACAAATAGAGAAAAAATGACACTTTACAGATTTACACTTTTAGGCAATACACAAATTTATGTATATGCTGGCACGTTTGAAG
ATGCTCTCAGGACATTTCGTAAATCATATGGAGATACGGGATTCAAGTCAATTGAAGAGCTTCCTGAATTTAGAGATAACATAC
TTATACAACTAGATTGATTGAAACAAACGTCAATTACCCACCACTGAAGTAGTGGGTTTCTTTGCAGTGATTTTATGAAAACGA
TAGAAGACAGAGCAGACATAGCAAGCGATATTGCTAAAAGAGAATTTGAAGAAGATAGTTATTGGAGTCATTACGCAGACGATA
TGGTAACATCTGCTTTTGTTGAAGGATGCTATAAAGGCTATATTTCAGGTGCGACA (SEQ ID NO: 116)
>SRR5678926_1309611_3|P
AAGGAGATAGATTATGACAGGGAAGGTAATATCACAAATATATATCTTTACTATGAGTCAGATAGTTTATGGAATGAAAAATTT
GAATTTATATTAACATTAGATGGTTATGAATTAAAGATACCTATTTTTATAGTAAGTGTAAGATAGTTTTGGCACGGAAATTGC
AGTAATGTTTTCCTGTCAAGAACAAATAAAATAAAAAATATGAAAAAATCAATTAAATTCAAAGTAAAAGGAAATTGTCCAATA
ACCAAAGATGTTATAAATGAATATAAAGAATATTATAATAAATGCAGTGATTGGATTAAGAATAATTTAACAAGCATAACTATT
GGGGAAATGGCAAAATTTCTCAATGAAGTGTGGAGAGAAATATTTTGTACAAGGCCTAAAAAGGCAGAATATAACGTTCCATCG
TTGGATACAACAAAAAAAGGACCATCTGCAATATTGCATATGTTGAAAAAAATCGAGGCAATTAAAATATTAGAAACAGAAAAG
TAGTGACTATAGATATAAACTTCTATGATAGATATCTGTTTTTTAATTCTATTATGCAATATAATATATTGAAATATAAACAAT
TATAAATAAAACGGGTGTATACAACAAGTTTTTTGTTTTTCTTATTCATTATCTGTATATTTGTATTATAAACAAATACAAATA
TGTATAATGAATCAGGAATATATTGCTATAAAAACAAAATAAACGGAAAATTATATATTGGACAGGCGCTAAATCTTAAAAGAA
GATATTTAAACTTTTTAAATATCAACCACAGATATGCGGGTCAAGTAATAGAAAACGCACGTAAAAAATATGGTGTAGATAACT
TTGAATATTCAATCCTTACTCACTGTCCAGTAGACGAATTAAATTATTGGGAAGCATTTTATGTAGAAAGATTAAATTGTGTCA
CACCCCACGGTTATAATATGACTAATGGGGGCGATTCAGTATATACTTCTACACAAGCATTTAAAGATGCACAAACTGAAAAGT
TGAAGCAAACTATTCTATCTAAGAATCCTAATCTTAATGTCAGCAAAGTAAAATATGAAGGTAATAGAATTTCAGTTATAATTA
CTTGCCCAATACATGGCACATTTAAAAAAACGCCTGATTACTTTAGAAATCCAGAAATAAATGATTTGTGTTGTCCTAAATGTG
TGAGGGAAGATATAAGACAAAAGACTGAAGATAGTTTCTTTAAACAAGCAACAAAGAAATGGGGAGATAAGTATGATTATTCTA
AAACTATAATAGTAGATAGAATTACCCCAGTTACAATTACTTGCCCTATACACGGAGATTTTACAGTATTACCAGGGAACCATG
TGTGTAAAGATAAAAATACTGGAGGATGCCAACAATGTAGTGAAGAAAGACAACATATTGAATCATTAGAAAAAGGTAGCGTGA
AGGTCATTAAGATGATAAAGAAAAAGTTTGGAAACAAATATTCATTAGATAAATTCGAATATAGGGGAGATAAAGAAAAAGTAA
TTCTTATTTGCCCTATTCATGGAGAATTTTCAATGACGCCAGGTAATTTAAGATATAGCAACGGTTGTCCACAATGCACTTTAG
AAAATGCTTATCGTATAAAAT (SEQ ID NO: 117)

### Example 3—Identification of Novel RNA Modulators of Enzymatic Activity

In addition to the effector protein and the crRNA, some CRISPR systems described herein may also include an additional small RNA to activate or modulate the effector activity, referred to herein as an RNA modulator.
RNA modulators are expected to occur within close proximity to CRISPR-associated genes or a CRISPR array. To identify and validate RNA modulators, non-coding sequences flanking CRISPR effectors or the CRISPR array can be isolated by cloning or gene synthesis for direct experimental validation.
Experimental validation of RNA modulators can be performed using small RNA sequencing of the host organism for a CRISPR system or synthetic sequences expressed heterologously in non-native species. Alignment of small RNA sequences to the originating genomic locus can be used to identify expressed RNA products containing DR homology regions and sterotyped processing.
Candidate RNA modulators identified by RNA sequencing can be validated in vitro or in vivo by expressing a crRNA and an effector in combination with or without the candidate RNA modulator and monitoring alterations in effector enzymatic activity.
In engineered constructs, RNA modulators can be driven by promoters including U6, U1, and H1 promoters for expression in mammalian cells, or J23119 promoter for expression in bacteria.
In some instances, the RNA modulators can be artificially fused with either a crRNA, a tracrRNA, or both and expressed as a single RNA element.

### Example 4—Functional Validation of Engineered CLUST.091979 CRISPR-Cas Systems

Having identified components of CLUST.091979 CRISPR-Cas systems, loci from the metagenomic source designated AUXO013988882 (SEQ ID NO: 1) and from the metagenomic source designated SRR3181151 (SEQ ID NO: 4) were selected for functional validation.

### DNA Synthesis and Effector Library Cloning

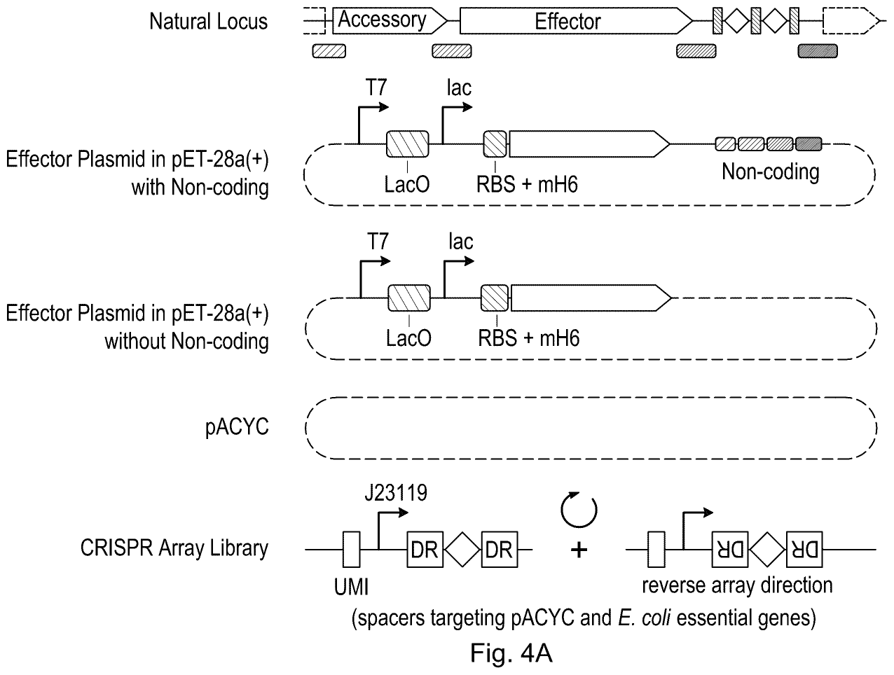

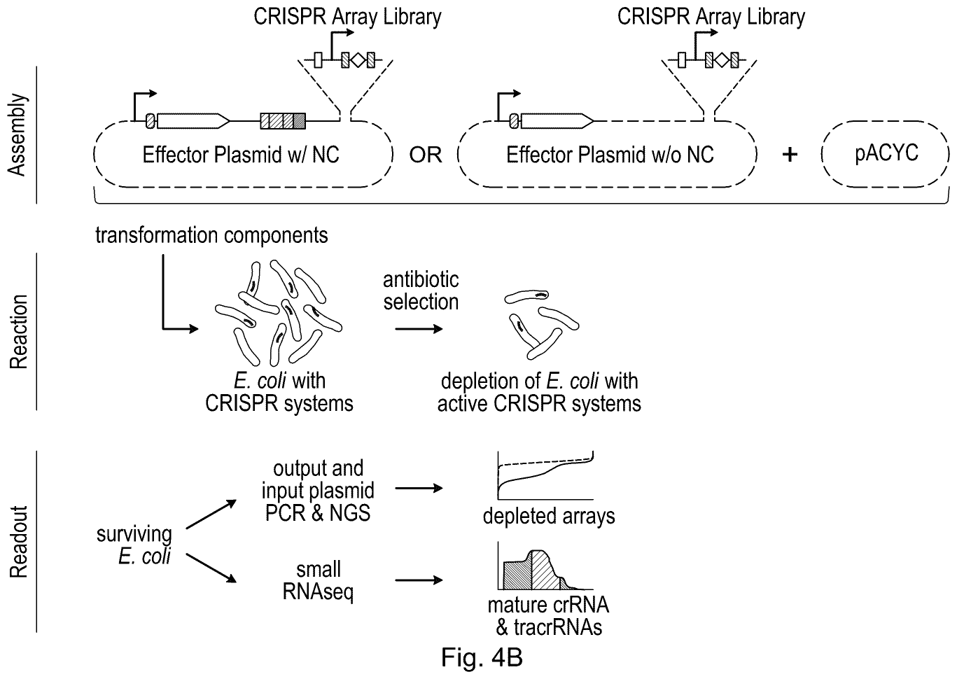

To test the activity of the exemplary CLUST.091979 CRISPR-Cas systems, systems were designed and synthesized using a pET28a(+) vector. Briefly, an E. coli codon-optimized nucleic acid sequence encoding the CLUST.091979 AUXO013988882 effector (SEQ ID NO: 1 shown in TABLE 6) and an E. coli codon-optimized nucleic acid sequence encoding the CLUST.091979 SRR3181151 effector (SEQ ID NO: 4 shown in TABLE 6) were synthesized (Genscript) and individually cloned into a custom expression system derived from pET-28a(+) (EMD-Millipore). The vectors included the nucleic acid encoding the CLUST.091979 effector under the control of a lac promoter and an E. coli ribosome binding sequence. The vector also included an acceptor site for a CRISPR array library driven by a J23119 promoter following the open reading frame for the CLUST.091979 effector. The non-coding sequence used for the CLUST.091979 AUXO013988882 effector (SEQ ID NO: 1) is set forth in SEQ ID NO: 98, and the non-coding sequence used for the CLUST.091979 SRR3181151 effector (SEQ ID NO: 4) is set forth in SEQ ID NO: 99, as shown in TABLE 9. Additional conditions were tested, wherein the CLUST.091979 effectors were individually cloned into pET28a(+) without a non-coding sequence. See FIG. 4A.

An oligonucleotide library synthesis (OLS) pool containing “repeat-spacer-repeat” sequences was computationally designed, where “repeat” represents the consensus direct repeat sequence found in the CRISPR array associated with the effector, and “spacer” represents sequences tiling the pACYC184 plasmid or E. coli essential genes. In particular, the repeat sequence used for the CLUST.091979 AUXO013988882 effector (SEQ ID NO: 1) is set forth in SEQ ID NO: 57, and the repeat sequence used for the CLUST.091979 SRR3181151 effector (SEQ ID NO: 4) is set forth in SEQ ID NO: 60, as shown in TABLE 8. The spacer length was determined by the mode of the spacer lengths found in the endogenous CRISPR array. The repeat-spacer-repeat sequence was appended with restriction sites enabling the bi-directional cloning of the fragment into the aforementioned CRISPR array library acceptor site, as well as unique PCR priming sites to enable specific amplification of a specific repeat-spacer-repeat library from a larger pool.

Next, the repeat-spacer-repeat library was cloned into the plasmid using the Golden Gate assembly method. Briefly, each repeat-spacer-repeat was first amplified from the OLS pool (Agilent Genomics) using unique PCR primers and pre-linearized the plasmid backbone using BsaI to reduce potential background. Both DNA fragments were purified with Ampure XP (Beckman Coulter) prior to addition to Golden Gate Assembly Master Mix (New England Biolabs) and incubated per the manufacturer's instructions. The Golden Gate reaction was further purified and concentrated to enable maximum transformation efficiency in the subsequent steps of the bacterial screen.

The plasmid library containing the distinct repeat-spacer-repeat elements and CRISPR effectors was electroporated into E. Cloni electrocompetent E. coli (Lucigen) using a Gene Pulser Xcell® (Bio-rad) following the protocol recommended by Lucigen. The library was either co-transformed with purified pACYC184 plasmid or directly transformed into pACYC184-containing E. Cloni electrocompetent E. coli (Lucigen), plated onto agar containing chloramphenicol (Fisher), tetracycline (Alfa Aesar), and kanamycin (Alfa Aesar) in BioAssay® dishes (Thermo Fisher), and incubated for 10-12 hours at 37° C. After estimation of approximate colony count to ensure sufficient library representation on the bacterial plate, the bacteria were harvested, and plasmid DNA WAS extracted using a QIAprep Spin Miniprep® Kit (Qiagen) to create an “output library.” By performing a PCR using custom primers containing barcodes and sites compatible with Illumina sequencing chemistry, a barcoded next generation sequencing library was generated from both the pre-transformation “input library” and the post-harvest “output library,” which were then pooled and loaded onto a Nextseq 550 (Illumina) to evaluate the effectors. At least two independent biological replicates were performed for each screen to ensure consistency. See FIG. 4B.

### Bacterial Screen Sequencing Analysis

Next generation sequencing data for screen input and output libraries were demultiplexed using Illumina bc12fastq. Reads in resulting fastq files for each sample contained the CRISPR array elements for the screening plasmid library. The direct repeat sequence of the CRISPR array was used to determine the array orientation, and the spacer sequence was mapped to the source (pACYC184 or E. Cloni) or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

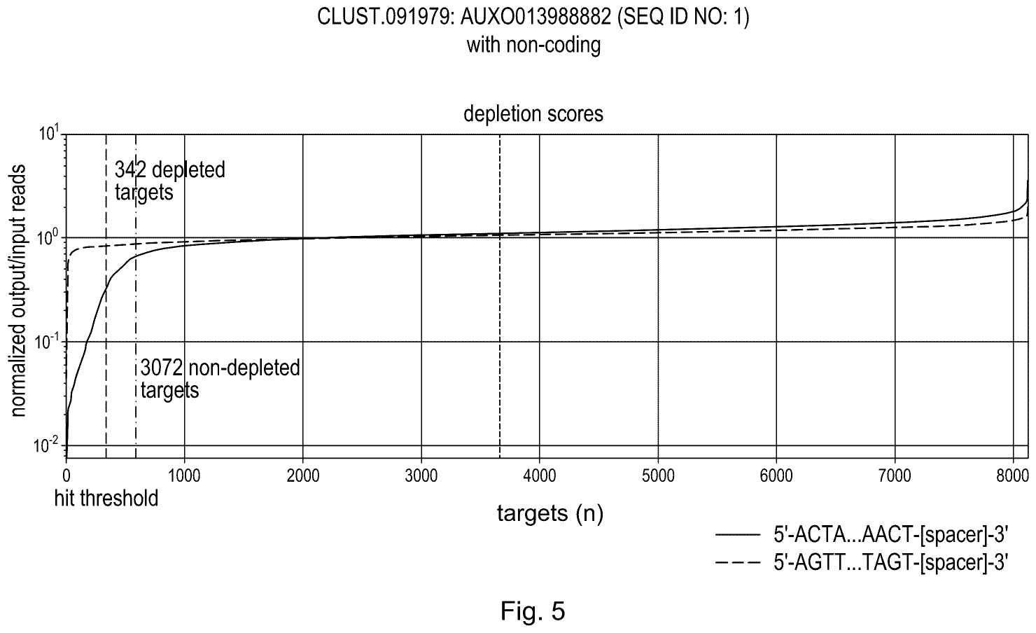

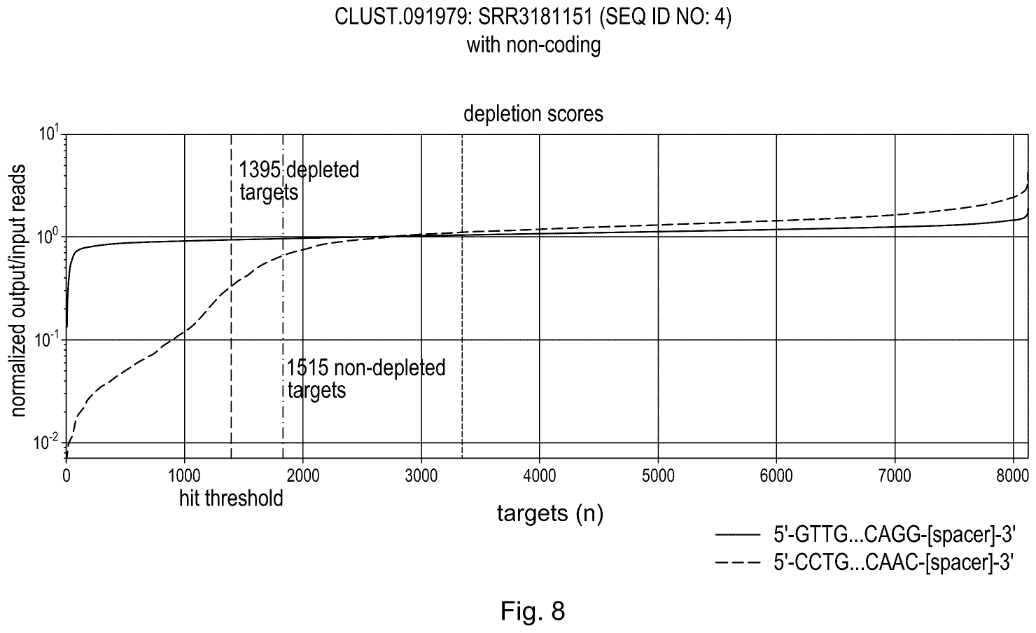

To identify specific parameters resulting in enzymatic activity and bacterial cell death, next generation sequencing (NGS) was used to quantify and compare the representation of individual CRISPR arrays (i.e., repeat-spacer-repeat) in the PCR product of the input and output plasmid libraries. The array depletion ratio was defined as the normalized output read count divided by the normalized input read count. An array was considered to be “strongly depleted” if the depletion ratio was less than 0.3 (more than 3-fold depletion), depicted by the dashed line in FIG. 5 and FIG. 8. When calculating the array depletion ratio across biological replicates, the maximum depletion ratio value for a given CRISPR array was taken across all experiments (i.e. a strongly depleted array must be strongly depleted in all biological replicates). A matrix including array depletion ratios and the following features were generated for each spacer target: target strand, transcript targeting, ORI targeting, target sequence motifs, flanking sequence motifs, and target secondary structure. The degree to which different features in this matrix explained target depletion for CLUST.091979 systems was investigated.

FIG. 5 and FIG. 8 show the degree of interference activity of the engineered CLUST.091979 compositions, with a non-coding sequence, by plotting for a given target the normalized ratio of sequencing reads in the screen output versus the screen input. The results are plotted for each DR transcriptional orientation. In the functional screen for the composition, an active effector complexed with an active RNA guide will interfere with the ability of the pACYC184 to confer E. coli resistance to chloramphenicol and tetracycline, resulting in cell death and depletion of the spacer element within the pool. Comparison of the results of deep sequencing the initial DNA library (screen input) versus the surviving transformed E. coli (screen output) suggests specific target sequences and DR transcriptional orientations that enable an active, programmable CRISPR system. The screen also indicates that the effector complex is only active with one orientation of the DR. As such, the screen indicated that the CLUST.091979 AUXO013988882 effector was active in the “forward” orientation (5′-ACTA . . . AACT-[spacer]-3′) of the DR (FIG. 5) and that the CLUST.091979 SRR3181151 effector was active in the “reverse” orientation (5′-CCTG . . . CAAC-[spacer]-3′) of the DR (FIG. 8).

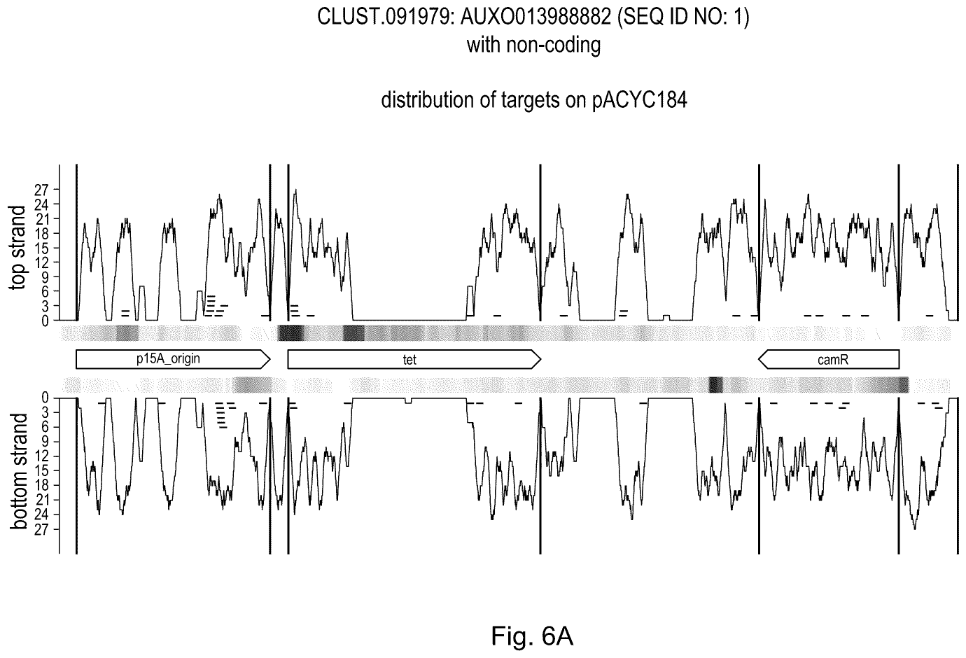

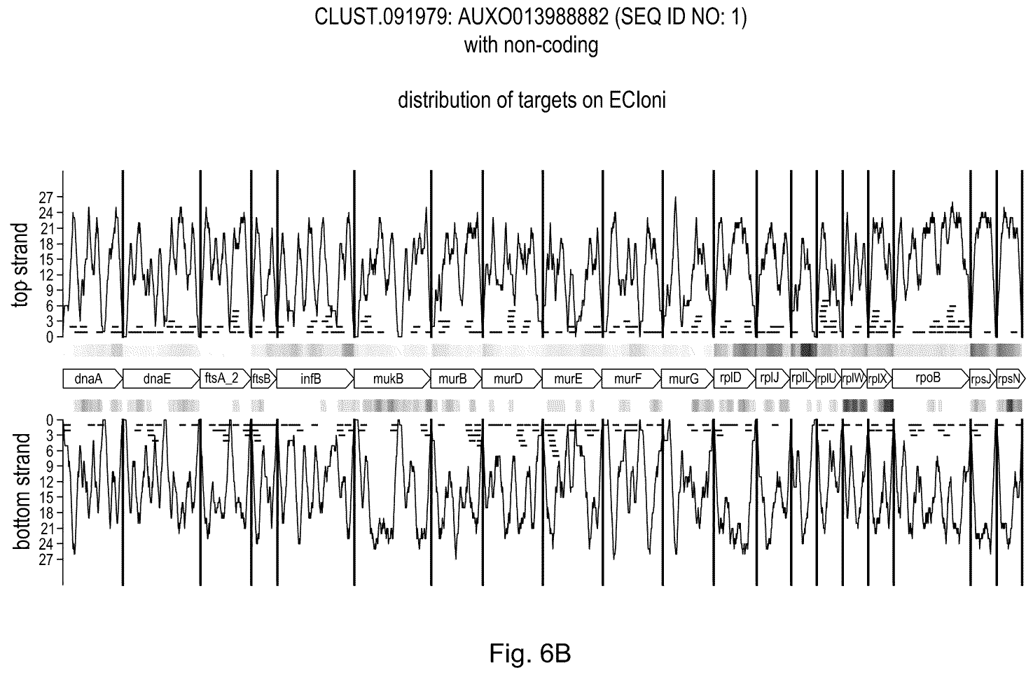

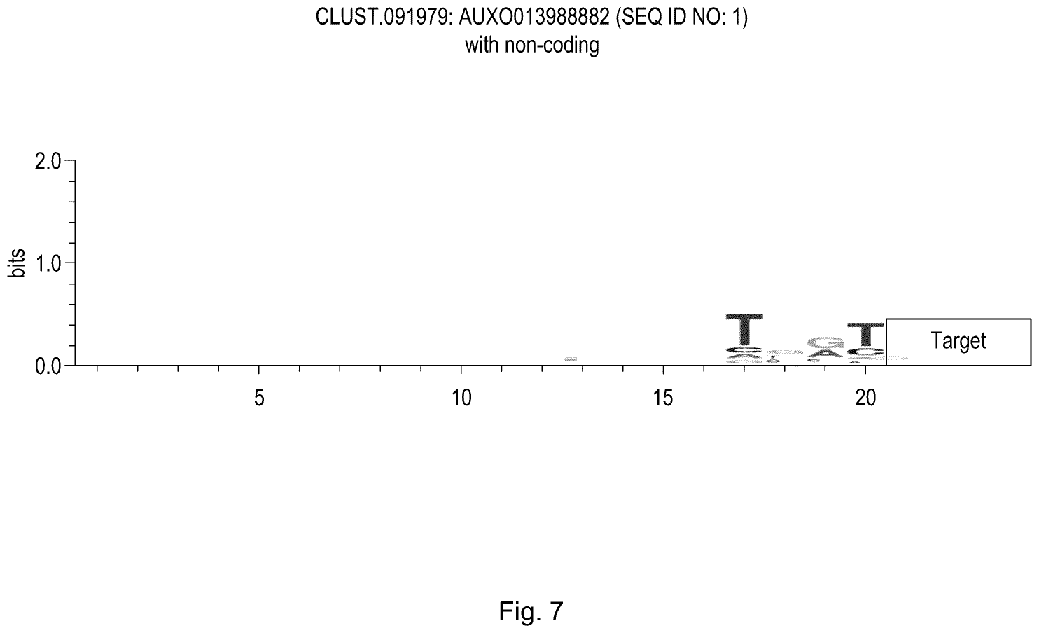

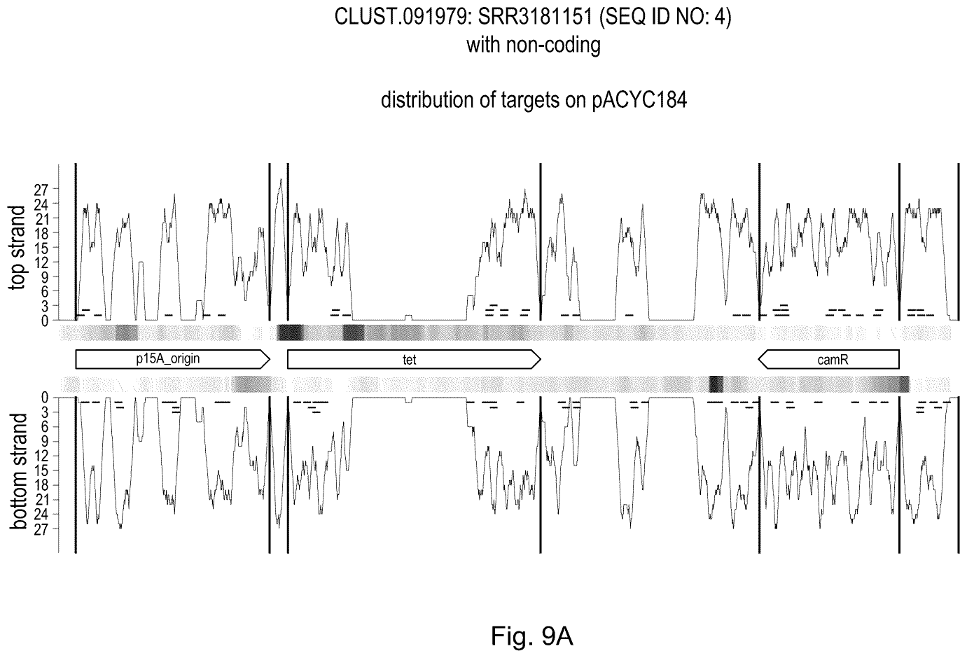

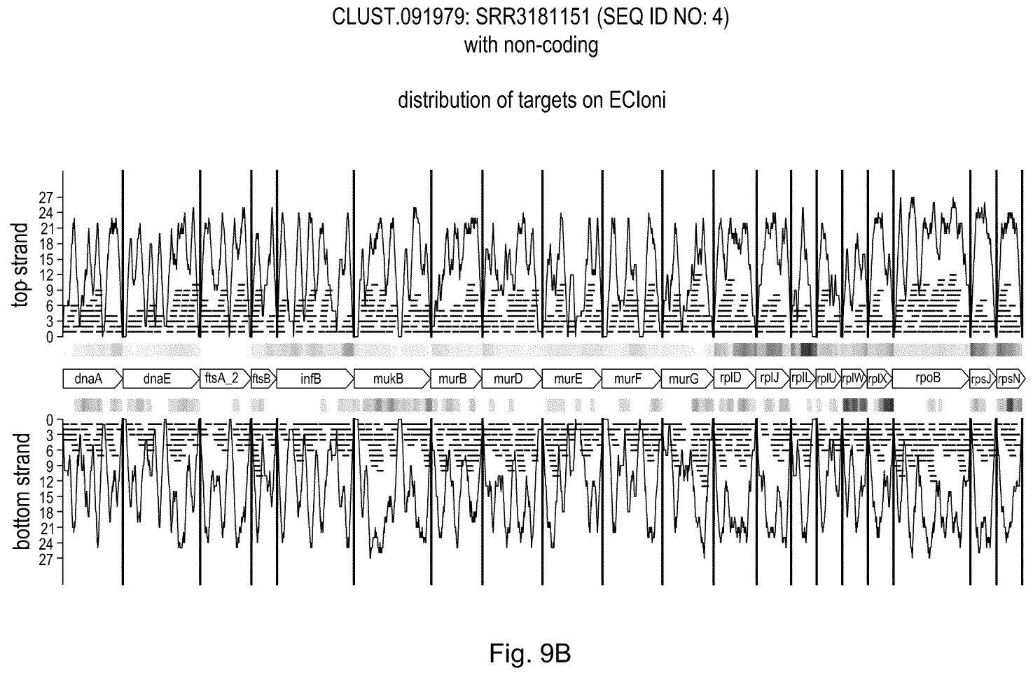

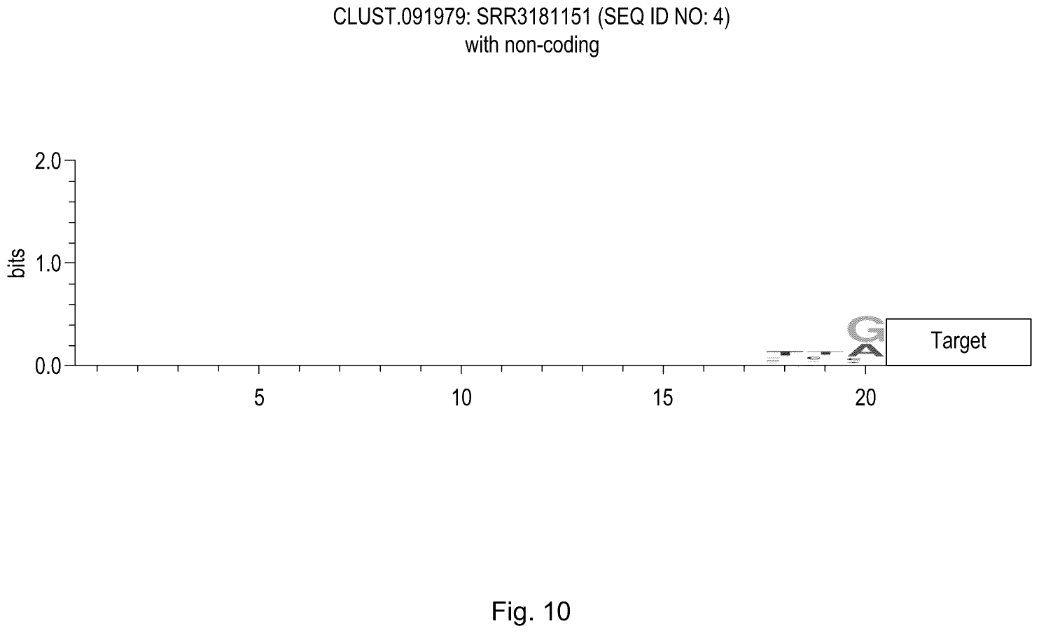

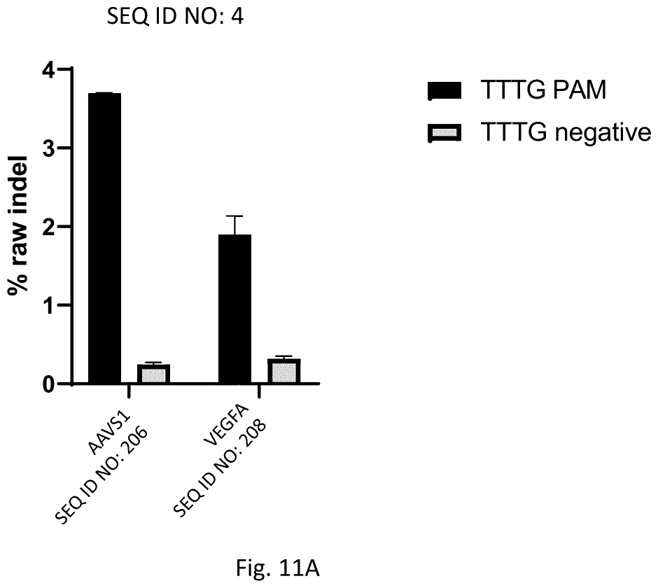

FIG. 6A and FIG. 6B depict the location of strongly depleted targets for the CLUST.091979 AUXO013988882 effector (plus non-coding sequence) targeting pACYC184 and E. coli E. Cloni essential genes, respectively. Likewise, FIG. 9A and FIG. 9B show the location of strongly depleted targets for the CLUST.091979 SRR3181151 effector targeting pACYC184 and E. coli E. Cloni essential genes, respectively. Flanking sequences of depleted targets were analyzed to determine the PAM sequences for CLUST.091979 AUXO013988882 and CLUST.091979 SRR3181151. WebLogo representations (Crooks et al., Genome Research 14: 1188-90, 2004) of the PAM sequences for CLUST.091979 AUXO013988882 and CLUST.091979 SRR3181151 are shown in FIG. 7 and FIG. 10, respectively, wherein the “20” position corresponds to the nucleotide adjacent to the 5′ end of the target.

Thus, multiple effectors of CLUST.091979 CRISPR-Cas show activity in vivo.

### Example 5— Targeting of Mammalian Genes by CLUST.091979

This Example describes indel assessment on multiple targets using nucleases from CLUST.091979 introduced into mammalian cells by transient transfection.

The effectors of SEQ ID NO: 4 and SEQ ID NO: 10 were cloned into a pcda3.1 backbone (Invitrogen). The plasmids were then maxi-prepped and diluted to 1 μg/μL. For RNA guide preparation, a dsDNA fragment encoding a crRNA was derived by ultramers containing the target sequence scaffold, and the U6 promoter. Ultramers were resuspended in 10 mM Tris·HCl at a pH of 7.5 to a final stock concentration of 100 μM. Working stocks were subsequently diluted to 10 μM, again using 10 mM Tris·HCl to serve as the template for the PCR reaction. The amplification of the crRNA was done in 50 μL reactions with the following components: 0.02 μl of aforementioned template, 2.5 μl forward primer, 2.5 μl reverse primer, 25 μL NEB HiFi Polymerase, and 20 μl water. Cycling conditions were: 1×(30s at 98° C.), 30×(10s at 98° C., 15s at 67° C.), 1×(2 min at 72° C.). PCR products were cleaned up with a 1.8×SPRI treatment and normalized to 25 ng/μL. The prepared crRNA sequences and their corresponding target sequences are shown in TABLE 10. The direct repeat sequence of the mature crRNAs of SEQ ID NO: 205, SEQ ID NO: 207, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, and SEQ ID NO: 276 is set forth in SEQ ID NO: 60. The direct repeat of the mature crRNAs of SEQ ID NO: 209 and SEQ ID NO: 214 is set forth in SEQ ID NO: 62. The direct repeat of the mature crRNAs of SEQ ID NO: 211, SEQ ID NO: 278, SEQ ID NO: 280, SEQ ID NO: 282, SEQ ID NO: 284, SEQ ID NO: 286, and SEQ ID NO: 288 is set forth in SEQ ID NO: 213.

TABLE 10
RNA guide and Target Sequences for Transient Transfection Assay.

| Effector | | | PAM |
|---|---|---|---|
| Sequence | mature crRNA Sequence | Target Sequence | Sequence |
| SEQ ID | CCTGTTGTGAATACTCTTTTATA | AAVS1: | 5’-TTTG-3’ |
| NO: 4 | GGTATCAAACAACGGAAGTGGT | GGAAGTGGTTGGTCAGCAT | |
| | TGGTCAGCATGGATTA | GGATTA (SEQ ID NO: 206) | |
| | (SEQ ID NO: SEQ ID NO: 205) | | |
| SEQ ID | CCTGTTGTGAATACTCTTTTATA | VEGFA: | 5-TTTG-3’ |
| NO: 4 | GGTATCAAACAACTGTGAAGTG | TGTGAAGTGACCTGGGAGCT | |
| | ACCTGGGAGCTAACTG | AACTG (SEQ ID NO: 208) | |
| | (SEQ ID NO: 207) | | |
| SEQ ID | CCTGTTGTGAATACTCTTTTATA | AAVS1: | 5-TTTG-3’ |
| NO: 4 | GGTATCAAACAACGAGAGGTG | GAGAGGTGAGGGACTTGGG | |
| | AGGGACTTGGGGGGTAA (SEQ | GGGTAA (SEQ ID NO: 253) | |
| | ID NO: 252) | | |
| SEQ ID | CCTGTTGTGAATACTCTTTTATA | AAVS1: | 5-TTTG-3’ |
| NO: 4 | GGTATCAAACAACTGAGAATGG | TGAGAATGGTGCGTCCTAGG | |
| | TGCGTCCTAGGTGTTC (SEQ ID | TGTTC (SEQ ID NO: 255) | |
| | NO: 254) | | |
| SEQ ID | CCTGTTGTGAATACTCTTTTATA | AAVS1: | 5-TTTG-3’ |
| NO: 4 | GGTATCAAACAACGCAGCCTGT | GCAGCCTGTGCTGACCCATG | |
| | GCTGACCCATGCAGTC (SEQ ID | CAGTC (SEQ ID NO: 257) | |
| | NO: 256) | | |
| SEQ ID | CCTGTTGTGAATACTCTTTTATA | AAVS1: | 5-TTTG-3’ |
| NO: 4 | GGTATCAAACAACGGAAGTGGT | GGAAGTGGTTGGTCAGCAT | |
| | TGGTCAGCATGGATTA (SEQ ID | GGATTA (SEQ ID NO: 259) | |
| | NO: 258) | | |
| SEQ ID | CCTGTTGTGAATACTCTTTTATA | EMX1: | 5-TTTG-3’ |
| NO: 4 | GGTATCAAACAACAGCCAGTGT | AGCCAGTGTTGCTAGTCAAG | |
| | TGCTAGTCAAGGGCAG (SEQ ID | GGCAG (SEQ ID NO: 261) | |
| | NO: 260) | | |
| SEQ ID | CCTGTTGTGAATACTCTTTTATA | VEGFA: | 5-TTTG-3’ |
| NO: 4 | GGTATCAAACAACTTGACATTG | TTGACATFGTCCACACCTGG | |
| | TCCACACCTGGAATCG (SEQ ID | AATCG (SEQ ID NO: 263) | |
| | NO: 262) | | |
| SEQ ID | CCTGTTGTGAATACTCTTTTATA | VEGFA: | 5-TTTG-3’ |
| NO: 4 | GGTATCAAACAACGAAATCTAT | GAAATCTATTGAGGCTCTGG | |
| | TGAGGCTCTGGAGAGA (SEQ ID | AGAGA (SEQ ID NO: 265) | |
| | NO: 264) | | |
| SEQ ID | CCTGTTGTGAATACTCTTTTATA | VEGFA: | 5-TTTG-3’ |
| NO: 4 | GGTATCAAACAACGGAAGCTGG | GGAAGCTGGATGAGCCTGG | |
| | ATGAGCCTGGTCCATG (SEQ ID | TCCATG (SEQ ID NO: 267) | |
| | NO: 266) | | |
| SEQ ID | CCTGTTGTGAATACTCTTTTATA | VEGFA: | 5-TTTG-3’ |
| NO: 4 | GGTATCAAACAACCCCATACTG | CCCATACTGGGGACCAAGG | |
| | GGGACCAAGGAAGTGT (SEQ ID | AAGTGT (SEQ ID NO: 269) | |
| | NO: 268) | | |
| SEQ ID | CCTGTTGTGAATACTCTTTTATA | VEGFA: | 5-TTTG-3’ |
| NO: 4 | GGTATCAAACAACATGATGCTT | ATGATGCTTTGCCGTAACCC | |
| | TGCCGTAACCCTTCGT (SEQ ID | TTCGT (SEQ ID NO: 271) | |
| | NO: 270) | | |
| SEQ ID | CCTGTTGTGAATACTCTTTTATA | VEGFA: | 5-TTTG-3’ |
| NO: 4 | GGTATCAAACAACAAGAGTCAT | AAGAGTCATTGCCCCACTTT | |
| | TGCCCCACTTTACCCT (SEQ ID | ACCCT (SEQ ID NO: 273) | |
| | NO: 272) | | |
| SEQ ID | CCTGTTGTGAATACTCTTTTATA | AAVS1: | 5-TTTG-3’ |
| NO: 4 | GGTATCAAACAACGAGAGGTG | GAGAGGTGAGGGACTTGGG | |
| | AGGGACTTGGGGGGTAA (SEQ | GGGTAA (SEQ ID NO: 275) | |
| | ID NO: 274) | | |
| SEQ ID | CCTGTTGTGAATACTCTTTTATA | VEGFA: | 5-TTTG-3’ |
| NO: 4 | GGTATCAAACAACGTGAAGTTC | GTGAAGTTCTAAACTTCATA | |
| | TAAACTTCATATTACC (SEQ ID | TTACC (SEQ ID NO: 277) | |
| | NO: 276) | | |
| SEQ ID | ATTGTTGTAGACACCTTTTTATA | AAVS1: | 5-ATTG-3’ |
| NO: 10 | AGGATTGAACAACAACCCCCGT | AACCCCCGTCTACCTGCCCA | |
| | CTACCTGCCCACAGGG | CAGGG(SEQ ID NO: 210) | |
| | (SEQ ID NO: 209) | | |
| SEQ ID | CTTGTTGTATATGTCCTTTTATA | AAVS1: | 5’-GTTA-3’ |
| NO: 10 | GGTATTAAACAACGTAGAGGGA | GTAGAGGGAGAAATGGAAT | |
| | GAAATGGAATCCATAT | CCATAT (SEQ ID NO: 212) | |
| | (SEQ ID NO: 211) | | |
| SEQ ID | ATTGTTGTAGACACCTTTTTATA | VEGFA: | 5-ATTG-3’ |
| NO: 10 | AGGATTGAACAACGCACCAACG | GCACCAACGGGTAGATTTG | |
| | GGTAGATTTGGTGGTG | GTGGTG (SEQ ID NO: 215) | |
| | (SEQ ID NO: 214) | | |
| SEQ ID | CTTGTTGTATATGTCCTTTTATA | AAVS1: | 5’-GTTA-3’ |
| NO: 10 | GGTATTAAACAACGTAGAGGGA | GTAGAGGGAGAAATGGAAT | |
| | GAAATGGAATCCATAT (SEQ ID | CCATAT (SEQ ID NO: 279) | |
| | NO: 278) | | |
| SEQ ID | CTTGTTGTATATGTCCTTTTATA | AAVS1: | 5-ATTG-3’ |
| NO: 10 | GGTATTAAACAACGAGTCGCTT | GAGTCGCTTFAACTGGCCCT | |
| | TAACTGGCCCTGGCTT (SEQ ID | GGCTT (SEQ ID NO: 281) | |
| | NO: 280) | | |
| SEQ ID | CTTGTTGTATATGTCCTTTTATA | VEGFA: | 5-ATTG-3’ |
| NO: 10 | GGTATTAAACAACTCCACACCT | TCCACACCTGGAATCGGCTT | |
| | GGAATCGGCTTTCAGC (SEQ ID | TCAGC (SEQ ID NO: 283) | |
| | NO: 282) | | |
| SEQ ID | CTTGTTGTATATGTCCTTTTATA | AAVS1: | 5-ATTG-3’ |
| NO: 10 | GGTATTAAACAACAACCCCCGT | AACCCCCGTCTACCTGCCCA | |
| | CTACCTGCCCACAGGG (SEQ ID | CAGGG (SEQ ID NO: 285) | |
| | NO: 284) | | |
| SEQ ID | CTTGTTGTATATGTCCTTTTATA | AAVS1: | 5’-GTTA-3’ |
| NO: 10 | GGTATTAAACAACGTAGAGGGA | GTAGAGGGAGAAATGGAAT | |
| | GAAATGGAATCCATAT (SEQ ID | CCATAT (SEQ ID NO: 287) | |
| | NO: 286) | | |
| SEQ ID | CTTGTTGTATATGTCCTTTTATA | EMX1: | 5’-GTTA-3’ |
| NO: 10 | GGTATTAAACAACGACCCATGG | GACCCATGGGAGCAGCTGG | |
| | GAGCAGCTGGTCAGAG (SEQ ID | TCAGAG (SEQ ID NO: 289) | |
| | NO: 288) | | |

Approximately 16 hours prior to transfection, 100 μl of 25,000 HEK293T cells in DMEM/10% FBS+Pen/Strep were plated into each well of a 96-well plate. On the day of transfection, the cells were 70-90% confluent. For each well to be transfected, a mixture of 0.5 μl of Lipofectamine 2000 and 9.5 μl of Opti-MEM was prepared and then incubated at room temperature for 5-20 minutes (Solution 1). After incubation, the lipofectamine:OptiMEM mixture was added to a separate mixture containing 182 ng of effector plasmid and 14 ng of crRNA and water up to 10 μL (Solution 2). In the case of negative controls, the crRNA was not included in Solution 2. The solution 1 and solution 2 mixtures were mixed by pipetting up and down and then incubated at room temperature for 25 minutes. Following incubation, 20 μL of the Solution 1 and Solution 2 mixture were added dropwise to each well of a 96 well plate containing the cells. 72 hours post transfection, cells are trypsinized by adding 10 μL of TrypLE to the center of each well and incubated for approximately 5 minutes. 100 μL of D10 media was then added to each well and mixed to resuspend cells. The cells were then spun down at 500 g for 10 minutes, and the supernatant was discarded. QuickExtract buffer was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit.

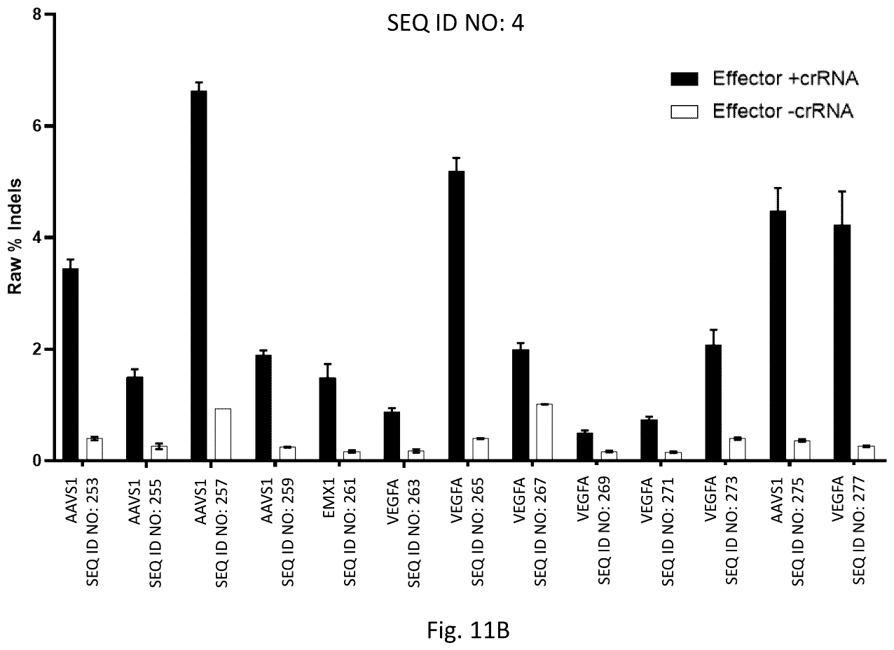

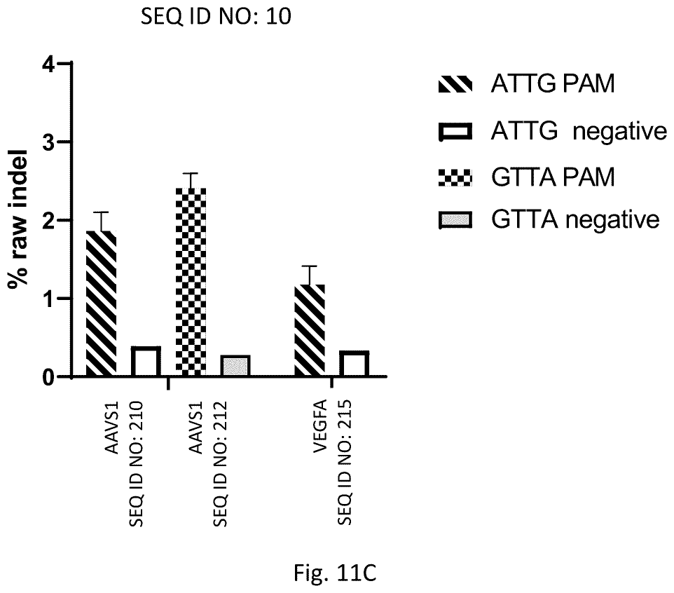

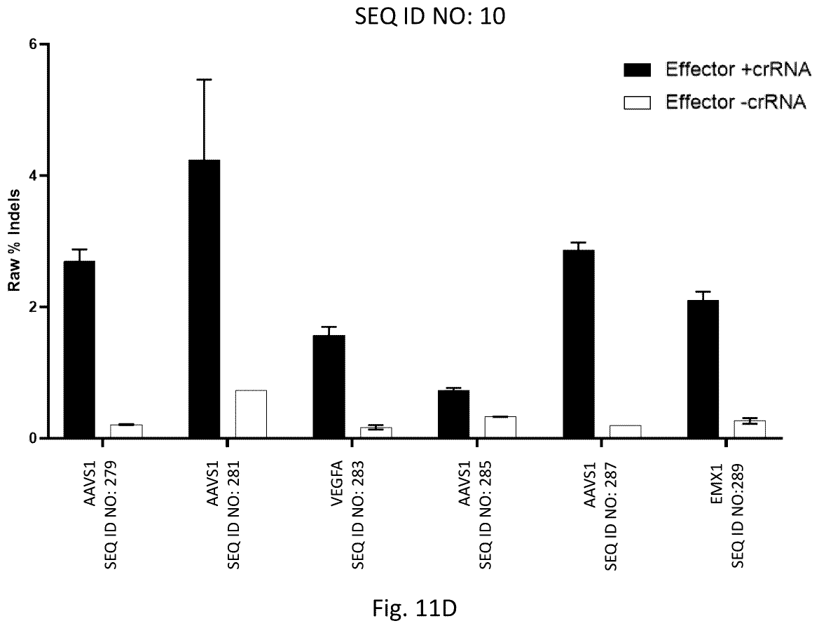

FIG. 11A, FIG. 11B, FIG. 11C, and FIG. 11D show percent indels in AAVS1, VEGFA, and EMX₁ target loci in HEK293T cells following transfection with the effectors of SEQ ID NO: 4 or SEQ ID NO: 10, respectively. The bars reflect the mean percent indels measured in two bioreplicates. For the effectors of SEQ ID NO: 4 and SEQ ID NO: 10, the percent indels were higher than the percent indels of the negative control at each of the targets.

As shown in FIG. 11A, a complex formed by the effector of SEQ ID NO: 4 and the crRNA of SEQ ID NO: 205 was active at the AAV target of SEQ ID NO: 206, and a complex formed by the effector of SEQ ID NO: 4 and the crRNA of SEQ ID NO: 207 was active at the VEGFA target of SEQ ID NO: 208. As shown in FIG. 11B, a complex formed by the effector of SEQ ID NO: 4 and the crRNA of SEQ ID NO: 252 was active at the AAVS1 target of SEQ ID NO: 253, a complex formed by the effector of SEQ ID NO: 4 and the crRNA of SEQ ID NO: 254 was active at the AAVS1 target of SEQ ID NO: 255, a complex formed by the effector of SEQ ID NO: 4 and the crRNA of SEQ ID NO: 256 was active at the AAVS1 target of SEQ ID NO: 257, a complex formed by the effector of SEQ ID NO: 4 and the crRNA of SEQ ID NO: 258 was active at the AAVS1 target of SEQ ID NO: 259, and a complex formed by the effector of SEQ ID NO: 4 and the crRNA of SEQ ID NO: 274 was active at the AAVS1 target of SEQ ID NO: 275. Also as shown in FIG. 11B, a complex formed by the effector of SEQ ID NO: 4 and the crRNA of SEQ ID NO: 260 was active at the EMX₁ target of SEQ ID NO: 261. Also as shown in FIG. 11B, a complex formed by the effector of SEQ ID NO: 4 and the crRNA of SEQ ID NO: 262 was active at the VEGFA1 target of SEQ ID NO: 263, a complex formed by the effector of SEQ ID NO: 4 and the crRNA of SEQ ID NO: 264 was active at the VEGFA1 target of SEQ ID NO: 265, a complex formed by the effector of SEQ ID NO: 4 and the crRNA of SEQ ID NO: 266 was active at the VEGFA1 target of SEQ ID NO: 267, a complex formed by the effector of SEQ ID NO: 4 and the crRNA of SEQ ID NO: 268 was active at the VEGFA1 target of SEQ ID NO: 269, a complex formed by the effector of SEQ ID NO: 4 and the crRNA of SEQ ID NO: 270 was active at the VEGFA1 target of SEQ ID NO: 271, a complex formed by the effector of SEQ ID NO: 4 and the crRNA of SEQ ID NO: 272 was active at the VEGFA1 target of SEQ ID NO: 273, and a complex formed by the effector of SEQ ID NO: 4 and the crRNA of SEQ ID NO: 274 was active at the VEGFA1 target of SEQ ID NO: 275. The effector of SEQ ID NO: 4 utilized a 5′-TTTG-3′ PAM for each of the targets in FIG. 11A and FIG. 11B.

As shown in FIG. 11C, a complex formed by the effector of SEQ ID NO: 10 and the crRNA of SEQ ID NO: 209 was active at the AAVS1 target of SEQ ID NO: 210, a complex formed by the effector of SEQ ID NO: 10 and the crRNA of SEQ ID NO: 211 was active at the AAVS1 target of SEQ ID NO: 212, and a complex formed by the effector of SEQ ID NO: 10 and the crRNA of SEQ ID NO: 214 was active at the VEGFA target of SEQ ID NO: 215. As shown in FIG. 11D, a complex formed by the effector of SEQ ID NO: 10 and the crRNA of SEQ ID NO: 278 was active at the AAVS1 target of SEQ ID NO: 279, a complex formed by the effector of SEQ ID NO: 10 and the crRNA of SEQ ID NO: 280 was active at the AAVS1 target of SEQ ID NO: 281, a complex formed by the effector of SEQ ID NO: 10 and the crRNA of SEQ ID NO: 284 was active at the AAVS1 target of SEQ ID NO: 285, and a complex formed by the effector of SEQ ID NO: 10 and the crRNA of SEQ ID NO: 286 was active at the AAVS1 target of SEQ ID NO: 287. Also as shown in FIG. 11D, a complex formed by the effector of SEQ ID NO: 10 and the crRNA of SEQ ID NO: 288 was active at the EMX₁ target of SEQ ID NO: 289, and a complex formed by the effector of SEQ ID NO: 10 and the crRNA of SEQ ID NO: 282 was active at the VEGFA target of SEQ ID NO: 283. The effector of SEQ ID NO: 10 utilized a 5′-ATTG-3′ PAM and a 5′-GTTA-3′ PAM for the targets in FIG. 11C and FIG. 11D.

This Example suggests that nucleases in the CLUST.091979 family have activity in mammalian cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered, non-naturally occurring Clustered Regularly Interspaced Short Palindromic Repeat (CRISPR)—associated (Cas) system comprising:
(a) an RNA guide or a nucleic acid encoding the RNA guide, wherein the RNA guide comprises a direct repeat sequence and a spacer sequence; and
(b) a CRISPR-Cas effector protein or a nucleic acid encoding the CRISPR-Cas effector protein, wherein the CRISPR-Cas effector protein comprises an amino acid sequence with at least 95% identity to SEQ ID NO: 4,
wherein the CRISPR-Cas effector protein binds to the RNA guide, and wherein the spacer sequence binds to a target nucleic acid.

2. The system of claim 1, wherein the CRISPR-Cas effector protein comprises an amino acid sequence with at least 99% identity to SEQ ID NO: 4.

3. The system of claim 1, wherein the direct repeat sequence comprises a nucleotide sequence with at least 95% sequence identity to SEQ ID NO: 291.

4. The system of claim 3, wherein the direct repeat sequence comprises the nucleotide sequence of SEQ ID NO: 291.

5. The system of claim 1, wherein the spacer sequence comprises between 15 and 55 nucleotides in length.

6. The system of claim 5, wherein the spacer sequence comprises between 20 and 45 nucleotides in length.

7. The system of claim 1, wherein the target nucleic acid comprises a sequence complementary to a nucleotide sequence in the spacer sequence.

8. The system of claim 1, wherein the system does not include a tracrRNA.

9. The system of claim 1, wherein the CRISPR-Cas effector protein recognizes a protospacer adjacent motif (PAM) sequence, wherein the PAM sequence comprises a nucleotide sequence set forth as 5′-NTTN-3′, 5′-NTTR-3′, or 5′-NNR-3′, wherein “N” is any nucleotide and “R” is A or G.

10. The system of claim 1, wherein the CRISPR-Cas effector protein further comprises at least one nuclear localization signal (NLS), at least one nuclear export signal (NES), or at least one NLS and at least one NES.

11. The system of claim 1, wherein the CRISPR-Cas effector protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

12. The system of claim 1, wherein the nucleic acid encoding the CRISPR-Cas effector protein is codon-optimized for expression in a cell.

13. The system of claim 1, wherein the nucleic acid encoding the CRISPR-Cas effector protein is operably linked to a promoter.

14. The system of claim 1, wherein the nucleic acid encoding the CRISPR-Cas effector protein is in a vector.

15. The system of claim 14, wherein the vector is a retroviral vector, a lentiviral vector, a phage vector, an adenoviral vector, an adeno-associated vector, or a herpes simplex vector.

16. The system of claim 1, wherein the system is present in a delivery system comprising a nanoparticle, a liposome, an exosome, a microvesicle, or a gene-gun.

17. An isolated cell comprising the system of claim 1.

18. The cell of claim 17, wherein the cell is a prokaryotic cell or a eukaryotic cell.

19. The cell of claim 17, wherein the cell is a mammalian cell or a plant cell.

20. The cell of claim 19, wherein the cell is an isolated human cell.

21. A method of binding the system of claim 1 to the target nucleic acid of claim 1 comprising:
(a) providing the system of claim 1; and
(b) delivering the system to a cell,
wherein the cell comprises the target nucleic acid, wherein the CRISPR-Cas effector protein binds to the RNA guide, and wherein the spacer sequence binds to the target nucleic acid.

22. The method of claim 21, wherein the target nucleic acid is a single-stranded DNA or a double-stranded DNA.

23. The method of claim 21, wherein binding the system to the target nucleic acid results in cleavage of the target nucleic acid.

24. The method of claim 23, wherein cleavage of the target nucleic acid results in formation of an insertion or a deletion in the target nucleic acid.


SEQUENCE LISTING (291 sequences)

SEQ ID NO: 1 - protein, 775 aa, unidentified
  REGION 1..775: note = Description of Unknown:gut metagenome sequence
MGNTTKKGNLTKTYLFKANLSEQDFKLWRSIVEEYQRYKEVLSKWVCDHLTTMKIGDILPYIDRYSKKIDNKTGEYPENTYYSLCEEHKDEPLYKIFQFDSNCRNNALYEVIRKINCDLYTGNILNLGETYYRRNGFVKRVLANYATKISGMKPSVRKRKVTSDSTEEEIRNQVVYEIFNNNIKNEKDFKGVLEYAESKCKTNEAYVERIRLLYDFYIKHTDEIKEYVEYICVEQLKEFCGVKVNRSKSSMNINIQNFSITRVDGKCTYILHLPIGKKVYDIKLWGNRQVVLNVDGTPVDIIDIINRHGESIDIIFKNGDIYFSFVVSEDFKKDDFEIGNVVGVDVNTKHMLIQTNIVDNGNVDGFFNIYKELVNDKEFSECVSKEDLELFKELSKYVSFCPIECQFLFTRYAEQKGILVYEKLRLAEKILTSVLDRSFEKYNGIDCNIANYISNVRMLRSKCKSYFTLKMKYKELQHKYDNEMGYVDTFSDSCVEMDSRRKENPFVQTNEAMELIGKMESVAQDIIGCRDNIITYAYNVFRRNGYDTVGLENLESSQFERFSSVRSPKSLLNYHHLKGKHIDFIDSDECSVKVNKDLYNFTLEDDGTISDITLSDKGKYRNDLSMFYNQIIKTIHFADIKDKFIQLGNNGNVQTVLVPSYFTSQMNSKTHKIYVVNVKNERTGKTEQKLANKNMVRLGQERHINGLNADVNASMNIAYIVENKEMRNAMCTNPKSETGYSVPFLTSRIKKQNIMVVELKKMGMVEVLNEKSTEI

SEQ ID NO: 2 - protein, 786 aa, unidentified
  REGION 1..786: note = Description of Unknown:bovine gut metagenome sequence
MAQHKSNNEESAINKTFIFKAKCDKNDVISLWEPAAKEYCDYYNKVSKWIADNLITMKIGDLAQYITNQNSKYYTAVTNKKKKDLPLYRIFQKGFSSQCADNALYCAIKSINPENYKGNSLGIGESDYRRFGYIQSVVSNFRTKMSSLKATVKWKKFDVNNVDDETLKIQTIYDVDKYGIETAKEFKELIETLKTRVETPQLNDTIARLECLCDYYSKNEKAINNEIETMAIADLQKFGGCQRKSLNAFTIHKQDSLMEKVGNTSFRLQLPFRKKTYVINLLGNRQVVNFVNGKRVDLIDIAENHGDLVTFNIKNGVLFVHLTSPIVFDKDVRDIRNVVGIDVNIKHSMLATSIKDVGNVKGYINLYKELLNDDEFVSTCNESELALYRQMSENVNFGILETDSLFERIVNQSKGGCLKNKLIRRELAMQKVFERITKTNKDQNIVDYVNYVKMMRAKCKASYILKEKYDEKQKEYYVKMGFTDESTESKETMDKRREEFPFVNTDTAKELLVKQNNIRQDIIGCRDNIVTYAFNVFKNNEYDTLSVEYLDSSQFDKRRIATPKSLLKYHKFEGKTKDEVENMMKSEKLSNAYYTFKYENDVVSDIDYSDEGNLRRSKLNFGNWIIKSIHFADIKDKFVQLSNNNKMNIVFCPSAFSSQMDSITHTLYYVEKITKNKKGKEKKKYVLANKKMVRTQQEKHINGLNADYNSACNLKYIALNDELRDKMTDRFKASKKIKTMYNIPAYNIKSNFKKNLSAKTIQTFRELGHYRDGKINEDGMFVENLE

SEQ ID NO: 3 - protein, 774 aa, unidentified
  REGION 1..774: note = Description of Unknown:gut metagenome sequence
MLNIKNNGESVDMNTIELAMKEYNRYYNICSDWICNNLMTPIGSLYQYIDDKCKNNAYAQNLIAEEWKDKPLYYMFYKGYNANNCANAICCAIRSQVPEVNKAENILNLSYTYYFRNGVIKSVISNYASKMRILSDKQIKYCIVSENTPDKILIEQCILELKRRHEDLKDWEENLKYLILKGNESAITRFTILKDFYSKNIERVKEEREIMAIAELKDFGGCRRKDDKLSMCIQSAGNSKDIKVSRVKTTHNYTELVDDYTENFNIKFSALDFNVMGRRDVVKTKLNKTEDDSNTWGGTELLVDIINNHGCSLTFKLVDDKLYVDIPIDTEHINKTTDFKKSVGIDVNLKHSLLNTDILDNGGINGYINIYKKLLADDAFMSACTKADLVNYIDIAKTVTFCPIEADFIISNVVEKYLHMKDNTNKMEIAFSSVLMNIRKELEIKLLHSSKEESPLIRKQIIYINCIICLRNELKQYAIAKHRYYKKQQEYDTLCDTLHGVDYKQIHPYAQSKEGAEQMKKMKTIENNLIANRNNIIEYAYTVFELNNFDLIALENITKDIMEDKKKRKSFPSINSLLKYHKVINCTEDNINDNETYQKFAKYYNVSYENGKVTGATLSQEGNKVKLKDDFYDKLLKVLHFTSIKDYFTTLSNKRKIAVAHVPAYYTSQIDSIDNKICMIKSTDKNGKSTYKIADKTIVRPTQEKHINGLNADYNAARNINFIVADEKWRKKFVRPTNTNKPLYNSPVFSPAVKSEGGTIKNLQILSATKTIIL

SEQ ID NO: 4 - protein, 756 aa, unidentified
  REGION 1..756: note = Description of Unknown:bovine gut metagenome sequence
MTTKQVKSIVLKVKNTNECPITKDVINEYKKYYNICSEWIKDNLTSITIGDIASFLKEATNKDTIPTYINMGLSEEWKYKPIYHLFTDDYHEKSANNLLYAYFKEKNLDCYNGNILNLSETYYRRNGYFKSVVGNYRTKIRTLNYKIKRKNVDENSTNEDIELQVMYEIAKRKLNIKKDWENYISYIENVENINIKNIDRYNLLYKHFCENESTINCKMELLSVEQLKEFGGCVMKQHINSMTINIQDFKIENKENSLGFILNLPLNKKKYQIELWGNRQIKKGNKDNYKTLVDFINTYGQNIIFTIKNNKIYVVFSYECELKEKEINFDKIVGIDVNFKHALFVASERDKNPLQDNNQLKGYINLYKYLLEHNEFTSLLTKEELDIYKEIAKGVTFCPLEYNLLFTRIENKGGKSNDKEQVLSKLLYSLQIKLKNENKIQEYIYVSCVNKLRAKYVSYFILKEKYYEKQKEYDIEMGFTDDSTESKESMDKRRLEFPFRNTQIANGFLEKLSNVQQDINGCLKNIINYAYKVFEQNGFGVIALENLENSNFEKTQVLPTIKSLLEYHKLENQNINNINASDKVKEYIEKEYYELTTNENNEIVDAKYTKKGIIKVKKANFFNLMMKSLHFASNKDEFILLSNNGKTQIALVPSEYTSQMDSIEHCLYVDKNGKKVDKKKVRQKQETHINGLNADFNAANNIKYIIENENLRKLFCGKLKVSGYNTPILDATKKGQFNILAELKKQNKIKIFEIEK

SEQ ID NO: 5 - protein, 746 aa, unidentified
  REGION 1..746: note = Description of Unknown:bovine gut metagenome sequence
MASHKKTESNQIIKTFPFKLKNANGLSLDVLNDAITEYQNYYNICSDWIKDHLTMKISELYKYIPDEKKNSGYALTLISDEWKDKPMYMMFKKGYPANNRDNAIYETLNTCNTEHYTGNILNFPDTYYRRFGYVASTISNYVTKISKMSTGSRSKNISNDSDVDTIMEQVIYEMEHNGWTSVKDWENQMEYLESKTDSNPNFVYRMTTLYEFYKSHIDEVNSKMETMSIDLLIKFGGCRRKDSKKSMYIMGGSNTPFDITQIGDNSLNIKFSKNLNVDVFGRYDVIKDNTLLVDIINGHGASFVLKIINDEIYIDINVSVPFDKKIATTNKVVGIDVNIKHMLLATNILDDGNVKGYVNIYKEVINDSDFKKVCNSTVMKYFTDFSKFVTFCPLEFDFLFSRVCNQKGIYNDNSVMEKSFSDVLNKLKWNFIETGDNTKRIYIENVMKLRTQMKAYAIVKNAYYKQQSEYDFGKSEEFIQEHPFSNTDKGIEILHKLDNISKKILGCRNNIIQYSYNLFEINGYDMISLEKLTSSQFKKKSFPTVNSLLKYHKILGCTQEEMEKKDIYSVIKKGYYDIIFDNDVVTDAKLSTKGELSKFKDDFFNLMIKSIHFADIKDYFITLSNNGTAGVSLVPSFFTSQMDSIDHKIYFVQDNKSGKLKLANKHKVRSSQEKHINGLNADYNAARNIAYIMENTECRNMFMKQSRTDKSLYNKPSYETFIKTQGSAVAKLKKEGFMKILDEASV

SEQ ID NO: 6 - protein, 733 aa, unidentified
  REGION 1..733: note = Description of Unknown:bovine gut metagenome sequence
MAHKKNIGAEIVKTYSFKVKNTNGITMEKLMNAIDEYQSYYNLCSDWICKNLTTMTIGDLDRYIPEKAKDNIYATVLLDEVWKNQPLYKIFGKKYSSNNRNNALYCALSSVIDMTKENVLGFSKTHYIRNGYILNVISNYASKLSKLNTGVKSRAIKETSDEATIIEQVIYEMEHNKWESIEDWKNQIEYLNSKTDYNPTYMERMKTLSAYYSTHKSEVDAKMQEMAVENLVKFGGCRRNNSKKSMFIMGSNTTNYTISYIGDNCFNINFANILNFDVYGRRDVVKNGEVLVDIMANHGDSIVLKIVNGELYADVPCSVTLNKVESNFDKVVGIDVNMKHMLLSTSVTDNGSSDFVNIYKEMSNNAEFMALCPEKDRKYYKDISQYVTFAPLELDLLFSRISKQGEVKMEKAYSEILESLKWKFFANGDNKNRIYVESIQKIRQQIKALCVIKNAYYEQQSAYDIDKTQEYIETHPFSLTEKGMSIKSKMDKICQTIIGCRNNIIDLAYSFFERNGYSIIGLEKLTSSQFKNTKSMPTCKSLLNLHKVLGHTLSELETLPINDIVKYYTFTTDNEGRITDASLSEKGKIRKMKDRFLNQAIKAIHFADVKDYFATLSNNGQTGIFFVPSQFTSQMDSNTHNLYFEVDKNGGLKMASKDKTRPKQEYHRNGLPADYNAARNIAYIGLDETMRNTFLKKVNSNKSLYNQPIYDTGIKKTAGVFSRMKKLKRYEII

SEQ ID NO: 7 - protein, 744 aa, unidentified
  REGION 1..744: note = Description of Unknown:bovine gut metagenome sequence
MIKSIKLKVKGDCPITKDVINEYKEYYNRCSDWIKNNLTSITIGEIGKFLQDVTGKTTGYIEVALSDKWKDKPMYYLFTDQYDTNHANNLLYSFIQENNLDGYDGNSLNISGTYYRKQGYFKLVSSNYRTKIRTLNCKIKRKKVDVDSTSEDIESQVMYEIINRSLNKKSDWDSFISYIENVENPNIDSINRYTLLRDYFCDNEDVIKNKIELLSIEQLKDFGGCIMKQHINTMSLNIQHFKIEEKENSLGFILYLPLNKKQYQIELWGHRQIKKGSKESCETLVDFINTYGENIVFTINNDELYVVFSYESEFGKEETNFEKSVGLDINFKHALFVTSELDNDQFDGYINLYKYILSHSEFTNLLTEDERKDYEELSKVVTFCPFENQLLFARYDKMSKFCKKEQVLSKLLYSLQKKLKNENRTKEYIYVSCVNKLRAKYISYFILREKYDEKNKEYDIEMGFVDDSTESKESMDKRRFENPFRNTLVANELLAKMSKVQQDINGCMSNIINYVYKVFEQNGYNIIALENLENSNFEKRQVLPTIKSLLKYHKLENQNINDIKASDKIKEYIENGYYSFTTNENNEIVDAKYTAKGDIKVKNAKFFNLMMKILHFASIKDEFVLLSNNGKSQIALVPPEYTSQMDSIDHCIYMTENDKGKIVKVDKRKVRTKQERHINGLNADFNAANNIKYIVSNEKWRNVFCTPKKAKYNTPALDATKKGQFRILDDMKKLNATKLLEIEK

SEQ ID NO: 8 - protein, 754 aa, unidentified
  REGION 1..754: note = Description of Unknown:bovine gut metagenome sequence
MYQLNQYIMASHKKTESNQIIKTFSFKIKNANGLSLDVLNDAITEYQNYYNICSDWIKDHLTMKISELYKYIPDEKKNSGYALTLISDEWKDKPMYMMFKKGYPANNRDNAIYETLNTCNTEHYTGNILNFSDTYYRRFGYVASAISNYVTKISKMSTGSRYKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINDEIYIDINVSVPFDKKIATTNKVVGIDVNIKHMLLATNILDDGNVKGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILHKLDNISKKILGCRNNIIQYSYNLFEINGYDMISLEKLTSSQFKKKPFPTVNSLLKYHKILGCTQEEMEKKDIYSVIKKGYYDIIFDNGVVIDAKLSAKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVSKLKKDGFVKILDEASV

SEQ ID NO: 9 - protein, 746 aa, unidentified
  REGION 1..746: note = Description of Unknown:bovine gut metagenome sequence
MASHKKTESNQIIKTFSFKIKNANGLSLDVLNDAITEYQNYYNICSDWIKDHLTMKISELYKYIPDEKKNSGYALTLISDEWKDKPMYMMFKKGYPANNRDNAIYETLNTCNTEHYTGNILNFSDTYYRRFGYVASAISNYVTKISKMSTGSRYKNISNDSDVDTIMEQVIYEMEHNGWTSVKDWENQMEYLESKTDSNPNFVYRMTTLYEFYKSHIDEVNSKMETMSIDSLIKFGGCRRKDSKKSMYIMGGSNTPFDITQIGGNSLNIKFSKNLNVDVFGRYDVIKDNTLLVDIINGHGASFVLKIINDEIYIDINVSVPFDKKIATTNKVVGIDVNIKHMLLATNILDDGNVKGYVNIYKEVINDSDFKKVCNSTVMKYFTDFSKFVTFCPLEFDFLFSRVCNQKGIYNDNSAMEKSFSDVLNKLKWNFIETGDNTKRIYIENVMKLRSQMKAYAIVKNAYYKQQSEYDFGKSEEFIQEHPFSNTDKGIEILHKLDNISKKILGCRNNIIQYSYNLFEINGYDMISLEKLTSSQFKKKPFPTVNSLLKYHKILGCTQEEMEKKDIYSVIKKGYYDIIFDNGVVIDAKLSAKGELSKFKDDFFNLMIKSIHFADIKDYFITLSNNGTAGVSLVPSYFTSQMDSIDHKIYFVQDNKSGKLKLANKHKVRSSQEKHINGLNADYNAARNIAYIMENTECRNMFMKQSRTDKSLYNKPSYETFIKTQGSAVSKLKKDGFVKILDEASV

SEQ ID NO: 10 - protein, 745 aa, unidentified
  REGION 1..745: note = Description of Unknown:bovine gut metagenome sequence
MIKSIQLKVKGECPITKDVINEYKEYYNNCSDWIKNNLTSITIGEMAKFLQSLSDKEVAYISMGLSDEWKDKPLYHLFTKKYHTKNADNLLYYYIKEKNLDGYKGNTLNISNTSFRQFGYFKLVVSNYRTKIRTLNCKIKRKKIDADSTSEDIEMQVMYEIIKYSLNKKSDWDNFISYIENVENPNIDNINRYKLLRECFCENENMIKNKLELLSVEQLKKFGGCIMKPHINSMTINIQDFKIEEKENSLGFILHLPLNKKQYQIELLGNRQIKKGTKEIHETLVDITNTHGENIVFTIKNDNLYIVFSYESEFEKEEVNFAKTVGLDVNFKHAFFVTSEKDNCHLDGYINLYKYLLEHDEFTNLLTEDERKDYEELSKVVTFCPFENQLLFARYNKMSKFCKKEQVLSKLLYALQKKLKDENRTKEYIYVSCVNKLRAKYVSYFILKEKYYEKQKEYDIEMGFVDDSTESKESMDKRRTEYPFRNTPVANELLSKLNNVQQDINGCLKNIINYIYKIFEQNGYKVVALENLENSNFEKKQVLPTIKSLLKYHKLENQNVNDIKASDKVKEYIENGYYELMTNENNEIVDAKYTEKGAMKVKNANFFNLMMKSLHFASVKDEFVLLSNNGKTQIALVPSEFTSQMDSTDHCLYMKKNDKGKLVKADKKEVRTKQERHINGLNADFNAANNIKYIVENEVWRGIFCTRPKKTEYNVPSLDTTKKGPSAILNMLKKIEAIKVLETEK

SEQ ID NO: 11 - protein, 744 aa, unidentified
  REGION 1..744: note = Description of Unknown:bovine gut metagenome sequence
MIKSIVFKVKGDCPITKDVIKEYKEYYNRCSEWIKNNLTSITIGEIGKFLQDTMGKTHGYIKVALSDEWKDKPMYYLFTEKYDTKHANNLLYYFIQENNLDRYEGNSLNIPSYYYKREGYFKLVTSNYRTKIRTLNCKIKRKKIDVDSTCVDIENQVIYEIIKKGLNKKSDWDNYISYIENIEMPNIDSINRYKLLRDYFCENENVIKNKIELLSIEQLKNFGGCIMKQHINTMILNIKRLKIEEKENSLGFILHLPLNKKQYQIELWGNRQIKKGTKESNETLVDFINTYGEDVVFTIKKNELYAKFSYECEFEKEETNFEKSVGLDINFKHALFVTSELDDDQFYGYINLYKYILSHSEFTNLLTEDEKKDYEDLSNAITFCPFENQLLFTRYDKKSKLYKKEQVLSKILYSLQKKLKDENRKQEYIYVSCVNKLRAKYVSYFILKEKYNEKQKEYDIEMGFVDDSTESKESMDKRRYEYPFRNTPVANELLEKMNNVQQDISGCLKNIINYAYKVFEQNGYNIVALENLENSNFEKRNVLPTIKSLLKYHKLENQNITDIKASDKIKEYIENGYYELITNENNEIIDAKYTENGDIKVKNARFFNLMMKSLHFASIKDEFVLLSNNGKSQIALVPSEYTSQMDSTDHCIYMTENDKGKLVKVDKRKVRTKQERHINGLNADFNAANNIKYIVENEKWRKVFCAPQKAKYNTPTLDATKKGQFRILEDLKKLKATKLLEIGK

SEQ ID NO: 12 - protein, 745 aa, unidentified
  REGION 1..745: note = Description of Unknown:bovine gut metagenome sequence
MIKSIQLKVKGECPITKDVINEYKEYYNNCSDWIKNNLTSITIGEMAKFLQSLSDKEVAYISMGLSDEWKDKPLYHLFTKKYHTKNADNLLYYYIKEKNLDGYKGNTLNISNTSFRQFGYFKLVVSNYRTKIRTLNCKIKRKKIDADSTSEDIEMQVMYEIIKYSLNKKSDWDNFISYIENVENPNIDNINRYKLLRECFCENENMIKNKLELLSVEQLKKFGGCIMKPHINSMTINIQDFKIEEKENSLGFILHLPLNKKQYQIELLGNRQIKKGTKESHETLVDITNTHGENIVFTIKNDNLYIVFSYESEFEKEEVNFAKTVGLDVNFKHAFFVTSEKDNCHLDGYINLYKYLLEHDEFTNLLTEDERKDYEELSKVVTFCPFENQLLFARYNKMSKFCKKEQVLSKLLYALQKKLKDENRTKEYIYVSCVNKLRAKYVSYFILKEKYYEKQKEYDIEMGFVDDSTESKESMDKRRTEYPFRNTPVANELLSKLNNVQQDINGCLKNIINYIYKIFEQNGYKVVALENLENSNFEKKQVLPTIKSLLKYHKLENQNVNDIKASDKVKEYIENGYYELMTNENNEIVDAKYTEKGAMKVKNANFFNLMMKSLHFASVKDEFVLLSNNGKTQIALVPSEFTSQMDSTDHCLYMKKNDKGKLVKADKKEVRTKQERHINGLNADFNAANNIKYIVENEVWRGIFCTRPKKTEYNVPSLDTTKKGPSAILNMLKKIEAVKILETEK

SEQ ID NO: 13 - protein, 712 aa, unidentified
  REGION 1..712: note = Description of Unknown:bovine gut metagenome sequence
MKNNLTTVTIGEMAKFLQETTGKNVTYITMGLSEEWKDKPLYHLFYGKYHTKNADNLLYYFIKAKKLDEYDGNMLNLGDTYYRQFGYFKLVVSNYRTKIRTLNLNVKRKRVDVDSTSEDIESQVMYEIVKRNLNTISDWENYISYIEDVETPNIDNINRYKFLQNYFCENEEDIKNKIEFLSIEQLKDFGGCIMKPHINSMTINIQDFKIEEIENSLGFVLQLPLNKKYHQIELYGNRQVKKGTKENYKTLVDIINTHGENIVFTIENNELYVVFSYEYELKKKDINFEKMAGIDVNFKHALFVTSETDNNQLNHYINLYKHILEHNEFTTLLTDSERKDYEEIAKTVTFCPFEYQLLFTRFDKNSNANVKEQALSKILYDLQKKLKSQNKIKEYIYVSCVNKLRAKYVSYFILKEKYYEKQKEYDIQMGFVDDSTESKSSMVKRRVEYPFRNTPVANALLAIVNNVQQDINGCLKNIINYAYKVFELNDYNVVALENLENANFEKKQVIPTIKSLLKYHKLEMQNINDIKANDTIKKYIENEYYQLITNENNEIVNAIYTPKGITKLKYANFFNLLMKSLHFASIKDEFILLSNNGNTNIALVPHEYTSQMDSIDHCIYMVQNDKGNLVKAHKTKVRTKQEKHINGLNADFNAANNIKYIVENEKWRNIFCKIPKKIEYNTPVLDVTKKGQSNIIKTLKNLNATKILEIKK

SEQ ID NO: 14 - protein, 741 aa, unidentified
  REGION 1..741: note = Description of Unknown:terrestrial metagenome sequence
MKKSIKFKVKGNCPITKDVINEYKEYYNKCSDWIKNNLTSITIGEMAKFLQETLGKDVAYISMGLSDEWKDKPLYHLFTKKYHTNNADNLLYYYIKEKNLDGYKGNTLNIGNTFFRQFGYFKLVVSNYRTKIRTLNCEIKRKKIDADSTSEDIEMQTMYEIIKHNLNKKTDWDEFISYIENVENPNIDNINRYKLLRKCFCENENMIKNKLELLSIEQLKNFGGCIMKQHINSMTLIIQHFKIEEKENSLGFILNLPLNKKQYQIELWGNRQVNKGTKERDAFLNTYGENIVFIINNDELYVVFSYEYELEKEEANFVKTVGLDVNFKHAFFVTSEKDNCHLDGYINLYKYLLEHDEFTNLLTNDEKKDYEELSKVVTFCPFENQLLFARYNKMSKFCKKEQVLSKLLYALQKQLKDENRTKEYIYVSCVNKLRAKYVSYFILKEKYYEKQKEYDIEMGFVDDSTESKESMDKRRTEFPFRNTPVANELLSKLNNVQQDINGCLKNIINYIYKIFEQNGYKIVALENLENSNFEKKQVLPTIKSLLKYHKLENQNVNDIKASDKVKEYIENGYYELITNENNEIVDAKYTEKGAMKVKNANFFNLMMKSLHFASVKDEFVLLSNNGKTQIALVPSEFTSQMDSTDHCLYMKKNDKGKLVKADKKEVRTKQEKHINGLNADFNAANNIKYIVENEVWREIFCTRPKKAEYNVPSLDTTKKGPSAILHMLKKIEAIKILETEK

SEQ ID NO: 15 - protein, 752 aa, unidentified
  REGION 1..752: note = Description of Unknown:feces metagenome sequence
MAKSIMKKSIKFKVKGNSPINEDIINEYKGYYNTCSNWINNNLTSITIGEMGKFLKDVMRKTTGYIDVALSDEWKDKPMYYLFTKKYNPKHANNLLYYFIKEKKLDKFNGNILNVPEYYYRKEGYFKLVAGNYRTKINTLNFKIKSKKVDANSLSEDIEMQTIYEIVKRGLNKKSDWDSYISYIECVQNPNIDNINRYKLLRDYFCENEDVIKNKIEILSIEQIKEFGGCIMKPHINSMTFGIQKFKIEEIENSLGFTFNLPLNKNNYKIELWGHRQLKKGNKESNVNVSLDDFINTYGQNVVFTIKRKKLYIVFSYDYEFERGECNFEKSVGLDVNFKHSLFVTSEIDNNQFDGYINLYKYILSNNEFTSLLTDSERKDYEDLANIVTFCPFEYQLLFSRYDKLSKISEKEKVLSKILYSLQKKLKNEKRTKEYIYVSCVNKLRAKYVSYFKLKQKYNEKQKEYDIEMGFVDDSTESKESMDKRRFENPFINTPVAKELLEKMNNVKQDINGCKKNIVVYAYKVLEQNGYNIIALENLENSNFEKIRVLPKIKSLLEYHKFENKNINDIKNSDKYKEFIEPGYFELITNENNEIIDAKYTQKGDIKIKNADFINIMIKALNFASIKDEFILLSHNGKSQIALVPAEYTSQMDSIDHCIYMTKNDKGKLVKVDKRKVRTKQERHINGLNADFNAACNIKYIVTNEDWRKVFCIKPKKEDYNTPLLDATKNGQFRILDKLKKLNATKLLEMEK

SEQ ID NO: 16 - protein, 766 aa, unidentified
  REGION 1..766: note = Description of Unknown:feces metagenome sequence
MANKKFKLTKNEVVKSFVLKVANQKKCAITNETLQEYKNYYNKVSQWINNNLTKMTIGDLIQYAPTVSKKGKKQPDGTMVYDTPLYVTYAMSDEWKNKPLYYIFKKEYNTNNANNLLYEAIRNLNVDEYDGNQLNFNSTYYRTQGYVNRVFSNYRTKINTLDIKIKKSKVDENSDVETLELQTMYEINKLNLKTNKDWEERLQYLTMQENPNQNTIDRTKILFNYFINNNDTIFQKMEELSIKQLTEFGGCKMKDNTTSMTINIQDFKIKRKENSIGYIMTIPFNKKNVDVELYGHKQTIKGHKNSYTEIVDIVNKHGNTITFKIKNNQLFAIITSDTEVTKPEPQYEKIVGVDVNIKHTLMVTSEKDNGKLKGYINLYKEVLKNDEFKKLLNKTELDNFKSLSQIVTFCPIEYDFLFSRIFDDENTKKELAFSNVLYDIQKQLKNTNNILQYNYIACVNKLRAKYKAYFVLKMSYMKQQKIYDTNMGFFDISTESKETMDQRRSLYPFINTEIAQNIITKMNNVQQDINGCLKNIFKYTYTVFENNNYDTIVLENLENANFEKHNPLPNITSLLKYHKVQGLTIQEAEQHEKVGNLIQNDNYIFQLNEDNKIINADYSQKAYYKVCKALFFNQAIKTLHFASVKDEMIKLSNNNKVCVAIIPPEYTSQIDSNTHKLYFINKDGKLLKADKKTVRKTQEKHINGLNADFNAASNIKYIVQNETWRNLFTNKTNNTYGLPILTPSKKGQSNIITQLMKINATQELVV

SEQ ID NO: 17 - protein, 784 aa, unidentified
  REGION 1..784: note = Description of Unknown:sheep gut metagenome sequence
MYNSKKKGEGDIQKSFKFKVKTDKETVELFRKAAVEYSEYYKRLTTFLCERLTDMTWGEVASFIPEKYRKNEYYKYLIKEENKDLPLYKMFTKAASSMFIDHSIERYVEALNPEGNTGNILGFCKSSYVRGGYLKNVVSNIRTKFATLKTGIKYKKFNPAEDDEETILGQTVFEMEKRGLEFKCDFEKTIKYLNEKGKTQEAERLQCLMEYFSTNTDKINEYRESLVLDDIRKFGGCNRSKSNSFSVTLEKADIKEDGLTGYTMKVSKKLKEIHLLGHRRVVEVVNGRRVNLVDICGDKSGDSKVFVVDGDNLYVCISAPVKFSKNGMEAKKYIGVDMNMKHSIISVSDNASDMKGFLNIYKELLKDEGFRKTLNATELEKYEKLAEGVNIGIIEYDGLYERIVKQKKENSVDGLKVQAEKKLIEREAAIERVLDKLRKGTSDTDTENYINYNKILRAKIKSAYILKDKYYEMLGKYDSERAGSGDLSEENKIKYKDEFNETEKGKEILGKLNNVYKDIIGCRDNIVTYAVNLFIRNGYDTVALEYLESSQMKARRIPSTGGLLKGHKLEGKPEGEVTAYLKANKIPKSYYSFEYDGNGMLTDVKYSDMGEKARGRNRFKNLVPKFLRWASIKDKFVQLSNYKDIQMVYVPSPYTSQTDSRTHSLYYIETVKVDEKTGKEKKEHIVAPKESVRTEQESFVNGMNADTNSANNIKYIFENETLRDKFLKRTKDGTEMYNRPAFDLKECYKKNSNVSVFNTLKKTLGAIYGKLDENGNFIENECNK

SEQ ID NO: 18 - protein, 782 aa, unidentified
  REGION 1..782: note = Description of Unknown:gut metagenome sequence
MAGHSKIKENHIMKAFLMKVKETRKKQWQSNFIRSEIAKFTNYYNGLSKFIADRLLDDMVTTLAPLIEEKKRNSEYYKYLTNGDWDGKPLYFIFKEGFNSTNADNILANSLVRVYCEQNYTGNGFGLSYSYYVVIGFAKEVIANYRSSFQKPKVKIKKKKLSENPTEDELIEQCIYTIYYEFNEKKDIQKWKDEIKFLKERGESKETRLKRIQTLFEFYKDKSHKELVDERVANLVVDNIKEFGGCKRDIDCPSMGIQIQHNFDISINEKRNGYTICFGPNKKNLTKLEVFGNRMVLLNGEEIVDLPNTHGEKLTLIDRGNAIYAAITAQVPFEKHMPDGNKTVGIDLNLKHSVFATSIVDNGKLAGYISIYKELLKDDEFVKYCPKDLLRFMKDASKYVFFAPIEIELLRSRVIYNKGYACVENYENVYKAEVAFVNVIKRLQSQCEANGDAQGALYMSYLSKMRAQLKNYINLKLAYYDHQSAYDLKMGFTDISTESKETMDERRKLFPFNKEKEAQEILAKMKNISNVIIACRNNIAVYMYKMFERNGYDFIGLEKLESSQMKKRQSRSFPTVKSLLNYHKLAGMTMDEIKKQEVSSNIKKGFYDLEFDADGKLYGAKYSNKGNVHFIEDEFYISGLKAIHFADMKDYFVRLSNNGKVSVALVPPSFTSQMDSVEHKFFMKKNANGKLIVADKKDVRSCQEKHKINGLNADYNAACNIGFIVEDDYMRESLLGSPTGGTYDTAYFDTKIQGSKGVYDKIKENGETYIAVLSDDVITAEV

SEQ ID NO: 19 - protein, 735 aa, unidentified
  REGION 1..735: note = Description of Unknown:human gut metagenome sequence
MAHKKNVGAEIVKTYSFKVKNTNGITMEKLMNAIDEFQSYYNLCSDWICKNLTTMTIGDLDQYIPEKAKGNTYATVLLDEAWKNQPLYKIFGKKYSSNNRNNALYCALSSVIDMTKENVLGFSKTHYIRNDYILNVISNYASKLSKLNTGVKSRAIKETSDEATIIEQVIYEMEHNKWESIEDWKNQIEYLNSKTDYNPTYMERMKTLSAYYSTHKSEVDAKMQEMAVENLVKFGGCRRNNSKKSMFIMGSNTTNYTISYIGGNSFNINFANILNFDVYGRRDVVKNGEVLVDIMANHGDSIVLKIVNGELYADVPCSVTLNKVESNFDKVVGIDVNMKHMLLSTSITDNGSSDFLNIYKEMSNNAEFMALCPEEDRKYYKDISKYVTFAPLELDLLFSRISKQGKVKMEKVYSEILEALKWKFFANGDNKNRIYVESIQKIRQQIKALCVIKNAYYEQQSAYDIDKTQEYIETHPFSLTEKGMSIKSKMDKICQTIIGCRNNIIDYAYSFFERNGYSIIGLEKLTSSQFEKTKSMPTCKSLLNFHKVLGHTLSELETLPINDVVKKGYYTFTTDNEGKITDASLSEKGKVRKMKDDFFNQAIKAIHFADVKDYFATLSNNGQTGIFFVPSQFTSQMDSNTHNLYFENAKNGGLKLAPKYKVRQTQEYHLNGLPADYNAARNIAYIGLDETMRNTFLKKANSNKSLYNQPIYDTGIKKTAGVFSRMKKLKRYEII

SEQ ID NO: 20 - protein, 774 aa, unidentified; identical to SEQ ID NO: 3
  REGION 1..774: note = Description of Unknown:mammals-digestive system-asian elephant fecal-elephas maximus sequence

SEQ ID NO: 21 - protein, 755 aa, unidentified
  REGION 1..755: note = Description of Unknown:mammals-digestive system-cattle and sheep rumen sequence
MAHVRTKNEGNMAKTYSFKVRETNLKKDVMIEYNEYYNRLSDWICGNLTKMTIGELAELVPEKKRNTSYYLAATDEKWINEPMYKLFTDEYTKKSSFTDPLVANSNNCDNLILTATDVLNPEGYEGNLLSLCKSTYRTFGYAKQIISNMKTKIGALKPNVKRRVLGENPTYDEKMIQVLYEMYNNGIADVTGFNDRIKYLKKQETPNEKLISRMKMLRDFFKENRNDIMDKCRIMAVEQLVSFGGCKRNINGASMTLRNQCISVKRKDGCQGYVVAIPVGTKNSIVFDLYGRRDVIKDGVELVDVCGKHTDTITIKSVNGELFLDMPVAINFEKKSGKCTKTVGIDVNTKHMLIQTSVKDNGKFDYYVNLYKIFAEDEELNKILGDDEVMVNIKKNAENLSFLPLEMDLLYSRILDGPQKYKLAEDRITELLKQWGINFDAGCMSQERIYVQCVRKLRGNLKRLLYLQNKYYEAQQEYDKKMGFDDKSTDSKETMDKRRWESPFRNTEEGTKLYDEINTYQNRIIGIRNSIIDYAYLVLEYNGYDNLSLEYLTSSQFKVNKTFPTTNSLLKYHKLQGKTKTEAEKCDAYISHKSKYKLSLKDGVIDSIDYSAEGLKQIKKDRSRNIIIKAIHFADVKDRFVLSSNNGNASVTFVPSYHTSQIDSTDHKMFVTNKGKIVDKRKVRQIQETHVNGLNSDFNAARNIQYISENEEWRNALCKPTENMYNEPIYVPLVKSQNGMFKAIKKLGATKIWQE

SEQ ID NO: 22 - protein, 789 aa, unidentified
  REGION 1..789: note = Description of Unknown:mammals-digestive system-cattle and sheep rumen sequence
MAHRNKNLAENCINKTFSFKVKAEKEEINSKWIPAIKEYTAYYNRISDWICDRLTNTTVGELIGIIGYKTDKKGNALAYIKDGSSEKYRNLPLYCMFKKNFPATTADNIMYQVIEKLGVDKYNGNSLGLSGTYYRRIGYIANVIGNYRTKVRGMKASVKYRNFDPNDVTEDVLENQTIFEINKNGFECKGDFEKHIEYLKNRELTDRLNKLILRMECLYNYYVEHEDAVKAKMENYAIESFKTFGGCHRNSNRSMSIQFTNNSPLEIKKVGKTSFDLYMPINGEVACLQLMGNKQAVCVGENGERCDLVDIVNSHSKTITIKIINGEMYVDIPCVVNFEKKDEDTIKSVGVDVNIKHEILATSVIDNGQLNGYFNIYKELINNKEFVDTFNGDIKAFEAFKDNAAYVTFGLLEPDLLFTRFYERSGFEKDDRHIKLRERERILTGILKRIGQEHSDVDVRNYVRFVNMLRSKYESYFVLKNKYYEKMQEFDSTQNYVDVSTASKETMDKRRFDNPFRNTEVANELLGKIDNVLGDIKGCMANIITYAFKVLQKNGYNTIGLEYLDSSQFENMRTLTPTSILKYHKMEGKSVDAVESWIKENKIPSNRYDFIYEDNHLTDVLLNSNGIAYQKKNLFMNLVIKAISFADIKNKFVQLSNNTNVSILFAPAAFTSQMDSNRHVIYTVKNNKGKLALVDKKRVRPNQEKHINGLHSGYNAACNVKFICDNEFFRNTMTISNKGKNLYSQPTYDIKEAYKKNAGCKVINDFIKNGNAVICCIENNKLIETNGRQ

SEQ ID NO: 23 - protein, 766 aa, unidentified
  REGION 1..766: note = Description of Unknown:mammals-digestive system-fecal sequence
MANKKFKLTKNEVVKSFVLKVANQKKCAITNETLQEYKNYYNKVSQWINNNLTKMTIGDLIQYAPTVSKKGKKQPDGTMVYDTPLYVTYAMSDEWKNKPLYYIFKKEYNTNNANNLLYEAIRNLNVDEYDGNQLNFNSTYYRTQGYVNRVFSNYRTKINTLDIKIKKSKVDENSDVETLEPQTMYEINKLNLKTNKDWEERLQYLTMQENPNQNTIDRTKILFNYFINNNDTIFQKMEELSIKQLTEFGGCKMKDNTTSMTINIQDFKIKRKENSIGYIMTIPFNKKNVDVELYGHKQTIKGHKNSYTEIVDIVNKHGNTITFKIKNNQLFAIITSDTEVTKPEPQYEKIVGVDVNIKHTLMVTSEKDNGKLKGYINLYKEVLKNDEFKKLLNKTELDNFKSLSQIVTFCPIEYDFLFSRIFDDENTKKELAFSNVLYDIQKQLKNTNNILQYNYIACVNKLRAKYKAYFVLKMSYMKQQKIYDTNMGFFDISTESKETMDQRRSLYPFINTEIAQNIITKMNNVQQDINGCLKNIFKYTYTVFENNNYDTIVLENLENANFEKHNPLPNITSLLKYHKVQGLTIQEAEQHEKVGNLIQNDNYIFQLNEDNKIINADYSQKAYYKVCKALFFNQAIKTLHFASVKDEMIKLSNNNKVCVAIIPPEYTSQIDSNTHKLYFINKDGKLLKADKKTVRKTQEKHINGLNADFNAASNIKYIVQNETWRNLFTNKTNNTYGLPILTPSKKGQSNIITQLMKINATQELVV

SEQ ID NO: 24 - protein, 752 aa, unidentified; identical to SEQ ID NO: 15
  REGION 1..752: note = Description of Unknown:mammals-digestive system-fecal sequence

SEQ ID NO: 25 - protein, 814 aa, unidentified
  REGION 1..814: note = Description of Unknown:mammals-digestive system-rumen-bos taurus sequence
MVKVFINVFLSEKNQITTNIFDTEKISNSYINHINHQFMATHKKTDNQTIVKAYVMKAKMSKHDIERVWKPTIDEYINYYNKLSDWICKNLTSVTIGDLLKYVGEKQINKGVGYYTYFIDEQKTDLPLYTLFTDCPKTHADNLLFEAVRKINPENYNGNLLSLFETGYRRNGYFDNVISNYRTKMTTLKINPKYKRFSSENMPTDEVLLEQTVYEVTKNDFKNDDDWKKSIDYMKQKSEPNTALIFRMETLFDYWKDHKQDVEQYINQKRVECLKDFGGCKRRADGLSMVILLNKKLTKIEADGLTSYKLTTNLFGGKYMINIFGHRALVSVCNGERAENENIDICNKHGERFTFKIENGNLFVALTADYNYEKQPNLPKNIVGVDINIKHSMLNSSIEDKGKVKGYVNLYKEFLSDKNFRKTITSDEELNQYIELSKYATFGITELDSLFARATDTEKSILCKRELAMQDVFEKLEKRYKDDHKIKFYLGSTQKLRAQYISYFKIKEAYNRKQQEYDLAHGKTDNPDEVYKSDFINEPSAKEMLVKLNRIERKIIGCRNNIVTYAFNVIKNNGYDTIGVEYLTSSQFEKKRRLPSIKSLLNYRKLLGKPKDEWNLKEWNDVYMCYRPELDDAGNIMNFTITNEGIKRNKESTFYNSFIKAIHFADVKDKFAQLTNNNTMNTVFIPSSFTSQIDSKTRKLYLLEYTEKCDNGKTKKVVKFINKRVLRKIQEQHLNGMNADNNAARNIRDITKNLRDVFTKKQTDKNCYNSAEFMIQTKFKKRLPQATVFGELNRNGYVKVLTQEEYDELTKSAK

SEQ ID NO: 26 - protein, 776 aa, unidentified
  REGION 1..776: note = Description of Unknown:mammals-digestive system-rumen-bos taurus sequence
MATHKKTDNQTIVKAYVMKAKMSKHDIERVWKPTIDEYINYYNKLSDWICKNLTSVTIGDLLKYVGEKQINKGVGYYTYFIDEQKTDLPLYTLFTDCPKTHADNLLFEAVRKINPENYNGNLLSLFETGYRRNGYFDNVISNYRTKMTTLKINPKYKRFSSENMPTDEVLLEQTVYEVTKNDFKNDDDWKKSIDYMKQKSEPNTALIFRMETLFDYWKDHKQDVEQYINQKRVECLKDFGGCKRRADGLSMVILLNKKLTKIEADGLTSYKLTTNLFGGKYMINIFGHRALVSVCNGERAENENIDICNKHGERFTFKIENGNLFVALTADYNYEKQPNLPKNIVGVDINIKHSMLNSSIEDKGKVKGYVNLYKEFLSDKNFRKTITSDEELNQYIELSKYATFGITELDSLFARATDTEKSILCKRELAMQDVFEKLEKRYKDDHKIKFYLGSTQKLRAQYISYFKIKEAYNRKQQEYDLAHGKTDNPDEVYKSDFINEPSAKEMLVKLNRIERKIIGCRNNIVTYAFNVIKNNGYDTIGVEYLTSSQFEKKRRLPSIKSLLNYRKLLGKPKDEWNLKEWNDVYMCYRPELDDAGNIMNFTITNEGIKRNKESTFYNSFIKAIHFADVKDKFAQLTNNNTMNTVFIPSSFTSQIDSKTRKLYLLEYTEKCDNGKTKKVVKFINKRVLRKIQEQHLNGMNADNNAARNIRDITKNLRDVFTKKQTDKNCYNSAEFMIQTKFKKRLPQATVFGELNRNGYVKVLTQEEYDELTKSAK

SEQ ID NO: 27 - protein, 778 aa, unidentified
  REGION 1..778: note = Description of Unknown:mammals-digestive system-rumen-bos taurus sequence
MAHKGEKEGYQIKTLKFKVRSHDIGKSLYDIVNEYTNYYNKVSKWICDNLDTPIGELSKNISEKRHNSKYYRATNDPNWKNEPMWKIFTKKFSNGETFSEQGKNDKLANLSNCDNILSYSIIDYNIDGYTGNILGLTDTSYRLNGYISNCISNYKTKIRTAKPKVRSTAITEHSTVEEKTNNTIYEMVRKGFMSPNDFKNQIKYLTEKENPNDKLIDRLSILHSFYTENEEDVNNAFSRMSVEMLKNNNGCTRNGDKKTLNISSIDYKVTRKEGCDGYILSFGSRNQKYNIDLWGRRDTISNGKELIDLSEHGEPLTITSENGDYYVCMTVDVPFEKKSTGSTEKVASVDVNTKHTMLSTDVIDDGTLKGYLNIYKKLLLDTELTSLLHKQDFDDMKELSHNVCFGPIEYNFLLSRILDLDAYEKKVEDRITHSMKEMLKTETDERNKMYLGSVIKMRALLKVYISTKNRYHKEQQSYDESMGFTDTSTASKDTMDKRRFENPFSETETGKKLNNDLSALSKKIIGCRDNIVRYAYTTLQDNGYTMIGVEDLNSSTFANTRNPFPTIKSLLNYHHLSGKTPEEARNIDTYSKFSDHYTLTTDEEGKITDAKYTKKAETKIKKKRARDTIIKAIHFAEVKDVMCVMSNNGTASVAFEPSYFSSQMDSATHKVYTTRNKKGKDVIASKETVRPRQEKHINGMNCDINSPKNLSYLITNEEFREMFLTPTKNGYNEPFYKSRVKSAASMMSGLKKLGATMPLTDENAIFSTPKPKKNIGKQ

SEQ ID NO: 28 - protein, 772 aa, unidentified
  REGION 1..772: note = Description of Unknown:mammals-digestive system-rumen-bos taurus sequence
MGNKVQSNETIVKTYTFKVREFISGATHEIMKSAIKQYIEDSNNLSDWINNQLTNKTICEVGALIPIEKRETSYYKSTVDELWANKPCFKMFTNDFTKEENFATRNIGNGKNCKNIITSAYKSTVNPSFRNVLDLTEKVYFSDGYGANVCSNYKTKLRTLKPAKIKLVSSLSDCDDNTLTEQVIREKQKYGYSTPKDFEKRIEYLNEKEKSEQNSKIIERLQKLYEFYDNNTKLVEEKELELSVKSLVEFGGCRRGEKTMTLNLPDIGYEIQRKDDKYGYIFTLKCSKKRKIIIDVWGSKATIDSNGNDKVDIINTHGKSINFKIINNEMYIDITVDVPFAKRKLGIKKVVGIDVNTKHMLMATNIKVTDSIKGYVNLYKEFLNSKEIMDVASPETKKNFEDMSMFVNFCPIEYNTMFALIFKLNNGDIRTEQAIRRTLHQLSKKFSDGNHETERIYVQNVFSIREQLKHFILLSNRYYSEQSDYDTKMGFIDENTTSNATMDKRRFDKSLMFRYTQRGRQLYEERIECGRKITEIRDNIITYARNVFVLNGYDTIALEYLTNATIQKPTRPTSPKSLLDYFKLKGKPVVEAEKNERITKNRKYYNLIPDENDNVINIEYTEEGKVAIKKSIARDHIMKAVHFAEVKDKFIQLSNNGKTQVALVPSNYTSQMNSETHTVYLMKNPKTKKLVIMDKDKVRPIQEKYKLNGLNADFNSARNIAYIVENEILRNSFLKEETKKYTYNTPLFTPRLKSSEKIITELKKLGMTTVIE

SEQ ID NO: 29 - protein, 781 aa, unidentified
  REGION 1..781: note = Description of Unknown:mammals-digestive system-rumen-bos taurus sequence
MANKSTKGNLPKTIIMKANLSPDGFTQWERVVKEYQAYKDTLSKWVAQNLTAMKIGDLLPYLDKYSKKTNKETGERPVNVYYQLCEQHKDEPLYKLFTYDSNSRNNAMYEIIRKTNCDGYKGNILGISETHYRRNGFVKNILANYTTKISTLELSERKRKIDSDSPEDLIRSQVVYEMQKNNIKDAKGFKSIIEYLKSKKEVNIQYLERLQILYEYFKNHENEIKEYITLAAVEQLKSFGGVRVNNEKSSMNLEIQGFSITRVDGACTYILHLPINGKIHGIKLWGNRQVVVNKDGTPVDILDLTNQHGSTINITIKNGEIYFAFTVTSDFVKPEHQIKNVVGVDVNTKHMLMQSNITDNGNVKGYFNIYKVLVEDRRFTSLLSEEQLKYFCELANIVSFCPIETEFLFARYAEYKKMSNNAEMRQIEKVFSDILDEQYKKYKDIDTSIANYISYVRKLRSQCCAYFKLKMKYKELQRQFDKEQDYKDLSTESKETMDKRRWENPFRNTPEASKLIKKMDNVSRQLIGCRDNIITYAYRVFEKNGYDTISLENLESSQFENNDHVIAPKSLLEYHHLKGKTMNYLLSDECKVRITTKDGKVKEWYHVELNDKDEIDNIFLTPEGETEKEKNLFNNMVIKIVHFADIKDKFIQLGNYNKLQTVLVPSYFTSQMDSKTHSVYVVETANTKTSKKELKLVSKKRVRRQQEWHINGLNADYNAACNIAHIAKNIELRQIMCKTPQTKNGYSSPVLTSKVKSQVEMVRELKKMGKTILYSNDSLPF

SEQ ID NO: 30 - protein, 798 aa, unidentified
  REGION 1..798: note = Description of Unknown:mammals-digestive system-rumen-bos taurus sequence
MAHRKKKDDEATLSYKFKVKVIEGDLTADDITKCIAENAEQGNHFSEFIHKNLTSKTIGEFASQLPVEKRQFGYYQYAIGGTMPAKKNASDEDKPKGELIDWSKKPFYVLFSKGYSATHAVNLIFNVYLNSEEGKAFSAKNSMNLSKSQFAYSGFVQIVCANYASMLANARPDKIKFEEITEATDDGTKKMQVVREMAERYLMKPKNFASRIEYLEANNTKGKFDKTIQRLRLLQPFFEKNEEGITELYYDLSVKALEHSGQCTYKGGRTISILEIGDIRISRKENAKGYLLTIPINRKSVVFDLYGRKDTIGGDGRDLIDIMNTHGSSLQFTADGNDIYLTITATKNFIKEKPTFNEDTVLGGDVNIKHSYTVFSTSPKDIPDFVNFYEYFAKDGEIMKLAPKPMWDYIVAAATKFLTILPIETPAISATVYGKRTEEGISRATFRETQKLIALEKAIERVMKQVFDKYNDGKHPLEAIYIGNAIKYRRLIKGYLAQKKKYYSAHSEYDKAMGYTDDDTDRKENMDERRFDDSKKFRYTPEAQALLDTMHTIEKKIVGCVSNAISYAYHKFDENGFNVIALENLTSATFAKKYKSDKPESIKKLLNFDKLLGKTLDEAKASKSISKHPNWYELVADENGCVSDIRITDEGQSATYRSLVTETIMKVSHFAETKDRFIGLANSGRLQVGLVPSQYTSYIDSTTHTLYAVIEDGKTVLAPKEVVRASQERHINGLNADYNSALNLKYMITDENFRKTFTSETSADKFGWGKPMFSPTTRSQDEVFSAIKKIGAITVLED

SEQ ID NO: 31 - protein, 786 aa, unidentified
  REGION 1..786: note = Description of Unknown:mammals-digestive system-rumen-bos taurus sequence
MAQHKSNNEESAINKTFIFKAKCEKNDVISLWEPAAKEYGDYYNKVSKWIADNLITMKIGDLAQYITNQNSKYYTAVTNKKKKDLPLYRIFQKGFSSQCADNALYCAIKSINPENYKGNSLGIGESDYRRFGYIQSVVSNFRTKMSSLKVSVKYKKFDVSNVDDETLKIQTIYDVDKYGIETAKEFKELIETLKTRVETPQLNDTIARLKCLCDYYSKNEKAINNEIETMAIADLQKFGGCQRKSLNAFTIHKQDSLMEKVGNTSFRLQLSFRKKTYVINLLGNRQVVNFVNGKRVDLIDIAENHGDLITFNIKNGELFLHITSPIVFDKDVRDIRNVVGIDVNIKHSMLATSIKDDGNVKGYINLYKELLNDDVFVSTCNESELALYRQMSENVNFGILETDSLFERIVNQSKGGCLKNKLIRRELAMQKVFERITKTNKDQNIVDYVNYVKMMRAKCKASYILKEKYDEKQKEYYVKMGFTDESTESKETMDKRREEFPFVNTDTAKELLVKQNNIRQDIIGCRDNIVTYAFNVFKNNEYDTLSVEYLDSSQFDKRRIPTPKSLLKYHKFEGKTKDEVENMMKSEKLSNAYYTFKYENDVVSDIDYSDEGNLRRSKLNFGNWIIKAIHFADIKDKFVQLSNNNKMNIVFCPSAFSSQMDSITHTLYYVEKITKNKKGKEKKKYVLANKKMVRTQQETHINGLNADYNSACNLKYIALNYELRDKMTDRFKASKKIKTMYNIPAYNIKSNFKKNLSAKTIQTFRELGHYRDGKINEDGMFVEILE

SEQ ID NO: 32 - protein, 781 aa, unidentified
  REGION 1..781: note = Description of Unknown:mammals-digestive system-rumen-bos taurus sequence
MAHKNSDGENTINKTFIFKVKCEKNDIISFWKPAAEEYCNYYNKLSEWIGKNLISMKIGDLAKYIDNPKSKYYLSVTDENKKDLPLYKIFQKGFSSIDADNALYCAIDKLNPEGYNGNILGVGKSDYRRNGYVSSVIGNFRTKMVSLKANVRWKKIDIGNVDEETLRRQTICDVEKYRIESEKDFRDLIDILKAREETPRLKEKISRLELLYDYYSKNTKTIKSEMENMAISDLQKFGGCVRKSLNTITIHKQDSKIEKEGNTSFRLHMVFNKKPYTITLLGNRQVVKYIDGKRVDIVNIVEKHGDWITFNIKNGELFVHLTKCVEFSKGQKEIKKAAGVDVNIKHAMLAASIVDDGQLKGYVNLYRELIEDDDFVSTFGDSDSGKTELGMYQKMAKTVFFGVLEVESLFERVVNQQSGWKLDNQLIRRERAMEKVFDRIVKTTSNKHIIDYVNYVKMLRAKYKAYFILDEKYHEKQREYDLSMGFTDESDERRELYPFINTETAKEILGKKRNVEQDLIGCRDNIVTYAFNVLRNNGYDTISVEYLDSSQFDKRRMPTPKSLLEYHKFKGKTQDEVERLMSEKKFAKTNYDIHYDGENKVDGIVYSKEGELRQKKLNFMNLVIKAIHFADIKDKFAQLCNNNDVNVVFGPSAFTSQMDSETHSLYYVEKETNGKNGKTGKKFVLADKKSVRRRQETHINGLNADFNAARNLEYIASNPELLERMTKRTKSGKDMYNTPSWNIRQEFKKNLSVRTINTFRELGNVKYGKINNEGLFVEDDV

SEQ ID NO: 33 - protein, 798 aa, unidentified
  REGION 1..798: note = Description of Unknown:mammals-digestive system-rumen-bos taurus sequence
MAHRKKKDDEATLSYKFKVKVIEGDLTADDITKCIAENAEQGNHFSEFIHKNLTSKTIGEFASQLPAEKRQFGYYQYAIGGTMPAKKNASDEDKPKGELIDWSKKPFYVLFSKGYSATHAVNLIFNVYLNSEEGKAFSAKNSMNLSKSQFAYSGFVQIVCANYASMLANARPDKIKFEEITEATDDGTKKMQVVREMAERYLMKPKNFASRIEYLEANNTKGKFDKTIQRLRLLQPFFEKNEESITELYYDLSVKALEHSGQCTYKGGRTISILEIGDIRISRKENAKGYLLTIPINRKSVVFDLYGRKDTIGGDGRDLIDIMNTHGSSLQFTADENDIYLTITATKNFIKEKPTFNEDTVLGGDVNIKHSYTVFSASPKDIPDFVNFYEYFAKDGEIMKLAPKPMWDYIVAAATKFLTILPIETPAISATVYGKRTEEGISRATFRETQKLIALEKAIERVMKQVFDKYNDGKHPLEAIYIGNAIKYRRLIKGYLAQKKKYYSAHSEYDKAMGYTDDDTDRKENMDERRFDDSKKFRYTPEAQALLDTMHTIEKKIVGCVSNAISYAYHKFDENGFNVIALENLTSATFAKKYKSDKPESIKKLLNFDKLLGKTLDEAKASKSISKHPNWYELVADENGCVSDIRITDEGQSATYRSLVTETIMKVSHFAETKDRFIGLANSGRLQVGLVPSQYTSYIDSTTHTLYAVIEDGKTVLAPKEVVRASQERHINGLNADYNSALNLKYMITDENFRKTFTSETSADKFGWGKPMFSPTTRSQDEVFSAIKKIGAITVLED

SEQ ID NO: 34 - protein, 724 aa, unidentified
  REGION 1..724: note = Description of Unknown:mammals-digestive system-rumen-bos taurus sequence
MVTTLAPLIEEKKRDSEYYKYLTNGDWDGKPLYFIFKEGFNSTNADNILANSLVRVYCEQNYTGNGFGLSYSYYVVIGFAKEVIANYRSSFQKPKVKIKKKKLSENPTEDELIEQCIYTIYYEFNEKKDIKKWKDEIKFLKERGESKETRLKRIQTLFEFYKDKNHKELVDERVANLVVDNIKEFGGCKRDIGCPSMGIQIQHNFDISINEKRNGYTICFGPNKKNLTKLEVFGNRMVLLNGEEIVDLPNTHGEKLTLIDRGNAIYAALTAQVPFEKHMPDGNKTVGIDLNLKHSVFATSIVDNGKLAGYISIYKELLKDDEFVKYCPKDLLRFMKDASKYVFFAPIEIELLRSRVIYNKGYACVENYENVYKAEVAFVNVIKRLQSQCEANGDAQGALYMSYLSKMRAQLKNYINLKLAYYDHQSAYDLKMGFNDISAESKETIDERRKLFPFSKEKEAQEILAKMKNISNVIIACRNNIAVYMYKMFERNGYDFIGLEKLESSQMKKRQSRSFPTVKSLLNYHKLAGMTMDEIKKQEVSSNIKKGFYDLEFDADGKLYGAKYSNKGNVHFIEDEFYISGLKAIHFADMKDYFVRLSNNGKVSVALVPPSFTSQMDSVEHKFFMKKNANGKLIVADKKDVRSCQEKHKINGLNADYNAACNIGFIVEDDYMRESLLGSPTGGTYDTAYFDTKIQGSKGVYDKIKENGETYIAVLSDDVITAEE

SEQ ID NO: 35 - protein, 772 aa, unidentified; identical to SEQ ID NO: 28
  REGION 1..772: note = Description of Unknown:mammals-digestive system-rumen-bos taurus sequence

SEQ ID NO: 36 - protein, 781 aa, unidentified; identical to SEQ ID NO: 29
  REGION 1..781: note = Description of Unknown:mammals-digestive system-rumen-bos taurus sequence

SEQ ID NO: 37 - protein, 798 aa, unidentified; identical to SEQ ID NO: 30
  REGION 1..798: note = Description of Unknown:mammals-digestive system-rumen-bos taurus sequence

SEQ ID NO: 38 - protein, 781 aa, unidentified; identical to SEQ ID NO: 32
  REGION 1..781: note = Description of Unknown:mammals-digestive system-rumen-bos taurus sequence

SEQ ID NO: 39 - protein, 786 aa, unidentified; identical to SEQ ID NO: 31
  REGION 1..786: note = Description of Unknown:mammals-digestive system-rumen-bos taurus sequence

SEQ ID NO: 40 - protein, 798 aa, unidentified; identical to SEQ ID NO: 33
  REGION 1..798: note = Description of Unknown:mammals-digestive system-rumen-bos taurus sequence

SEQ ID NO: 41 - protein, 771 aa, unidentified
  REGION 1..771: note = Description of Unknown:mammals-digestive system-rumen-ovis aries sequence
MANKRTDTTINLNKTVIMLTNMLPEVRAMFQAGIRQAQAYADLVNKWICSNLTNKIGEVLLPYIDNKNCVYYELCYKYKEAPLYTIFMKGKFDLNSRNNALYCAVVAQNIDNYSGNIFGFSQSDYRRNGYCKVVFSNYATKMSSLKPSIKKVTINEESTEETIQSQVIYEMFTNGRQWGKPEYFAEHLKYLEMKDNVSDKLMFRMKTLCEYYQTHTDLIDTMAMNAGVEALKQFEGLKLNRDKFSMTITTNSTSPYTLTRVAGTCAYNLHIPCRKRSYDIRLWGNRQTVRWVNGELVDIADIINQHGQTIIFTIKNGNVYVHIPYGLNFEKTEHEIKNVVGVDVNTKHMLMQTSIKDNGWVKGYVNIYKALVEDEEFVKYISKSDLKLYKDLSKYVSFCPLELNLLYTRYLSKKGLPFNEADNNAEKCVEKVLNNLVKQYEGDDVHVVNYIHNVKKLRALCKASFVLYKKYAELQKAFDDAQGYNDQSTETKETMDKRRWENPFIQTREAQELIAKMDNAVAGIIGCRDNIITYAYKVFGDNNYDTVGLENLTTSQFDNYSTVKSPKSLLSYYGLLGQQVDSDKYNAVMTESNKDWYDFKTDGDGNITDITLTAAGEAQKAKSLFNNKVLKNIHFADVKDKFIQLGNNGSIQTVLVPPSYTSQMDSKTHTIYVKETVDPKNKNKKKLKLVDKKLVRHGQEYHKNGLNADINAALNIAYIVENQEMREVMCLHPSKKDGVYDQPFLKATTKYPATVAGILLKMGKTTNWGEK

SEQ ID NO: 42 - protein, 764 aa, unidentified
  REGION 1..764: note = Description of Unknown:mammals-digestive system-rumen-ovis aries sequence
MNKSYVFKSNVAIDDIMSLFEPAIEEYINYYNRTSDFICDNLTSMKIGDLANYIKNKENVYCKFVLNDDIKDLPLYKIFSLNLNSSQKKNADNALYEAIKVLNADGYKGKNILGLGDTYFRRNGYVKNVISNYRTKFVTLKPNVKYSKIDINSVTEQLIKTQTIFEVVNKKIESETDFENLITYFKNRETPNDEKIKRLELLFDYYTKHKNEINEEIEKHAVESLKSFNGCRRNGNRKTMTVQMQKMLLKKHGLTSYILHLVLDKKPYDINLMGNRQTVKVDNNGNRVDLVDISSKHGYDLTFEVKGKTLFFTFSSEKDFSKKEQEIKNILGIDINTKHSMLATSITDNGKVKGYINIYVELLKNKDFVSTLNKEELAYYTEMAKFVSFGLLEIPSLFERVSNQYDKKNNVSITDETLLKREIAISQTLDNLAKKYRDKNCKIASYIDYTKMLRSKYKSYFILKQKYYEKNHEYDDKMGFSDISTNSKETMDPRRFENPFINTDIAKGLIVKLENVKCDIVGCRDNIIKYAYDVIVLNGFDTIGLEYLDSSNFERDRLPFPTAKSLMTYYGFEGKKYSEIDKSVFNTKYYNFIFNENETIKDISYSVYGLKEIQKKRFKNLVIKAIGFADIKDKFVQLSNNTNMNVIFVPAAFTSQMDSNTHKIYVKEIMDKNNKKQLQLIDKRKVRTKQEFHINGLNADFNAANNIKYIAENNDLLLTMCTKTKENNRYGNPLYNIKDTFKKKIPSSILNIFKKKDMYQIICD

SEQ ID NO: 43 - protein, 768 aa, unidentified
  REGION 1..768: note = Description of Unknown:mammals-digestive system-rumen-ovis aries sequence
MFRIFAALKLTNMGHVRLQKREGEVYKTYKLKVKSFSGNVDIKAGIVEYDQKFNNVSQWIADHLTSMTIGEAASRISPHKMDSQYAMTSLSDEWKDQPLYKIFTRGFGGMNADNLIIECTKTEENCKYDKEKSLGFSESVFRTFGFAANASSDMKSRMTQAKVKIGRKNIDEDSADDEKCLQAIYEIQKNELLTDDNWKDRIGYLEMKGDQERELERTTILYDYYRANRTTVLDKLDNLKVETLSKFRGSKRKSDRKILTLNGISYDIKRKEGCQGFELKFSVDKNHMEFDLLGHRALIKNGEMLVDIENCHGSQLSLEIDGDDMYAIISMRTFCEKNESKLEKIIGADVNIKHMFLMTSEKDDGNTKCYVNLYRELLSDSDFTDVLNKEEYEIFSELSKYVMFGLIETPYLGSRVIGTTQHEKIVEDKITSGMKKIAIRLFQEGKVRERIYVQNVLKIRALLKALFSTKLAYSNEQKIYDNLMRFGEKDDRRKDEGFHTTCRGTSLRSEMDMLSKKILACRDNIVEYGYYVIGLNGFDGISLENLESSTFMDVKISYPSCNSMLDHFKLKGKTIEEAENHETVGKFIKKGYYVMTLVNGKINDINYSEKAVMLHKKNLLYDTVIKSTHFADVKDKFVELSNNGKVSVVIVPPYFSSQMDSVTHKVFTEEIVVQKKSSNGKVRKTKKTVLVDKRKVRKTQESHINGLNADYNAALNLKYIAETIDWRSTLCFKTWNTYGSPQWDSKIKNQKTMIDRLDSLGAIELKNW

SEQ ID NO: 44 - protein, 789 aa, unidentified
  REGION 1..789: note = Description of Unknown:mammals-digestive system-rumen-ovis aries sequence
MSHEFNKNKGENEISKTFIFKTKCGKNDITSLWVPAMEEYCTYYNRVSKWICDNLTEMRIGDLAQYIDNHGSAYYSAVTDITKKDLPLYKIFKKGFSGLCADNALYCAIAKLNPEGYDGNMFGLSETYYRRQGYIANVFGNYRTKMNAGLKVGCAKWKKFDTNDVDDEILMEQVIVDVVKYDIDSKNEFKEYIEVLKCREENPKLLETIERLECLYGYYSQHEEDIKKKIEELVVEELKTFGGCVRKSMTSCTITVQDFVMERIGNTGYRINLTFNKKPYVLGLLGNRQVVRYVDGDRVELVDIVNNHGNQITFNLKNGELFVHLTSGVDFSKEESSMENIVGVDVNIKHSMLASSIVDDGNVNGYINIYKELVNDDEFVSTFGDSESGLNELELYRQMAESVNFGLMETDSLFERYVEQWKGSDSDSRLARRERVVGKVFDRIVKTNGDVHVVNYIHAVKMLRAKCKAYFVLKQKYYEKQKEYDDAHGYTDESTASKETMDKRRFENPFVETDVAKELLGKLACVEQDIIGCRDNIVTYAFNVFRRNGYDTISLEYLDSSQFKKIGMGAPTPKSLLKYHKLEGKTVEEVESIISEKGLKKNLYVFKFGDNGLLSDIEYSDEGLIRKKKADFGNIITKAIHFADIKDKFVQLTNNSDMGVVFCPSAFTSQMDSKTHRLYFVEGLDGNGKNKYVLANKWSVRRQQERHINGLNADFNSACNCQHIAYDPILRDAMTIKVEAGKGMYNKPSYDIRKKFKKNLSAATLKTFIKLGNTVKGMIVNGQFVEMES

SEQ ID NO: 45 - protein, 784 aa, unidentified; identical to SEQ ID NO: 17
  REGION 1..784: note = Description of Unknown:mammals-digestive system-rumen-ovis aries sequence

SEQ ID NO: 46 - protein, 764 aa, unidentified; identical to SEQ ID NO: 42
  REGION 1..764: note = Description of Unknown:mammals-digestive system-rumen-ovis aries sequence

SEQ ID NO: 47 - protein, 758 aa, unidentified
  REGION 1..758: note = Description of Unknown:mammals-digestive system-rumen-ovis aries sequence
MAHKTKESEKLVKSFKLKVDISNCEIEKKWIPSFEEYTNYYNGVSNWICENLISMKIGDLGQYIKNTESVYYKFITDESISNLPLYKIFTLKQTQNVDNALFCAIKEINPEKYNGNSIGLGETDYRRFGYVQCVISNYRTKIGTMKASIKYKTLPENQSYDVIFEQTMYEMIDKSLEKKEDWENIISNYKAKQTENTSKINRMETLYSFFIEHSEEIIEKSNLVAIEQLALFNGCKRKSLSTMTIHSQHSKLQKNGLTSFVFCINQKIGSINLFGNRQLVSVDENGNRNDIIDICNNYGDFITFQIKNGKMFIILTAKVDFDKENIEIKNVVGADVNIKHNMIASSIIDNGNVFGYINIYKELLNDEDFCSSCTNEELDIYKEISKSVNFGLLECESLFSRVSAQIYKENESISKLDDRFLRREKSIENVLNRLSKQYRYKDCKIATYIDYTKIMRDSYKSYFIIKEKYYEKQKEYDISMGYVDESTNSKKTMDKRRFENPFIETETAKNILSKLNRIESRLIGCRNNITNYAFDVFKNNGFDTIALEYLDSSQFDKTKVLTPISMLKYHKFEGKSIEEVKTLNVKFSMDNYEFEFDNNGKITNISFSQLGKREVMKTNFFNLIIKAIHFAEIKDKFIQLSNNKPINIVLVPSAFSSQMDSKDHKLYVDENGKLINKRKVRKQQERHINGLNADFNAACNLSYLAKNNELLEKVCLKRKKFGKASYSVPYWNVKDAFKKNVSSNMIATIKKMNMVKVF

SEQ ID NO: 48 - protein, 785 aa, unidentified
  REGION 1..785: note = Description of Unknown:mammals-digestive system-rumen-ovis aries sequence
MAHKTNNGENTINKTFIFKAKCEKNDIISLWKPAAEEYCNYYNKLSKWIGDSLTTMKIGDLAQYITNQNSAYYLAVTNDSKKDLPLYKIFQKGFSSQCADNALYSAIKAINPENYNGNSLEIGETDYRRFGYVQSVIGNFRTKMSSLKVSVKYKKFDVNDVDEETLKTQTIYDVDKYGIESIKDFNEFIEVLKLREETPQLNEKITRLECLCGYYSKNEENIKNEIETMAISDLQKFGGCQRKSLNTLTIHKQNSLMEKVGNTSFTLQLSFNKKPYTINLLGNRQVVKFVDGKRVDLIDITEKHGDWVTFNIKNDELFVHLTSPIDFEKEVCEIKNAVGVDVNIKHNMLATSIKDDGNVKGYINLYKELVNDCDFISTCNEDEFDLYRQMSESVNFGILETDSLFERVVNQSKGGCLNNKFIRRELAMQKVFDNITKTNKDQNIVDYVNYVKMLRAKYKAYFILKEKYYEKQKEYDIKMGFTDVSTESKETMDKRRMEFPFVNTDTAKELLAKLNNIEQDLIGCRDNIVTYAFNIFKNNGYDTLAVEYLDSAQFDKRRMPTPTSLLKYHKFEGKTKDEVEDMMKSKKFSNAYYTFKFENDVVSNIEYSNDGIWKQKQLNFGNLIIKAIHFADIKDKFVQLCNNNKMNIVFCPSAFTSQMDSITHTLYYVEKITKKKNGKEEKKYVLANKKMVRTQQETHINGLNADYNSACNLKYIALNDELRNEMTDTFKVTNRQKTMYGIPAYNIKRGFKKNLSAKTINTFRKLGHYRDGKINEDGMFVETLA

SEQ ID NO: 49 - protein, 805 aa, unidentified
  REGION 1..805: note = Description of Unknown:mammals-digestive system-rumen-ovis aries sequence
MAHKTNNGENTINKTFIFKAKCDNNDIISLWKPAMEEYCTYYNKLSQWICNNLTSMKVKDLFAYLDDKQKTKPCVDKKTGETKIGVGYYRYFIENNKEDMPLYWLFTKNCSSSHADNLLFEFVRKVNHEEYNGNSLGMGETDYRRFGYFQNVISNFRTKMSSLKATTKWKKFDVNDVDEDTLKNQTIYDVDKYGIESVNDFNERIDILKIREETEQTKDKIARLECLCKYYKEHEEDIKNEIATMAIADLQKFGGCQRKSMNTLTIHKQDSPMEKVGNTSFNLRLTFNKKPYTLNLLGNRQVVKFVGGKRIDLINITENHGDWITFNIKNNELFVHMTSPVDFEKEVCEIKNAVGVDVNIKHMMLATSIVDDGNVKGYINLYRELVNNNDFIATFGNSKNGHQGLEIYEQMAENVNFGILETESLFERVVNQSNGGELNNQLIRREIAMQKVFDNITKTNNDKNIVNYVNYVKMLRAKYKAYFILKEKYYEKQKEYDDMMGFNDESTENKEMMDKRRFEFSFINTDTAQELLIKLNKVEQDLIGCRDNIVTYAFNVFKTNGYDTLAVEYLDSAQFDKAKMPTPKSLLKYHKFEGKTIDEVKEMMNNKNFTNAYYNFKFENEIVKDIEYSTDGIWRQKKLNFMNLIIKAIHFADIKDKFVQLCNNNSMNVVFCPSAFTSQMDSITHSLYYIEKTSKTKNGKEKKQYVLANKKMVRTQQEKHINGLNADFNSACNLKYIALDEELRNAMTDEFNPKKQKTMYGVPAYNIKNGFKKNLSTKTINTFRTLGHYRDGKINEDGVFVENLA

SEQ ID NO: 50 - protein, 784 aa, unidentified; identical to SEQ ID NO: 17
  REGION 1..784: note = Description of Unknown:mammals-digestive system-rumen-ovis aries sequence

SEQ ID NO: 51 - protein, 764 aa, unidentified; identical to SEQ ID NO: 42
  REGION 1..764: note = Description of Unknown:mammals-digestive system-rumen-ovis aries sequence

SEQ ID NO: 52 - protein, 768 aa, unidentified; identical to SEQ ID NO: 43
  REGION 1..768: note = Description of Unknown:mammals-digestive system-rumen-ovis aries sequence

SEQ ID NO: 53 - protein, 764 aa, unidentified; identical to SEQ ID NO: 42
  REGION 1..764: note = Description of Unknown:mammals-digestive system-rumen-ovis aries sequence

SEQ ID NO: 54 - protein, 805 aa, unidentified; identical to SEQ ID NO: 49
  REGION 1..805: note = Description of Unknown:mammals-digestive system-rumen-ovis aries sequence

SEQ ID NO: 55 - protein, 785 aa, unidentified; identical to SEQ ID NO: 48
  REGION 1..785: note = Description of Unknown:mammals-digestive system-rumen-ovis aries sequence

SEQ ID NO: 56 - protein, 735 aa, unidentified
  REGION 1..735: note = Description of Unknown:pig gut metagenome sequence
MAHKKNIGAEIVKTYSFKVKNTNGITMEKLMAAIDEYQSYYNLCSDWICKNLTTMTIGDLDRYIPEKSKDNIYATVLLDEVWKNQPLYKIFGKKYSANNRNNALYCALSSVIDMNKENVLGFSKTHYVRNGYILNVISNYASKLSKLNTGVKSRAIKETSDEATIIEQVIYEMEHNKWESIEDWKNQIEYLNSKTDYNPTYMERMKTLSAYYSEHKSEIDAKMQEMAVENLVKFGGCRRNNSKKSMFIMGSNHTNYTISYIGENCFNINFANILNFDVYGRRDVVKNGEVLVDIMANHGDSIVLKIVNGELYADVPCSVTLNKVESNFDKVVGIDVNMKHMLLSTSVTDNGSLDFLNIYKEMSNNAEFMALCPEKDRKYYKDISQYVTFAPLELDLLFSRISKQDKVKMEKAYSEILEALKWKFFANGDNKNRIYVESIQKIRQQIKALCVIKNAYYEQQSAYDIDKTQEYIETHPFSLTEKGMSIKSKMDKICQTIIGCRNNIIDYAYSFFERNGYTIIGLEKLTSSQFEKTKSMPTCKSLLNFHKVLGHTLSELETLPINDVVKKGYYAFTTDNEGRITDASLSEKGKVRKMKDDFFNQAIKAIHFADVKDYFATLSNNGQTGIFFVPSQFTSQMDSNTHNLYFENAKNGGLKLASKSKVRKSQEYHLNGLPADYNAARNIAYIGLDEIMRNTFLKKANSNKSLYNQPIYDTGIKKTAGVFSRMKKLKKYKVI

SEQ ID NO: 57 - DNA, 37 bp, synthetic construct
  misc_feature 1..37: note = Description of Artificial Sequence: Syntheticoligonucleotide
  source 1..37: mol_type = other DNA
actatgttggaatacatttttataggtatttacaact

SEQ ID NO: 58 - DNA, 36 bp, synthetic construct
  misc_feature 1..36: note = Description of Artificial Sequence: Syntheticoligonucleotide
  source 1..36: mol_type = other DNA
attgttggaatatcacttttgtagggtattcacaac

SEQ ID NO: 59 - DNA, 19 bp, synthetic construct
  misc_feature 1..19: note = Description of Artificial Sequence: Syntheticoligonucleotide
  source 1..19: mol_type = other DNA
aatgttgttcacccttttt

SEQ ID NO: 60 - DNA, 36 bp, synthetic construct
  misc_feature 1..36: note = Description of Artificial Sequence: Syntheticoligonucleotide
  source 1..36: mol_type = other DNA
cctgttgtgaatactcttttataggtatcaaacaac

SEQ ID NO: 61 - DNA, 36 bp, synthetic construct
  misc_feature 1..36: note = Description of Artificial Sequence: Syntheticoligonucleotide
  source 1..36: mol_type = other DNA
attgttgtaactcttattttgtatggagtaaacaac

SEQ ID NO: 62 - DNA, 36 bp, synthetic construct
  misc_feature 1..36: note = Description of Artificial Sequence: Syntheticoligonucleotide
  source 1..36: mol_type = other DNA
attgttgtagacacctttttataaggattgaacaac

SEQ ID NO: 63 - DNA, 36 bp, synthetic construct
  misc_feature 1..36: note = Description of Artificial Sequence: Syntheticoligonucleotide
  source 1..36: mol_type = other DNA
cttgttgtatatactcttttataggtattaaacaac

SEQ ID NO: 64 - DNA, 29 bp, synthetic construct
  misc_feature 1..29: note = Description of Artificial Sequence: Syntheticoligonucleotide
  source 1..29: mol_type = other DNA
cttgttgtatatgtccttttataggtatt

SEQ ID NO: 65 - DNA, 36 bp, synthetic construct
  misc_feature 1..36: note = Description of Artificial Sequence: Syntheticoligonucleotide
  source 1..36: mol_type = other DNA
cttgttgtatatgtctttttataggtattgaacaac

SEQ ID NO: 66 - DNA, 25 bp, synthetic construct
  misc_feature 1..25: note = Description of Artificial Sequence: Syntheticoligonucleotide
  source 1..25: mol_type = other DNA
tactcttttttaggtaatgaacaac

SEQ ID NO: 67 - DNA, 36 bp, synthetic construct
  misc_feature 1..36: note = Description of Artificial Sequence: Syntheticoligonucleotide
  source 1..36: mol_type = other DNA
cttgttgtatatattcttttataggtattaaacaac

SEQ ID NO: 68 - DNA, 36 bp, synthetic construct
  misc_feature 1..36: note = Description of Artificial Sequence: Syntheticoligonucleotide
  source 1..36: mol_type = other DNA
catgttgtacatactattttttaagtattaaacaac

SEQ ID NO: 69 - DNA, 36 bp, synthetic construct
  misc_feature 1..36: note = Description of Artificial Sequence: Syntheticoligonucleotide
  source 1..36: mol_type = other DNA
gatgttggacactatgttttatacggtggatacaac

SEQ ID NO: 70 - DNA, 36 bp, synthetic construct
  misc_feature 1..36: note = Description of Artificial Sequence: Syntheticoligonucleotide
  source 1..36: mol_type = other DNA
gatgttgttatgctgtttttgtaagtaataaacaac

SEQ ID NO: 71 - DNA, 36 bp, synthetic construct
  misc_feature 1..36: note = Description of Artificial Sequence: Syntheticoligonucleotide
  source 1..36: mol_type = other DNA
attgttgtagacctctttttataaggattgaacaac

SEQ ID NO: 72 - DNA, 36 bp, synthetic construct
  misc_feature 1..36: note = Description of Artificial Sequence: Syntheticoligonucleotide
  source 1..36: mol_type = other DNA
attgttgtacgaaccattttatatggtaataacaac

SEQ ID NO: 73 - DNA, 39 bp, synthetic construct
  misc_feature 1..39: note = Description of Artificial Sequence: Syntheticoligonucleotide
  source 1..39: mol_type = other DNA
actgtaaaacccctgcagatgaaaggaaagtacaacagt

SEQ ID NO: 74 - DNA, 40 bp, synthetic construct
  misc_feature 1..40: note = Description of Artificial Sequence: Syntheticoligonucleotide
  source 1..40: mol_type = other DNA
atcatgttgtacatactattttttaagtattaaacaacta

SEQ ID NO: 75 - DNA, 36 bp, synthetic construct
  misc_feature 1..36: note = Description of Artificial Sequence: Syntheticoligonucleotide
  source 1..36: mol_type = other DNA
attgttgaatggctatgtttgtatgctatttacaac

SEQ ID NO: 76 - DNA, 36 bp, synthetic construct
  misc_feature 1..36: note = Description of Artificial Sequence: Syntheticoligonucleotide
  source 1..36: mol_type = other DNA
attgttggggtacttcttttatagggtactcacaac

SEQ ID NO: 77 - DNA, 37 bp, synthetic construct
  misc_feature 1..37: note = Description of Artificial Sequence: Syntheticoligonucleotide
  source 1..37: mol_type = other DNA
attgttgtagaccttgtgttttaggggtctaacaacg

SEQ ID NO: 78 - DNA, 36 bp, synthetic construct
  misc_feature 1..36: note = Description of Artificial Sequence: Syntheticoligonucleotide
  source 1..36: mol_type = other DNA
actgtgttggaatacaatatgagatgtatttacaac

SEQ ID NO: 79 - DNA, 36 bp, synthetic construct
  misc_feature 1..36: note = Description of Artificial Sequence: Syntheticoligonucleotide
  source 1..36: mol_type = other DNA
attgttgtggcataccgcaaggcggatgctgacaac

SEQ ID NO: 80 - DNA, 36 bp, synthetic construct
  misc_feature 1..36: note = Description of Artificial Sequence: Syntheticoligonucleotide
  source 1..36: mol_type = other DNA
aattgttgagataccgttttttatggtattggcaac

SEQ ID NO: 81 - DNA, 35 bp, synthetic construct
  misc_feature 1..35: note = Description of Artificial Sequence: Syntheticoligonucleotide
  source 1..35: mol_type = other DNA
attgttgtggcataccgtattacgggtgctgacaa

SEQ ID NO: 82 - DNA, 36 bp, synthetic construct
  misc_feature 1..36: note = Description of Artificial Sequence: Syntheticoligonucleotide
  source 1..36: mol_type = other DNA
attgttgtggcataccgtattacgggtgctgacaac

SEQ ID NO: 83 - DNA, 36 bp, synthetic construct
  misc_feature 1..36: note = Description of Artificial Sequence: Syntheticoligonucleotide
  source 1..36: mol_type = other DNA
attgtgttgggatacacttttataggtatttacaac

SEQ ID NO: 84 - DNA, 37 bp, synthetic construct
  misc_feature 1..37: note = Description of Artificial Sequence: Syntheticoligonucleotide
  source 1..37: mol_type = other DNA
tattgttgaatacctttcttataaaggtaattacaac

SEQ ID NO: 85 - DNA, 36 bp, synthetic construct
  misc_feature 1..36: note = Description of Artificial Sequence: Syntheticoligonucleotide
  source 1..36: mol_type = other DNA
tgttgtaaatggctttttatgggcaacgaacaactc

SEQ ID NO: 86 - DNA, 36 bp, synthetic construct
  misc_feature 1..36: note = Description of Artificial Sequence: Syntheticoligonucleotide
  source 1..36: mol_type = other DNA
attgttgaatgtattcttttttaggacagatacaac

SEQ ID NO: 87 - DNA, 37 bp, synthetic construct
  misc_feature 1..37: note = Description of Artificial Sequence: Syntheticoligonucleotide
  source 1..37: mol_type = other DNA
attgttgaatggtatcttttatagactgattacaact

SEQ ID NO: 88 - DNA, 36 bp, synthetic construct
  misc_feature 1..36: note = Description of Artificial Sequence: Syntheticoligonucleotide
  source 1..36: mol_type = other DNA
attgttggataataggttttttatcttaattacaac

SEQ ID NO: 89 - DNA, 36 bp, synthetic construct
  misc_feature 1..36: note = Description of Artificial Sequence: Syntheticoligonucleotide
  source 1..36: mol_type = other DNA
actgttgaatagttgattttatatcctatttacaac

SEQ ID NO: 90 - DNA, 36 bp, synthetic construct
  misc_feature 1..36: note = Description of Artificial Sequence: Syntheticoligonucleotide
  source 1..36: mol_type = other DNA
attgttgtagatacctttttgtaaggattgaacaac

SEQ ID NO: 91 - DNA, 644 bp, unidentified
  misc_feature 1..644: note = Description of Unknown:mammals-digestive system-rumen-bos taurus sequence
  source 1..644: mol_type = unassigned DNA
tatatcgtggccgaatatgttaacgcggacgacgtccgtcttgtgaagtttcaggacgaggatttcgacaggcttcttgacaaggttagagaatggaacaagaaacatcttgttgttggaaatcggaacttcgaagaaaaatttgcgtaatccaaaaattttccgtatatttgcggcgtgaaattaaaaatatgtttaactaaaaacaaagattatggcacacaagaatcctgatggggagaacaccatcaacaaaacttttattttcaaagtgaaatgcgagaagaatgatattatatcgttctggaaacccgcagctgaagagtattgcaactattacaacaaacttagcgaatggattggcaaagatatgtataacacgccgtcatggaacatccggcaagagttcaagaagaatttaagtgttagaaccataaacacgtttcgtgagcttggcaatgtgaaatacggcaaaatcaacaatgaagggctttttgtcgaagacgatgtgtaaacattaagatttccatacgacaggattcaaaaaaacgttctttgaaatattggattggtggcaagaggctgttttttttaggctaaaaagttgtgtaaatagcagaaacacagaacataacataaaatct

SEQ ID NO: 92 - DNA, 264 bp, unidentified
  misc_feature 1..264: note = Description of Unknown:mammals-digestive system-rumen-bos taurus sequence
  source 1..264: mol_type = unassigned DNA
aactgctacaattctgccgagtttatgattcagacaaaattcaaaaaaagacttccgcaagcaaccgtttttggtgaattgaacagaaacgggtatgttaaagtattgacccaagaagaatatgacgaactcacaaaatcagcaaaataatttattactgattgaaaaataaagcgttctttgacatattgtataacaaacaagcatttttgtaagagataacccatttcattttattgatatacaatgaaatgaaaagaatat

SEQ ID NO: 93 - DNA, 614 bp, unidentified
  misc_feature 1..614: note = Description of Unknown:bovine gut metagenome sequence
  source 1..614: mol_type = unassigned DNA
gataaatttgcccgtaatgttatcgggttcaagtcatatcacgaactgcttgataatgctatcataaaagaaaaattacaacgggaatttggttatgaagatgctccgaaaacgtggttgttcggacaacaaaaaaatgaatgtttctaatgtattaaaacaataattcaattacaattttaagattatggcacaacacaaatcaaacaacgaagaatcagcaatcaacaagactttcattttcaaggcaaaatgcgataagaacgatgtcatatcgttatgggaaccagcggcaaaggaatactgcgactattataacaaagtgagcaagtggattaaaactatgtataacatacccgcatataacattaagtccaatttcaagaaaaatttgagcgccaaaacaattcaaacttttagagaacttggacactaccgtgacggaaaaataaatgaggatggtatgtttgttgaaaacttggaataattctgtatataccaattagaattgaaaaaaaaacgctctttgacatattgttttctacataaaaacaagattttacacaacgcaatacatcataaagtgttgcgttataacaaataacaaaaattct

SEQ ID NO: 94 - DNA, 1041 bp, unidentified
  misc_feature 1..1041: note = Description of Unknown:mammals-digestive system-cattle and sheep rumen sequence
  source 1..1041: mol_type = unassigned DNA
tttattcaatgcgaaccagaggtcttgacgcatgaatctggctatacatatcgttatgcgaccgacgaagagaaaatattgattaaaagatgcaaatattgaataggcaattttaaattgtgaaaaaaaaaatgattgaatataagtttacgtttgaactggatggacatctatcggcgtacgattttgttacgttgcaagaacggtttgaaagggaattgaatccttattttgatgatgggagcatatctggtactctttcttatgcaaatgatgattaatatgcaaataatatggcacatgtaagaacaaaaaatgaaggaaacatggcaaaaacatattcttttaaggtcagagaaacaaaccttaaaaaggatgtgatgattgaatataacgaatattataacaggttatccgattggatatgtggcaatttaaccaaaatctcggaaaatgaagaatggaggaatgccttatgcaaaccaacagaaaacatgtacaacgaaccgatttacgttcccttggttaaatcacagaacggaatgttcaaggcaattaaaaaattgggcgcaacgaagatatggcaagaatagaaagaccgatttttaaatctgaaatcacttctaacgaattgtatactaaagaaatataaagaatatacatcttttatgacattatgatattgttgtatgcatcatttcacatggtaataacaacgaagagaaacaccgagcgacccacaaacctattgtcgtacgcatcatttcacatgataataacaacgaatattcctgcaagcatgatttaacaatttttaagaacctggtggtttctccgttgggttctttttagtatctttgccttgttgaaacaaataaaacaaattgaattatgatttataaaggcaaagaaatagacgaaagttaccacatcaataaatgggaagatgaagagatttactctggtccaacccattatgaatcattcgaagccgatgaaataaaagagttctacctcaaggcacttgcaaaggaaaaggaa

SEQ ID NO: 95 - DNA, 1545 bp, unidentified
  misc_feature 1..1545: note = Description of Unknown:gut metagenome sequence
  source 1..1545: mol_type = unassigned DNA
gtgcgcatatacactcaattcgccgatgaccgtgtgtacgcgaaggattgtatcgacggattctttagtataagacaagataccgaaatgcgcctcgtgtataaaaatgagatagcacgcgggcttgagtgtatcaatattgtaagatagtagttttctgttattttacatattgatgtgttttggcatggtttttgttaaaatataatctagcagtattgagactgcggagtaacgtgtctaactgtttcattataagcagtaaagactaatatttttatatcttaaacttatttttattatggctggtcacagcaaaatcaaagaaaatcacattatgaaggcgtttcttatgaaagtaaaagaaacgcgaaaaaaacagtggcaatcaaattttattagaagtgagattgctaagtttacaaattattacaatgggctgtcaaagttccttcttggaagcccgactggagggacatatgacactgcatattttgatacaaagattcaaggctccaagggggtatatgataagattaaagaaaacggagaaacttatattgcagtattaagtgatgacgttattacggcagaggtgtaaaatcctctgccaacatcgcaagtaactcattgaaaattagttaaatgcgaatgccaacaaaagtgaacgaactgacttgtaaagcaggatgttgttatatctttttgtagataataagcaacaagatacaatcaatcgcgagtttatactgaaatgttgttacactgtttttgtaagtgttaaacaaccttgcacaaatgtcatctaccagtacaatagatgttgttatactgttttgtaggtattaaacaaccattgcgcagactgacagagtaacctttcctgatatgttgttacacatttttgtaagtgttaaacaactgacgcattgatattgccttgtctattaagaatgttgttatgctctttttattggtataaacaaccgagcaactggtactcaaattttaaatactgtcgcgctatgttatgtacatcgaacagctaccactcaatggctttgtttgcaaccgtgattaattcaatcgcggttgcatttgttttatgatgtgtttttgtatatattatgtatatatggaaaaggaaaacagggtatcggagttatggagcaagttctctgatattgacttgcgccgaagccaaatgacatatatgccaataagaggtagtaaaagatacggcagaagaataaaacgtagtgacatcgagtacgagtacagatatctgtatagagcaaacaaacattggtaatatgaccgtagctaaattatcaagtaatcataagccagcgtgccttggacgaatctcagctttaaacaccccgattagatttgagtgtcgggctggtaatagtataaggcctggcaacatagagtatagctataaaagatggaaaacgtcgtaatttcaactatgcacaacccgcatacgctggcttattaccaaggtaagctggctcctatgcatttcagacaagatacagg

SEQ ID NO: 96 - DNA, 1380 bp, unidentified
  misc_feature 1..1380: note = Description of Unknown:mammals-digestive system-cattle and sheep rumen sequence
  source 1..1380: mol_type = unassigned DNA
agcctgtatacagggacaaggttaagtacaacaccaaggctgaggcaaagaagagggctgatgatatgaacaaacagaatagggtcatacaccagctgtctgtttatttgtgtcctaaatgtcataagtggcatataggtaggagcagtgtggagagtgtgcgcagggaagggtactttagtcagatttgaaattaattgttatatggcgcatagaaataaaaacctagcagaaaactgcattaacaaaacattcagttttaaagtcaaagccgaaaaagaggagataaattcaaaatggattccagccattaaagaatatactgcttattataacaggataagtgactggataaacctgtattcacagcctacttatgatattaaggaagtttataagaaaaacgctggttgcaaagtgataaacgacttcattaaaaacggtaacgccgttatatgttgtatcgaaaataacaaactaattgagacaaatggaagacaatagttcaaattttaaatgtaaaacagtcattaatgtattaatatataatacatagcaaaaatccagatgttgaatacatttcttttaagtgtacttacaacgcggtggcattgctaaaatatagtcctgtggatgttgaatacatttcttttaagtgtacttacaaccaacgctgtacacattgctaatggatgatgacgatatagaggtgttgaactaccttaatgaaaactacaccaatgaaaacattgagtatatacgcggttggtggatggatgacgacgataaactccagacacttgacaggtttttgaaaaatttttcaatatagacctgtcactgttgcggctataagaagaccgatttgacactgaaagaccgatactgggtttgccccgaatgcggtgcaaaactagaccgcgataccaatgcaggaataaacattaagaatgagacaattagactgataaacaaagaataatgagaactataatagggaggtgtacccccgaatttaagccagtggagaaccatacaaacctatcatataggggttcaatgaatctggaatttctgacaaaaacagggtttaacagccagtgtaccaatgactaacacaggacatataaagacaaatctaacaataaaaaaaaatattgaccaattctgcagaaaaaacaggttggtttcggttatgttggtgaataaagacagttagattaattttatatggaaatgaaaatagagacaaaagacgagaacatctacgtattcatctatgccaagtccgcctacttcggcaatacatttgaatatggcggcacattttccgtcggcaaggacgacaactggaacgatgtgagaggccacgttaccgaa

SEQ ID NO: 97 - DNA, 853 bp, unidentified
  misc_feature 1..853: note = Description of Unknown:mammals-digestive system-rumen-ovis aries sequence
  source 1..853: mol_type = unassigned DNA
gacaacatcctggtcaagaccgaggttaacagaaggtactgccgccttatgaccgacgagaacggagtgtggctcctgaggaaaaacgacaaacatccaacatattttatctaccagaacggaacactctatcaatatgaggaagattgattagttgatgttttcataataattttatctggaatttgaaaagattccagattttttttttatttcgactgtacaaaaaacaggttccgttgcgttatataggtgtaaattaaaaattcagtcaaacaaaaattggaataaaatatggctaacaagagaacagacacaacaatcaaccttaacaaaaccgttataatgttaacgaacatgctgccagaagtacgggcaatgtttcaggcgggaatacgccaggctcaagtttatgcagacttggtgaacaagtggatatgttcacaggaaatgagagaggttatgtgtctccatccgtcaaaaaaggacggggtgtacgaccaaccgttcctgaaagctacaaccaaatacccagccacggtagctggtatcctgcttaagatgggaaaaacaaccaattggggtgagaaataatacccacccgccccatttttttacactgattagttctttgacttattgatttatattggtttacacaaattatcgacacaataaataaaaaaaattgtatattagtagtatgatgacagaagaaacacggaagacaatagagagcgtcatagtggttctcggcatagcaatcatgctggcagccgccgtccgaataatgacgcagaacaaagcaattgtgaaatatgatgaacaggttgaaaccatgcaaacttgcata

SEQ ID NO: 98 - DNA, 795 bp, unidentified
  misc_feature 1..795: note = Description of Unknown:gut metagenome sequence
  source 1..795: mol_type = unassigned DNA
atggaagttgtacgtggtggaaatcaatgggaggtttatgacaattacgatgagactatgaaagcatcaaaaaatgtaaggtctgtattgggacttccggaagtaaaatatccacctgaggattttaggacatataatttctaataaaaatgaacggaaaaatttccgttcattttttttttgtttattggtgaaaaaatagtatctttgtaaaaaataaatgttaaaatattttttatgggaaatactacaaaaaaaggaaatttgacgaagacttatttattcaaagccaatctttcagaacaagactttaaattatggaggtctattgttgaagagtatcaaagatataaggaagtgttgagtaaatgggtatgtgaccatcttagaaatgcaatgtgtacgaacccgaaaagtgagactggatattctgtaccgttcttgacttcaagaatcaagaaacagaacattatggttgtagaattgaaaaaaatgggcatggttgaagtcttgaatgaaaaatcaacagaaatttaagaaaaaaatatttatataatgtactgaaaataagtaaataataaatattgtgtaaaaaacttgatattttttttttgttatctttataatataaaataaaatgtaaatatgaaaaatctgttaaaactcaaagaacaaatcaaggattacaaacatcttcagtttgtgttggagaaagaagatgaatctgaactccattatagatgtatgactgaagatttttcgttcaaggtatctgaagaaaaagacggaacactt

SEQ ID NO: 99 - DNA, 420 bp, unidentified
  misc_feature 1..420: note = Description of Unknown:bovine gut metagenome sequence
  source 1..420: mol_type = unassigned DNA
ttataaacatctaaaaagaaagacttatgacaacaaaacaagttaaatcaatcgttttaaaagtaaaaaacactaatgaatgccctattacaaaagatgtaataaatgaatataaaaaatattataatatatgtagtgaatggattaaagataatctaacaagtattactattggaaacgaaaatttacgaaaattattttgtggtaaacttaaagtaagtggatataatacaccaatattagacgcaacaaaaaaaggtcaatttaatatattggcagaattaaaaaaacagaataaaattaaaatatttgaaatagaaaaataagtcttatgattacaaaaataatagatttcaaacattttttttaattctattttattgactaattcattgaaatataaataattacaaataaccc

SEQ ID NO: 100 - DNA, 1058 bp, unidentified
  misc_feature 1..1058: note = Description of Unknown:mammals-digestive system-rumen-ovis aries sequence
  source 1..1058: mol_type = unassigned DNA
gatagatatagtattgcagcatttctggcttgcgaatcatcagcaatgcaaaaatgtgactattggaacaatgatgatgcccaagattacataagaaactacaaagaggcttatagtaatgcagtaagacttgcgttttttaatgattaagcaacacgcttaacattgtcaaatgtaacgacattaagtgcgtgtttcataagggcagcgaacctttcgccgcccttctttttttgttgctgtaacggaattatgtttacttttgtgccatcaagtatatagttcccttaataaattgtatattaattaaaagtttggcacaatatttgatgcgtacaaattaaaataaaaacattttgaattttaaaatttaatttgtaattttaaataagaaagttttatttaactaaaataaaaaaaatgaataaatcttatgtttttaagtcgaatgtggctattgatgacattatgtctttatttgaaccggcaattgaagagtacataaactattacaatagaaccagcgatttcatttgtgataatcttacatcaatgaaaatcggagatttgttgcttctaacaatgtgtactaagacaaaagaaaataatagatacggtaaccccctctataatatcaaagatacttttaaaaagaaaataccatcttcaatacttaatatattcaaaaaaaaggatatgtatcaaataatatgtgattaattatgcctttttttaataaaaaattgttaaataatactttgtttattaataaattataaatatcacagtaaactattagggatttgtaaaatttatggaaattatatacatgatggcactaagatttggttattaagaaatttttctgtataagtataataacctatttataattataattgaataaaatgtataatatggaaaacacaggcttttatacagtttcaaatattgaaacttctcataagccaaccgaaaattctaatgacgaaattcttaggattttcaataaaagaaggccttattgcccttcagactttaagaagcaacattttatt

SEQ ID NO: 101 - DNA, 554 bp, unidentified
  misc_feature 1..554: note = Description of Unknown:mammals-digestive system-rumen-ovis aries sequence
  source 1..554: mol_type = unassigned DNA
aggctcaacctcctcaacccgatttatcttgagatcgccaagtacggacacttcgggaggaagagctatgtgaaggacggcatcaagtacttcccgtgggaggatttggatttggttgaagacatcagaaaaattttcgaaatggaatagagggaaccggaattttttccggtttttctttgtcctttcgaaaataaatagtatctttgtaaaaaaacaacagattatgtacaatagtaagaagaagggggagggtgacattcagaagtcgttcaagttcaaggtcaaaacggacaaggagacggtcgaattattcagaaaggccgcagtcgaatactcggaatactacaagaggctgacaacattcctctgtgagatgtataacagaccagcgtttgacttgaaggagtgctacaagaaaaattccaatgtaagtgtcttcaacacattgaagaaaactctcggtgcaatatatggaaagctcgatgaaaacggaaattttattgagaatgaatgtaataagtaactggaataaaagaaattagacagagtaa

SEQ ID NO: 102 - DNA, 1039 bp, unidentified
  misc_feature 1..1039: note = Description of Unknown:mammals-digestive system-rumen-bos taurus sequence
  source 1..1039: mol_type = unassigned DNA
ttgtattggttgctgtatggcgacggaagtgacatatatgatgacgggtggtttgactgtgttcataattttgcccgtaatgttatcgggtttcagtcatatcacgaactgcttgataatgctattataaaagaaaaattacaacggtaatttggttatgaagatgctccgaaaacgtggttgttcggacaacaaaaaaatgaatgtttctaatgtattaaaacaataattcaattacaattttaagattatggcacaacacaaatcaaacaacgaagaatcagcaatcaacaagactttcattttcaaggcaaaatgcgagaagaacgatgtcatatcgttatgggaaccagcagcaaaggaatacggcgactattataacaaagtgagcaagtggattaaaactatgtataacatacccgcatataacattaagtccaatttcaagaaaaatttgagcgccaaaacaattcaaacttttagagaacttggacactaccgtgacggaaaaataaatgaggatggtatgtttgttgaaattttggaataattctgtatataccaattagaattgaaaaaaaaacgctctttgacatattgttttctacataaaaacaagattttacacaacgcaatacatcataaagtgttgcgttataacaaataacaaaaattctggacgggaaaggaagatgtcagacgtttttattgttggaatactcgttttttacggtatttacaactgccccgtagcggaatcaaaataccaccgcattgttggagtacaagttttacacggtattcacagtacgaacaccgaatgaactgaaaaaaataaacccgaccttgcaaccgtagatataaataaagcaatacaaaatttgaaactatggcacacattaaaaaaattgacgaaatggcaagtcaaactgtttcactccgttctgacgcattgttcaaaaaagcgtttgaggaatttgaaaaggagttgaaagaagttctcaaatcgcacaacaatatcatttattgtggaggtgat

SEQ ID NO: 103 - DNA, 1252 bp, unidentified
  misc_feature 1..1252: note = Description of Unknown:mammals-digestive system-rumen-bos taurus sequence
  source 1..1252: mol_type = unassigned DNA
ctcatcaaattgtacaagtcgttgacggacactgaatttgacaagaagaaaatcatcaatgatgtctacgacggcacttttgagataatcctcaaatacccaaagaagaagaacgggacattcgtgttctggaaacattacaagaagtaacacaatgatacacagtatgttgtaagaaataagatttaggctttaattttaatatatgaaaatatggcacacaaaggagaaaaggaaggctaccaaatcaagacactgaagttcaaggtacgctcgcatgacatcgggaaatcactttatgatattgtcaacgaatacaccaactactataacaaagtaagcaaatggatatgtgacaaccttggttacaacgagccattctacaagtcaagggtgaaaagcgccgcctccatgatgtcaggattgaaaaaactgggcgccaccatgccattgacggatgaaaatgccattttttcaacaccaaaaccgaagaaaaacattggaaaacaataatttacacaaagtctacggcgggaatcgtgataaaaatgaacgagattgttgggatataccttttataggattttcacaacatctgagttgtttgatgttaaaaactttaactaataaggcaagaagtcccattccttcaggtgggggtagttcatttgttgggatactcgtttcacacggtattcacaacttccaaccaaccattaaaaaaccttcaaatattgttggagtacccgttttatacggtgcaaagcctccccgacgatttcaagttcctgtacgaagatgtcaattttggatagcaactgttaccaataaacatattcaaaagtaatcaaatatattcaaaaacaactcgtataaatatataaagttcgtgatatttattataaagaagccgaaggagagagcggtttccgaacaataaagatatacagaggttttattcttgacggcactctctcctttagccgcaagtttaattcctcttttttattgcactatggtcatcgacagcaaatataccaagacattcaagtcaaacggactgacccatcagaaatatgacgagttgctctcgtttgcttctatgctgcgtgaccataagaacaccatctccgaatatgtcaatgccaaccttgaacactacctcgaatactcaaaactcgacttccttaaggaaatgcgtgcgaggtacaaggatgtcgttccgagttcgtttgacgctcaactctacacg

SEQ ID NO: 104 - DNA, 1131 bp, unidentified
  misc_feature 1..1131: note = Description of Unknown:pig gut metagenome sequence
  source 1..1131: mol_type = unassigned DNA
agaatctgtcctatatgtgggaaacattgcgaatatgaggaaatggagggcgaccacattgttccatggtcaaagggcggtaaaaccgatataggcaacctccaaatgctatgcaagaagtgcaatcacgaaaagtccaatagatattagtggcgtaatcaaaaatttgtttgtgttgaggaaaagcagtgaaaaaaaacattgtttttcctcaatttttatttgcataattcaaataattttttattttataggataatagagctaacaagcattaacaattattaaaacgatttatattgaaaataaattttgtgggaatatttatttttactacctttgcatcgtaatacaattaaacaaatttttgattatggcacacaaaaagaacataggagcagagatagtaaaaacttactcttttaaggtgaagaataccaatggtatcacaatggaaaaattaatggccgccattgatgagtatcagtcgtactataacctttgcagtgattggatatgcaagggtcttgacgaaataatgaggaatacttttctgaaaaaagcaaatagcaataaatcattgtataatcagccaatctacgatacgggtatcaagaaaaccgcaggtgtgtttcctagaatgaaaaaattaaagaaatataaagttatctgaaataaaatatgtatttttctttgtggaaatacctattaatagactgatttctaataagttataagaaatactgtatgtagtaaataagatatcatatttttgcggagaggcacatggagtatgctatagggtttttgctaccgagcagaaagcaaaagaaaaaatgcagggatgatatcatttcattcttgcattttgcttatacatattcaatcaagtatcattttctgtttttactattatcctataaaataaaattttcctcaacatttccaaatttaatttgcaataattttttttgataaaaagtgcaaataaattttatagattcaaaacttttgattaactttgtaacaagaaaaacattaaggattatgggttacacatattttagggttactgatgaaagggcaagggatgttatgccaaaggcggctgaaatcataaaggatattttc

SEQ ID NO: 105 - DNA, 3677 bp, unidentified
  misc_feature 1..3677: note = Description of Unknown:mammals-digestive system-rumen-bos taurus sequence
  source 1..3677: mol_type = unassigned DNA
cttcacctcgtacagccgacaataagtttcgcttggactgaacttatgtgcgcctgcgcattcatagcgggtggcgtatcaggctatctcatcaagggcaagatgccaaacgacgggaacaagtaccagtcggtagagggaaaggaataggacaaaaaaaaacacatcacccccagcgcatcgggcgcggaggtcgggtgtgcatataacggtgtctgtggcgcaactggtagcgcagtggattgtggttccaaaggttgcgagttcgagcctcgccagacacccattatcacacggaagcattggatggaagtgcaagtacctactgggaacttcctgaaagcgcaagcaaagtcgaggtctaacggtacttatgaccgaggtaatggcggggcgttggttcgagtccaacacaatgtttccatttacacggagagttgcaggagtggtaactggtcagattgctaatctgaagcccacctcgttgtggcaggggtccgaatcccttactctccgccaagcaacatacccgcagagtagtcgcgtatattctgtcggtgtggtcagaaagaagtgaatgtgatgcgaacgcgcgaaaccatcgcatttagagtccgaatctcctctgcggtagccagtccgcatagtttaatcaggttaaaacattctgacgcttttttaaatcgcgggagtagttcagtggtagaacatcggcttcccaagccgagggtcgcgggttcgagtcccgtttcccgctcaacacataggctgtggacaaggtgggcgaaagtattttttccatagttttacaccaacgcccgccttttcctaaacgcattggagagatagaggacttgccttctaaacaagcagtacgggggaacttgcatccgacctccgtttcaatgcggtagaactccgctcccgtgacagcgacgaatgatgcaatagcggttcacgagatacctcaagaaacttcatttttcaaaagccacaatagttcaactggtagaacggcggtatcgtaaaccgcaggttgctggttcaattcctgcttgtggctcaacaatttcgggggcttgcaacgctgccactgcgggtggaagccagcgacaagaacttgtgtgaagccgaaacgcagtccttcgggagaggggcgaaggggcaagcgagatgtgtcccacttttttaaagtaacaggctttaataaatatttatcattcccgaaaggctgtgcggaacagcctctcggcttttacggggatttagttcagttggtagaacatctggttcgcaatcagaaggtcgcgggttcgactcccgcaatctccacaaatataaatatagtattgccctgtggtgcaatcggtaacacaccagattctgaatctggaatttcgagttcgagcctcggtggggcaacacaataggcagccgtactgccgaatacaagcctgtggagaacccaaccgtggatgaccgttgcctatgcaacctaaaaagcggtggttctgtgaagcaggaagcggaaatacaatattccgcatacggtggtggtgtaatcggtaacataacaatatccgaaaagtttaaaccatacacccgacgattatttttattcattgttagcgaccgccgtgaggcggacgcaggctggcggtcggataatgacgcataatggcggttgtgaaagccgacggaaagcactacatcgttaagtgccagccaccataataggcagccgtactgccgaatttaagcctgtggagaacccaaccgtggatgaccgttgcgtaagcaacctaaaaagcgatggttctgcgaagcaggaaggaaatgcccaatttattaggtttttccatacggtatgacagcctctaactgtagcgcattacaaaacaaacgctaccattacataaatggtcagaggcataacgccgagcgcaggtatggtatgcgttcaagtcgcagtcacggaagccccagataaaaatgggaggtgcttgcggtcaagcgagtggtcagcgggcttgcactcggtgtggcaacaatggtcgtttccgaacttacgaccattcaaaaagataaggtagtggcttgtgagtgaaaagaaactctcgatacgctcctttcgtctaacggtcaggacgcgagattctcaatctcgtaatgcgggttcgattcccgcagggagtacaatggcgaacacacgacaatccaaactgaaggggaactggaaaaccctcgctccgagataacatcagcgcagagaggttggtgaggcaaccgtaaaagtaatcctgtgtgcaagcaagaaggaagttcgggttcaagtcccgatgaggattattgttgaagagggatatgattcaaccatagcacttatggtgctgtgcaagggttataggcagccgtactgccgaatacaagcctgtggagaacccaacagtggatgaccgttgcctatgcaacctaaaaagcggtggttctgcgaagcaggaaggaaatgcccaatttattaggtttttccatacggtatcactactcgcggtggatgtggaaataaccgcgatttggtcagttggtgaagttggttatcatacctgcctgtcacgcaggtgttcacgagttcgagcctcgtactgaccgcagacaaagacaaagaacgagaggacttgtatgacttgcaaatgtcacggactcaaacaagaaaagtttataggctattagaggatgactgtttctttaatttgttttcttgtactgaaggtcatcactgccgtgccaccaagccgtgcaagtccaaatggtgcgttagttcagttggttagaatgccagcctgtcacgctggaggtcgcgggttcgattcccgcacgcaccgcaataatctggatataggcaaattacacatatcatatgtcgccccgcgtaatcatagacgacactgcggacgacagcggcgagaatgtcgaaaggctcgacagcataatgacattcgacatcaccgacaccccgatatacgaaggcggggaggaacttgagataaacgcaaaattcaacagatagaaataattaaaacaaacggcaatggcacacagaaaaaagaaagatgacgaagcaacgctatcgtacaagttcaaggtaaaggtcatagagggcgacctgacggcagacgacataacgaagtgtatcgcggaaaacgcggagcagggcaaccatttctccgagttcatacacgatgagaatttcaggaagaccttcacatccgagatcagcgcggacaagttcggatggggcaagccgatgttcagcccgaccaccagaagtcaggacgaagtgttctccgcgataaagaaaatcggggcgataaccgtgctggaagattagcgcatattattctcatatctaaaattggaaggacacctgcggacgcgggtgtccttttttcttaaaatgccaatttataaataatatataacttatatttattgtactttttttgtttaactaaaacacatagacaaatatggaaattcaacagattaggtttataaacccagttgattttgaagaaacaatcgttaatgtacccacggagaagggcgaaagattcctgagaacaaaaatctatacggacgagtattcacccgaaacattcataaaactctgcgagaag

SEQ ID NO: 106 - DNA, 831 bp, unidentified
  misc_feature 1..831: note = Description of Unknown:mammals-digestive system-rumen-ovis aries sequence
  source 1..831: mol_type = unassigned DNA
tggcgattattcttacggcaaaggccttatccatgcatacataaatcgagacatcaaaagtttttgcttgccaaacactttaatatgtgaatgccatataccaaaacataccagatatattactgattactcaggtacaaatatagccgcaaagaaaatcatcatcgacaaagttgtctgggagaaggtatgtataaaaacataatggtattaggggagaaattttcttggacggaatgaatataatttcataccaacaccgtgcattgattaaactaaattaaattatcaagcataaaaagtttggcacggtttttgatatagtaaatttgtatttaaaatttttaatatggcacacaaaactaaagaatcagaaaaattagtaaagtctttcaaattaaaagtagacattagcaattgcgaaattgaaaagaaatggattccttcttttgaagaatacacaaattattataatggagtaagtaattggatttgtgaactattagaaaaagtttgcctgaaaagaaaaaaatttggaaaggcttcttattcagtaccatattggaacgttaaagacgcatttaagaaaaacgttagctcaaacatgattgctacaattaaaaaaatgaatatggtaaaggttttttaatgcgtgattatggcgttttttaaacataaaatcatttataatatattgaaaaacattttattatataaaatatgcatcttagtgaaaccgtgttttcgtatagattgctggattatacttttttataggataattacagctcgaacttctttgatggcattaataagatattgttggattat

SEQ ID NO: 107 - DNA, 634 bp, unidentified
  misc_feature 1..634: note = Description of Unknown:mammals-digestive system-rumen-ovis aries sequence
  source 1..634: mol_type = unassigned DNA
atcatggctgaaagcgtccgcctgattgcagagcaaaccgcaagcccgaaggttgtcatcaagagccgttacgctctggtcgacgcaggtttctatcctgagttgaactatgtgaccttcttcgtgaacactccagatcaactggtttaatcactgcgggtagcaagcgattgactacggaaggccgattcgatagagtcggtcttcttttttttttgtatattttctttttttggtttggaaatgttccgtatatttgcagcactaaaactaaccaatatgggacatgtacgtttgcaaaaaagagagggagaggtttataagacctacaaacttaaagtaaagagcttttctggcaatgtagacattaaagctggtatcgttgaatacgatatcgccgaaacaattgattggagaagtacgctttgtttcaagacatggaatacgtatggttctcctcaatgggactcgaagatcaagaaccagaaaacgatgatcgatcgactggattcgttgggtgcaatagaattgaaaaactggtgattttgatcatggttttgaaacaaaatattgatttttcgttctttgacatgcttgttaaaaattgagtatcagtttaatataaagaatatat

SEQ ID NO: 108 - DNA, 1154 bp, unidentified
  misc_feature 1..1154: note = Description of Unknown:human gut metagenome sequence
  source 1..1154: mol_type = unassigned DNA
ggaaacaattataacgatgcctacaaaacgttaattcaaatgagagacaaaggaattttaacgcaggaagttgtaaatgtatttaccctattgaaagggcggtatattaaagaaaaagaatacggaacacaatataatactatcaattaaattttttggtagtttcatttggaattgccaattatttttttattttatagaataatagagccaacaagcattagcaattattaaatcgatttatattgaaaataaattttgtgggaatatttatttttactatctttgcatcgtaagataattacaaaacattaacaacatttattaaacaattaaacaaattttaattatggcgcacaaaaagaacgtaggagcagagatagtaaaaacttactcttttaaggtaaagaataccaatggtatcacaatggaaaaattgatgaacgccattgacgagtttcagtcatactataacctttgtagcgattggatatgcaagggtcttgacgaaacaatgaggaacacttttctgaaaaaagcaaatagcaataaatcattgtataatcagccaatctacgatacgggtatcaagaagaccgcaggtgtgttttccagaatgaaaaaattaaagagatatgaaattatctaaaataaaatatgaatttttctttgcggaaataccttttaatagattgatttctaataagttataagaaatacaatagatactgaaggaaaatcaaagtgtaatcaaaaatttgtttgtgttgaggaagcagtgaagaaatttcattgtttcctcaatttttatttgcataatccaaaaagttttttattttataggataataagactaacaaatctcaacgactattaaaacgatttatataaaaaaagttttgcagttccaatcttttttgctatctttgcagtgttgaaagacaacaaagatttaagtttaacaaacaaatactttttattacatattttaatttttttgtattatgacaatagaagaaaaagcaagggaagaatacccttatataaccccatctgatgggtatgaatgccatgattataatgaagccgctaaagacggttttattgagggggcaaaatggatgcttgaaaaagccgctgaatggtttaagaat

SEQ ID NO: 109 - DNA, 1048 bp, unidentified
  misc_feature 1..1048: note = Description of Unknown:mammals-digestive system-rumen-ovis aries sequence
  source 1..1048: mol_type = unassigned DNA
atatgggcaaagcgtgataaaattgaaaacaaatatgtcaaagaaccattaaaacgagtcaatgaagatatgtggtggatgtactatgtttatgaatggaatgtgttttatgtgcttgaagaaaatgtccatccatatatgaaaaaataaattttaccacacatattattattcgtgtcatgccgatgaggtttggcacgatttttgtttatatggagagacataatgtcagtcaatacatgacaacttgtcacaataactgacattaaaagtttggcacaatatttgcttataagaaaaacgaacaagtaaaattaaaattttatagattatggcacacaaaacaaacaacggagaaaacaccatcaacaaaactttcatcttcaaagcaaaatgcgagaagaacgatattatatcgttatggaaacccgcagcagaagagtattgcaactattataacaaattgagcaaatggattggtaaaacaatgtacggcattcctgcatataacatcaaaagaggttttaagaagaatttaagtgccaaaactataaacacatttagaaaacttggacactatcgtgatggaaaaataaatgaggatggcatgtttgttgaaactttggcatagaatttgcatataccaattagaattgaaaaaatcgctctttgacacactgaaacatacaaaaacaccacaattttttaatccttttctatttgtattttattgaaataaaatgtattatagtaatatatctgctaaggtcatatttttcattgttctcaaattgttggataatgttttgtgtgtttcatttttgtcattgtgtcaccttaactgacaaggtggcacattttttatgtcaatatgtcagttgaggttttggcataatttttgtataatggtaaatggataagaattgaaattacaatgacaacaaaacaaaggttaataaagagaataaacaaggcattcggatttgaattaacggatgcaacaccttgtttccaccatcaaggtagaagatggggaagcggtggtttc

SEQ ID NO: 110 - DNA, 968 bp, unidentified
  misc_feature 1..968: note = Description of Unknown:mammals-digestive system-rumen-ovis aries sequence
  source 1..968: mol_type = unassigned DNA
gaaggcggcgcgtttgaaatcgctaacgtaattgaaaatgccaagaagcagaatctcggggagggtggatacaaggaattgtgcaatgatttcctgaaacatgcgagggaaacgtttttcagtgggaaatacgaacaccattcttggtagtggatttgttattttggtaaatataattaacgcggcattgtcgtcagtgaatataatattgcatttcgacagtattttataagtattttgacttataaacagtatttataagttattcggcttataggttaattagcctatagatgttgtttataggttggatgacctatagtgccaagttttgaagaaatcgttatagtcatcgttctgccctattagatattccgtatttctttaagactgttataatacaaatatactacaaatcatgcaatttttgatttttaacaaaaattaagaaatagggtattattgtgtattgttttttgttatatatttgtcctgttaggttaaatcaccgcgcctgatgacgaagtcggtggtagaattagactaatattaaatatgtctcatgaatttaacaagaataaaggtgagaatgagattagcaagacctttattttcaaaacaaaatgcgggaagaatgatattacatcattatgggttcccgcgatggaggagtattgcacgtattacaacagggtaagcaaatgggggaaaggtatgtacaacaagccgtcatatgacatacggaagaaattcaagaagaacttgagtgcggctactttgaaaactttcattaagttgggaaacacggtgaaagggatgattgtcaacggacagtttgttgaaatggaatcataggttgacagaaacggaaaatcggtttgtttgttagaagaatatttgttgaaattcatttttcttttgctaacgtatatacaaataactgtaatagaatatcttatataagatat

SEQ ID NO: 111 - DNA, 1542 bp, unidentified
  misc_feature 1..1542: note = Description of Unknown:mammals-digestive system-fecal sequence
  source 1..1542: mol_type = unassigned DNA
acaaatgaaattatgggacaagtaaaacttaataaacctcttctgtatatcaaaatattgactatctttagacataaccttgtcaaataataaatctaaattactcttttccttttcttttttaaataatttcatattaaatattcccataatttattaatatatttttttttcattacttatttctctgttatataaatagttacataaaaaaattaaaactattttttaaaaagtcttgtgtatataaaaaaaatatagtacctttgcacccgaaatcaagatttaatcctgttttcatattatatttatcaattttatactaattaataaacttatggcaaataaaaaatttaaacttacaaaaaatgaagtcgtgaaatcattcgtactcaaagttgctaaccaaaaaaaatgtgctatcactaacgaaacacttcaagaatataaaaactattataataaggtaagtcagtggattaataacatcgtacaaaatgaaacgtggagaaatctatttactaacaaaaccaataatacatatggattacctatactaacaccttcaaaaaaaggacaatctaatatcattacacaattaatgaaaattaatgcaacacaagaacttgttgtataatataatctatttttaaatttataatactaatataattcattgataattaaataattatataaaattcctatatacaatagaaagactttccacagacatgttgtacatacatttttttaagtattaaacaacgcatacccaccaatggtacacgaaaattttcatgttgtacatactatttttaggtattaaacaactcactgttttgacgattaatataggcatgttgtacatactctttttagatattaacaacctgtaaacaataacaatatttacaacaataatccatttttgaaataatgaaaaattttctggaaaaattttttaacaagtctgtttttgaaataatgaaaaaatttctggaaaaatttttttaacaaacccatttttgattggttcattttttattggaaaattagtgtgtggaactacccacccgtatatgagcaagtgttatggggtgtaacgtggggagggttacataggggggtctttggtagggggtacataggtagggtaataatggggtctttggtagggggtacataggtagtccccatatattattataaaaagtaaaataaatgatatatgcaagagtttttgaaaatttatttttattttgctacttagactttacaaaaagtagatatatagtattttcttttcaaaatattttgtagtttggaaaaaaagcagtacctttgcacacggaaacgaaaaacaagtttaacctattaaatttttagtttatggcaataaacattttgacttattctgctatggcagaaaaatcttgggaaaattttatgcgtgaaaattgcggttacgagcgcattagtacattttatagtgatttcactattgcagaccattgtggtggtgtaaacgcaataaaagac

SEQ ID NO: 112 - DNA, 920 bp, unidentified
  misc_feature 1..920: note = Description of Unknown:mammals-digestive system-fecal sequence
  source 1..920: mol_type = unassigned DNA
gatgtgaatgaagaatttcttggtggcttgcgaagcactatgacatatcttggagcaaagagattgaaagatattccgaaatgttgcgttttctatcgtgtaaatcatcagttgaatacaatttatgagaatacaacgataggaaaataatataaattttatattattttgagaaaaagagtctaaatttgggctcttttttcgttttttatgaaaaaatatgaaaaaagtttgtaaaaaatttgtaatattgaaaaaatagtattatatttgtatcaaatttaaaaataaaatataaatatggcaaaatcaataatgaaaaaatcaattaaattcaaagtaaaaggaaatagtccaataaacgaagatattataaatgagtataaaggttattataatacctgtagtaattggattaataataatttaacaagcataactattggtgaaaatgaagactggagaaaagtgttttgtatcaaaccaaaaaaagaagattacaatacacctttattggatgctacgaaaaatggtcaatttagaatacttgacaagttgaaaaaattaaatgctactaaattattagaaatggaaaaataataaatatatacaataaatttatataattttgtctatttttaattttagttcattagataatatgttcataaattcattgacatataattataaataaatatatatgcaataaaattcgagagacatttcatcagagatgtctcttttttattttttgttatatttatattatgaatattagattggaactcataaagacaaaggataaacagaacattgcaaagcgtatagtggaaagcaatcactcatatgttccaacctggcgtagtgtaggacgaaggatagattatcttatttatttggataatgatgttgtcgga

SEQ ID NO: 113 - DNA, 1217 bp, unidentified
  misc_feature 1..1217: note = Description of Unknown:mammals-digestive system-rumen-ovis aries sequence
  source 1..1217: mol_type = unassigned DNA
gtgaactatatctacgaatcaatcgaaggaatattgacaaaaacaatgaatccaaccactttacaggatatcatccttaacggaatcacatatacaccagtggaagacaacacaacaacatgcgacggatgtgaatttaaagacacataaggccaatgtatgctaacacacctattcgataacgacatggtccaaaactgcctcaaggaaaaaaacggcgttgcagatatcatatatgtcaaaaaagaaaattaatcggaatcttgatttggattttaatattatttgttgtataattacaatagaaagaaaattttgtatattttaaaatttgtaaattaaaatttagaaaaatggcacacaaaacaaacaacggagaaaatacaatcaataaaacttttattttcaaagcaaagtgcgataataacgatattatatcgttatggaaacccgcaatggaagagtattgtacttattacaataaattaagccaatggatttgcaagacaatgtatggagtaccagcttacaacattaaaaacggtttcaaaaaaaatctgagcacaaagacaatcaatacgtttagaacgcttggccactatcgtgacggaaaaataaacgaagacggcgtattcgttgaaaacctggcataataaggagtaaaaaaatgttctttgatattctgacacaaatgaaaaaacaatcaaaaatttatttctgttttgcttgtaatttattgaaataaaatgtattatatagaaatatgtcggtggataatagtcaaatagtctgttgactgttgaatagtaagttttttactctattgacaacaggtgatgtggatggaacatacaaagtttattgttgagtaataggttttacacttttaccacaactttagtgattttatgtataaaataattaaaatcatatataaaaatttttccagaaagtagtacttattgaattaaaattatattgtgaaaaatggtttttgattttaattttatttgttgtataattgaaatgtaatttaatttagaattgtataaataaaaaacgtaaaaatgagactgccaacagaaatttatgagtcaggcacaatggttagtaagatatcggaaaaaccatttaaatcaggtttaagggttaatactgtaaagtctgtagttgaacatccacataagattgacccgaatactaataagggtgttcca

SEQ ID NO: 114 - DNA, 930 bp, unidentified
  misc_feature 1..930: note = Description of Unknown:mammals-digestive system-rumen-bos taurus sequence
  source 1..930: mol_type = unassigned DNA
gactacgactggttctcaaatgtgtacggcgccatcagggaggaacgtgagaaaatgagaagggaagaggaggaacgcaggaagaacgaacccaagacggtgaaaaccaaagaggttgacttgttcggggatgatgacctgccgttctaataaaaaaaaaaacaaacctctccgaaattgaacgtatcaacttcggagaggttatatagggtgatggaaatgttaaataaaaagtttaaaaataactatgggaaacaaagtacaaagtaatgaaacaatagttaagacttatacatttaaagtgcgtggattcataagtggtgctacccacgaaataatgaaatcagccataaaacaatatatagaagattctaacaatctatcagattggattaatgtagagaatgaaatacttaggaactctttccttaaagaagagactaaaaaatacacttataatacaccattattcactcccagacttaagtcatcggaaaaaataataacagaattgaaaaaattgggtatgactacggttatagaataaccattacacatttttttcataacaaacgttctttaacatattggaaaataagaaaatacgatattcatataaaaatccgtcccacacaaaattaatgtaatatcttagttttgttacatcaacactatataattaaaaaaataaaaaaatattttgtggattcaaaaaatcattatatatttgcgtccgaaaattaacacttatgtcaaacaaatttaaaatgtaaaagaactatgcaaacagaaacacagaatttcacaggcgagttgagagcaatcaacacaacaatgggttcaagcaagagctacaagacaatctgccgttgcgcacttgacatcctcaagggatatatcgttacgcacgacattagggacaacttctca

SEQ ID NO: 115 - DNA, 1087 bp, unidentified
  misc_feature 1..1087: note = Description of Unknown:mammals-digestive system-rumen-bos taurus sequence
  source 1..1087: mol_type = unassigned DNA
acagagggtgtatggataggcatgaaccaccaaggcaaaatactgatggcttgcagggaggctttgtgtaacaactgtgaacccccgattgattacaaggcactgaacgatgccgagatatatttttatggaaaagaagttaaattttaaaaattaaaagatatggcgaacaaaagcacaaaaggaaacctgcccaagacaatcataatgaaggcaaaccttagccccgatggtttcactcaatgggaaagggttgtaaaagaataccaagcctacaaagacacgttgagtaaatgggtagcccaaaatctcagacaaataatgtgcaagacaccgcagacaaagaacggctactcatcacctgtgctcacctcaaaggttaaaagccaagtggaaatggtaagagaattgaaaaaaatgggaaaaaccattctttattccaatgattcacttcctttttgaaactaaaatgtcttatgtgtatttgaattataggctaatataaagattgtactgtgttgagatacacttttagaggtatttacaacaaaatgcgtgatatggaaatgaagaaataactgtgttgagatacacttttagaggtatttacaacaccatataaacctgaccatctcctgaatctcgcccgacacggataatgttagatatgttcacaatacaactgcatgtgctattcaagaaaaaatagtatatttacaatatgttggtgcataatattagatgtgcttacacaacgcagacctgaaaagccaggataaaagtatgcgggattgtgtttttagaacactgttcaatccgctgtatgtcgcttgaagcgtcagtaacctatgtcgaaacaatccttttagaggtgtttacgaccgaccagaaacagcaagacctgtatttatgttggtatacggttctttttaggggattagtagttgaatcccttttcacccttggtgttcacgggttgtgagacattcttcatacccatgcgtgtcttctcagccatcttaccgaaagttataggcacaatatgttcaatgcctgcctgctgagcattgtagcatatatcagacag

SEQ ID NO: 116 - DNA, 1064 bp, unidentified
  misc_feature 1..1064: note = Description of Unknown:gut metagenome sequence
  source 1..1064: mol_type = unassigned DNA
agaatgctttccccaattgaatgtgaaagactacagacactgccagataactataccgaaggtgttagcaaatgcgcaagatataaggcaatcggaaacggatggacagttgatgtaatttcacatatttttaagaatttgaaaaattaatttggtattttgaaatatttgacttatttttgcaacataaaatttaaaacaaatttatatggcacacgcgaaaaaaaaattttgacaaaggaaagcaaataacaaaaacgttctctttcaaggtgttaaatattaagaacaatggcgaatcagttgatatgaatactatagaattagccatgaaagagtacaataggtattataacatttgtagtgattggatttgcaacaatctaatgacgccaattggttccctatatcaatacatagatgatgagaaatggagaaaaaaatttgttcgcccaacaaacactaataaaccgttgtataactctccagttttctcccctgctgtaaaatctgaaggtggtactattaaaaatctccaaattttaagcgcaacaaagaccataattctttgatttaattattaatacatatatcgttcgtaaatttaatacaaccacaaccaaatatgataatttgcataattaaaaaaattcacatatctttgtagcataaaaacaaatagagaaaaaatgacactttacagatttacacttttaggcaatacacaaatttatgtatatgctggcacgtttgaagatgctctcaggacatttcgtaaatcatatggagatacgggattcaagtcaattgaagagcttcctgaatttagagataacatacttatacaactagattgattgaaacaaacgtcaattacccaccactgaagtagtgggtttctttgcagtgattttatgaaaacgatagaagacagagcagacatagcaagcgatattgctaaaagagaatttgaagaagatagttattggagtcattacgcagacgatatggtaacatctgcttttgttgaaggatgctataaaggctatatttcaggtgcgaca

SEQ ID NO: 117 - DNA, 1617 bp, unidentified
  misc_feature 1..1617: note = Description of Unknown:terrestrial metagenome sequence
  source 1..1617: mol_type = unassigned DNA
aaggagatagattatgacagggaaggtaatatcacaaatatatatctttactatgagtcagatagtttatggaatgaaaaatttgaatttatattaacattagatggttatgaattaaagatacctatttttatagtaagtgtaagatagttttggcacggaaattgcagtaatgttttcctgtcaagaacaaataaaataaaaaatatgaaaaaatcaattaaattcaaagtaaaaggaaattgtccaataaccaaagatgttataaatgaatataaagaatattataataaatgcagtgattggattaagaataatttaacaagcataactattggggaaatggcaaaatttctcaatgaagtgtggagagaaatattttgtacaaggcctaaaaaggcagaatataacgttccatcgttggatacaacaaaaaaaggaccatctgcaatattgcatatgttgaaaaaaatcgaggcaattaaaatattagaaacagaaaagtagtgactatagatataaacttctatgatagatatctgttttttaattctattatgcaatataatatattgaaatataaacaattataaataaaacgggtgtatacaacaagttttttgtttttcttattcattatctgtatatttgtattataaacaaatacaaatatgtataatgaatcaggaatatattgctataaaaacaaaataaacggaaaattatatattggacaggcgctaaatcttaaaagaagatatttaaactttttaaatatcaaccacagatatgcgggtcaagtaatagaaaacgcacgtaaaaaatatggtgtagataactttgaatattcaatccttactcactgtccagtagacgaattaaattattgggaagcattttatgtagaaagattaaattgtgtcacaccccacggttataatatgactaatgggggcgattcagtatatacttctacacaagcatttaaagatgcacaaactgaaaagttgaagcaaactattctatctaagaatcctaatcttaatgtcagcaaagtaaaatatgaaggtaatagaatttcagttataattacttgcccaatacatggcacatttaaaaaaacgcctgattactttagaaatccagaaataaatgatttgtgttgtcctaaatgtgtgagggaagatataagacaaaagactgaagatagtttctttaaacaagcaacaaagaaatggggagataagtatgattattctaaaactataatagtagatagaattaccccagttacaattacttgccctatacacggagattttacagtattaccagggaaccatgtgtgtaaagataaaaatactggaggatgccaacaatgtagtgaagaaagacaacatattgaatcattagaaaaaggtagcgtgaaggtcattaagatgataaagaaaaagtttggaaacaaatattcattagataaattcgaatataggggagataaagaaaaagtaattcttatttgccctattcatggagaattttcaatgacgccaggtaatttaagatatagcaacggttgtccacaatgcactttagaaaatgcttatcgtataaaat

SEQ ID NO: 118 - DNA, 37 bp, synthetic construct
  misc_feature 1..37: note = Description of Artificial Sequence: Syntheticoligonucleotide
  source 1..37: mol_type = other DNA
agttgtaaatacctataaaaatgtattccaacatagt

SEQ ID NO: 119 - DNA, 36 bp, synthetic construct
  misc_feature 1..36: note = Description of Artificial Sequence: Syntheticoligonucleotide
  source 1..36: mol_type = other DNA
gttgtgaataccctacaaaagtgatattccaacaat

SEQ ID NO: 120 - DNA, 19 bp, synthetic construct
  misc_feature 1..19: note = Description of Artificial Sequence: Syntheticoligonucleotide
  source 1..19: mol_type = other DNA
aaaaagggtgaacaacatt

SEQ ID NO: 121 - DNA, 36 bp, synthetic construct
  misc_feature 1..36: note = Description of Artificial Sequence: Syntheticoligonucleotide
  source 1..36: mol_type = other DNA
gttgtttgatacctataaaagagtattcacaacagg

SEQ ID NO: 122 - DNA, 36 bp, synthetic construct
  misc_feature 1..36: note = Description of Artificial Sequence: Syntheticoligonucleotide
  source 1..36: mol_type = other DNA
gttgtttactccatacaaaataagagttacaacaat

SEQ ID NO: 123 - DNA, 36 bp, synthetic construct
  misc_feature 1..36: note = Description of Artificial Sequence: Syntheticoligonucleotide
  source 1..36: mol_type = other DNA
gttgttcaatccttataaaaaggtgtctacaacaat

SEQ ID NO: 124 - DNA, 36 bp, synthetic construct
  misc_feature 1..36: note = Description of Artificial Sequence: Syntheticoligonucleotide
  source 1..36: mol_type = other DNA
gttgtttaatacctataaaagagtatatacaacaag

SEQ ID NO: 125 - DNA, 29 bp, synthetic construct
  misc_feature 1..29: note = Description of Artificial Sequence: Syntheticoligonucleotide
  source 1..29: mol_type = other DNA
aatacctataaaaggacatatacaacaag

SEQ ID NO: 126 - DNA, 36 bp, synthetic construct
  misc_feature 1..36: note = Description of Artificial Sequence: Syntheticoligonucleotide
  source 1..36: mol_type = other DNA
gttgttcaatacctataaaaagacatatacaacaag

SEQ ID NO: 127 - DNA, 25 bp, synthetic construct
  misc_feature 1..25: note = Description of Artificial Sequence: Syntheticoligonucleotide
  source 1..25: mol_type = other DNA
gttgttcattacctaaaaaagagta

SEQ ID NO: 128 - DNA, 36 bp, synthetic construct
  misc_feature 1..36: note = Description of Artificial Sequence: Syntheticoligonucleotide
  source 1..36: mol_type = other DNA
gttgtttaatacctataaaagaatatatacaacaag

SEQ ID NO: 129 - DNA, 36 bp, synthetic construct
  misc_feature 1..36: note = Description of Artificial Sequence: Syntheticoligonucleotide
  source 1..36: mol_type = other DNA
gttgtttaatacttaaaaaatagtatgtacaacatg

SEQ ID NO: 130 - DNA, 36 bp, synthetic construct
  misc_feature 1..36: note = Description of Artificial Sequence: Syntheticoligonucleotide
  source 1..36: mol_type = other DNA
gttgtatccaccgtataaaacatagtgtccaacatc

SEQ ID NO: 131 - DNA, 36 bp, synthetic construct
  misc_feature 1..36: note = Description of Artificial Sequence: Syntheticoligonucleotide
  source 1..36: mol_type = other DNA
gttgtttattacttacaaaaacagcataacaacatc

SEQ ID NO: 132 - DNA, 36 bp, synthetic construct
  misc_feature 1..36: note = Description of Artificial Sequence: Syntheticoligonucleotide
  source 1..36: mol_type = other DNA
gttgttcaatccttataaaaagaggtctacaacaat

SEQ ID NO: 133 - DNA, 36 bp, synthetic construct
  misc_feature 1..36: note = Description of Artificial Sequence: Syntheticoligonucleotide
  source 1..36: mol_type = other DNA
gttgttattaccatataaaatggttcgtacaacaat

SEQ ID NO: 134 - DNA, 39 bp, synthetic construct
  misc_feature 1..39: note = Description of Artificial Sequence: Syntheticoligonucleotide
  source 1..39: mol_type = other DNA
actgttgtactttcctttcatctgcaggggttttacagt

SEQ ID NO: 135 - DNA, 40 bp, synthetic construct
  misc_feature 1..40: note = Description of Artificial Sequence: Syntheticoligonucleotide
  source 1..40: mol_type = other DNA
tagttgtttaatacttaaaaaatagtatgtacaacatgat

SEQ ID NO: 136 - DNA, 36 bp, synthetic construct
  misc_feature 1..36: note = Description of Artificial Sequence: Syntheticoligonucleotide
  source 1..36: mol_type = other DNA
gttgtaaatagcatacaaacatagccattcaacaat

SEQ ID NO: 137 - DNA, 36 bp, synthetic construct
  misc_feature 1..36: note = Description of Artificial Sequence: Syntheticoligonucleotide
  source 1..36: mol_type = other DNA
gttgtgagtaccctataaaagaagtaccccaacaat

SEQ ID NO: 138 - DNA, 37 bp, synthetic construct
  misc_feature 1..37: note = Description of Artificial Sequence: Syntheticoligonucleotide
  source 1..37: mol_type = other DNA
cgttgttagacccctaaaacacaaggtctacaacaat

SEQ ID NO: 139 - DNA, 36 bp, synthetic construct
  misc_feature 1..36: note = Description of Artificial Sequence: Syntheticoligonucleotide
  source 1..36: mol_type = other DNA
gttgtaaatacatctcatattgtattccaacacagt

SEQ ID NO: 140 - DNA, 36 bp, synthetic construct
  misc_feature 1..36: note = Description of Artificial Sequence: Syntheticoligonucleotide
  source 1..36: mol_type = other DNA
gttgtcagcatccgccttgcggtatgccacaacaat

SEQ ID NO: 141 - DNA, 36 bp, synthetic construct
  misc_feature 1..36: note = Description of Artificial Sequence: Syntheticoligonucleotide
  source 1..36: mol_type = other DNA
gttgccaataccataaaaaacggtatctcaacaatt

SEQ ID NO: 142 - DNA, 35 bp, synthetic construct
  misc_feature 1..35: note = Description of Artificial Sequence: Syntheticoligonucleotide
  source 1..35: mol_type = other DNA
ttgtcagcacccgtaatacggtatgccacaacaat

SEQ ID NO: 143 - DNA, 36 bp, synthetic construct
  misc_feature 1..36: note = Description of Artificial Sequence: Syntheticoligonucleotide
  source 1..36: mol_type = other DNA
gttgtcagcacccgtaatacggtatgccacaacaat

SEQ ID NO: 144 - DNA, 36 bp, synthetic construct
  misc_feature 1..36: note = Description of Artificial Sequence: Syntheticoligonucleotide
  source 1..36: mol_type = other DNA
gttgtaaatacctataaaagtgtatcccaacacaat

SEQ ID NO: 145 - DNA, 37 bp, synthetic construct
  misc_feature 1..37: note = Description of Artificial Sequence: Syntheticoligonucleotide
  source 1..37: mol_type = other DNA
gttgtaattacctttataagaaaggtattcaacaata

SEQ ID NO: 146 - DNA, 36 bp, synthetic construct
  misc_feature 1..36: note = Description of Artificial Sequence: Syntheticoligonucleotide
  source 1..36: mol_type = other DNA
gagttgttcgttgcccataaaaagccatttacaaca

SEQ ID NO: 147 - DNA, 36 bp, synthetic construct
  misc_feature 1..36: note = Description of Artificial Sequence: Syntheticoligonucleotide
  source 1..36: mol_type = other DNA
gttgtatctgtcctaaaaaagaatacattcaacaat

SEQ ID NO: 148 - DNA, 37 bp, synthetic construct
  misc_feature 1..37: note = Description of Artificial Sequence: Syntheticoligonucleotide
  source 1..37: mol_type = other DNA
agttgtaatcagtctataaaagataccattcaacaat

SEQ ID NO: 149 - DNA, 36 bp, synthetic construct
  misc_feature 1..36: note = Description of Artificial Sequence: Syntheticoligonucleotide
  source 1..36: mol_type = other DNA
gttgtaattaagataaaaaacctattatccaacaat

SEQ ID NO: 150 - DNA, 36 bp, synthetic construct
  misc_feature 1..36: note = Description of Artificial Sequence: Syntheticoligonucleotide
  source 1..36: mol_type = other DNA
gttgtaaataggatataaaatcaactattcaacagt

SEQ ID NO: 151 - DNA, 36 bp, synthetic construct
  misc_feature 1..36: note = Description of Artificial Sequence: Syntheticoligonucleotide
  source 1..36: mol_type = other DNA
gttgttcaatccttacaaaaaggtatctacaacaat

SEQ ID NO: 152 - DNA, 103 bp, synthetic construct
  misc_feature 1..103: note = Description of Artificial Sequence: Syntheticpolynucleotide
  source 1..103: mol_type = other DNA
attgggacttccggaagtaaaatatccacctgaggattttaggacatataatttctaataaaaatgaacggaaaaatttccgttcattttttttttgtttatt

SEQ ID NO: 153 - DNA, 105 bp, synthetic construct
  misc_feature 1..105: note = Description of Artificial Sequence: Syntheticpolynucleotide
  source 1..105: mol_type = other DNA
tattgggacttccggaagtaaaatatccacctgaggattttaggacatataatttctaataaaaatgaacggaaaaatttccgttcattttttttttgtttattg

SEQ ID NO: 154 - DNA, 163 bp, synthetic construct
  misc_feature 1..163: note = Description of Artificial Sequence: Syntheticpolynucleotide
  source 1..163: mol_type = other DNA
gacgagaacggagtgtggctcctgaggaaaaacgacaaacatccaacatattttatctaccagaacggaacactctatcaatatgaggaagattgattagttgatgttttcataataattttatctggaatttgaaaagattccagattttttttttatttcg

SEQ ID NO: 155 - DNA, 66 bp, synthetic construct
  misc_feature 1..66: note = Description of Artificial Sequence: Syntheticoligonucleotide
  source 1..66: mol_type = other DNA
gcaatcaacaagactttcattttcaaggcaaaatgcgataagaacgatgtcatatcgttatgggaa

SEQ ID NO: 156 - DNA, 59 bp, synthetic construct
  misc_feature 1..59: note = Description of Artificial Sequence: Syntheticoligonucleotide
  source 1..59: mol_type = other DNA
gatgctccgaaaacgtggttgttcggacaacaaaaaaatgaatgtttctaatgtattaa

SEQ ID NO: 157 - DNA, 70 bp, synthetic construct
  misc_feature 1..70: note = Description of Artificial Sequence: Syntheticoligonucleotide
  source 1..70: mol_type = other DNA
gacggaaaaataaatgaggatggtatgtttgttgaaaacttggaataattctgtatataccaattagaat

SEQ ID NO: 158 - DNA, 55 bp, synthetic construct
  misc_feature 1..55: note = Description of Artificial Sequence: Syntheticoligonucleotide
  source 1..55: mol_type = other DNA
tgttgattgctgattcttcgttgtttgatttgtgttgtgccataatcttaaaatt

SEQ ID NO: 159 - DNA, 83 bp, synthetic construct
  misc_feature 1..83: note = Description of Artificial Sequence: Syntheticoligonucleotide
  source 1..83: mol_type = other DNA
cgcaagatataaggcaatcggaaacggatggacagttgatgtaatttcacatatttttaagaatttgaaaaattaatttggta

SEQ ID NO: 160 - DNA, 95 bp, synthetic construct
  misc_feature 1..95: note = Description of Artificial Sequence: Syntheticoligonucleotide
  source 1..95: mol_type = other DNA
ggacatttcgtaaatcatatggagatacggagttcaagtcaattgaagagcttcctgaatttagagataacatacttatacaactagattgattg

SEQ ID NO: 161 - DNA, 59 bp, synthetic construct
  misc_feature 1..59: note = Description of Artificial Sequence: Syntheticoligonucleotide
  source 1..59: mol_type = other DNA
atcaatacatagatgatgagaaatggagaaaaaaatttgttcgcccaacaaacactaat

SEQ ID NO: 162 - DNA, 80 bp, synthetic construct
  misc_feature 1..80: note = Description of Artificial Sequence: Syntheticoligonucleotide
  source 1..80: mol_type = other DNA
ctggtaatactgtaaaatctccgtgtatagggcaagtaattgtaactggggtaattctatctactattatagttttagaa

SEQ ID NO: 163 - DNA, 56 bp, synthetic construct
  misc_feature 1..56: note = Description of Artificial Sequence: Syntheticoligonucleotide
  source 1..56: mol_type = other DNA
cagaagtcgttcaagttcaaggtcaaaacggacaaggagacggtcgaattattcag

SEQ ID NO: 164 - DNA, 66 bp, synthetic construct
  misc_feature 1..66: note = Description of Artificial Sequence: Syntheticoligonucleotide
  source 1..66: mol_type = other DNA
gggagggtgacattcagaagtcgttcaagttcaaggtcaaaacggacaaggagacggtcgaattat

SEQ ID NO: 165 - DNA, 102 bp, synthetic construct
  misc_feature 1..102: note = Description of Artificial Sequence: Syntheticpolynucleotide
  source 1..102: mol_type = other DNA
aagtgtcttcaacacattgaagaaaactctcggtgcaatatatggaaagctcgatgaaaacggaaattttattgagaatgaatgtaataagtaactggaata

SEQ ID NO: 166 - DNA, 98 bp, synthetic construct
  misc_feature 1..98: note = Description of Artificial Sequence: Syntheticoligonucleotide
  source 1..98: mol_type = other DNA
ccgtgggaggatttggatttggttgaagacatcagaaaaattttcgaaatggaatagagggaaccggaattttttccggtttttctttgtcctttcga

SEQ ID NO: 167 - DNA, 82 bp, synthetic construct
  misc_feature 1..82: note = Description of Artificial Sequence: Syntheticoligonucleotide
  source 1..82: mol_type = other DNA
cagagtaacctttcctgatatgttgttacacatttttgtaagtgttaaacaactgacgcattgatattgccttgtctattaa

SEQ ID NO: 168 - DNA, 82 bp, synthetic construct
  misc_feature 1..82: note = Description of Artificial Sequence: Syntheticoligonucleotide
  source 1..82: mol_type = other DNA
caatcgcgagtttatactgaaatgttgttacactgtttttgtaagtgttaaacaaccttgcacaaatgtcatctaccagtac

SEQ ID NO: 169 - DNA, 78 bp, synthetic construct
  misc_feature 1..78: note = Description of Artificial Sequence: Syntheticoligonucleotide
  source 1..78: mol_type = other DNA
ccgagcgacccacaaacctattgtcgtacgcatcatttcacatgataataacaacgaatattcctgcaagcatgattt

SEQ ID NO: 170 - DNA, 77 bp, synthetic construct
  misc_feature 1..77: note = Description of Artificial Sequence: Syntheticoligonucleotide
  source 1..77: mol_type = other DNA
tatgacattatgatattgttgtatgcatcatttcacatggtaataacaacgaagagaaacaccgagcgacccacaaa

SEQ ID NO: 171 - DNA, 85 bp, synthetic construct
  misc_feature 1..85: note = Description of Artificial Sequence: Syntheticoligonucleotide
  source 1..85: mol_type = other DNA
acatcttttatgacattatgatattgttgtatgcatcatttcacatggtaataacaacgaagagaaacaccgagcgacccacaaa

SEQ ID NO: 172 - DNA, 82 bp, synthetic construct
  misc_feature 1..82: note = Description of Artificial Sequence: Syntheticoligonucleotide
  source 1..82: mol_type = other DNA
gctaaaatatagtcctgtggatgttgaatacatttcttttaagtgtacttacaaccaacgctgtacacattgctaatggatg

SEQ ID NO: 173 - DNA, 83 bp, synthetic construct
  misc_feature 1..83: note = Description of Artificial Sequence: Syntheticoligonucleotide
  source 1..83: mol_type = other DNA
tgctaaaatatagtcctgtggatgttgaatacatttcttttaagtgtacttacaaccaacgctgtacacattgctaatggatg

SEQ ID NO: 174 - DNA, 87 bp, synthetic construct
  misc_feature 1..87: note = Description of Artificial Sequence: Syntheticoligonucleotide
  source 1..87: mol_type = other DNA
caacaccaaggctgaggcaaagaagagggctgatgatatgaacaaacagaatagggtcatacaccagctgtctgtttatttgtgtcc

SEQ ID NO: 175 - DNA, 95 bp, synthetic construct
  misc_feature 1..95: note = Description of Artificial Sequence: Syntheticoligonucleotide
  source 1..95: mol_type = other DNA
aattagactgataaacaaagaataatgagaactataatagggaggtgtacccccgaatttaagccagtggagaaccatacaaacctatcatatag

SEQ ID NO: 176 - DNA, 72 bp, synthetic construct
  misc_feature 1..72: note = Description of Artificial Sequence: Syntheticoligonucleotide
  source 1..72: mol_type = other DNA
tgggtatgcgttgtttaatacttaaaaaaatgtatgtacaacatgtctgtggaaagtctttctattgtatat

SEQ ID NO: 177 - DNA, 68 bp, synthetic construct
  misc_feature 1..68: note = Description of Artificial Sequence: Syntheticoligonucleotide
  source 1..68: mol_type = other DNA
cgttgtttaatacttaaaaaaatgtatgtacaacatgtctgtggaaagtctttctattgtatatagga

SEQ ID NO: 178 - DNA, 118 bp, synthetic construct
  misc_feature 1..118: note = Description of Artificial Sequence: Syntheticpolynucleotide
  source 1..118: mol_type = other DNA
tgggtatgcgttgtttaatacttaaaaaaatgtatgtacaacatgtctgtggaaagtctttctattgtatataggaattttatataattatttaattatcaatgaattatattagtat

SEQ ID NO: 179 - DNA, 58 bp, synthetic construct
  misc_feature 1..58: note = Description of Artificial Sequence: Syntheticoligonucleotide
  source 1..58: mol_type = other DNA
ggtgggtatgcgttgtttaatacttaaaaaaatgtatgtacaacatgtctgtggaaag

SEQ ID NO: 180 - DNA, 73 bp, synthetic construct
  misc_feature 1..73: note = Description of Artificial Sequence: Syntheticoligonucleotide
  source 1..73: mol_type = other DNA
aatgaacgagattgttgggatataccttttataggattttcacaacatctgagttgtttgatgttaaaaactt

SEQ ID NO: 181 - DNA, 80 bp, synthetic construct
  misc_feature 1..80: note = Description of Artificial Sequence: Syntheticoligonucleotide
  source 1..80: mol_type = other DNA
gataaaaatgaacgagattgttgggatataccttttataggattttcacaacatctgagttgtttgatgttaaaaacttt

SEQ ID NO: 182 - DNA, 75 bp, synthetic construct
  misc_feature 1..75: note = Description of Artificial Sequence: Syntheticoligonucleotide
  source 1..75: mol_type = other DNA
gctaatataaagattgtactgtgttgagatacacttttagaggtatttacaacaaaatgcgtgatatggaaatga

SEQ ID NO: 183 - DNA, 90 bp, synthetic construct
  misc_feature 1..90: note = Description of Artificial Sequence: Syntheticoligonucleotide
  source 1..90: mol_type = other DNA
ataccaacataaatacaggtcttgctgtttctggtcggtcgtaaacacctctaaaaggattgtttcgacataggttactgacgcttcaag

SEQ ID NO: 184 - DNA, 72 bp, synthetic construct
  misc_feature 1..72: note = Description of Artificial Sequence: Syntheticoligonucleotide
  source 1..72: mol_type = other DNA
aatgaagaaataactgtgttgagatacacttttagaggtatttacaacaccatataaacctgaccatctcct

SEQ ID NO: 185 - DNA, 84 bp, synthetic construct
  misc_feature 1..84: note = Description of Artificial Sequence: Syntheticoligonucleotide
  source 1..84: mol_type = other DNA
aggaagatgtcagacgtttttattgttggaatactcgttttttacggtatttacaactgccccgtagcggaatcaaaataccac

SEQ ID NO: 186 - DNA, 76 bp, synthetic construct
  misc_feature 1..76: note = Description of Artificial Sequence: Syntheticoligonucleotide
  source 1..76: mol_type = other DNA
atgtcagacgtttttattgttggaatactcgttttttacggtatttacaactgccccgtagcggaatcaaaatacc

SEQ ID NO: 187 - DNA, 99 bp, synthetic construct
  misc_feature 1..99: note = Description of Artificial Sequence: Syntheticoligonucleotide
  source 1..99: mol_type = other DNA
aaataacaaaaattctggacgggaaaggaagatgtcagacgtttttattgttggaatactcgttttttacggtatttacaactgccccgtagcggaatc

SEQ ID NO: 188 - DNA, 96 bp, synthetic construct
  misc_feature 1..96: note = Description of Artificial Sequence: Syntheticoligonucleotide
  source 1..96: mol_type = other DNA
ataacaaaaattctggacgggaaaggaagatgtcagacgtttttattgttggaatactcgttttttacggtatttacaactgccccgtagcggaat

SEQ ID NO: 189 - DNA, 60 bp, synthetic construct
  misc_feature 1..60: note = Description of Artificial Sequence: Syntheticoligonucleotide
  source 1..60: mol_type = other DNA
tattgcaactattacaacaaacttagcgaatggattggcaaagatatgtataacacgccg

SEQ ID NO: 190 - DNA, 59 bp, synthetic construct
  misc_feature 1..59: note = Description of Artificial Sequence: Syntheticoligonucleotide
  source 1..59: mol_type = other DNA
attgcaactattacaacaaacttagcgaatggattggcaaagatatgtataacacgccg

SEQ ID NO: 191 - DNA, 71 bp, synthetic construct
  misc_feature 1..71: note = Description of Artificial Sequence: Syntheticoligonucleotide
  source 1..71: mol_type = other DNA
gtatgatgacagaagaaacacggaagacaatagagagcgtcatagtggttctcggcatagcaatcatgctg

SEQ ID NO: 192 - DNA, 118 bp, synthetic construct
  misc_feature 1..118: note = Description of Artificial Sequence: Syntheticpolynucleotide
  source 1..118: mol_type = other DNA
atgatgacagaagaaacacggaagacaatagagagcgtcatagtggttctcggcatagcaatcatgctggcagccgccgtccgaataatgacgcagaacaaagcaattgtgaaatatg

SEQ ID NO: 193 - DNA, 57 bp, synthetic construct
  misc_feature 1..57: note = Description of Artificial Sequence: Syntheticoligonucleotide
  source 1..57: mol_type = other DNA
agaaggtactgccgccttatgaccgacgagaacggagtgtggctcctgaggaaaaac

SEQ ID NO: 194 - DNA, 163 bp, synthetic construct; identical to SEQ ID NO: 154
  misc_feature 1..163: note = Description of Artificial Sequence: Syntheticpolynucleotide
  source 1..163: mol_type = other DNA

SEQ ID NO: 195 - DNA, 92 bp, synthetic construct
  misc_feature 1..92: note = Description of Artificial Sequence: Syntheticoligonucleotide
  source 1..92: mol_type = other DNA
tttttgttatatatttgtcctgttaggttaaatcaccgcgcctgatgacgaagtcggtggtagaattagactaatattaaatatgtctcatg

SEQ ID NO: 196 - DNA, 82 bp, synthetic construct
  misc_feature 1..82: note = Description of Artificial Sequence: Syntheticoligonucleotide
  source 1..82: mol_type = other DNA
cctattagatattccgtatttctttaagactgttataatacaaatatactacaaatcatgcaatttttgatttttaacaaaa

SEQ ID NO: 197 - DNA, 103 bp, synthetic construct
  misc_feature 1..103: note = Description of Artificial Sequence: Syntheticpolynucleotide
  source 1..103: mol_type = other DNA
tcgttgaatacgatatcgccgaaacaattgattggagaagtacgctttgtttcaagacatggaatacgtatggttctcctcaatgggactcgaagatcaagaa

SEQ ID NO: 198 - DNA, 108 bp, synthetic construct
  misc_feature 1..108: note = Description of Artificial Sequence: Syntheticpolynucleotide
  source 1..108: mol_type = other DNA
atcgttgaatacgatatcgccgaaacaattgattggagaagtacgctttgtttcaagacatggaatacgtatggttctcctcaatgggactcgaagatcaagaaccag

SEQ ID NO: 199 - DNA, 73 bp, synthetic construct
  misc_feature 1..73: note = Description of Artificial Sequence: Syntheticoligonucleotide
  source 1..73: mol_type = other DNA
gagcttttctggcaatgtagacattaaagctggtatcgttgaatacgatatcgccgaaacaattgattggaga

SEQ ID NO: 200 - DNA, 98 bp, synthetic construct
  misc_feature 1..98: note = Description of Artificial Sequence: Syntheticoligonucleotide
  source 1..98: mol_type = other DNA
tttttcattgttctcaaattgttggataatgttttgtgtgtttcatttttgtcattgtgtcaccttaactgacaaggtggcacattttttatgtcaat

SEQ ID NO: 201 - DNA, 98 bp, synthetic construct
  misc_feature 1..98: note = Description of Artificial Sequence: Syntheticoligonucleotide
  source 1..98: mol_type = other DNA
ttttcattgttctcaaattgttggataatgttttgtgtgtttcatttttgtcattgtgtcaccttaactgacaaggtggcacattttttatgtcaata

SEQ ID NO: 202 - DNA, 122 bp, synthetic construct
  misc_feature 1..122: note = Description of Artificial Sequence: Syntheticpolynucleotide
  source 1..122: mol_type = other DNA
aatatatctgctaaggtcatatttttcattgttctcaaattgttggataatgttttgtgtgtttcatttttgtcattgtgtcaccttaactgacaaggtggcacattttttatgtcaatatg

SEQ ID NO: 203 - DNA, 75 bp, synthetic construct
  misc_feature 1..75: note = Description of Artificial Sequence: Syntheticoligonucleotide
  source 1..75: mol_type = other DNA
acaaatttttgattatggcacacaaaaagaacataggagcagagatagtaaaaacttactcttttaaggtgaaga

SEQ ID NO: 204 - DNA, 136 bp, synthetic construct
  misc_feature 1..136: note = Description of Artificial Sequence: Syntheticpolynucleotide
  source 1..136: mol_type = other DNA
ttattttataggataatagagctaacaagcattaacaattattaaaacgatttatattgaaaataaattttgtgggaatatttatttttactacctttgcatcgtaatacaattaaacaaatttttgattatggca

SEQ ID NO: 205 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaacggaagtggttggtcagcatggatta

SEQ ID NO: 206 - DNA, 25 bp, unidentified
  misc_feature 1..25: note = Description of Unknown:target sequence
  source 1..25: mol_type = other DNA
ggaagtggttggtcagcatggatta

SEQ ID NO: 207 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaactgtgaagtgacctgggagctaactg

SEQ ID NO: 208 - DNA, 25 bp, unidentified
  misc_feature 1..25: note = Description of Unknown:target sequence
  source 1..25: mol_type = other DNA
tgtgaagtgacctgggagctaactg

SEQ ID NO: 209 - DNA, 61 bp, synthetic construct
  misc_feature 1..61: note = Description of Artificial Sequence: Syntheticoligonucleotide
  source 1..61: mol_type = other DNA
attgttgtagacacctttttataaggattgaacaacaacccccgtctacctgcccacaggg

SEQ ID NO: 210 - DNA, 25 bp, unidentified
  misc_feature 1..25: note = Description of Unknown:target sequence
  source 1..25: mol_type = other DNA
aacccccgtctacctgcccacaggg

SEQ ID NO: 211 - DNA, 61 bp, synthetic construct
  misc_feature 1..61: note = Description of Artificial Sequence: Syntheticoligonucleotide
  source 1..61: mol_type = other DNA
cttgttgtatatgtccttttataggtattaaacaacgtagagggagaaatggaatccatat

SEQ ID NO: 212 - DNA, 25 bp, unidentified
  misc_feature 1..25: note = Description of Unknown:target sequence
  source 1..25: mol_type = other DNA
gtagagggagaaatggaatccatat

SEQ ID NO: 213 - DNA, 36 bp, synthetic construct
  misc_feature 1..36: note = Description of Artificial Sequence: Syntheticoligonucleotide
  source 1..36: mol_type = other DNA
cttgttgtatatgtccttttataggtattaaacaac

SEQ ID NO: 214 - DNA, 61 bp, synthetic construct
  misc_feature 1..61: note = Description of Artificial Sequence: Syntheticoligonucleotide
  source 1..61: mol_type = other DNA
attgttgtagacacctttttataaggattgaacaacgcaccaacgggtagatttggtggtg

SEQ ID NO: 215 - DNA, 25 bp, unidentified
  misc_feature 1..25: note = Description of Unknown:target sequence
  source 1..25: mol_type = other DNA
gcaccaacgggtagatttggtggtg

SEQ ID NO: 216-218 - skipped (3 entries)

SEQ ID NO: 219 - protein, 10 aa, synthetic construct
  REGION 1..10: note = Description of Artificial Sequence: Syntheticpeptide
  VARIANT 2: note = T, I, N, A, S, F, or V
  VARIANT 3: note = I, V, L, or S
  VARIANT 4: note = H, S, G, or R
  VARIANT 7: note = D, S, or E
  VARIANT 8: note = I, V, M, T, or N
KXXXFAXXKD

SEQ ID NO: 220 - skipped

SEQ ID NO: 221 - protein, 10 aa, synthetic construct
  REGION 1..10: note = Description of Artificial Sequence: Syntheticpeptide
  VARIANT 2: note = S, P, or A
  VARIANT 3: note = Y, S, A, P, E, Y, Q, or N
  VARIANT 4: note = F, Y, or H
  VARIANT 5: note = T or S
  VARIANT 8: note = M, T, or I
PXXXXSQXDS

SEQ ID NO: 222 - protein, 11 aa, synthetic construct
  REGION 1..11: note = Description of Artificial Sequence: Syntheticpeptide
  VARIANT 2: note = N, K, W, R, E, T, or Y
  VARIANT 3: note = M, R, L, S, K, V, E, T, I, or D
  VARIANT 6: note = L, R, H, P, T, K, Q, P, S, or A
  VARIANT 7: note = G, Q, N, R, K, E, I, T, S, or C
  VARIANT 10: note = R, W, Y, K, T, F, S, or Q
KXXVRXXQEXH

SEQ ID NO: 223 - protein, 13 aa, synthetic construct
  REGION 1..13: note = Description of Artificial Sequence: Syntheticpeptide
  VARIANT 1: note = I, K, V, or L
  VARIANT 4: note = L or M
  VARIANT 5: note = N, H, or P
  VARIANT 6: note = A, S, or C
  VARIANT 8: note = V, Y, I, F, T, N, or Y
  VARIANT 10: note = A or S
  VARIANT 11: note = S, A, or P
  VARIANT 12: note = M, C, L, R, N, S, K, or L
XNGXXXDXNXXXN

SEQ ID NO: 224-229 - skipped (6 entries)

SEQ ID NO: 230 - DNA, 41 bp, synthetic construct
  misc_feature 1..41: note = Description of Artificial Sequence: Syntheticoligonucleotide
  misc_difference 18: note = a, c, t, g, unknown or other
  source 1..41: mol_type = other DNA
atattgttgdakrwwyynttttwtargkwwwwwacaacwrb

SEQ ID NO: 231 - protein, 8 aa, synthetic construct
  REGION 1..8: note = Description of Artificial Sequence: Syntheticpeptide
NLTSITIG

SEQ ID NO: 232 - protein, 10 aa, synthetic construct
  REGION 1..10: note = Description of Artificial Sequence: Syntheticpeptide
NYRTKIRTLN

SEQ ID NO: 233 - protein, 9 aa, synthetic construct
  REGION 1..9: note = Description of Artificial Sequence: Syntheticpeptide
ISYIENVEN

SEQ ID NO: 234 - protein, 9 aa, synthetic construct
  REGION 1..9: note = Description of Artificial Sequence: Syntheticpeptide
ELLSVEQLK

SEQ ID NO: 235 - protein, 15 aa, synthetic construct
  REGION 1..15: note = Description of Artificial Sequence: Syntheticpeptide
HINSMTINIQDFKIE

SEQ ID NO: 236 - protein, 9 aa, synthetic construct
  REGION 1..9: note = Description of Artificial Sequence: Syntheticpeptide
KENSLGFIL

SEQ ID NO: 237 - protein, 8 aa, synthetic construct
  REGION 1..8: note = Description of Artificial Sequence: Syntheticpeptide
GNRQIKKG

SEQ ID NO: 238 - protein, 7 aa, synthetic construct
  REGION 1..7: note = Description of Artificial Sequence: Syntheticpeptide
DVNFKHA

SEQ ID NO: 239 - protein, 12 aa, synthetic construct
  REGION 1..12: note = Description of Artificial Sequence: Syntheticpeptide
GYINLYKYLLEH

SEQ ID NO: 240 - protein, 10 aa, synthetic construct
  REGION 1..10: note = Description of Artificial Sequence: Syntheticpeptide
KEQVLSKLLY

SEQ ID NO: 241 - protein, 38 aa, synthetic construct
  REGION 1..38: note = Description of Artificial Sequence: Syntheticpolypeptide
EYIYVSCVNKLRAKYVSYFILKEKYYEKQKEYDIEMGF

SEQ ID NO: 242 - protein, 14 aa, synthetic construct
  REGION 1..14: note = Description of Artificial Sequence: Syntheticpeptide
DDSTESKESMDKRR

SEQ ID NO: 243 - protein, 16 aa, synthetic construct
  REGION 1..16: note = Description of Artificial Sequence: Syntheticpeptide
NVQQDINGCLKNIINY

SEQ ID NO: 244 - protein, 12 aa, synthetic construct
  REGION 1..12: note = Description of Artificial Sequence: Syntheticpeptide
ALENLENSNFEK

SEQ ID NO: 245 - protein, 10 aa, synthetic construct
  REGION 1..10: note = Description of Artificial Sequence: Syntheticpeptide
QVLPTIKSLL

SEQ ID NO: 246 - protein, 8 aa, synthetic construct
  REGION 1..8: note = Description of Artificial Sequence: Syntheticpeptide
YHKLENQN

SEQ ID NO: 247 - protein, 10 aa, synthetic construct
  REGION 1..10: note = Description of Artificial Sequence: Syntheticpeptide
ASDKVKEYIE

SEQ ID NO: 248 - protein, 13 aa, synthetic construct
  REGION 1..13: note = Description of Artificial Sequence: Syntheticpeptide
TNENNEIVDAKYT

SEQ ID NO: 249 - protein, 15 aa, synthetic construct
  REGION 1..15: note = Description of Artificial Sequence: Syntheticpeptide
ANFFNLMMKSLHFAS

SEQ ID NO: 250 - protein, 16 aa, synthetic construct
  REGION 1..16: note = Description of Artificial Sequence: Syntheticpeptide
LLSNNGKTQIALVPSE

SEQ ID NO: 251 - protein, 18 aa, synthetic construct
  REGION 1..18: note = Description of Artificial Sequence: Syntheticpeptide
HINGLNADFNAANNIKYI

SEQ ID NO: 252 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaacgagaggtgagggacttggggggtaa

SEQ ID NO: 253 - DNA, 25 bp, synthetic construct
  misc_feature 1..25: note = Description of Artificial Sequence: Syntheticoligonucleotide
  source 1..25: mol_type = other DNA
gagaggtgagggacttggggggtaa

SEQ ID NO: 254 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaactgagaatggtgcgtcctaggtgttc

SEQ ID NO: 255 - DNA, 25 bp, synthetic construct
  misc_feature 1..25: note = Description of Artificial Sequence: Syntheticoligonucleotide
  source 1..25: mol_type = other DNA
tgagaatggtgcgtcctaggtgttc

SEQ ID NO: 256 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaacgcagcctgtgctgacccatgcagtc

SEQ ID NO: 257 - DNA, 25 bp, synthetic construct
  misc_feature 1..25: note = Description of Artificial Sequence: Syntheticoligonucleotide
  source 1..25: mol_type = other DNA
gcagcctgtgctgacccatgcagtc

SEQ ID NO: 258 - DNA, 61 bp, synthetic construct; identical to SEQ ID NO: 205
  misc_feature 1..61: note = Description of Artificial Sequence: Syntheticoligonucleotide
  source 1..61: mol_type = other DNA

SEQ ID NO: 259 - DNA, 25 bp, synthetic construct
  misc_feature 1..25: note = Description of Artificial Sequence: Syntheticoligonucleotide
  source 1..25: mol_type = other DNA
ggaagtggttggtcagcatggatta

SEQ ID NO: 260 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaacagccagtgttgctagtcaagggcag

SEQ ID NO: 261 - DNA, 25 bp, synthetic construct
  misc_feature 1..25: note = Description of Artificial Sequence: Syntheticoligonucleotide
  source 1..25: mol_type = other DNA
agccagtgttgctagtcaagggcag

SEQ ID NO: 262 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaacttgacattgtccacacctggaatcg

SEQ ID NO: 263 - DNA, 25 bp, synthetic construct
  misc_feature 1..25: note = Description of Artificial Sequence: Syntheticoligonucleotide
  source 1..25: mol_type = other DNA
ttgacattgtccacacctggaatcg

SEQ ID NO: 264 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaacgaaatctattgaggctctggagaga

SEQ ID NO: 265 - DNA, 25 bp, synthetic construct
  misc_feature 1..25: note = Description of Artificial Sequence: Syntheticoligonucleotide
  source 1..25: mol_type = other DNA
gaaatctattgaggctctggagaga

SEQ ID NO: 266 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaacggaagctggatgagcctggtccatg

SEQ ID NO: 267 - DNA, 25 bp, synthetic construct
  misc_feature 1..25: note = Description of Artificial Sequence: Syntheticoligonucleotide
  source 1..25: mol_type = other DNA
ggaagctggatgagcctggtccatg

SEQ ID NO: 268 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaaccccatactggggaccaaggaagtgt

SEQ ID NO: 269 - DNA, 25 bp, synthetic construct
  misc_feature 1..25: note = Description of Artificial Sequence: Syntheticoligonucleotide
  source 1..25: mol_type = other DNA
cccatactggggaccaaggaagtgt

SEQ ID NO: 270 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaacatgatgctttgccgtaacccttcgt

SEQ ID NO: 271 - DNA, 25 bp, synthetic construct
  misc_feature 1..25: note = Description of Artificial Sequence: Syntheticoligonucleotide
  source 1..25: mol_type = other DNA
atgatgctttgccgtaacccttcgt

SEQ ID NO: 272 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaacaagagtcattgccccactttaccct

SEQ ID NO: 273 - DNA, 25 bp, synthetic construct
  misc_feature 1..25: note = Description of Artificial Sequence: Syntheticoligonucleotide
  source 1..25: mol_type = other DNA
aagagtcattgccccactttaccct

SEQ ID NO: 274 - DNA, 61 bp, synthetic construct; identical to SEQ ID NO: 252
  misc_feature 1..61: note = Description of Artificial Sequence: Syntheticoligonucleotide
  source 1..61: mol_type = other DNA

SEQ ID NO: 275 - DNA, 25 bp, synthetic construct
  misc_feature 1..25: note = Description of Artificial Sequence: Syntheticoligonucleotide
  source 1..25: mol_type = other DNA
gagaggtgagggacttggggggtaa

SEQ ID NO: 276 - DNA, 61 bp, synthetic construct
  misc_feature 1..61: note = Description of Artificial Sequence: Syntheticoligonucleotide
  source 1..61: mol_type = other DNA
cctgttgtgaatactcttttataggtatcaaacaacgtgaagttctaaacttcatattacc

SEQ ID NO: 277 - DNA, 25 bp, synthetic construct
  misc_feature 1..25: note = Description of Artificial Sequence: Syntheticoligonucleotide
  source 1..25: mol_type = other DNA
gtgaagttctaaacttcatattacc

SEQ ID NO: 278 - DNA, 61 bp, synthetic construct; identical to SEQ ID NO: 211
  misc_feature 1..61: note = Description of Artificial Sequence: Syntheticoligonucleotide
  source 1..61: mol_type = other DNA

SEQ ID NO: 279 - DNA, 25 bp, synthetic construct
  misc_feature 1..25: note = Description of Artificial Sequence: Syntheticoligonucleotide
  source 1..25: mol_type = other DNA
gtagagggagaaatggaatccatat

SEQ ID NO: 280 - DNA, 61 bp, synthetic construct
  misc_feature 1..61: note = Description of Artificial Sequence: Syntheticoligonucleotide
  source 1..61: mol_type = other DNA
cttgttgtatatgtccttttataggtattaaacaacgagtcgctttaactggccctggctt

SEQ ID NO: 281 - DNA, 25 bp, synthetic construct
  misc_feature 1..25: note = Description of Artificial Sequence: Syntheticoligonucleotide
  source 1..25: mol_type = other DNA
gagtcgctttaactggccctggctt

SEQ ID NO: 282 - DNA, 61 bp, synthetic construct
  misc_feature 1..61: note = Description of Artificial Sequence: Syntheticoligonucleotide
  source 1..61: mol_type = other DNA
cttgttgtatatgtccttttataggtattaaacaactccacacctggaatcggctttcagc

SEQ ID NO: 283 - DNA, 25 bp, synthetic construct
  misc_feature 1..25: note = Description of Artificial Sequence: Syntheticoligonucleotide
  source 1..25: mol_type = other DNA
tccacacctggaatcggctttcagc

SEQ ID NO: 284 - DNA, 61 bp, synthetic construct
  misc_feature 1..61: note = Description of Artificial Sequence: Syntheticoligonucleotide
  source 1..61: mol_type = other DNA
cttgttgtatatgtccttttataggtattaaacaacaacccccgtctacctgcccacaggg

SEQ ID NO: 285 - DNA, 25 bp, synthetic construct
  misc_feature 1..25: note = Description of Artificial Sequence: Syntheticoligonucleotide
  source 1..25: mol_type = other DNA
aacccccgtctacctgcccacaggg

SEQ ID NO: 286 - DNA, 61 bp, synthetic construct; identical to SEQ ID NO: 211
  misc_feature 1..61: note = Description of Artificial Sequence: Syntheticoligonucleotide
  source 1..61: mol_type = other DNA

SEQ ID NO: 287 - DNA, 25 bp, synthetic construct
  misc_feature 1..25: note = Description of Artificial Sequence: Syntheticoligonucleotide
  source 1..25: mol_type = other DNA
gtagagggagaaatggaatccatat

SEQ ID NO: 288 - DNA, 61 bp, synthetic construct
  misc_feature 1..61: note = Description of Artificial Sequence: Syntheticoligonucleotide
  source 1..61: mol_type = other DNA
cttgttgtatatgtccttttataggtattaaacaacgacccatgggagcagctggtcagag

SEQ ID NO: 289 - DNA, 25 bp, synthetic construct
  misc_feature 1..25: note = Description of Artificial Sequence: Syntheticoligonucleotide
  source 1..25: mol_type = other DNA
gacccatgggagcagctggtcagag

SEQ ID NO: 290 - protein, 13 aa, synthetic construct
  REGION 1..13: note = Description of Artificial Sequence: Syntheticpeptide
ECPITKDVINEYK

SEQ ID NO: 291 - RNA, 36 nt, synthetic construct
  misc_feature 1..36: note = Description of Artificial Sequence: Syntheticoligonucleotide
  source 1..36: mol_type = other RNA
cctgttgtgaatactcttttataggtatcaaacaac